(19) United States
(12) Patent Application Publication
(10) Pub. No.: US 2023/0116703 A1
(43) Pub. Date: Apr. 13, 2023

(54) NOVEL ERGOLINES AND METHODS OF TREATING MOOD DISORDERS

(71) Applicant: Gilgamesh Pharmaceuticals, Inc., New York, NY (US)

(72) Inventor: Andrew Carry Kruegel, New York, NY (US)

(73) Assignee: Gilgamesh Pharmaceuticals, Inc., New York, NY (US)

(21) Appl. No.: 17/973,119

(22) Filed: Oct. 25, 2022

Related U.S. Application Data
(63) Continuation-in-part of application No. PCT/US22/26186, filed on Apr. 25, 2022.
(60) Provisional application No. 63/179,053, filed on Apr. 23, 2021.
(60) Provisional application No. 63/179,055, filed on Apr. 23, 2021.
(60) Provisional application No. 63/308,376, filed on Feb. 9, 2022.
(60) Provisional application No. 63/308,379, filed on Feb. 9, 2022.

(51) Int. Cl. C07D 471/04; A61P 25/22

(52) U.S. Cl. CPC C07D 471/04; A61P 25/22

(57) ABSTRACT

The present disclosure provides ergoline compounds and their use in treating mood disorders. Pharmaceutical compositions and methods of making various ergoline compounds are provided.

### CROSS-REFERENCE TO RELATED APPLICATIONS

The present application is a continuation-in-part of international application US2022/026,186, filed on Apr. 25, 2022, which claims priority of provisional applications U.S. Ser. Nos. 63/179,053 and 63/179,055, both of which were filed on Apr. 23, 2021, and of provisional applications U.S. Ser. Nos. 63/308,376 and 63/308,379, both of which were filed on Feb. 9, 2022, the contents of all of which are incorporated by reference.

### FIELD OF THE DISCLOSURE

The present disclosure relates to ergoline compounds that can reliably be used for the treatment of mood disorders.

### BACKGROUND

Ergolines are a diverse class of alkaloids containing the structural scaffold of the natural alkaloid ergoline.

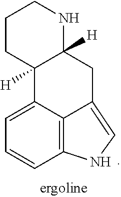 [SMILES: c1cc2c3c(c[nH]c3c1)C[C@H]1NCCC[C@H]21]

There are a significant number of ergoline compounds that include naturally occurring compounds, as well as synthetic and semi-synthetic chemical derivatives with similar structure. Ergolines are known to have diverse psychoactive and physiological effects. Some ergolines are serotonin 2a (5-HT_{2A}) receptor agonists and/or modulators of other serotonin receptors and are known to be psychoactive and/or induce vasoconstriction. In some cases, such compounds induce prolonged hallucinations. Other ergolines are agonists of dopamine receptors. Perhaps the most well-known ergoline is the psychedelic compound lysergic acid diethylamide (LSD). This compound is known to have significant effects on thought, perception, and behavior. However, it is currently classified as a Schedule I drug under the Controlled Substances Act due to its high abuse potential, no accepted medical use, and lack of established safety.

Accordingly, there remains a need for safe and effective ergoline compounds that can reliably be used for the treatment of mood disorders.

### SUMMARY

The present disclosure includes a compound of formula (I):

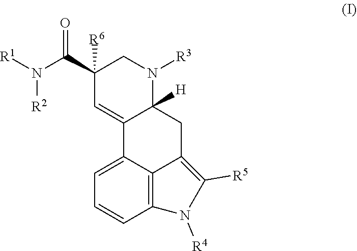

or a pharmaceutically acceptable salt thereof, wherein R¹, R², R³, R⁴, R⁵, and R⁶ are defined herein.

Additionally, the present disclosure includes methods of treating mood disorders comprising administering to a patient in need thereof a therapeutically effective amount of a compound of Formula (I).

### BRIEF DESCRIPTION OF THE DRAWINGS

FIG. 1 depicts the effect of Compound 1 in the mouse head twitch response assay as quantified by the number of head twitches recorded during a 20-minute observation period. Data points represent mean±SEM.

FIG. 2. depicts time immobile in the rat forced swim test 23.5 hours after administration of Compound 1. Data points represent mean±SEM. Comparisons to vehicle: **p<0.01, ****p<0.0001.

FIG. 3. depicts the total number of marbles buried during a 30-minute observation period in the mouse marble burying test. Data points represent mean±SEM. Comparisons to vehicle: *p<0.05, ****p<0.0001.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

“Treating” includes any effect, e.g., lessening, reducing, modulating, or eliminating, that results in the improvement of the condition, disease, disorder and the like.

The term “alkyl” as used herein refers to a saturated straight or branched hydrocarbon, having the number of carbon atoms specified herein, for example, 1 to 6 carbon atoms. Exemplary alkyl groups include, but are not limited to, straight or branched hydrocarbons of 1-6, 1-4, or 1-3 carbon atoms, referred to herein as C₁₋C₆ alkyl, C₁₋C₄ alkyl, and C₁₋C₃ alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

The term “alkenyl” as used herein is a branched or unbranched hydrocarbon group having a specified number of carbon atoms and containing at least one double bond as defined hereinbelow, for example, having 2-6 carbon atoms and 1-3 carbon-carbon double bonds. In some embodiments, alkenyl refers to a branched or unbranched saturated hydrocarbon group having three carbon atoms (C₃). In some embodiments, alkenyl refers to a branched or unbranched hydrocarbon group having six carbon atoms (C₆). In some embodiments, the term “alkenyl” includes, but is not limited to, vinyl or allyl.

The term “alkynyl” as used herein is a branched or unbranched hydrocarbon group having a specified number of carbon atoms and containing at least one triple bond as described hereinbelow, for example, having 2-6 carbon atoms, and 1-3 carbon-carbon triple bonds. In some embodiments, alkynyl refers to a branched or unbranched saturated hydrocarbon group having three carbon atoms (C₃). In some embodiments, alkynyl refers to a branched or unbranched hydrocarbon group having six carbon atoms (C₆). In some embodiments, the term “alkynyl” includes, but is not limited to, ethynyl or propargyl.

The term “cyano” as used herein refers to the radical —CN.

The terms “cycloalkyl” or a “carbocyclic group” as used herein refers to a saturated or partially unsaturated hydrocarbon group of, for example, 3-6, or 4-6 carbons, referred to herein as C₃₋C₆ cycloalkyl or C₄₋C₆ cycloalkyl, respectively. Exemplary cycloalkyl groups include, but are not limited to, cyclohexyl, cyclopentyl, cyclopentenyl, cyclobutyl or cyclopropyl.

The terms “halo” or “halogen” as used herein refer to F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring”. In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

The terms “heterocyclyl” or “heterocyclic group” are art-recognized and refer to saturated or partially unsaturated, 4-10 membered ring structures, including bridged or fused rings, and whose ring structures include one to three heteroatoms, such as nitrogen, oxygen, and sulfur. Where possible, heterocyclyl rings may be linked to the adjacent radical through carbon or nitrogen. Examples of heterocyclyl groups include, but are not limited to, pyrrolidine, piperidine, morpholine, thiomorpholine, piperazine, oxetane, azetidine, tetrahydrofuran or dihydrofuran etc.

The terms “hydroxy” and “hydroxyl” as used herein refers to the radical —OH.

“Pharmaceutically or pharmacologically acceptable” include molecular entities and compositions that do not produce an adverse, allergic, or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the present disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the present disclosure is desirably a mammal in which treatment of psychiatric disease or disorder is desired. “Modulation” includes antagonism (e.g., inhibition), agonism, partial antagonism and/or partial agonism.

In the present specification, the term “therapeutically effective amount” means the amount of the subject compound that will elicit the biological or medical response of a tissue, system, or animal, (e.g., mammal or human) that is being sought by the researcher, veterinarian, medical doctor, or other clinician. The compounds of the present disclosure are administered in therapeutically effective amounts to treat a disease. Alternatively, a therapeutically effective amount of a compound is the quantity required to achieve a desired therapeutic and/or prophylactic effect, such as an amount which results in a decrease in symptoms of a psychiatric disorder. As used herein, the term “prophylactic effect” refers to preventing the worsening of the condition, disease, disorder and the like.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt. In some embodiments the term “pharmaceutically acceptable salt(s)” as used herein refers to a hemitartrate salt. As used herein, a hemitartrate salt of a compound of Formula (I) is salt wherein the molar ratio of a compound of Formula (I) to tartaric acid is 2:1. In some embodiments the term “pharmaceutically acceptable salt(s)” as used herein refers to a tartrate salt. As used herein, a tartrate salt of a compound of Formula (I) is salt wherein the molar ratio of a compound of Formula (I) to tartaric acid is 1:1.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol [symbol] denotes a bond that may be a single, double, or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. Substituents around a carbocyclic or heterocyclic ring may also be referred to as “cis” or “trans,” where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diasteriomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations and may involve the use of chiral auxiliaries. For examples, see Carreira and Kvaerno, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like, and it is intended that the present disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The present disclosure also embraces isotopically labeled compounds of the present disclosure which are identical to those recited herein, except that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, sulfur, fluorine, and chlorine, such as ²H, ³H, ¹³C, ¹⁴C, ¹⁵N, ¹⁸O, ¹⁷O ³¹P, ³²P, ³⁵S, ¹⁸F, and ³⁶Cl, respectively. For example, a compound of the present disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present disclosure can generally be prepared by following procedures analogous to those disclosed in the examples herein by substituting an isotopically labeled reagent for a non-isotopically labeled reagent.

In the structural formulae depicted hereinbelow, the structures of compounds I and II are the same, what makes them different are the definitions of the variables, R¹, R², R³, R⁴, R⁵, and R⁶. The same is true with respect to the compounds of Formulae Ia and IIa, Ib and IIb, Ic and IIc, Id and IId, and Ie and IIe, respectively.

### I. Compounds

In some embodiments, the present disclosure provides a compound of Formula (I):

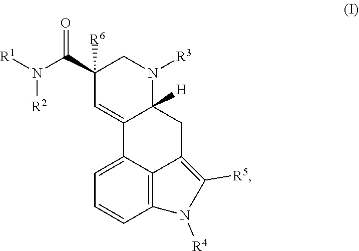

or a pharmaceutically acceptable salt thereof,

wherein
R¹ is C₁-C₆ alkyl or 3-7 membered carbocyclyl, wherein R¹ is optionally substituted with one or more halogen or C₁-C₆ alkyl;
R² is hydrogen or C₁-C₆ alkyl, wherein R² is optionally substituted with one or more halogen or C₁-C₆ alkyl; or
   wherein R¹ and R² can be taken together with the atom on which they are attached to form an optionally substituted 3-7 membered heterocyclyl comprising 1-3 heteroatoms selected from the group consisting of N, O, and S, wherein the heterocyclyl is optionally substituted with one or more fluoro or C₁-C₆ alkyl;
R³ is selected from the group consisting of C₂-C₆ alkyl, C₂-C₆ alkenyl, C₂-C₆ alkynyl, —CH₂-(cyclopropyl), and 3-7 membered cycloalkyl,
   wherein R³ may be substituted with one or more substituents each independently selected from the group consisting of fluoro, hydroxyl, and —OMe;
   or
   R³ is selected from the group consisting of —(C₁-C₂ alkyl)-phenyl and —(C₁-C₂ alkyl)-(6-membered heteroaryl),
   wherein C₁-C₂ alkyl is optionally substituted with one or more fluoro, hydroxyl, and —OMe, and wherein phenyl and 6-membered heteroaryl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₄ alkyl, C₃-C₅ cycloalkyl, and C₁-C₄ alkoxy;
R⁴ is hydrogen or —C(O)(C₁-C₈ alkyl);
R⁵ is hydrogen, Me, Et, —CH₂F, CHF₂, —CF₃ or halogen;
R⁶ is hydrogen or deuterium,

In some embodiments, the compound of the above Formula (I) does not include the following:
wherein when R¹ and R² are both ethyl, and R⁴ and R⁵ are both hydrogen, R³ is not unsubstituted linear C₂-C₆ alkyl, isopropyl, —CH₂CH═CH₂, —CH₂CH₂F, or —CH₂CH₂Ph;
wherein when R¹ and R² are both ethyl, R⁴ is —C(O)(C₂ alkyl), and R⁵ is hydrogen, R³ is not unsubstituted ethyl; and
wherein when R¹ is ethyl and R² is H, R³ is not unsubstituted ethyl, unsubstituted n-propyl, or —CH₂CH═CH₂.

The present disclosure describes, in some embodiments, a compound of Formula (II):

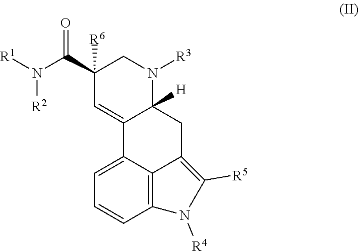

or a pharmaceutically acceptable salt thereof,

wherein
R¹ is C₁-C₆ alkyl or 3-7 membered carbocyclyl, wherein R¹ is optionally substituted with one or more halogen or C₁-C₆ alkyl;
R² is hydrogen or C₁-C₆ alkyl, wherein R² is optionally substituted with one or more halogen or C₁-C₆ alkyl; or
   wherein R¹ and R² can be taken together with the atom on which they are attached to form an optionally substituted 3-7 membered heterocyclyl comprising 1-3 heteroatoms selected from the group consisting of N, O, and S, wherein the heterocyclyl is optionally substituted with one or more fluoro or C₁-C₆ alkyl;
R³ is selected from the group consisting of C₁-C₆ alkyl, C₂-C₆ alkenyl, C₂-C₆ alkynyl, —CH₂-(cyclopropyl), and 3-7 membered cycloalkyl,
   wherein R³ may be substituted with one or more substituents each independently selected from the group consisting of fluoro, hydroxyl, and —OMe;
   or
   R³ is selected from the group consisting of —(C₁-C₂ alkyl)-phenyl and —(C₁-C₂ alkyl)-(6-membered heteroaryl),
   wherein C₁-C₂ alkyl is optionally substituted with one or more fluoro, hydroxyl, and —OMe, and wherein phenyl and 6-membered heteroaryl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₄ alkyl, C₃-C₅ cycloalkyl, and C₁-C₄ alkoxy;
R⁴ is hydrogen or —C(O)(C₁-C₈ alkyl);
R⁵ is Me, Et, —CH₂F, —CHF₂, —CF₃, or halogen;
R⁶ is hydrogen or deuterium.

In some embodiments, the following compounds are excluded from Formula (II):
(a) when R¹ and R² are both ethyl, R³ is methyl, and R⁴ is hydrogen, then R⁵ is not chloro, bromo, iodo, or unsubstituted methyl;
(b) when R² is hydrogen, R³ is methyl, R⁴ is hydrogen, and R⁵ is bromo, then R¹ is not ethyl, isopropyl, or propargyl; and
(c) when R² is methyl, R³ is methyl, R⁴ is hydrogen, and R⁵ is bromo, then R¹ is not propargyl or cyclopropyl.

In some embodiments, the present disclosure includes a compound of formula (Ia) or (IIa):

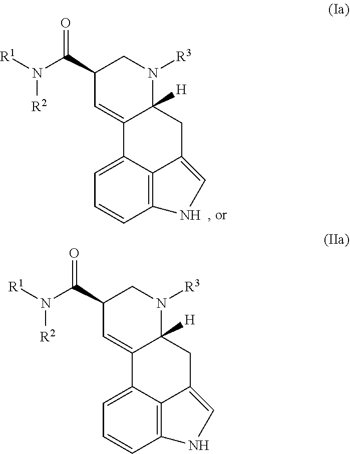

or a pharmaceutically acceptable salt thereof, wherein R¹, R², and R³ are defined above and in the classes and embodiments disclosed herein.

In some embodiments, the present disclosure includes a compound of formula (Ib):

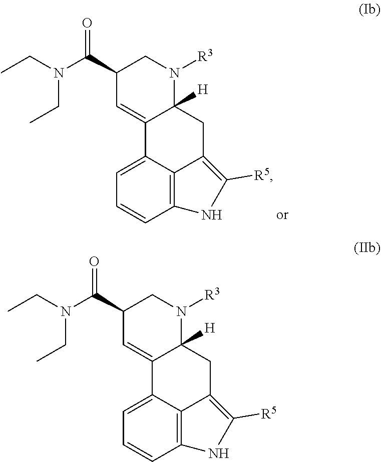

or a pharmaceutically acceptable salt thereof, wherein R³, and R⁵ are defined above and in the classes and embodiments disclosed herein.

In some embodiments, the present disclosure includes a compound of formula (Ic):

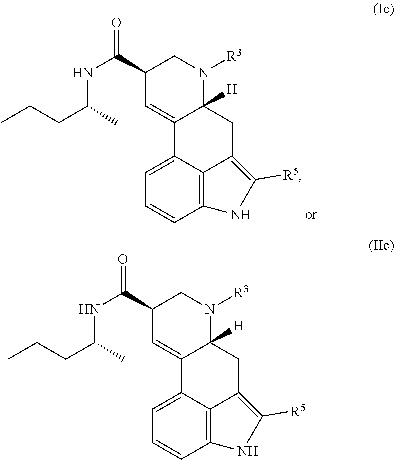

or a pharmaceutically acceptable salt thereof, wherein R³, and R⁵ are defined above and in the classes and embodiments disclosed herein.

In some embodiments, the present disclosure includes a compound of formula (Id):

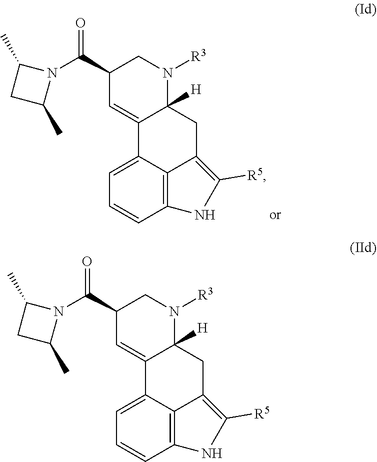

or a pharmaceutically acceptable salt thereof, wherein R³, and R⁵ are defined above and in the classes and embodiments disclosed herein.

In some embodiments, the present disclosure includes a compound of formula (Ie):

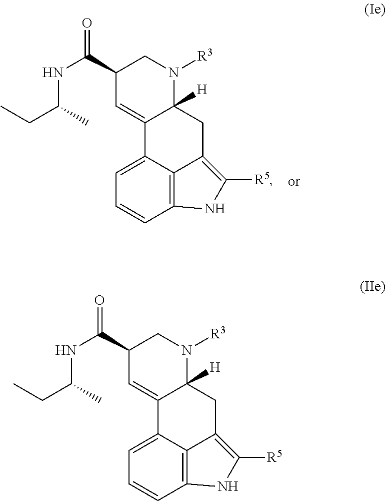

or a pharmaceutically acceptable salt thereof, wherein R³, and R⁵ are defined above and in the classes and embodiments disclosed herein.

In some embodiments, R¹ is C₁-C₆ alkyl. In some embodiments, R¹ is linear C₁-C₆ alkyl. In some embodiments, R¹ is branched C₁-C₆ alkyl. In some embodiments, R¹ is C₂-C₅ alkyl. In some embodiments, R¹ is selected from the group consisting of ethyl, sec-butyl, 2-pentyl, and 3-pentyl.

In some embodiments, R¹ is C₁-C₆ alkyl or 3-7 membered carbocyclyl, wherein R¹ is optionally substituted with one or more halogen or C₁-C₆ alkyl. In some embodiments, R¹ is C₁-C₆ alkyl or 3-5 membered carbocyclyl, wherein R¹ is optionally substituted with one or more fluoro or C₁-C₄ alkyl.

In some embodiments, R² is hydrogen or C₁-C₆ alkyl, wherein R² is optionally substituted with one or more halogen or C₁-C₆ alkyl. In some embodiments, R² is hydrogen or C₁-C₆ alkyl. In some embodiments, R² is hydrogen. In some embodiments, R² is C₁-C₆ alkyl. In some embodiments, R² is linear C₁-C₆ alkyl. In some embodiments, R² is branched C₁-C₆ alkyl. In some embodiments, R² is C₂-C₅ alkyl. In some embodiments, R² is selected from the group consisting of hydrogen, ethyl, sec-butyl, 2-pentyl, and 3-pentyl.

In some embodiments, R¹ and R² can be taken together with the atom on which they are attached to form an optionally substituted 3-7 membered heterocyclyl comprising 1-3 heteroatoms selected from the group consisting of N, O, and S. In some embodiments, R¹ and R² can be taken together with the atom on which they are attached to form an optionally substituted group selected from the group consisting of azetidinyl, pyrrolidinyl, piperidinyl, piperizinyl, and morpholinyl. In some embodiments, R¹ and R² can be taken together with the atom on which they are attached to form dimethylazetidinyl.

In some embodiments, R³ is selected from the group consisting of C₁-C₆ alkyl, C₂-C₆ alkenyl, C₂-C₆ alkynyl, and 3-7 membered cycloalkyl, wherein R³ may be substituted with one or more substituents each independently selected from the group consisting of fluoro, 3-7 membered cycloalkyl, and phenyl, wherein cycloalkyl or phenyl are optionally substituted with one, two, or three substitutents each independently selected from the group consisting of halogen, hydroxyl, C₁-C₄ alkyl, and C₁-C₄ alkoxy. In some embodiments, R³ is C₁-C₆ alkyl or C₂-C₆ alkenyl, wherein R³ may be substituted with one or more substituents each independently selected from the group consisting of fluoro, 3-7 membered cycloalkyl, and phenyl, wherein cycloalkyl or phenyl are optionally substituted with one, two, or three substitutents each independently selected from the group consisting of halogen, hydroxyl, C₁-C₄ alkyl, and C₁-C₄ alkoxy. In some embodiments, R³ is C₁-C₃ alkyl, or C₂-C₃ alkenyl, wherein R³ may be substituted with one or more substituents each independently selected from the group consisting of fluoro, 3-7 membered cycloalkyl, and phenyl, wherein cycloalkyl or phenyl are optionally substituted with one, two, or three substitutents each independently selected from the group consisting of halogen, hydroxyl, C₁-C₄ alkyl, and C₁-C₄ alkoxy. In some embodiments, R³ is selected from the group consisting of methyl, ethyl, n-propyl, and allyl, wherein R³ may be substituted with one to three substituents selected from the group consisting of fluoro, 2-methoxyphenyl, and 2-hydroxyphenyl.

R³ is selected from the group consisting of C₂-C₆ alkyl, C₂-C₆ alkenyl, C₂-C₆ alkynyl, —CH₂-(cyclopropyl), and 3-7 membered cycloalkyl, wherein R³ may be substituted with one or more substituents each independently selected from the group consisting of fluoro, hydroxyl, and —OMe; or R³ is selected from the group consisting of —(C₁-C₂ alkyl)-phenyl and —(C₁-C₂ alkyl)-(6-membered heteroaryl), wherein C₁-C₂ alkyl is optionally substituted with one or more fluoro, hydroxyl, and —OMe, and wherein phenyl and 6-membered heteroaryl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₄ alkyl, C₃-C₅ cycloalkyl, and C₁-C₄ alkoxy. In some embodiments, R³ is selected from the group consisting of C₂-C₆ alkyl, C₂-C₆ alkenyl, C₂-C₆ alkynyl, —CH₂-(cyclopropyl), and 3-7 membered cycloalkyl, wherein R³ may be substituted with one or more substituents each independently selected from the group consisting of fluoro, hydroxyl, and —OMe. In some embodiments, R³ is selected from the group consisting of —(C₁-C₂ alkyl)-phenyl and —(C₁-C₂ alkyl)-(6-membered heteroaryl), wherein C₁-C₂ alkyl is optionally substituted with one or more fluoro, hydroxyl, and —OMe, and wherein phenyl and 6-membered heteroaryl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₄ alkyl, C₃-C₅ cycloalkyl, and C₁-C₄ alkoxy.

In some embodiments, R³ is selected from the group consisting of C₂-C₄ alkyl, C₂-C₄ alkenyl, C₂-C₄ alkynyl, —CH₂-(cyclopropyl), and 3-5 membered cycloalkyl, wherein R³ may be substituted with one or more substituents each independently selected from the group consisting of fluoro, hydroxyl, and —OMe; or R³ is selected from the group consisting of —(C₁-C₂ alkyl)-phenyl and —(C₁-C₂ alkyl)-(6-membered heteroaryl), wherein C₁-C₂ alkyl is optionally substituted with one or more fluoro, and wherein phenyl and 6-membered heteroaryl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₃ alkyl, cyclopropyl, and C₁-C₃ alkoxy. In some embodiments, R³ is selected from the group consisting of C₂-C₄ alkyl, C₂-C₄ alkenyl, C₂-C₄ alkynyl, —CH₂-(cyclopropyl), and 3-5 membered cycloalkyl, wherein R³ may be substituted with one or more substituents each independently selected from the group consisting of fluoro, hydroxyl, and —OMe. In some embodiments, R³ is selected from the group consisting of —(C₁-C₂ alkyl)-phenyl and —(C₁-C₂ alkyl)-(6-membered heteroaryl), wherein C₁-C₂ alkyl is optionally substituted with one or more fluoro, and wherein phenyl and 6-membered heteroaryl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₃ alkyl, cyclopropyl, and C₁-C₃ alkoxy.

In some embodiments, R³ is selected from the group consisting of ethyl, n-propyl, —CH₂CH═CH₂, cyclopropyl, and —CH₂-(cyclopropyl), wherein R³ may be substituted with one to three instances of fluoro. In some embodiments, R³ is selected from the group consisting of ethyl, n-propyl, —CH₂CH═CH₂, cyclopropyl, —CH₂-(cyclopropyl), —CH₂CF₃, —CH₂CH₂CH₂F, and —CH₂CH₂CF₃. In some embodiments, R³ is selected from the group consisting of —(C₁-C₂ alkyl)-phenyl and —(C₁-C₂ alkyl)-(6-membered heteroaryl), wherein C₁-C₂ alkyl is optionally substituted with one or more fluoro, and wherein phenyl and 6-membered heteroaryl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₃ alkyl, cyclopropyl, and C₁-C₃ alkoxy. In some embodiments, R³ is selected from the group consisting of —(C₁-C₂ alkyl)-phenyl and —(C₁-C₂ alkyl)-pyridinyl, wherein phenyl and pyridinyl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₃ alkyl, cyclopropyl, and C₁-C₃ alkoxy.

In some embodiments, R³ is selected from the group consisting of

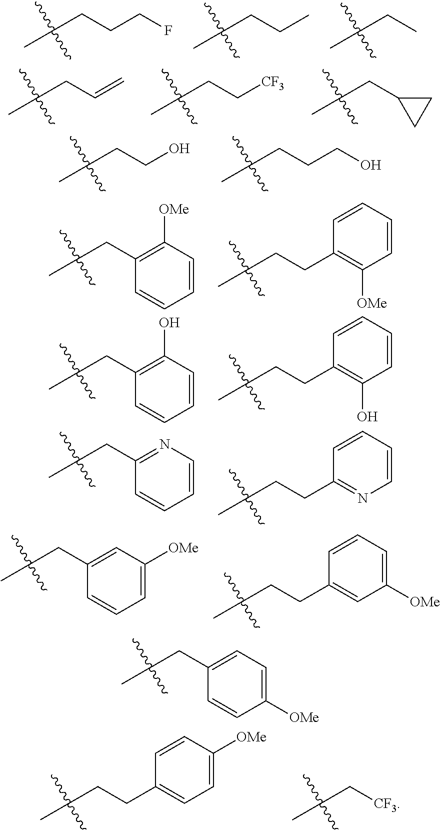

In some embodiments, R⁴ is hydrogen or —C(O)(C₁-C₈ alkyl). In some embodiments, R⁴ is hydrogen or —C(O)(C₁-C₃ alkyl). In some embodiments, R⁴ is hydrogen. In some embodiments, R⁴ is —C(O)(C₁-C₈ alkyl). In some embodiments, R⁴ is —C(O)(C₁-C₃ alkyl).

In some embodiments, R⁵ is Me, Et, —CH₂F, CHF₂, —CF₃, or halogen. In some embodiments, R⁵ is Me, Et, or halogen. In some embodiments, R⁵ is Me, Et, or bromo. In some embodiments, R⁵ is hydrogen or halogen. In some embodiments, R⁵ is hydrogen. In some embodiments, R⁵ is halogen. In some embodiments, R⁵ is hydrogen or bromo. In some embodiments, R⁵ is bromo. In some embodiments, R⁵ is hydrogen, Me, or Et. In some embodiments, R⁵ is Me or Et.

In some embodiments, the present disclosure includes a compound selected from the group consisting of:

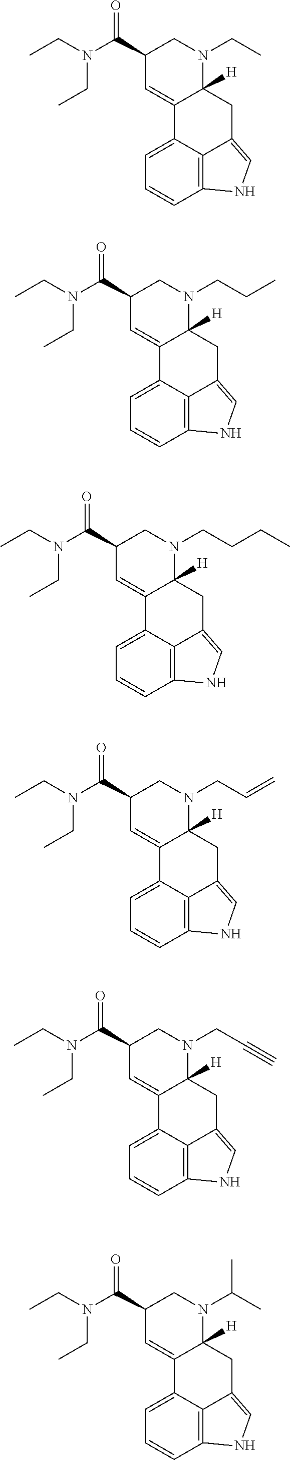

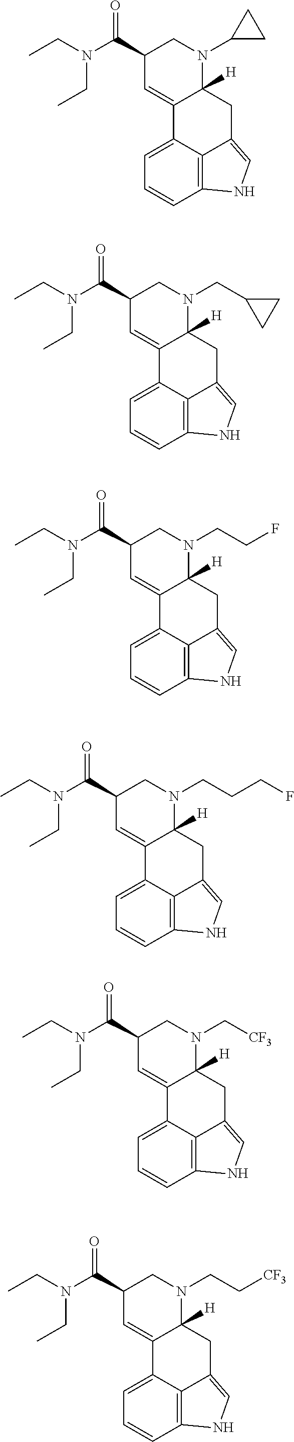 [SMILES: CCN(CC)C(=O)[C@@H]1C=C2c3cccc4[nH]cc(c34)C[C@H]2N(C2CC2)C1 ; CCN(CC)C(=O)[C@@H]1C=C2c3cccc4[nH]cc(c34)C[C@H]2N(CC2CC2)C1 ; CCN(CC)C(=O)[C@@H]1C=C2c3cccc4[nH]cc(c34)C[C@H]2N(CCF)C1 ; CCN(CC)C(=O)[C@@H]1C=C2c3cccc4[nH]cc(c34)C[C@H]2N(CCCF)C1 ; CCN(CC)C(=O)[C@@H]1C=C2c3cccc4[nH]cc(c34)C[C@H]2N(CC(F)(F)F)C1 ; CCN(CC)C(=O)[C@@H]1C=C2c3cccc4[nH]cc(c34)C[C@H]2N(CCC(F)(F)F)C1]

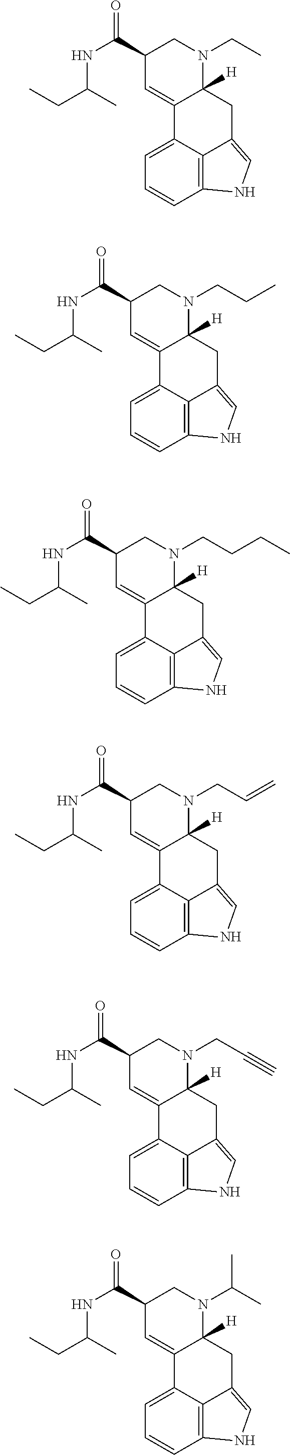

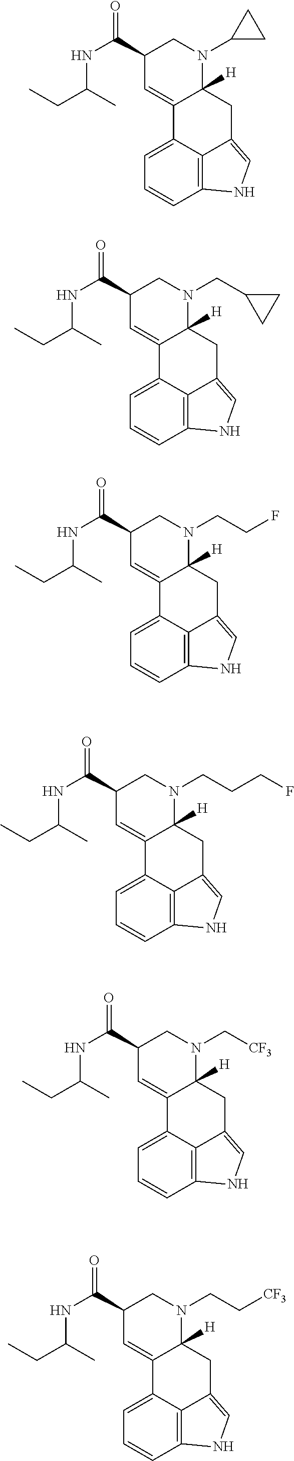 [SMILES: CCC(C)NC(=O)[C@@H]1C=C2c3cccc4[nH]cc(c34)C[C@H]2N(C2CC2)C1 ; CCC(C)NC(=O)[C@@H]1C=C2c3cccc4[nH]cc(c34)C[C@H]2N(CC2CC2)C1 ; CCC(C)NC(=O)[C@@H]1C=C2c3cccc4[nH]cc(c34)C[C@H]2N(CCF)C1 ; CCC(C)NC(=O)[C@@H]1C=C2c3cccc4[nH]cc(c34)C[C@H]2N(CCCF)C1 ; CCC(C)NC(=O)[C@@H]1C=C2c3cccc4[nH]cc(c34)C[C@H]2N(CC(F)(F)F)C1 ; CCC(C)NC(=O)[C@@H]1C=C2c3cccc4[nH]cc(c34)C[C@H]2N(CCC(F)(F)F)C1]

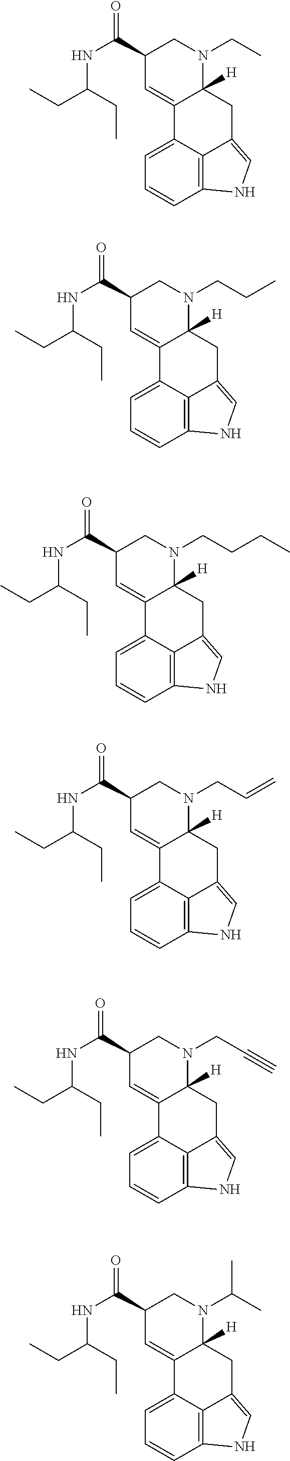

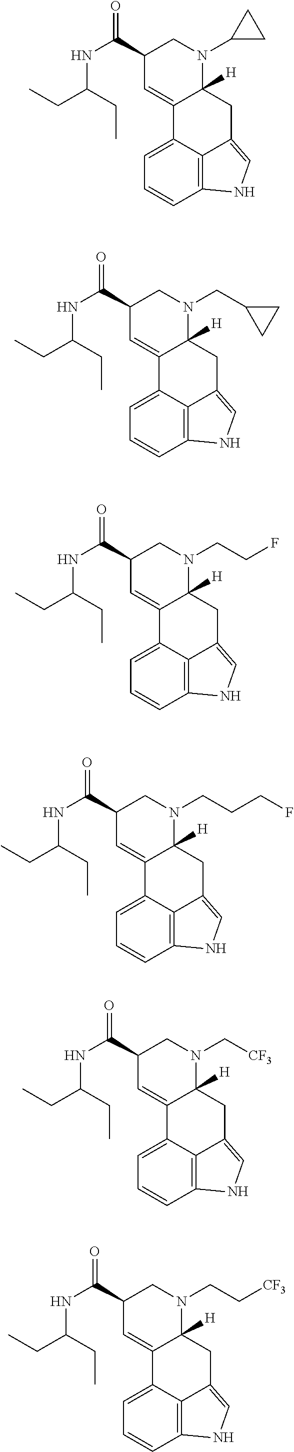 [SMILES: CCC(CC)NC(=O)[C@@H]1C=C2c3cccc4[nH]cc(c34)C[C@H]2N(C2CC2)C1 ; CCC(CC)NC(=O)[C@@H]1C=C2c3cccc4[nH]cc(c34)C[C@H]2N(CC2CC2)C1 ; CCC(CC)NC(=O)[C@@H]1C=C2c3cccc4[nH]cc(c34)C[C@H]2N(CCF)C1 ; CCC(CC)NC(=O)[C@@H]1C=C2c3cccc4[nH]cc(c34)C[C@H]2N(CCCF)C1 ; CCC(CC)NC(=O)[C@@H]1C=C2c3cccc4[nH]cc(c34)C[C@H]2N(CC(F)(F)F)C1 ; CCC(CC)NC(=O)[C@@H]1C=C2c3cccc4[nH]cc(c34)C[C@H]2N(CCC(F)(F)F)C1]

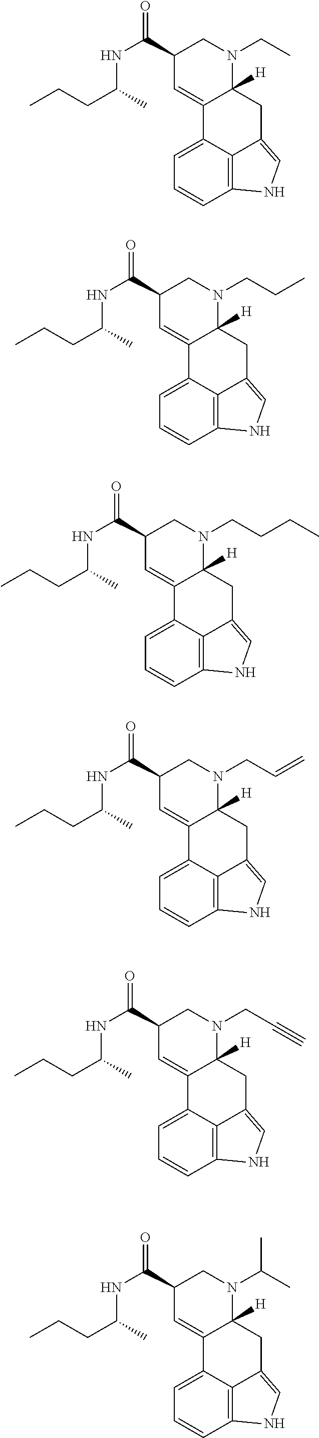

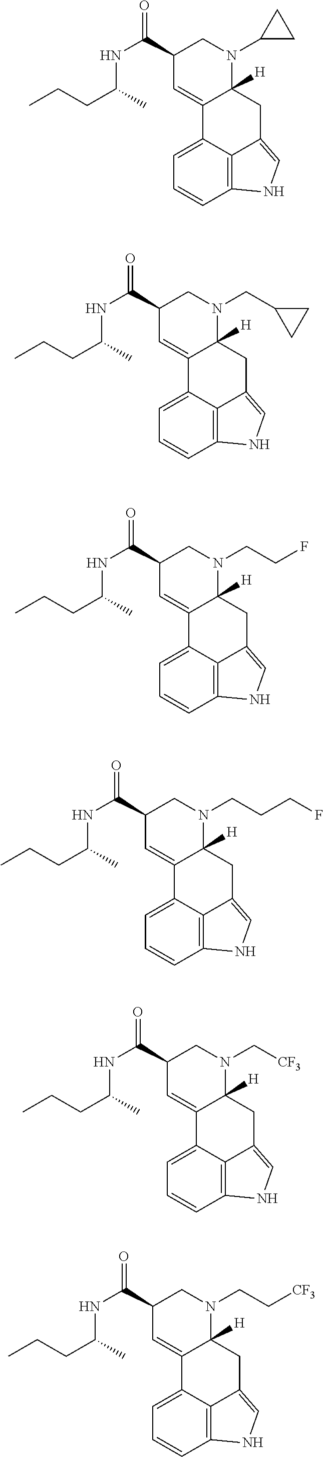 [SMILES: CCC[C@@H](C)NC(=O)[C@@H]1C=C2c3cccc4[nH]cc(c34)C[C@H]2N(C2CC2)C1 ; CCC[C@@H](C)NC(=O)[C@@H]1C=C2c3cccc4[nH]cc(c34)C[C@H]2N(CC2CC2)C1 ; CCC[C@@H](C)NC(=O)[C@@H]1C=C2c3cccc4[nH]cc(c34)C[C@H]2N(CCF)C1 ; CCC[C@@H](C)NC(=O)[C@@H]1C=C2c3cccc4[nH]cc(c34)C[C@H]2N(CCCF)C1 ; CCC[C@@H](C)NC(=O)[C@@H]1C=C2c3cccc4[nH]cc(c34)C[C@H]2N(CC(F)(F)F)C1 ; CCC[C@@H](C)NC(=O)[C@@H]1C=C2c3cccc4[nH]cc(c34)C[C@H]2N(CCC(F)(F)F)C1]

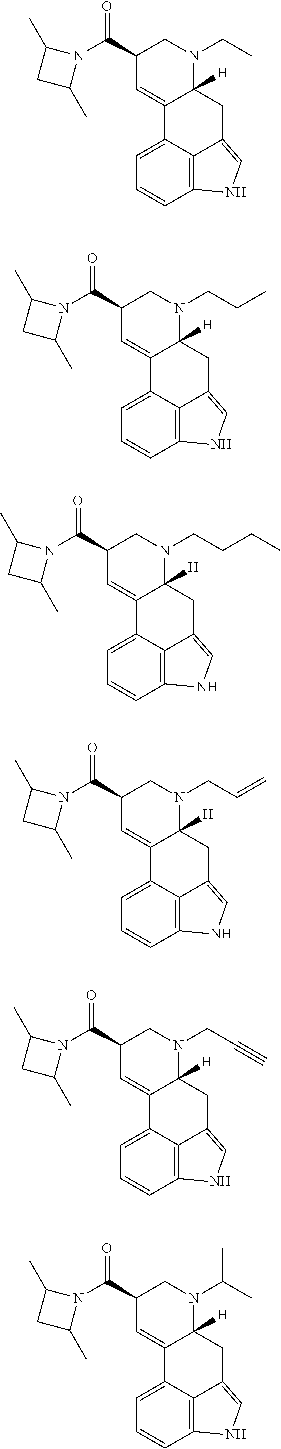

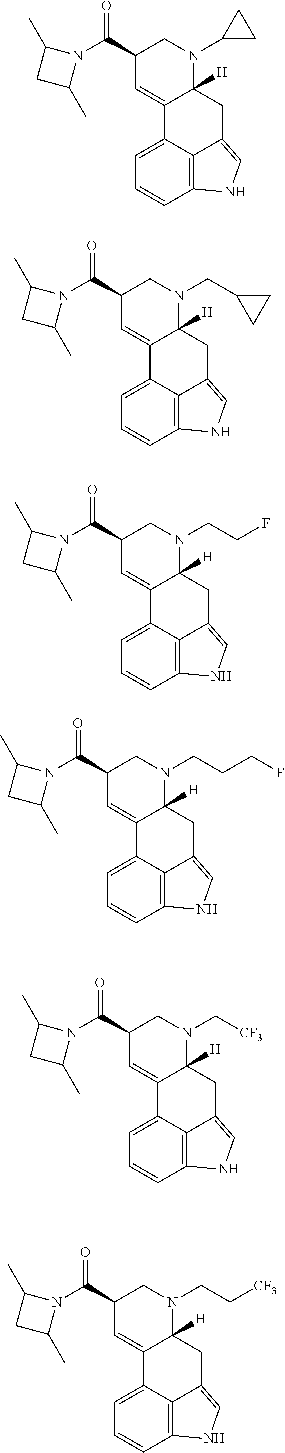 [SMILES: CC1CC(C)N1C(=O)[C@@H]1C=C2c3cccc4[nH]cc(c34)C[C@H]2N(C2CC2)C1 ; CC1CC(C)N1C(=O)[C@@H]1C=C2c3cccc4[nH]cc(c34)C[C@H]2N(CC2CC2)C1 ; CC1CC(C)N1C(=O)[C@@H]1C=C2c3cccc4[nH]cc(c34)C[C@H]2N(CCF)C1 ; CC1CC(C)N1C(=O)[C@@H]1C=C2c3cccc4[nH]cc(c34)C[C@H]2N(CCCF)C1 ; CC1CC(C)N1C(=O)[C@@H]1C=C2c3cccc4[nH]cc(c34)C[C@H]2N(CC(F)(F)F)C1 ; CC1CC(C)N1C(=O)[C@@H]1C=C2c3cccc4[nH]cc(c34)C[C@H]2N(CCC(F)(F)F)C1]

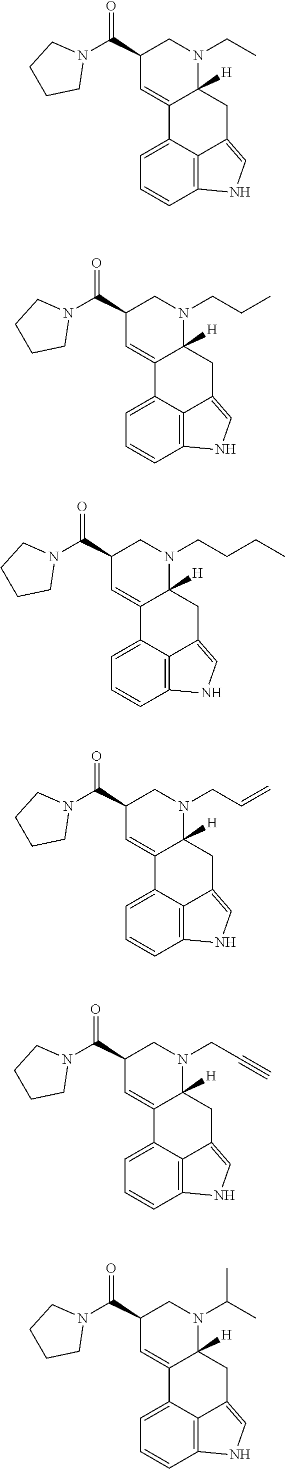 [SMILES: CCN1C[C@H](C(=O)N2CCCC2)C=C2c3cccc4[nH]cc(c34)C[C@H]21 ; CCCN1C[C@H](C(=O)N2CCCC2)C=C2c3cccc4[nH]cc(c34)C[C@H]21 ; CCCCN1C[C@H](C(=O)N2CCCC2)C=C2c3cccc4[nH]cc(c34)C[C@H]21 ; C=CCN1C[C@H](C(=O)N2CCCC2)C=C2c3cccc4[nH]cc(c34)C[C@H]21 ; C#CCN1C[C@H](C(=O)N2CCCC2)C=C2c3cccc4[nH]cc(c34)C[C@H]21 ; CC(C)N1C[C@H](C(=O)N2CCCC2)C=C2c3cccc4[nH]cc(c34)C[C@H]21]

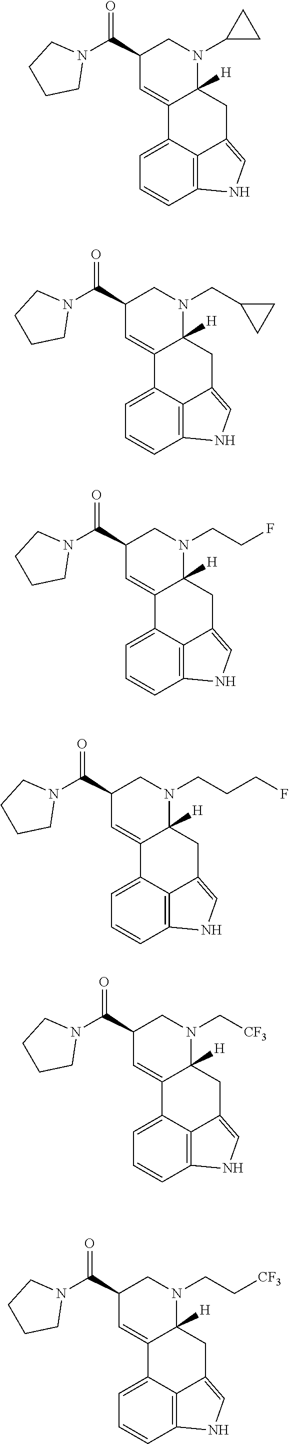 [SMILES: O=C([C@@H]1C=C2c3cccc4[nH]cc(c34)C[C@H]2N(C2CC2)C1)N1CCCC1 ; O=C([C@@H]1C=C2c3cccc4[nH]cc(c34)C[C@H]2N(CC2CC2)C1)N1CCCC1 ; O=C([C@@H]1C=C2c3cccc4[nH]cc(c34)C[C@H]2N(CCF)C1)N1CCCC1 ; O=C([C@@H]1C=C2c3cccc4[nH]cc(c34)C[C@H]2N(CCCF)C1)N1CCCC1 ; O=C([C@@H]1C=C2c3cccc4[nH]cc(c34)C[C@H]2N(CC(F)(F)F)C1)N1CCCC1 ; O=C([C@@H]1C=C2c3cccc4[nH]cc(c34)C[C@H]2N(CCC(F)(F)F)C1)N1CCCC1]

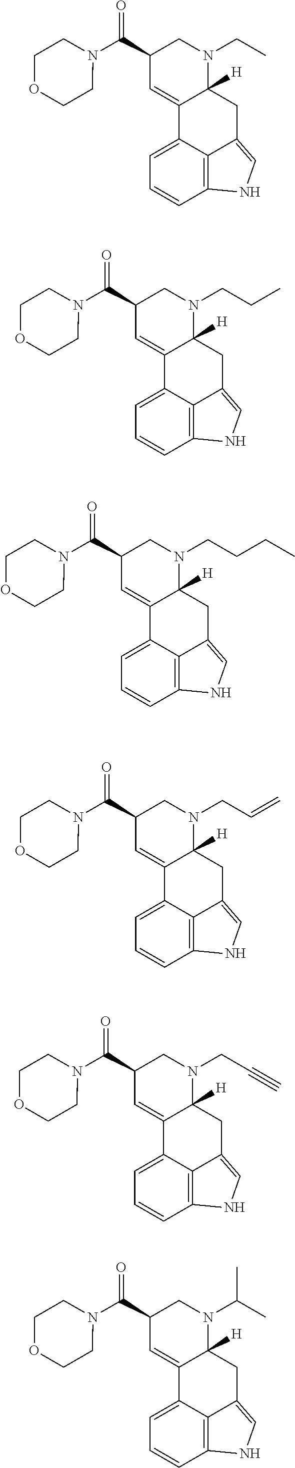 [SMILES: CCN1C[C@H](C(=O)N2CCOCC2)C=C2c3cccc4[nH]cc(c34)C[C@H]21 ; CCCN1C[C@H](C(=O)N2CCOCC2)C=C2c3cccc4[nH]cc(c34)C[C@H]21 ; CCCCN1C[C@H](C(=O)N2CCOCC2)C=C2c3cccc4[nH]cc(c34)C[C@H]21 ; C=CCN1C[C@H](C(=O)N2CCOCC2)C=C2c3cccc4[nH]cc(c34)C[C@H]21 ; C#CCN1C[C@H](C(=O)N2CCOCC2)C=C2c3cccc4[nH]cc(c34)C[C@H]21 ; CC(C)N1C[C@H](C(=O)N2CCOCC2)C=C2c3cccc4[nH]cc(c34)C[C@H]21]

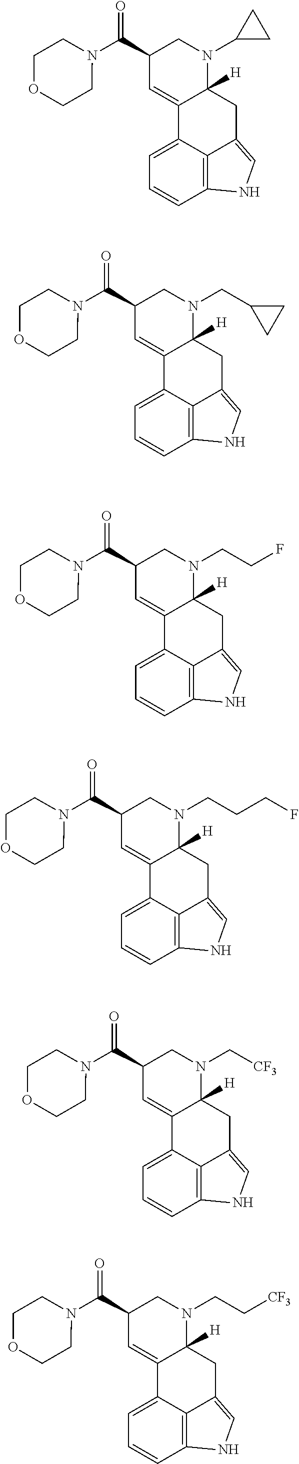 [SMILES: O=C([C@@H]1C=C2c3cccc4[nH]cc(c34)C[C@H]2N(C2CC2)C1)N1CCOCC1 ; O=C([C@@H]1C=C2c3cccc4[nH]cc(c34)C[C@H]2N(CC2CC2)C1)N1CCOCC1 ; O=C([C@@H]1C=C2c3cccc4[nH]cc(c34)C[C@H]2N(CCF)C1)N1CCOCC1 ; O=C([C@@H]1C=C2c3cccc4[nH]cc(c34)C[C@H]2N(CCCF)C1)N1CCOCC1 ; O=C([C@@H]1C=C2c3cccc4[nH]cc(c34)C[C@H]2N(CC(F)(F)F)C1)N1CCOCC1 ; O=C([C@@H]1C=C2c3cccc4[nH]cc(c34)C[C@H]2N(CCC(F)(F)F)C1)N1CCOCC1]

or a pharmaceutically acceptable salt thereof.

In some embodiments, the present disclosure includes a compound selected from the group consisting of:

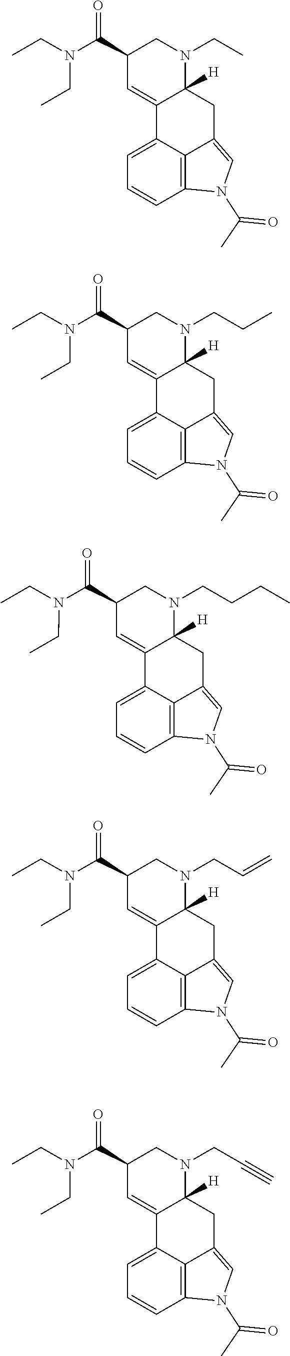

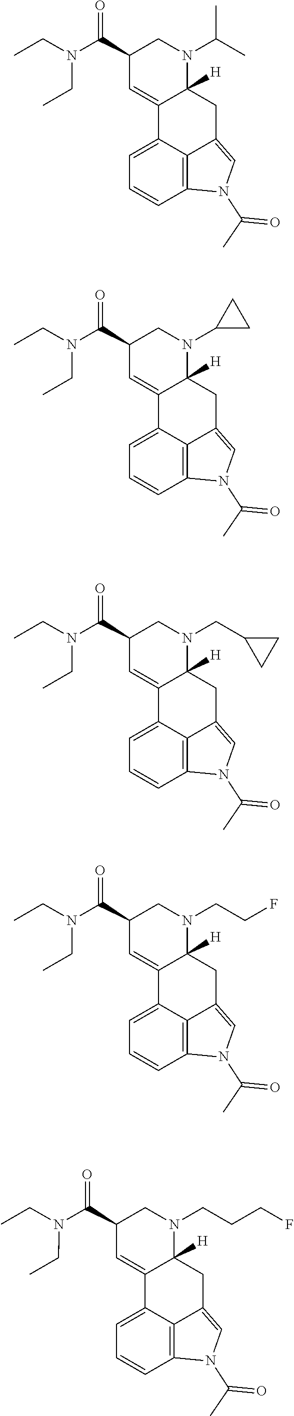 [SMILES: CCN(CC)C(=O)[C@@H]1C=C2c3cccc4c3c(cn4C(C)=O)C[C@H]2N(C(C)C)C1 ; CCN(CC)C(=O)[C@@H]1C=C2c3cccc4c3c(cn4C(C)=O)C[C@H]2N(C2CC2)C1 ; CCN(CC)C(=O)[C@@H]1C=C2c3cccc4c3c(cn4C(C)=O)C[C@H]2N(CC2CC2)C1 ; CCN(CC)C(=O)[C@@H]1C=C2c3cccc4c3c(cn4C(C)=O)C[C@H]2N(CCF)C1 ; CCN(CC)C(=O)[C@@H]1C=C2c3cccc4c3c(cn4C(C)=O)C[C@H]2N(CCCF)C1]

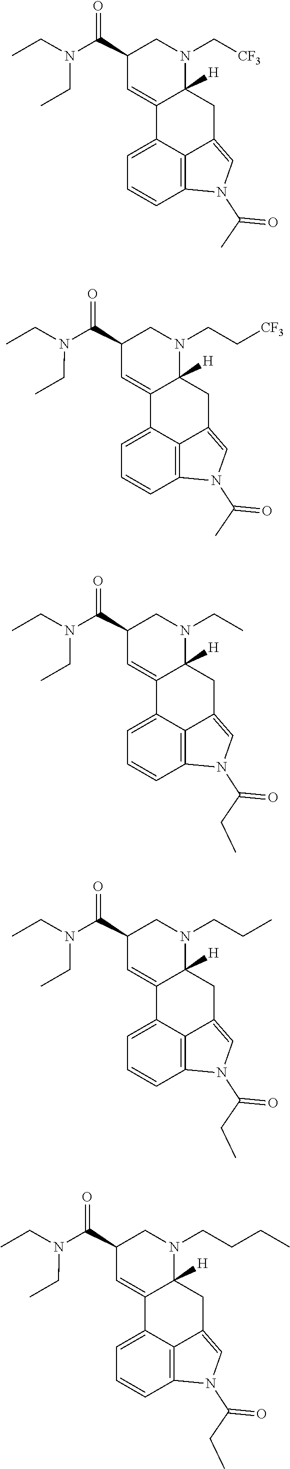 [SMILES: CCN(CC)C(=O)[C@@H]1C=C2c3cccc4c3c(cn4C(C)=O)C[C@H]2N(CC(F)(F)F)C1 ; CCN(CC)C(=O)[C@@H]1C=C2c3cccc4c3c(cn4C(C)=O)C[C@H]2N(CCC(F)(F)F)C1 ; CCC(=O)n1cc2c3c(cccc31)C1=C[C@@H](C(=O)N(CC)CC)CN(CC)[C@@H]1C2 ; CCCN1C[C@H](C(=O)N(CC)CC)C=C2c3cccc4c3c(cn4C(=O)CC)C[C@H]21 ; CCCCN1C[C@H](C(=O)N(CC)CC)C=C2c3cccc4c3c(cn4C(=O)CC)C[C@H]21]

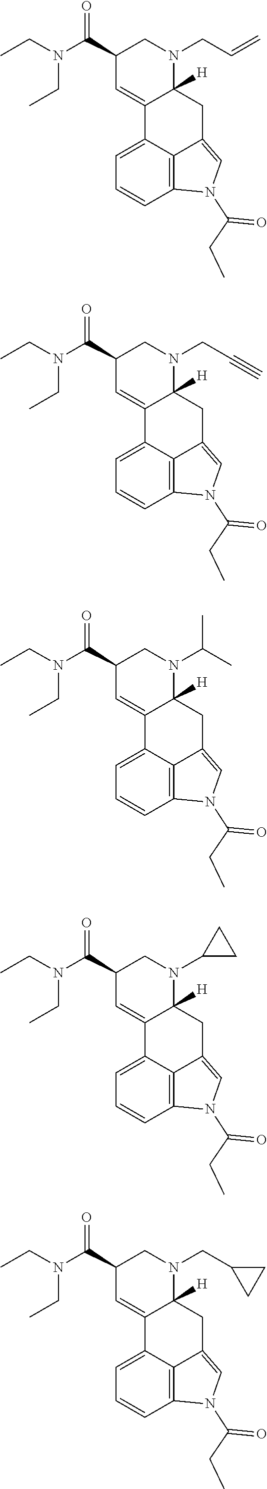

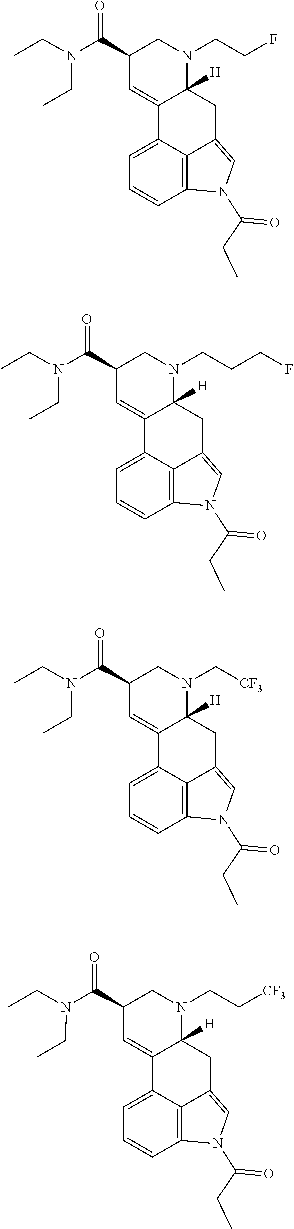 [SMILES: CCC(=O)n1cc2c3c(cccc31)C1=C[C@@H](C(=O)N(CC)CC)CN(CCF)[C@@H]1C2 ; CCC(=O)n1cc2c3c(cccc31)C1=C[C@@H](C(=O)N(CC)CC)CN(CCCF)[C@@H]1C2 ; CCC(=O)n1cc2c3c(cccc31)C1=C[C@@H](C(=O)N(CC)CC)CN(CC(F)(F)F)[C@@H]1C2 ; CCC(=O)n1cc2c3c(cccc31)C1=C[C@@H](C(=O)N(CC)CC)CN(CCC(F)(F)F)[C@@H]1C2]

or a pharmaceutically acceptable salt thereof.

In some embodiments, the present disclosure includes a compound selected from the group consisting of:

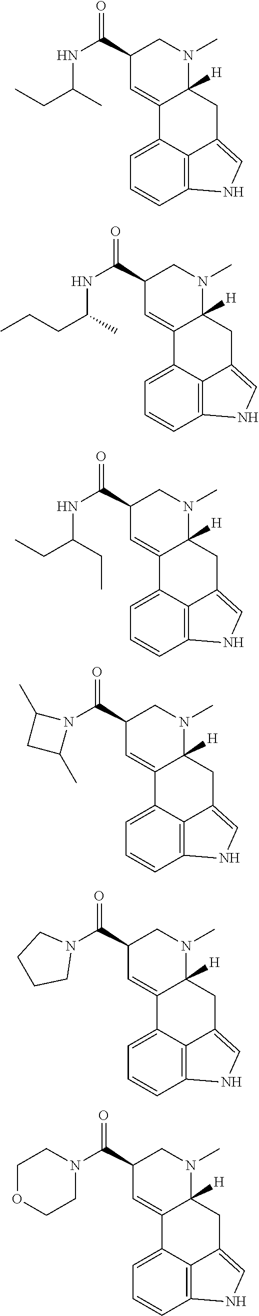

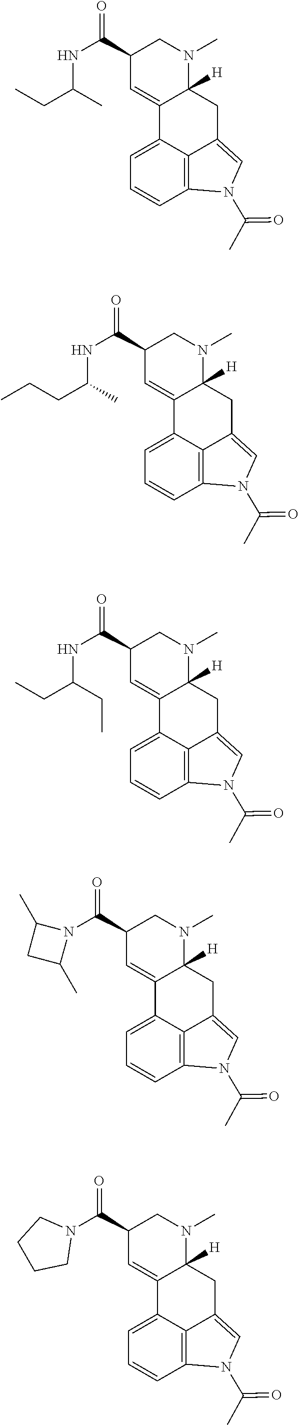 [SMILES: CCC(C)NC(=O)[C@@H]1C=C2c3cccc4c3c(cn4C(C)=O)C[C@H]2N(C)C1 ; CCC[C@@H](C)NC(=O)[C@@H]1C=C2c3cccc4c3c(cn4C(C)=O)C[C@H]2N(C)C1 ; CCC(CC)NC(=O)[C@@H]1C=C2c3cccc4c3c(cn4C(C)=O)C[C@H]2N(C)C1 ; CC(=O)n1cc2c3c(cccc31)C1=C[C@@H](C(=O)N3C(C)CC3C)CN(C)[C@@H]1C2 ; CC(=O)n1cc2c3c(cccc31)C1=C[C@@H](C(=O)N3CCCC3)CN(C)[C@@H]1C2]

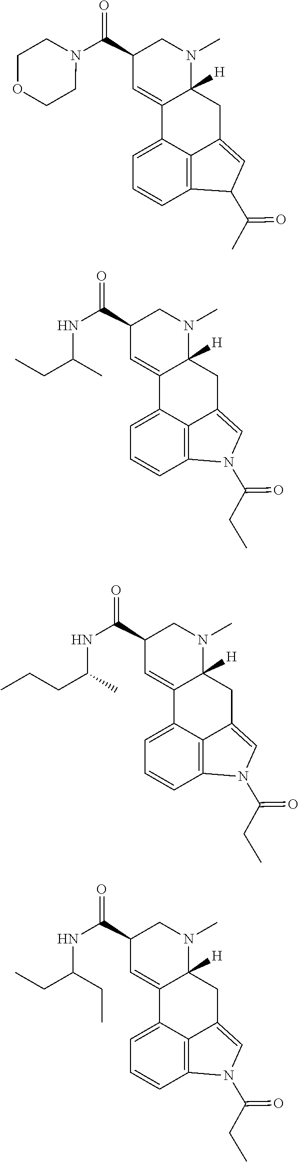 [SMILES: CC(=O)C1C=C2C[C@@H]3C(=C[C@@H](C(=O)N4CCOCC4)CN3C)c3cccc1c32 ; CCC(=O)n1cc2c3c(cccc31)C1=C[C@@H](C(=O)NC(C)CC)CN(C)[C@@H]1C2 ; CCC[C@@H](C)NC(=O)[C@@H]1C=C2c3cccc4c3c(cn4C(=O)CC)C[C@H]2N(C)C1 ; CCC(=O)n1cc2c3c(cccc31)C1=C[C@@H](C(=O)NC(CC)CC)CN(C)[C@@H]1C2]

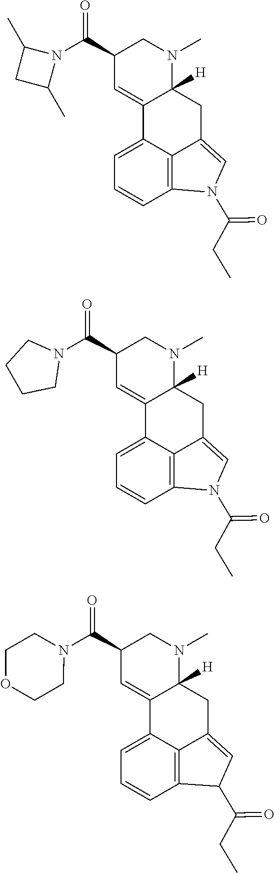 [SMILES: CCC(=O)n1cc2c3c(cccc31)C1=C[C@@H](C(=O)N3C(C)CC3C)CN(C)[C@@H]1C2 ; CCC(=O)n1cc2c3c(cccc31)C1=C[C@@H](C(=O)N3CCCC3)CN(C)[C@@H]1C2 ; CCC(=O)C1C=C2C[C@@H]3C(=C[C@@H](C(=O)N4CCOCC4)CN3C)c3cccc1c32]

or a pharmaceutically acceptable salt thereof.

In some embodiments, the present disclosure includes a compound selected from the group consisting of:

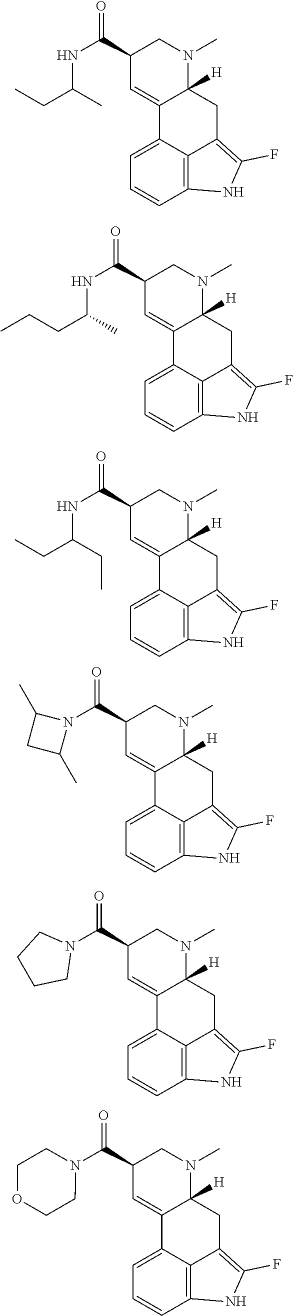

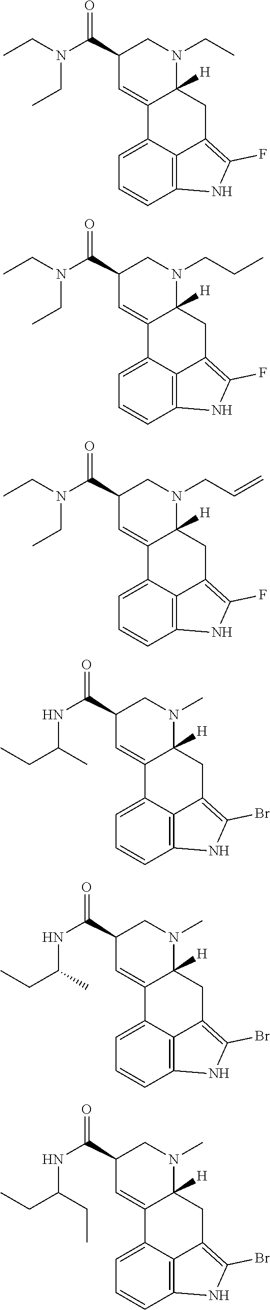

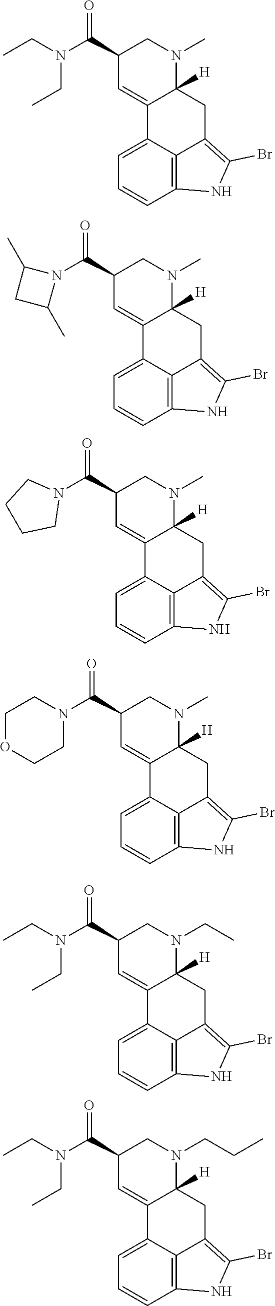

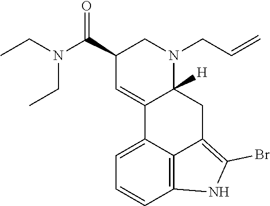 [SMILES: C=CCN1C[C@H](C(=O)N(CC)CC)C=C2c3cccc4[nH]c(Br)c(c34)C[C@H]21]

or a pharmaceutically acceptable salt thereof.

In other embodiments provided are methods and compositions for treating a mood disorder by administering to a subject in need thereof an effective amount of a compound selected from the group consisting of:

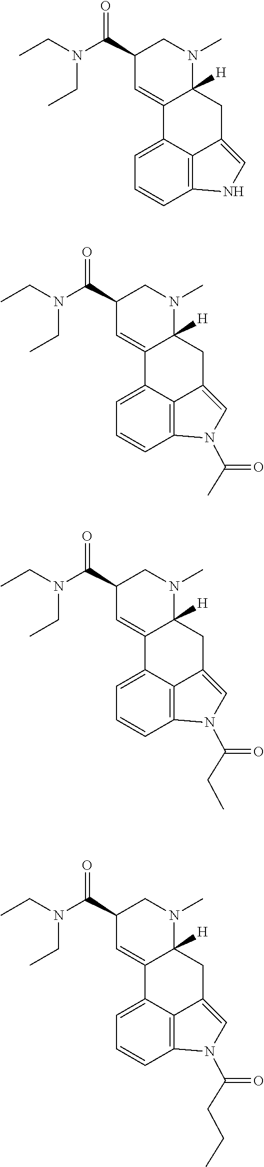 [SMILES: CCN(CC)C(=O)[C@@H]1C=C2c3cccc4[nH]cc(c34)C[C@H]2N(C)C1 ; CCN(CC)C(=O)[C@@H]1C=C2c3cccc4c3c(cn4C(C)=O)C[C@H]2N(C)C1 ; CCC(=O)n1cc2c3c(cccc31)C1=C[C@@H](C(=O)N(CC)CC)CN(C)[C@@H]1C2 ; CCCC(=O)n1cc2c3c(cccc31)C1=C[C@@H](C(=O)N(CC)CC)CN(C)[C@@H]1C2]

or a pharmaceutically acceptable salt thereof.

In some embodiments, the present disclosure includes a compound selected from the group consisting of:

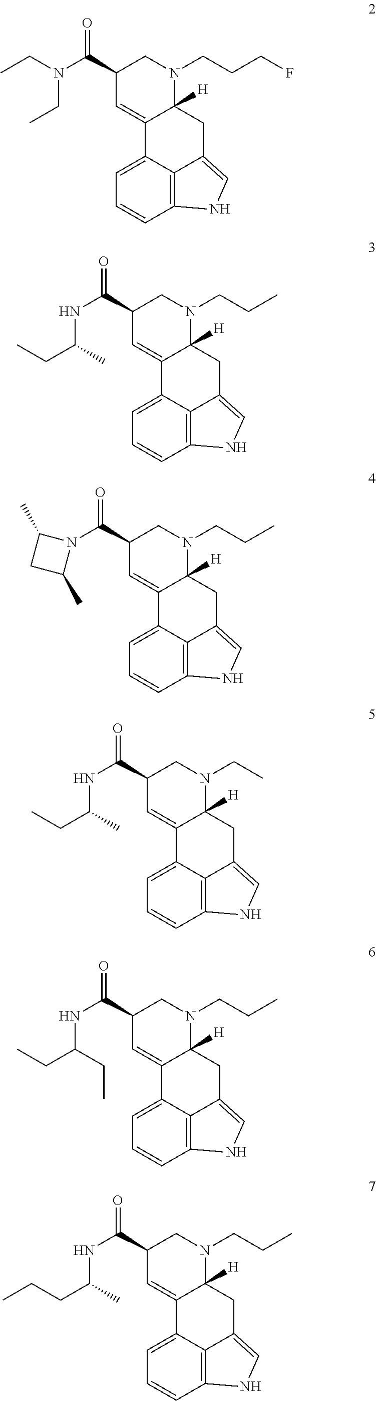

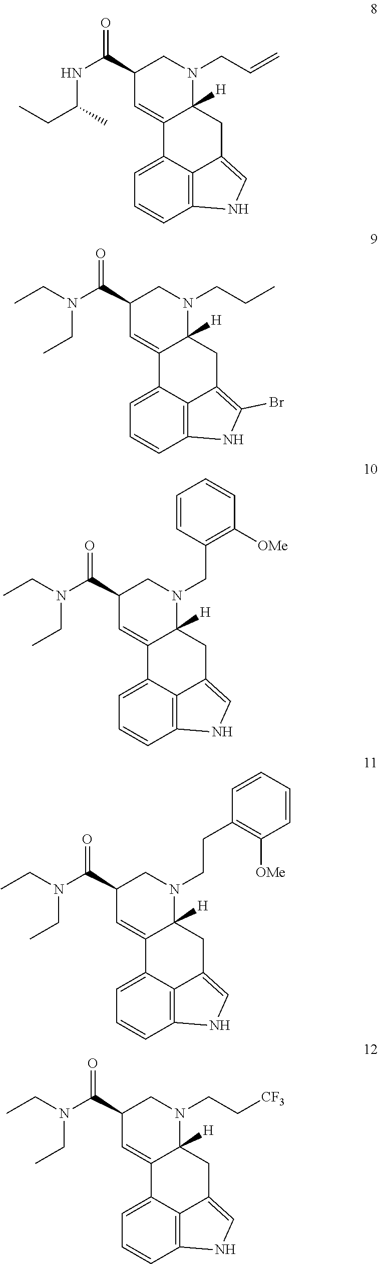

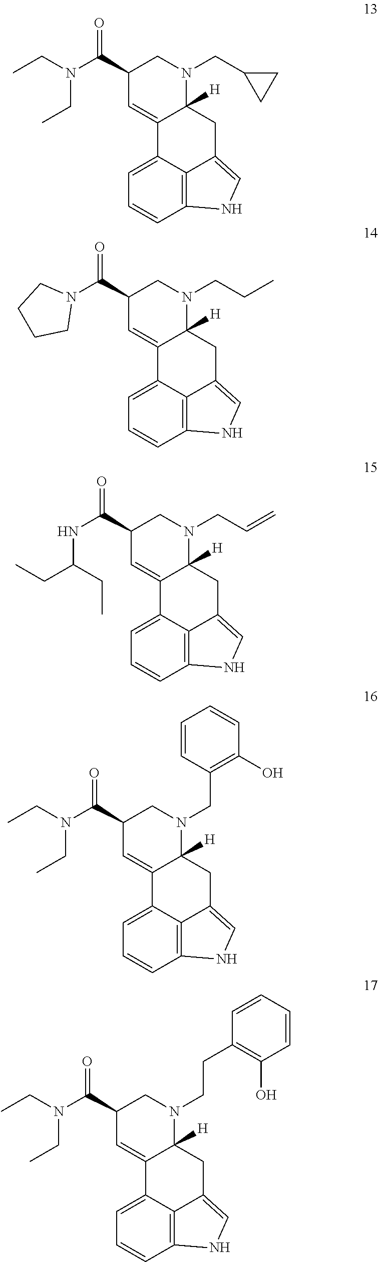

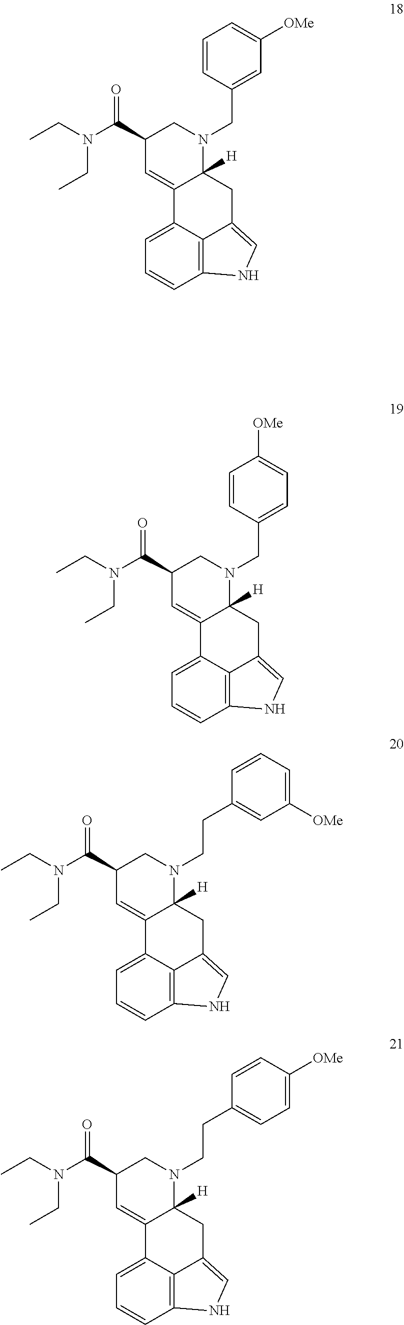

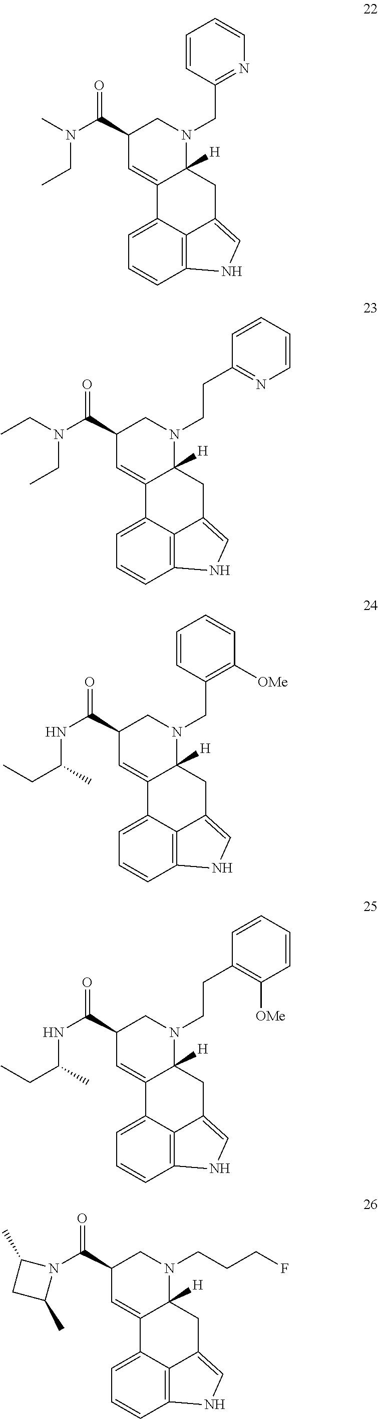

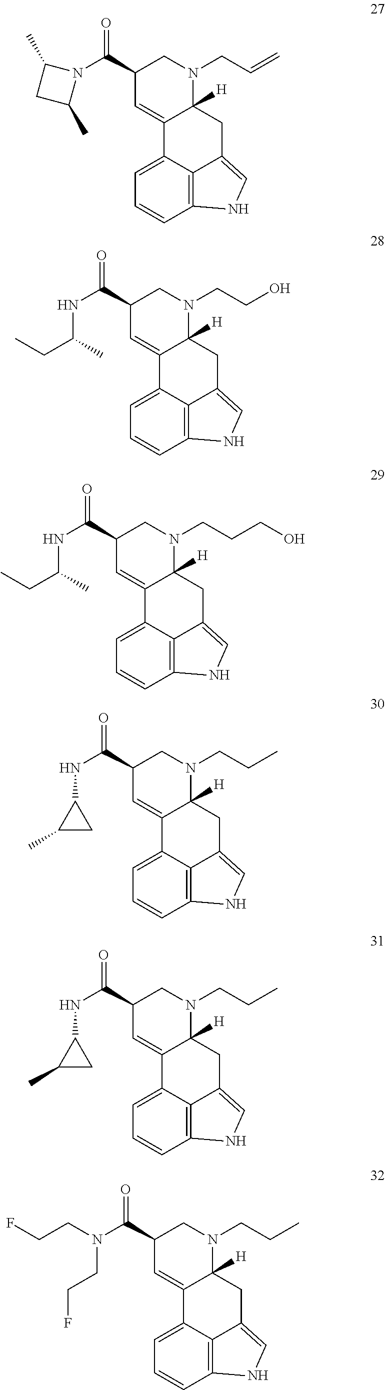

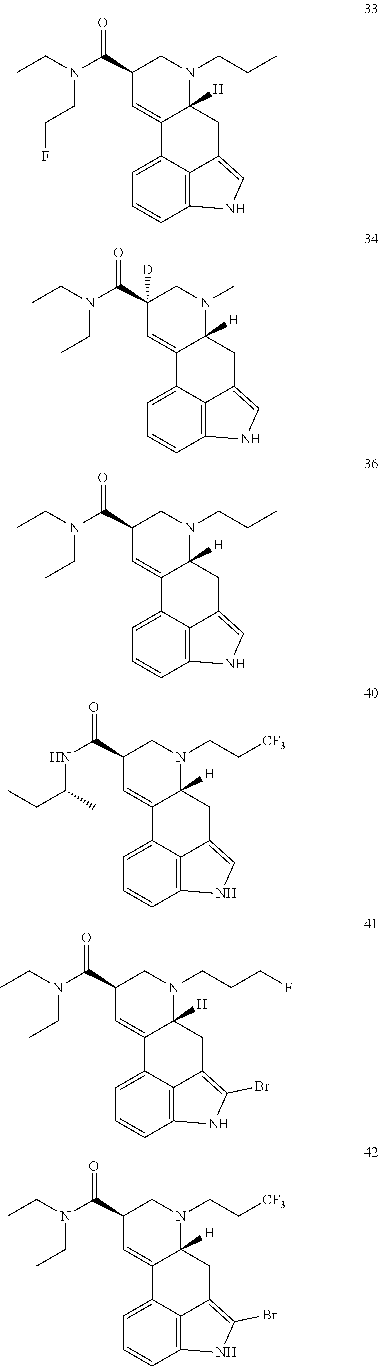

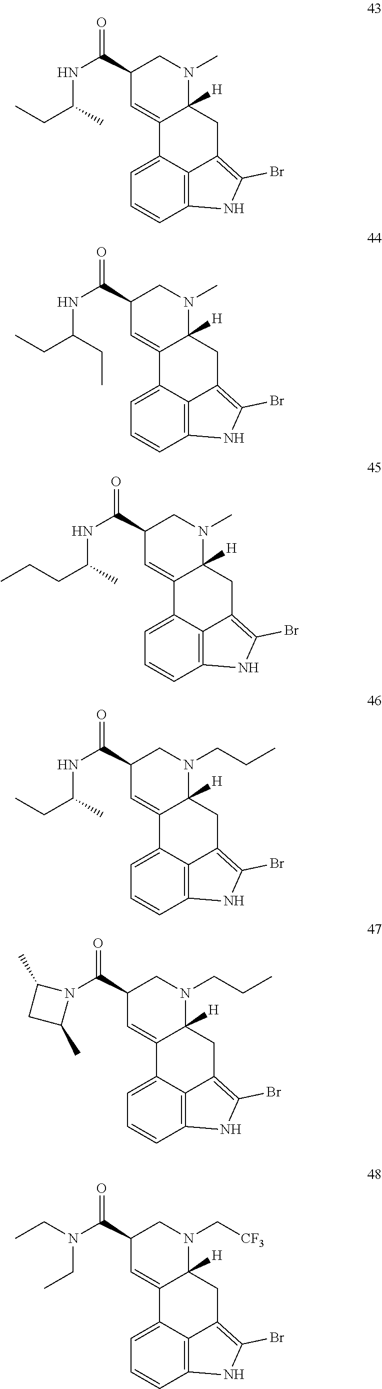

or a pharmaceutically acceptable salt thereof.

In some embodiments, the present disclosure includes a compound selected from the group consisting of

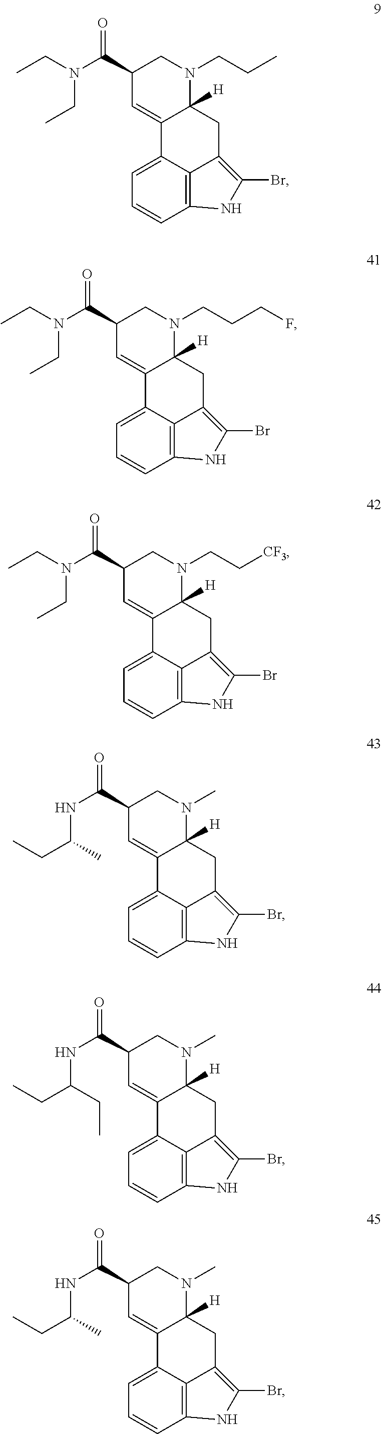

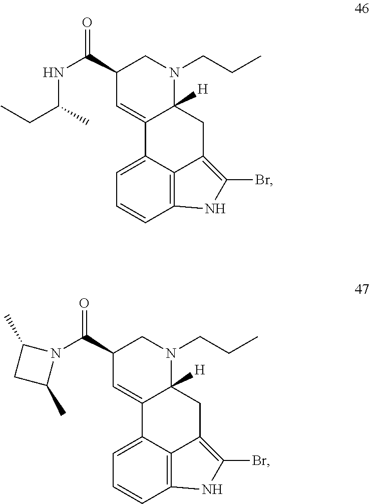

or a pharmaceutically acceptable salt thereof.

Salts of compounds of the present disclosure can be prepared by the reaction of a compound of the present disclosure with an appropriate acid or base in a suitable solvent, or mixture of solvents (such as an ether, for example, diethyl ether, or an alcohol, for example ethanol, or an aqueous solvent) using conventional procedures. Salts of compounds of General Formula I can be exchanged for other salts by treatment using conventional ion-exchange chromatography procedures. Preferred salts of compounds of the present disclosure include tartrate, fumarate, and maleate.

Where it is desired to obtain a particular enantiomer of a compound of the present disclosure, this may be produced from a corresponding mixture of enantiomers by employing any suitable conventional procedure for resolving enantiomers. For example, diastereomeric derivatives (such as salts) can be produced by reaction of a mixture of enantiomers of a compound the present disclosure (such a racemate) and an appropriate chiral compound (such as a chiral base). The diastereomers can then be separated by any conventional means such as crystallization, and the desired enantiomer recovered (such as by treatment with an acid in the instance where the diastereomer is a salt). Alternatively, a racemic mixture of esters can be resolved by kinetic hydrolysis using a variety of biocatalysts (for example, see Patel Stereoselective Biocatalysts, Marcel Decker; New York 2000).

In another resolution process a racemate of compounds of the present disclosure can be separated using chiral High Performance Liquid Chromatography. Alternatively, a particular enantiomer can be obtained by using an appropriate chiral intermediate in one of the processes described above. Chromatography, recrystallisation and other conventional separation procedures may also be used with intermediates or final products where it is desired to obtain a particular geometric isomer of the present disclosure.

### II. Methods

Described herein are methods and compositions for treating a mood disorder by administering to a patient in need thereof a compound of the present disclosure. Also provided are pharmaceutical compositions that include a compound of the present disclosure.

In embodiments, the methods, compounds, and compositions may be used to treat a mood disorder including Depressive Disorders, e.g., Major Depressive Disorder, Persistent Depressive Disorder, Postpartum Depression, Premenstrual Dysphoric Disorder, Seasonal Affective Disorder, Psychotic Depression, Disruptive Mood Dysregulation Disorder, Substance/Medication-Induced Depressive Disorder, or Depressive Disorder Due to Another Medical Condition.

In embodiments, the methods, compounds, and compositions may treat mood disorders that include Bipolar and Related Disorders. In embodiments, the methods, compounds, and compositions may treat mood disorders that include Substance-Related Disorders. In embodiments, the methods, compounds, and compositions may treat mood disorders that include Anxiety Disorders. In embodiments, the methods, compounds, and compositions may treat mood disorders that include Obsessive-Compulsive and Related Disorders. In embodiments, the methods, compounds, and compositions may treat mood disorders that include Trauma- and Stressor-Related Disorders. In embodiments, the methods, compounds, and compositions may treat mood disorders that include Feeding and Eating Disorders. In embodiments, the methods, compounds, and compositions may treat mood disorders that include Neurocognitive Disorders. In embodiments, the methods, compounds, and compositions may treat mood disorders that include Neurodevelopmental Disorders. In embodiments, the methods, compounds, and compositions may treat mood disorders that include Personality Disorders. In embodiments, the methods, compounds, and compositions may treat mood disorders that include Sexual Dysfunctions. In embodiments, the methods, compounds, and compositions may treat mood disorders that include Gender Dysphoria. In embodiments, the methods, compounds, and compositions may treat migraine or cluster headache.

Also provided herein are methods of treating refractory depression, e.g., patients suffering from a depressive disorder that does not, and/or has not, responded to adequate courses of at least one, or at least two, other antidepressant compounds or therapeutics. As used herein “depressive disorder” encompasses refractory depression.

In embodiments, the methods, compounds, and compositions may be used to treat a mood disorder including Bipolar and Related Disorders, e.g., Bipolar I Disorder, Bipolar II Disorder, Cyclothymic Disorder, Substance/Medication-Induced Bipolar and Related Disorder, Bipolar and Related Disorder Due to Another Medical Condition,

In embodiments, the methods, compounds, and compositions may be used to treat a mood disorder including Substance-Related Disorders, e.g., preventing a substance use craving, diminishing a substance use craving, and/or facilitating substance use cessation or withdrawal. Substance use disorders involve abuse of psychoactive compounds such as alcohol, caffeine, cannabis, inhalants, opioids, sedatives, hypnotics, anxiolytics, stimulants, nicotine, and tobacco. As used herein “substance” or “substances” are psychoactive compounds which can be addictive such as alcohol, caffeine, cannabis, hallucinogens, inhalants, opioids, sedatives, hypnotics, anxiolytics, stimulants, nicotine, and tobacco. For example, the methods, compounds, and compositions may be used to facilitate smoking cessation or cessation of opioid use.

In embodiments, the methods, compounds, and compositions may be used to treat a mood disorder including Anxiety Disorders, e.g., Separation Anxiety Disorder, Selective Mutism, Specific Phobia, Social Anxiety Disorder (Social Phobia), Panic Disorder, Panic Attack, Agoraphobia, Generalized Anxiety Disorder, Substance/Medication-Induced Anxiety Disorder, or Anxiety Disorder Due to Another Medical Condition.

In embodiments, the methods, compounds, and compositions may be used to treat a mood disorder including Obsessive-Compulsive and Related Disorders, e.g., Obsessive-Compulsive Disorder, Body Dysmorphic Disorder, Hoarding Disorder, Trichotillomania (Hair-Pulling Disorder), Excoriation (Skin-Picking) Disorder, Substance/Medication-Induced Obsessive-Compulsive, and Related Disorder, or Obsessive-Compulsive and Related Disorder Due to Another Medical Condition.

In embodiments, the methods, compounds, and compositions may be used to treat a mood disorder including Trauma- and Stressor-Related Disorders, e.g., Reactive Attachment Disorder, Disinhibited Social Engagement Disorder, Posttraumatic Stress Disorder, Acute Stress Disorder, or Adjustment Disorders.

In embodiments, the methods, compounds, and compositions may be used to treat a mood disorder including Feeding and Eating Disorders, e.g., Anorexia Nervosa, Bulimia Nervosa, Binge-Eating Disorder, Pica, Rumination Disorder, or Avoidant/Restrictive Food Intake Disorder.

In embodiments, the methods, compounds, and compositions may be used to treat a mood disorder including Neurocognitive Disorders, e.g., Delirium, Major Neurocognitive Disorder, Mild Neurocognitive Disorder, Major or Mild Neurocognitive Disorder Due to Alzheimer's Disease, Major or Mild Frontotemporal Neurocognitive Disorder, Major or Mild Neurocognitive Disorder With Lewy Bodies, Major or Mild Vascular Neurocognitive Disorder, Major or Mild Neurocognitive Disorder Due to Traumatic Brain Injury, Substance/Medication-Induced Major or Mild Neurocognitive Disorder, Major or Mild Neurocognitive Disorder Due to HIV Infection, Major or Mild Neurocognitive Disorder Due to Prion Disease, Major or Mild Neurocognitive Disorder Due to Parkinson's Disease, Major or Mild Neurocognitive Disorder Due to Huntington's Disease, Major or Mild Neurocognitive Disorder Due to Another Medical Condition, or Major or Mild Neurocognitive Disorder Due to Multiple Etiologies,

In embodiments, the methods, compounds, and compositions may be used to treat a mood disorder including Neurodevelopmental Disorders, e.g., Autism Spectrum Disorder, Attention-Deficit/Hyperactivity Disorder, Stereotypic Movement Disorder, Tic Disorders, Tourette's Disorder, Persistent (Chronic) Motor or Vocal Tic Disorder, or Provisional Tic Disorder,

In embodiments, the methods, compounds, and compositions may be used to treat a mood disorder including Personality Disorders, e.g., Borderline Personality Disorder.

In embodiments, the methods, compounds, and compositions may be used to treat a mood disorder including Sexual Dysfunctions, e.g., Delayed Ejaculation, Erectile Disorder, Female Orgasmic Disorder, Female Sexual Interest/Arousal Disorder, Genito-Pelvic Pain/Penetration Disorder, Male Hypoactive Sexual Desire Disorder, Premature (Early) Ejaculation, or Substance/Medication-Induced Sexual Dysfunction.

In embodiments, the methods, compounds, and compositions may be used to treat a mood disorder including Gender Dysphoria, e.g., Gender Dysphoria.

In embodiments provided are methods and compositions for treating a mood disorder by administering to a subject in need thereof an effective amount of (6aR,9R)—N,N-diethyl-7-propyl-4,6,6a,7,8,9-hexahydroindolo[4,3-fg]quinoline-9-carboxamide (1) or a pharmaceutically acceptable salt thereof.

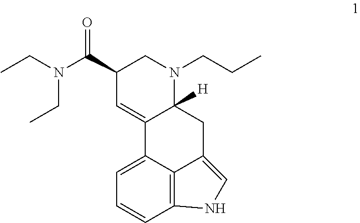 [SMILES: CCCN1C[C@H](C(=O)N(CC)CC)C=C2c3cccc4[nH]cc(c34)C[C@H]21]

In other embodiments, provided is a method of treating a mood disorder comprising administering to a patient in need thereof a pharmaceutical composition comprising an effective amount of a compound according to Formula I or II, as defined hereinabove, or pharmaceutically acceptable salts thereof, or Formulae Ia or IIa, or Ib or IIb or Ic or IIc, or Id or IId or Ie or IIe or pharmaceutically salts of Formulae Ia or IIa, or Ib or IIb or Ic or IIc, or Id or IId or Ie or IIe.

In other embodiments, provided is a method of treating a mood disorder comprising administering to a patient in need thereof a pharmaceutical composition comprising an effective amount of a compound according to Formula (III):

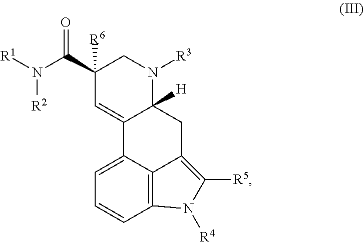

or a pharmaceutically acceptable salt thereof,

wherein
R¹ is C₁-C₆ alkyl or 3-7 membered carbocyclyl, wherein R¹ is optionally substituted with one or more halogen or C₁-C₆ alkyl;
R² is hydrogen or C₁-C₆ alkyl, wherein R² is optionally substituted with one or more halogen or C₁-C₆ alkyl; or
   wherein R¹ and R² can be taken together with the atom on which they are attached to form an optionally substituted 3-7 membered heterocyclyl comprising 1-3 heteroatoms selected from the group consisting of N, O, and S, wherein the heterocyclyl is optionally substituted with one or more fluoro or C₁-C₆ alkyl;
R³ is selected from the group consisting of C₁-C₆ alkyl, C₂-C₆ alkenyl, C₂-C₆ alkynyl, —CH₂-(cyclopropyl), and 3-7 membered cycloalkyl,
   wherein R³ may be substituted with one or more substituents each independently selected from the group consisting of fluoro, hydroxyl, and —OMe;
   or
   R³ is selected from the group consisting of —(C₁-C₂ alkyl)-phenyl and —(C₁-C₂ alkyl)-(6-membered heteroaryl),
   wherein C₁-C₂ alkyl is optionally substituted with one or more fluoro, hydroxyl, and —OMe, and wherein phenyl and 6-membered heteroaryl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₄ alkyl, C₃-C₅ cycloalkyl, and C₁-C₄ alkoxy;
R⁴ is hydrogen or —C(O)(C₁-C₈ alkyl);
R⁵ is hydrogen, Me, Et, —CH₂F, CHF₂, —CF₃, or halogen;
R⁶ is hydrogen or deuterium.

In other embodiments provided are methods and compositions for treating a mood disorder by administering to a subject in need thereof an effective amount of a compound selected from the group consisting of:

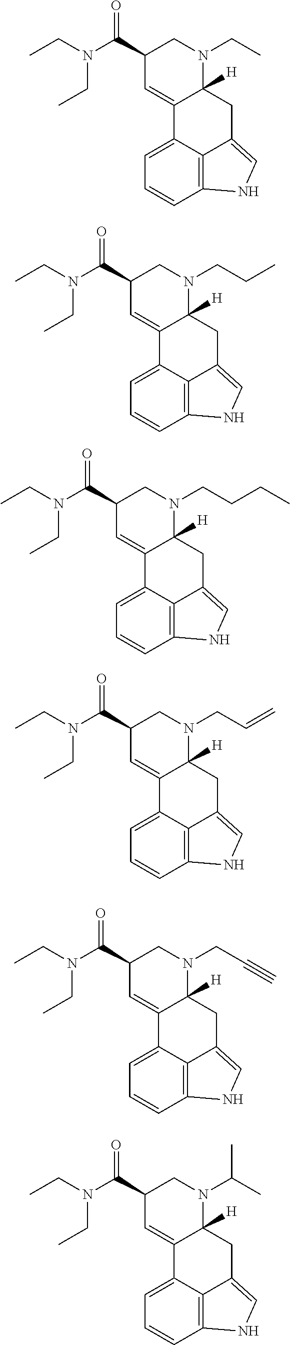

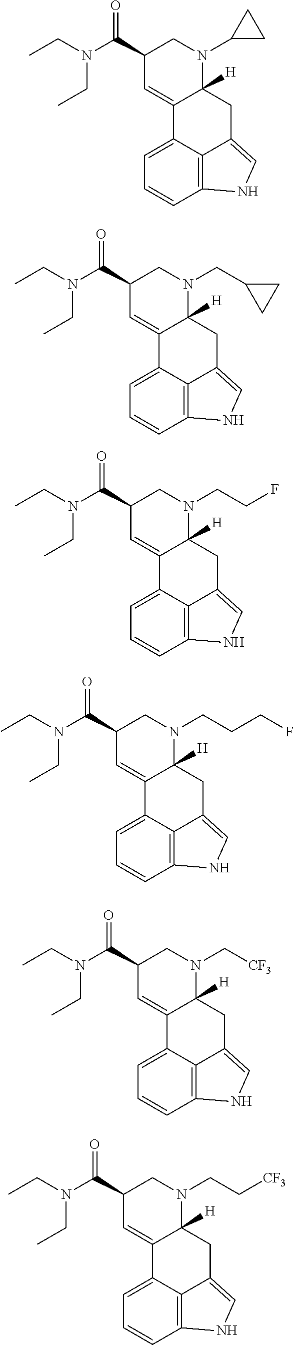 [SMILES: CCN(CC)C(=O)[C@@H]1C=C2c3cccc4[nH]cc(c34)C[C@H]2N(C2CC2)C1 ; CCN(CC)C(=O)[C@@H]1C=C2c3cccc4[nH]cc(c34)C[C@H]2N(CC2CC2)C1 ; CCN(CC)C(=O)[C@@H]1C=C2c3cccc4[nH]cc(c34)C[C@H]2N(CCF)C1 ; CCN(CC)C(=O)[C@@H]1C=C2c3cccc4[nH]cc(c34)C[C@H]2N(CCCF)C1 ; CCN(CC)C(=O)[C@@H]1C=C2c3cccc4[nH]cc(c34)C[C@H]2N(CC(F)(F)F)C1 ; CCN(CC)C(=O)[C@@H]1C=C2c3cccc4[nH]cc(c34)C[C@H]2N(CCC(F)(F)F)C1]

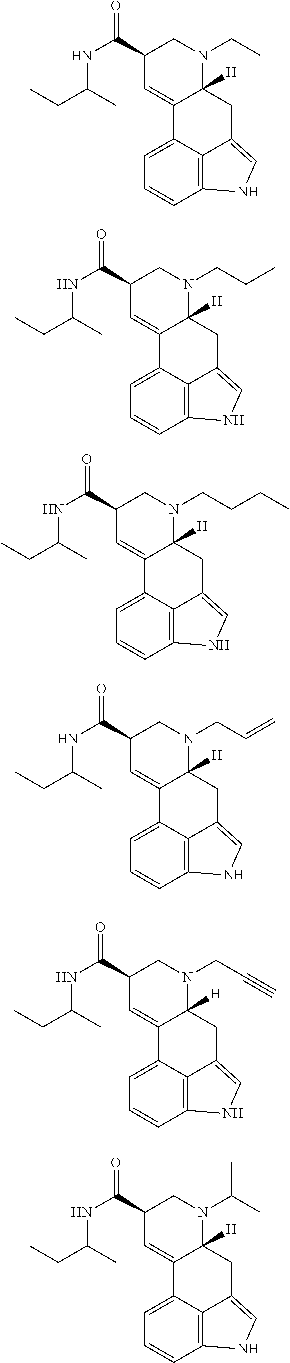

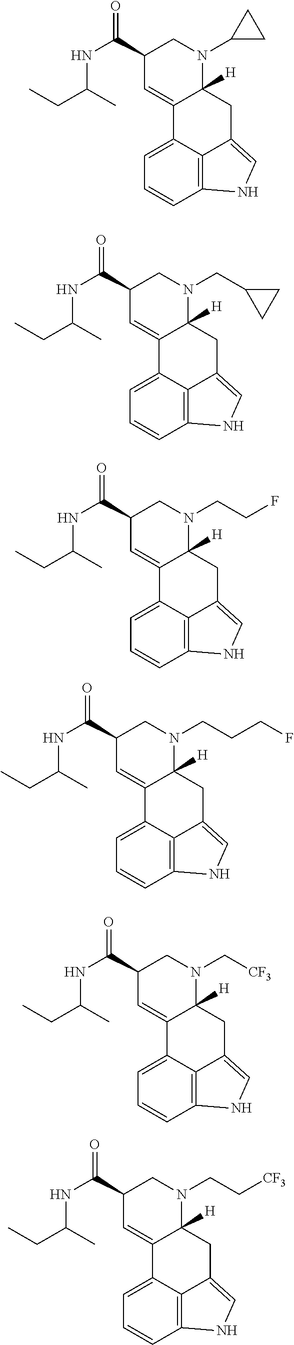 [SMILES: CCC(C)NC(=O)[C@@H]1C=C2c3cccc4[nH]cc(c34)C[C@H]2N(C2CC2)C1 ; CCC(C)NC(=O)[C@@H]1C=C2c3cccc4[nH]cc(c34)C[C@H]2N(CC2CC2)C1 ; CCC(C)NC(=O)[C@@H]1C=C2c3cccc4[nH]cc(c34)C[C@H]2N(CCF)C1 ; CCC(C)NC(=O)[C@@H]1C=C2c3cccc4[nH]cc(c34)C[C@H]2N(CCCF)C1 ; CCC(C)NC(=O)[C@@H]1C=C2c3cccc4[nH]cc(c34)C[C@H]2N(CC(F)(F)F)C1 ; CCC(C)NC(=O)[C@@H]1C=C2c3cccc4[nH]cc(c34)C[C@H]2N(CCC(F)(F)F)C1]

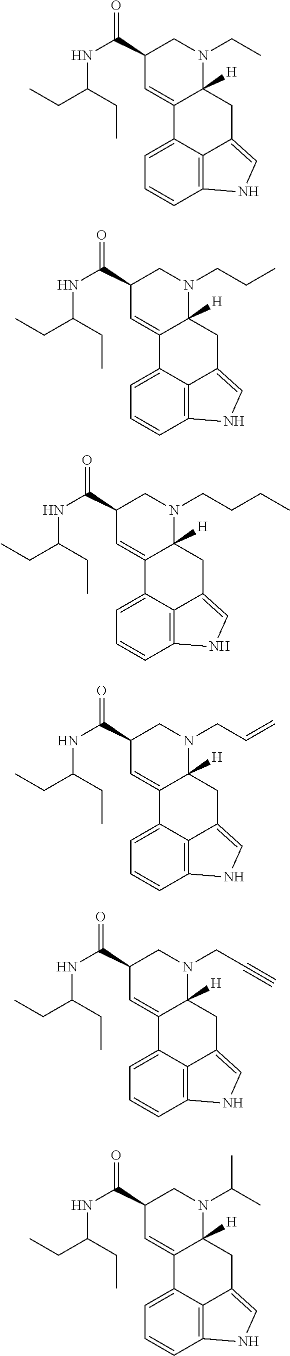

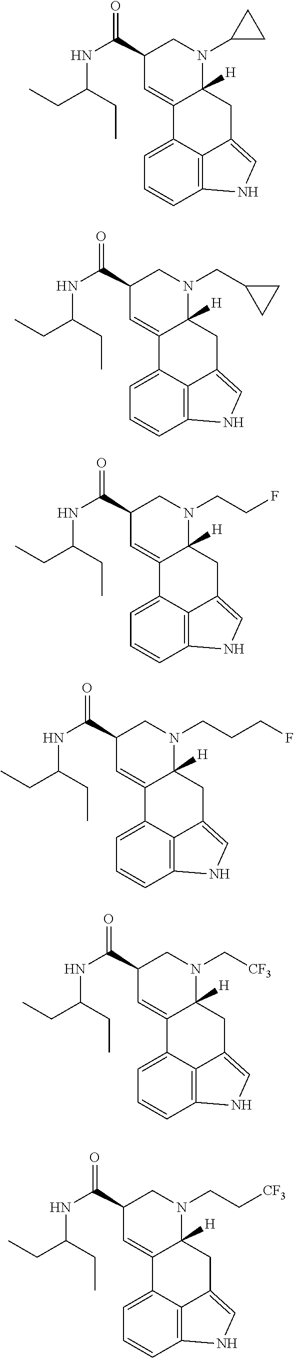 [SMILES: CCC(CC)NC(=O)[C@@H]1C=C2c3cccc4[nH]cc(c34)C[C@H]2N(C2CC2)C1 ; CCC(CC)NC(=O)[C@@H]1C=C2c3cccc4[nH]cc(c34)C[C@H]2N(CC2CC2)C1 ; CCC(CC)NC(=O)[C@@H]1C=C2c3cccc4[nH]cc(c34)C[C@H]2N(CCF)C1 ; CCC(CC)NC(=O)[C@@H]1C=C2c3cccc4[nH]cc(c34)C[C@H]2N(CCCF)C1 ; CCC(CC)NC(=O)[C@@H]1C=C2c3cccc4[nH]cc(c34)C[C@H]2N(CC(F)(F)F)C1 ; CCC(CC)NC(=O)[C@@H]1C=C2c3cccc4[nH]cc(c34)C[C@H]2N(CCC(F)(F)F)C1]

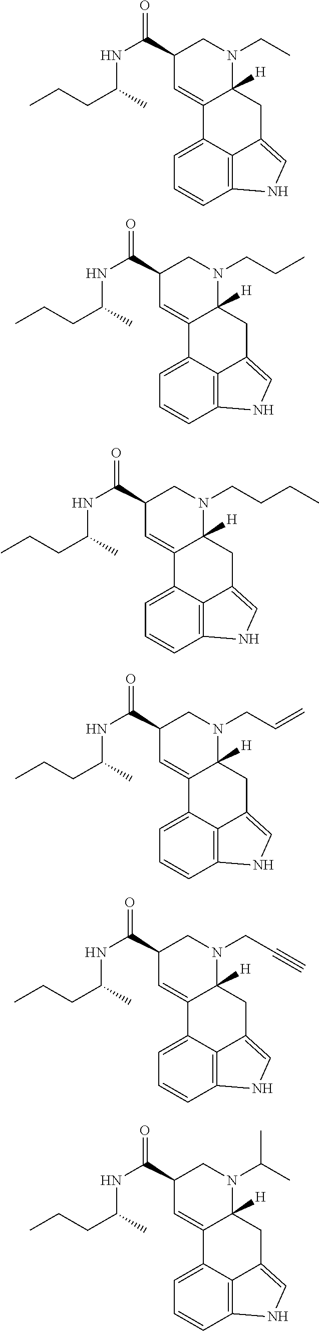

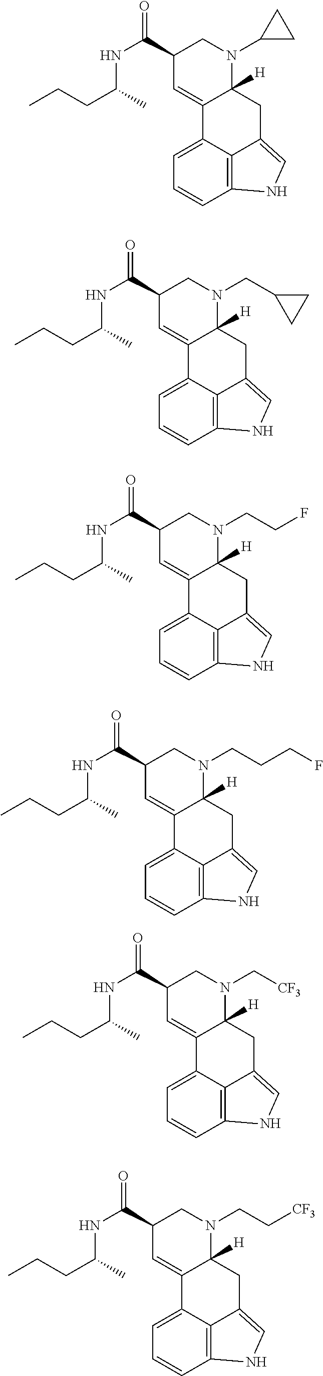

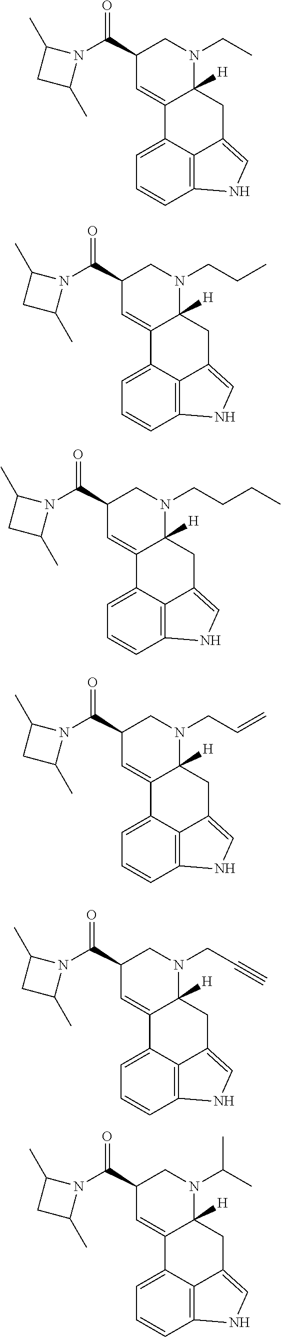

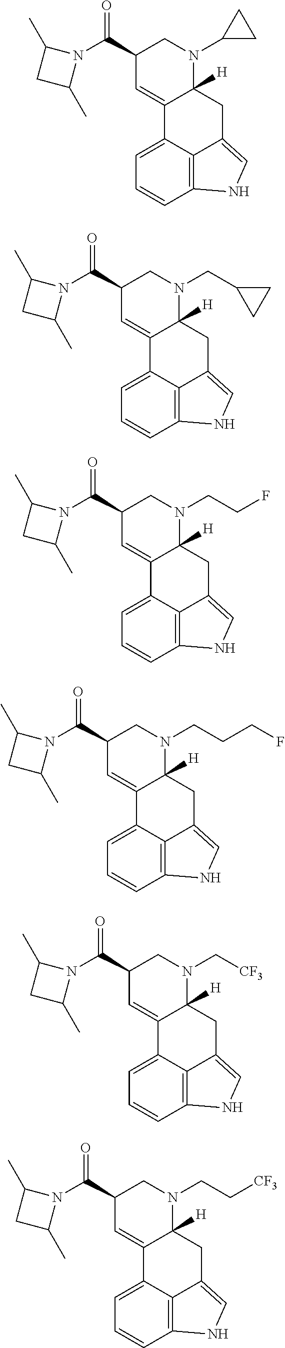

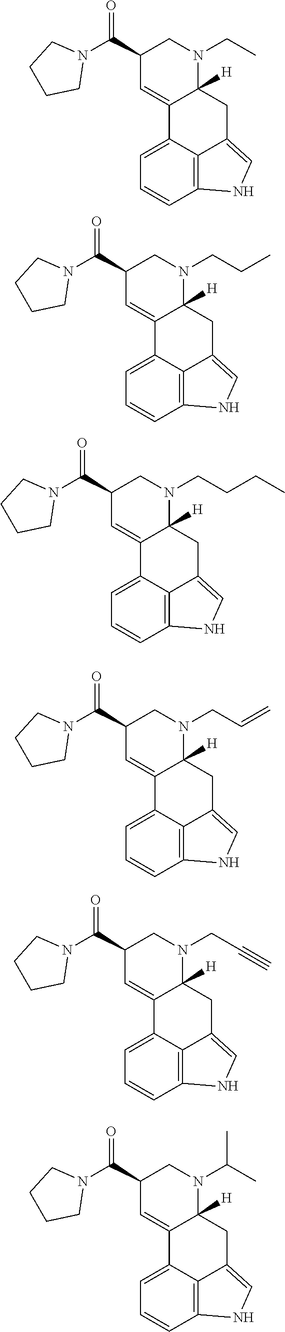

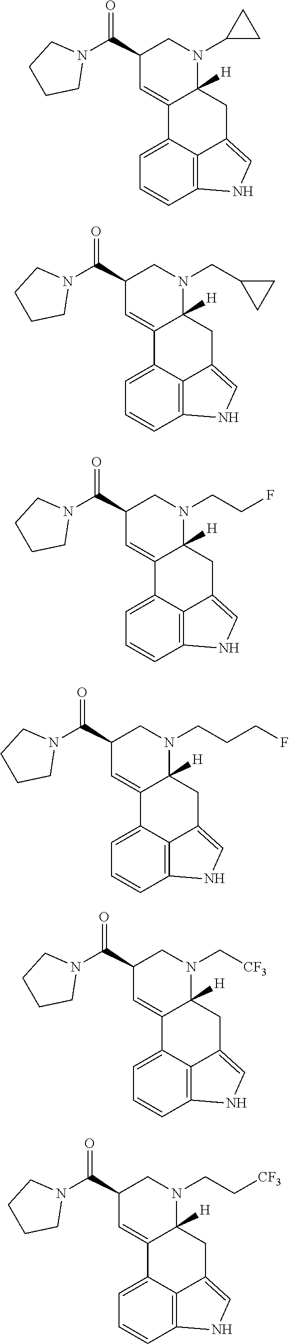

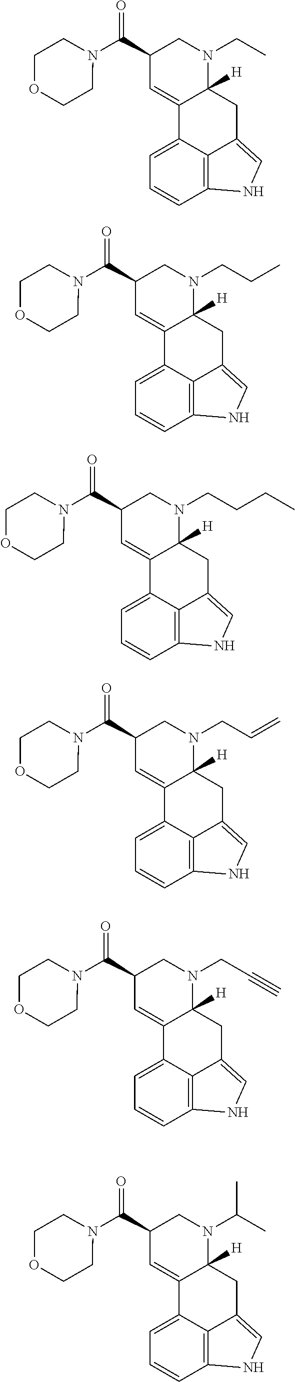 [SMILES: CCN1C[C@H](C(=O)N2CCOCC2)C=C2c3cccc4[nH]cc(c34)C[C@H]21 ; CCCN1C[C@H](C(=O)N2CCOCC2)C=C2c3cccc4[nH]cc(c34)C[C@H]21 ; CCCCN1C[C@H](C(=O)N2CCOCC2)C=C2c3cccc4[nH]cc(c34)C[C@H]21 ; C=CCN1C[C@H](C(=O)N2CCOCC2)C=C2c3cccc4[nH]cc(c34)C[C@H]21 ; C#CCN1C[C@H](C(=O)N2CCOCC2)C=C2c3cccc4[nH]cc(c34)C[C@H]21 ; CC(C)N1C[C@H](C(=O)N2CCOCC2)C=C2c3cccc4[nH]cc(c34)C[C@H]21]

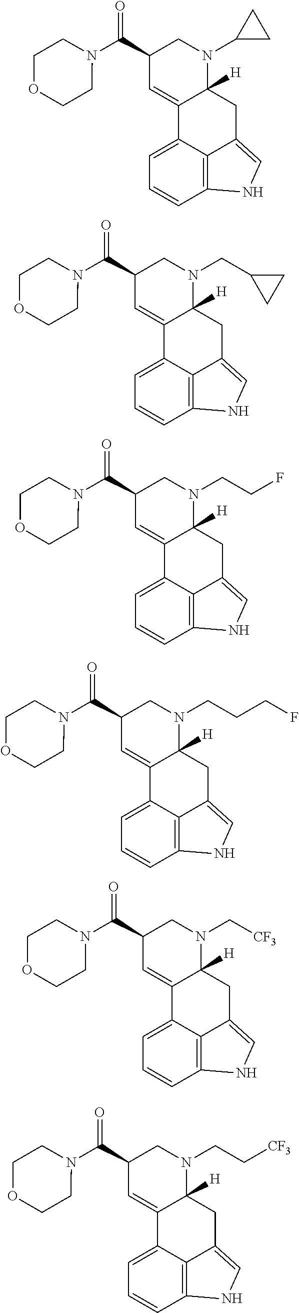

or a pharmaceutically acceptable salt thereof.

In other embodiments provided are methods and compositions for treating a mood disorder by administering to a subject in need thereof an effective amount of a compound selected from the group consisting of:

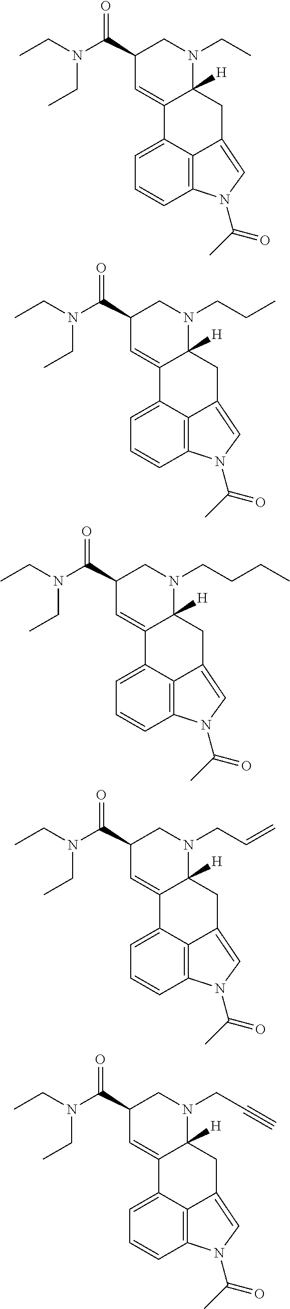

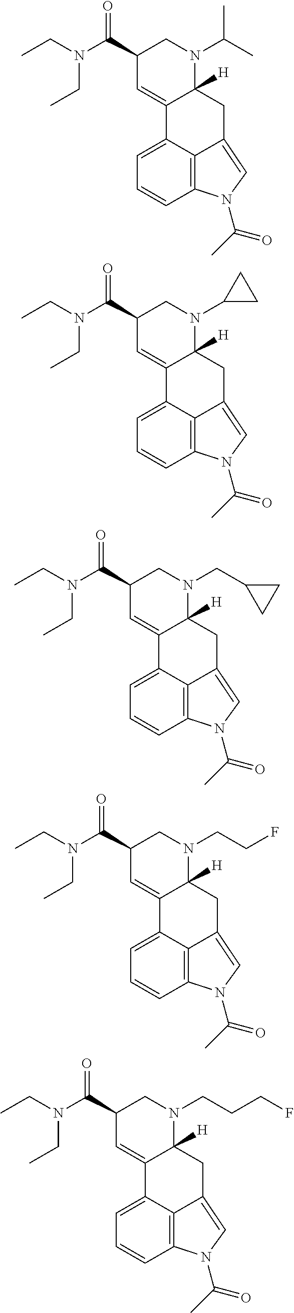

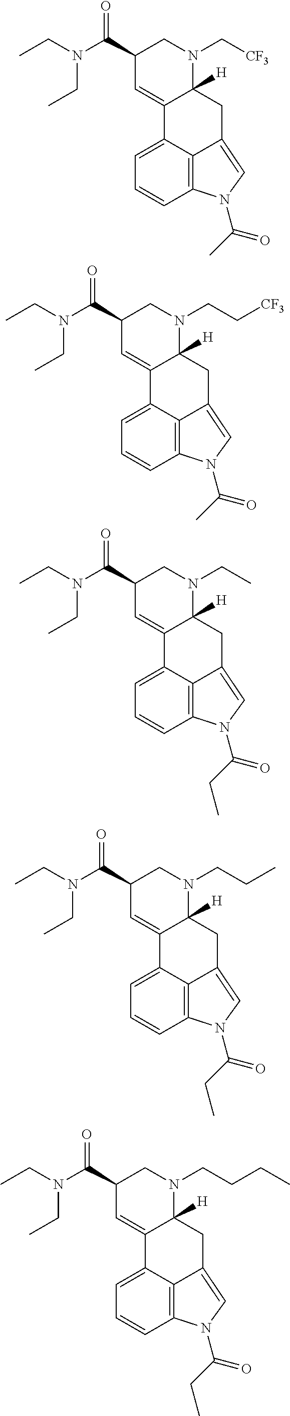

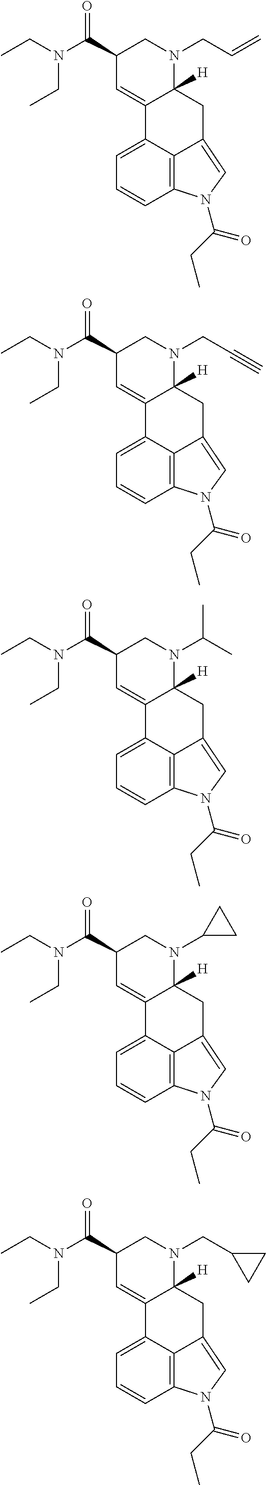

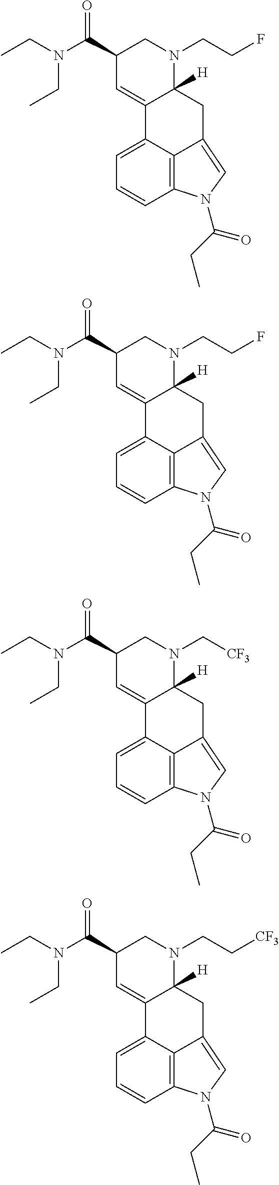 [SMILES: CCC(=O)n1cc2c3c(cccc31)C1=C[C@@H](C(=O)N(CC)CC)CN(CCF)[C@@H]1C2 ; CCC(=O)n1cc2c3c(cccc31)C1=C[C@@H](C(=O)N(CC)CC)CN(CCF)[C@@H]1C2 ; CCC(=O)n1cc2c3c(cccc31)C1=C[C@@H](C(=O)N(CC)CC)CN(CC(F)(F)F)[C@@H]1C2 ; CCC(=O)n1cc2c3c(cccc31)C1=C[C@@H](C(=O)N(CC)CC)CN(CCC(F)(F)F)[C@@H]1C2]

or a pharmaceutically acceptable salt thereof.

In other embodiments provided are methods and compositions for treating a mood disorder by administering to a subject in need thereof an effective amount of a compound selected from the group consisting of:

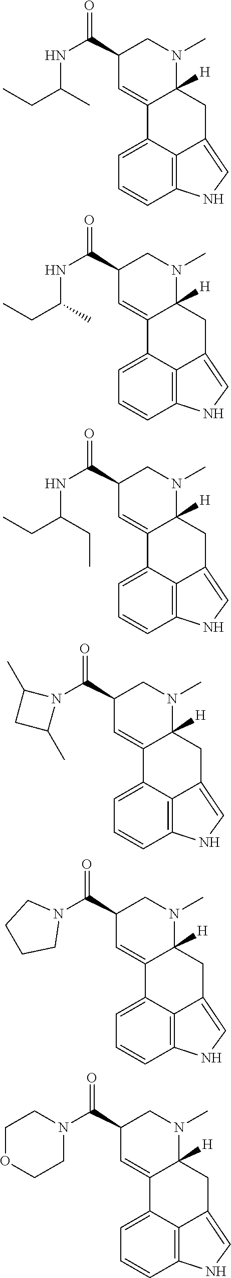

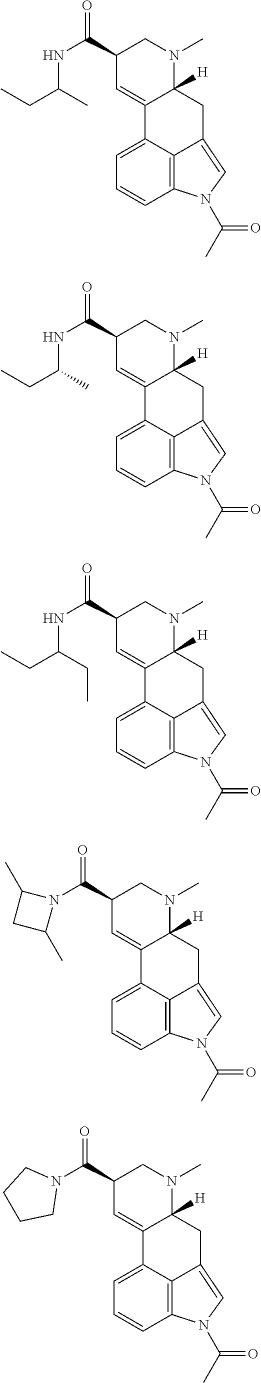 [SMILES: CCC(C)NC(=O)[C@@H]1C=C2c3cccc4c3c(cn4C(C)=O)C[C@H]2N(C)C1 ; CC[C@@H](C)NC(=O)[C@@H]1C=C2c3cccc4c3c(cn4C(C)=O)C[C@H]2N(C)C1 ; CCC(CC)NC(=O)[C@@H]1C=C2c3cccc4c3c(cn4C(C)=O)C[C@H]2N(C)C1 ; CC(=O)n1cc2c3c(cccc31)C1=C[C@@H](C(=O)N3C(C)CC3C)CN(C)[C@@H]1C2 ; CC(=O)n1cc2c3c(cccc31)C1=C[C@@H](C(=O)N3CCCC3)CN(C)[C@@H]1C2]

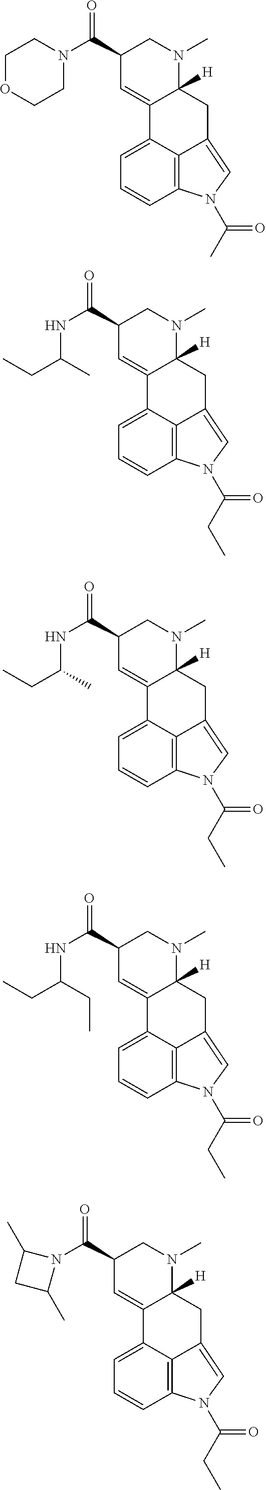 [SMILES: CC(=O)n1cc2c3c(cccc31)C1=C[C@@H](C(=O)N3CCOCC3)CN(C)[C@@H]1C2 ; CCC(=O)n1cc2c3c(cccc31)C1=C[C@@H](C(=O)NC(C)CC)CN(C)[C@@H]1C2 ; CCC(=O)n1cc2c3c(cccc31)C1=C[C@@H](C(=O)N[C@H](C)CC)CN(C)[C@@H]1C2 ; CCC(=O)n1cc2c3c(cccc31)C1=C[C@@H](C(=O)NC(CC)CC)CN(C)[C@@H]1C2 ; CCC(=O)n1cc2c3c(cccc31)C1=C[C@@H](C(=O)N3C(C)CC3C)CN(C)[C@@H]1C2]

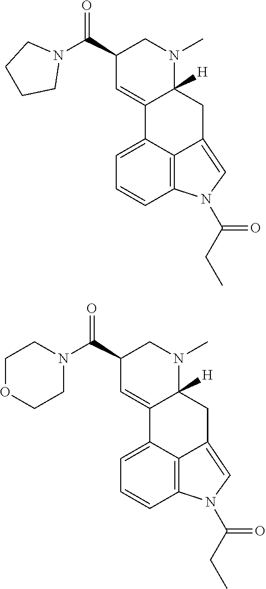 [SMILES: CCC(=O)n1cc2c3c(cccc31)C1=C[C@@H](C(=O)N3CCCC3)CN(C)[C@@H]1C2 ; CCC(=O)n1cc2c3c(cccc31)C1=C[C@@H](C(=O)N3CCOCC3)CN(C)[C@@H]1C2]

or a pharmaceutically acceptable salt thereof.

In other embodiments provided are methods and compositions for treating a mood disorder by administering to a subject in need thereof an effective amount of a compound selected from the group consisting of:

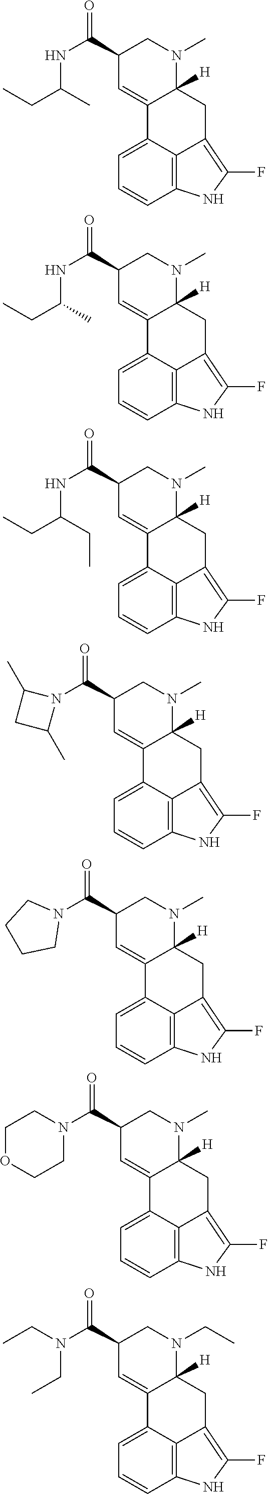

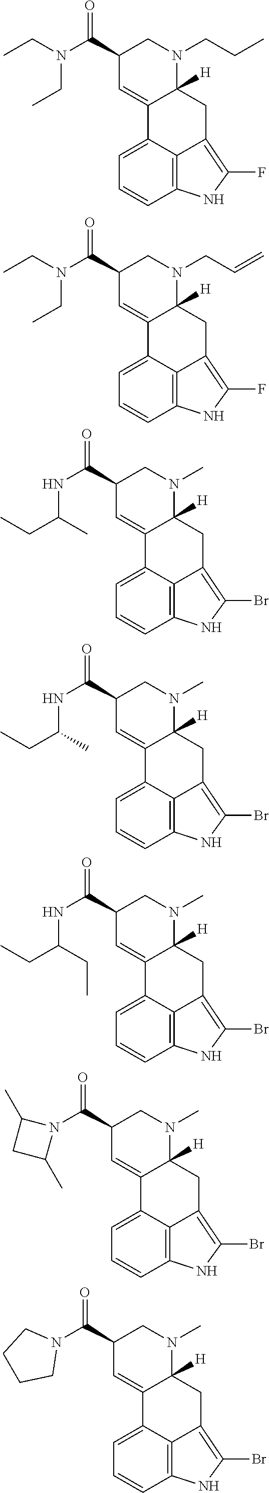

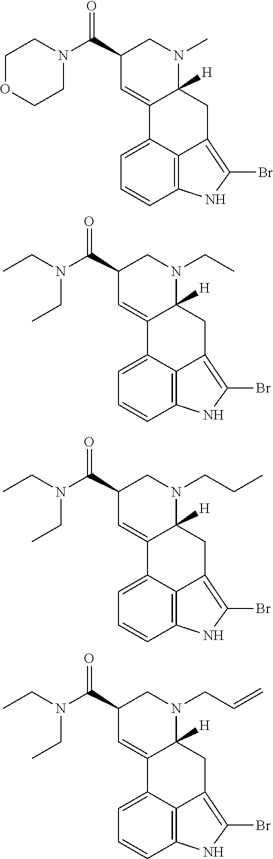

or a pharmaceutically acceptable salt thereof.

In other embodiments provided are methods and compositions for treating a mood disorder by administering to a subject in need thereof an effective amount of a compound selected from the group consisting of:

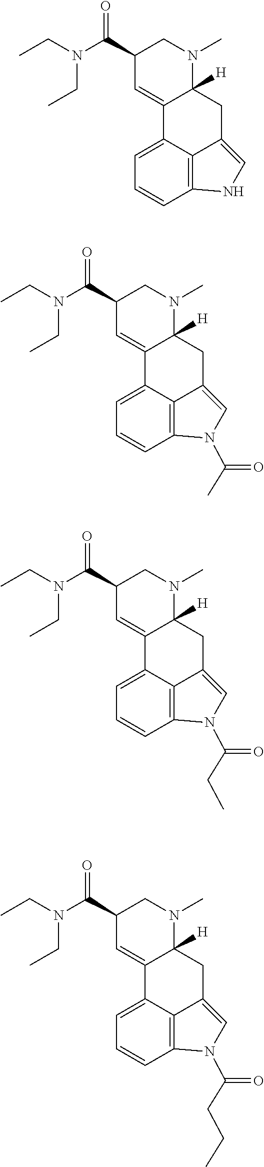 [SMILES: CCN(CC)C(=O)[C@@H]1C=C2c3cccc4[nH]cc(c34)C[C@H]2N(C)C1 ; CCN(CC)C(=O)[C@@H]1C=C2c3cccc4c3c(cn4C(C)=O)C[C@H]2N(C)C1 ; CCC(=O)n1cc2c3c(cccc31)C1=C[C@@H](C(=O)N(CC)CC)CN(C)[C@@H]1C2 ; CCCC(=O)n1cc2c3c(cccc31)C1=C[C@@H](C(=O)N(CC)CC)CN(C)[C@@H]1C2]

or a pharmaceutically acceptable salt thereof.

In other embodiments provided are methods and compositions for treating a mood disorder by administering to a subject in need thereof an effective amount of a compound selected from the group consisting of:

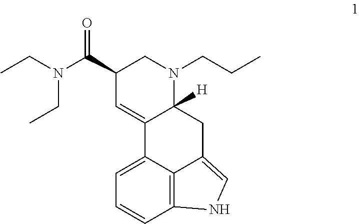 [SMILES: CCCN1C[C@H](C(=O)N(CC)CC)C=C2c3cccc4[nH]cc(c34)C[C@H]21]

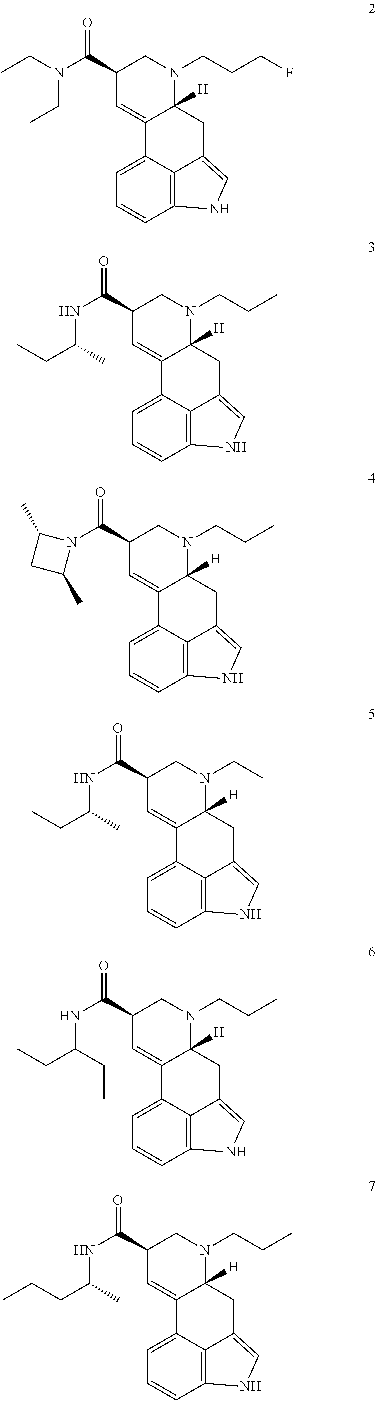

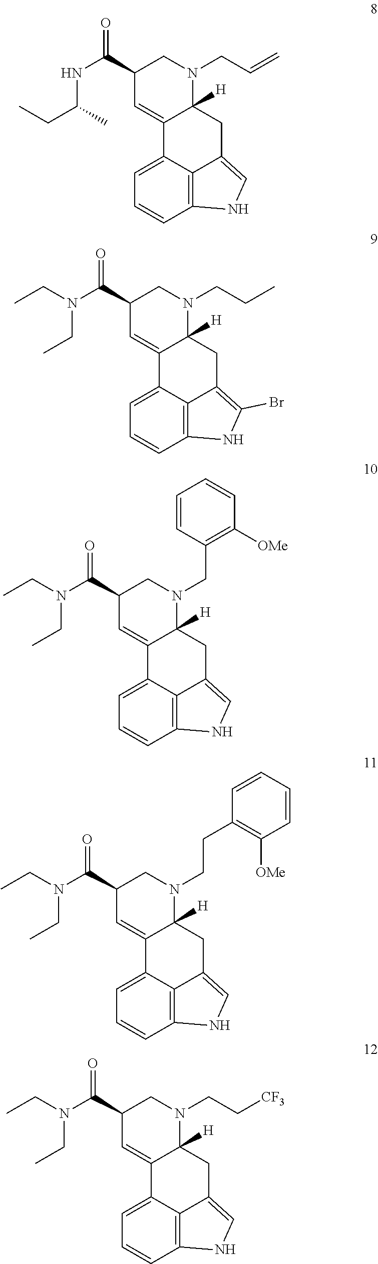

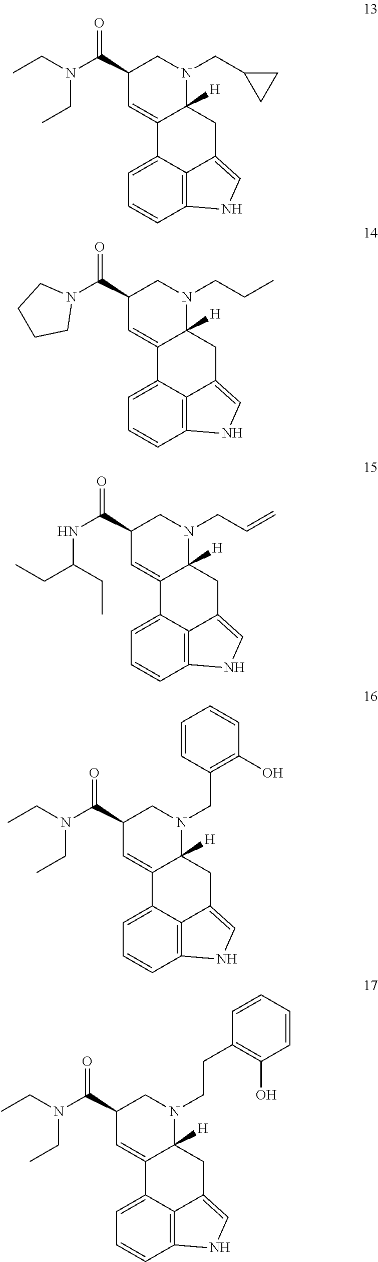

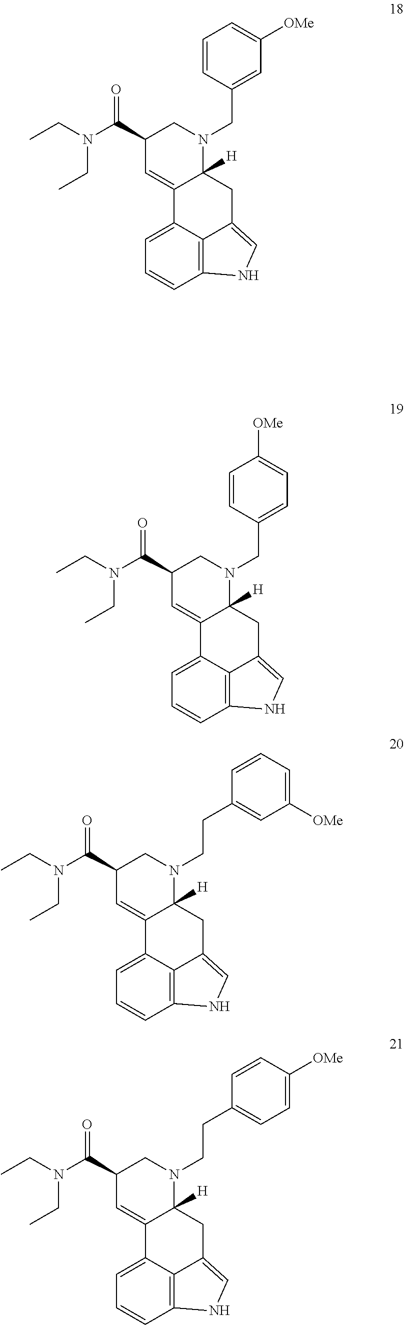

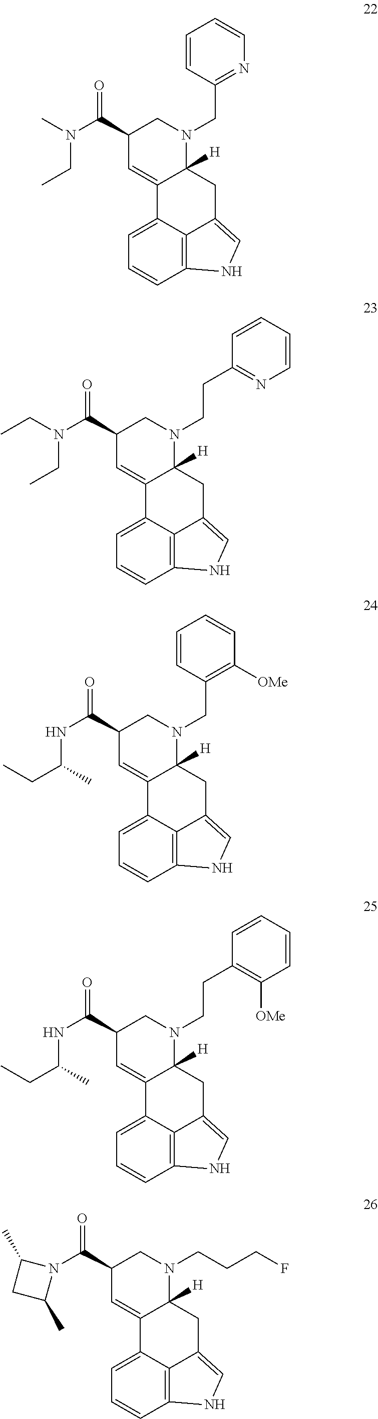

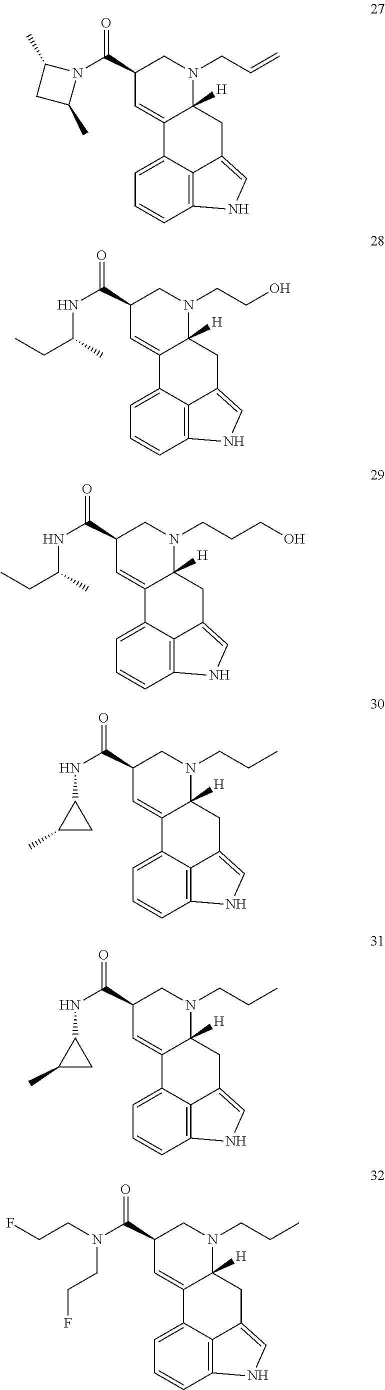

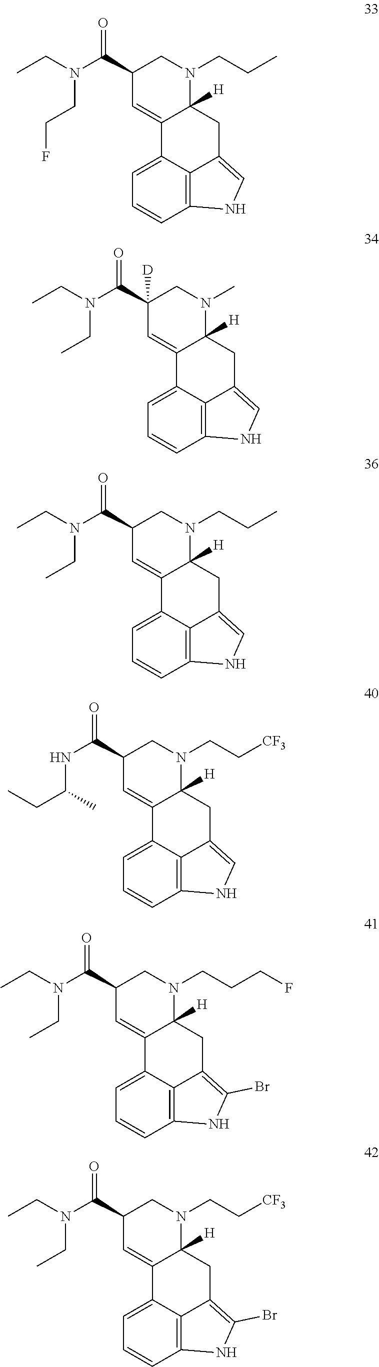

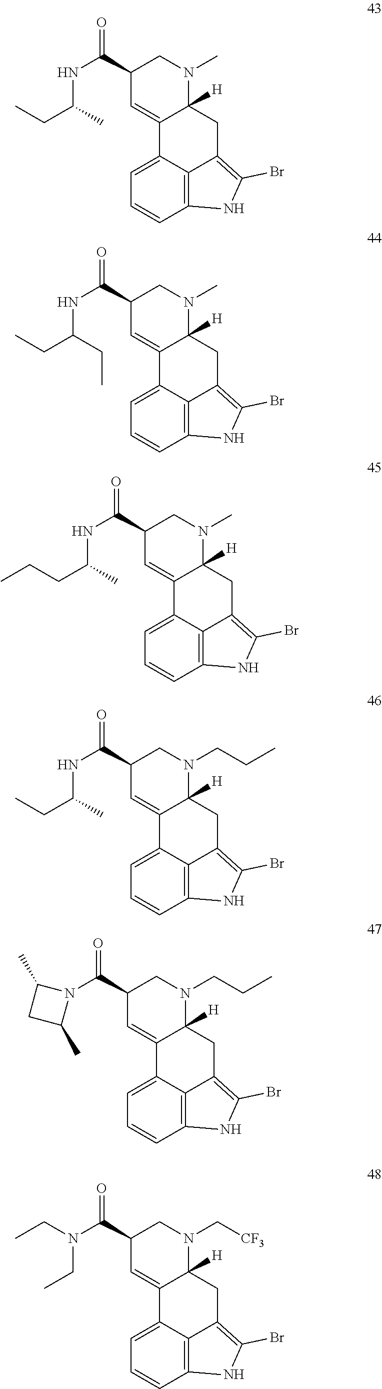

or a pharmaceutically acceptable salt thereof.

The compounds of the present disclosure are found to be agonists of 5-HT2A receptors. Moreover, compounds of the present disclosure, especially those compounds with substituents at the 2-position of the indole ring are non-hallucinogenic or less hallucinogenic agonists of 5-HT2A receptors than other agonists of 5-HT2A receptors. For example, compounds wherein the 2-substituent is halo or alkyl, such as Me, Et, —CH₂F, CHF₂, or —CF₃ are non-hallucinogenic or less hallucinogenic. In some embodiments, this reduced hallucinogenic effect can be demonstrated by an attenuated maximal response in the mouse head twitch assay compared to hallucinogenic 5-HT2A receptor agonists.

In other embodiments provided are methods and compositions for treating migraine or cluster headache by administering a therapeutically effective amount of a compound disclosed herein to a patient in need thereof.

In embodiments, methods include treating a mood disorder, e.g., a depressive disorder, by administering to a patient in need thereof a pharmaceutical composition including about 0.001 mg to about 20 mg of a compound disclosed herein. In embodiments, doses may be, e.g., in the range of about 0.001 to 20 mg, 0.001 to 10 mg, 0.001 to 5 mg, 0.001 to 2 mg, 0.001 to 1 mg, 0.001 to 0.5 mg, 0.001 to 0.25 mg, 0.001 to 0.15 mg, 0.001 to 0.1 mg, 0.001 to 0.075 mg, 0.001 to 0.05 mg, 0.001 to 0.025 mg, 0.001 to 0.015 mg, 0.001 to 0.01 mg, 0.01 to 5 mg, 0.01 to 2 mg, 0.01 to 1 mg, 0.01 to 0.5 mg, 0.01 to 0.25 mg, 0.01 to 0.15 mg, 0.01 to 0.1 mg, 0.01 to 0.075 mg, 0.01 to 0.05 mg, 0.01 to 0.025 mg, 0.01 to 0.015 mg, 0.025 to 2 mg, 0.025 to 1 mg, 0.025 to 0.5 mg, 0.025 to 0.25 mg, 0.025 to 0.15 mg, 0.025 to 0.1 mg, 0.025 to 0.075 mg, 0.025 to 0.05 mg, 0.05 to 2 mg, 0.05 to 1 mg, 0.05 to 0.5 mg, 0.05 to 0.25 mg, 0.05 to 0.15 mg, 0.05 to 0.1 mg, 0.05 to 0.075 mg, 0.1 to 2 mg, 0.1 to 1 mg, 0.1 to 0.5 mg, 0.1 to 0.25 mg, 0.1 to 0.15 mg, with doses of, e.g., about 0.001 mg, 0.0025 mg, 0.005 mg, 0.0075 mg, 0.01 mg, 0.015 mg, 0.02 mg, 0.025 mg, 0.03 mg, 0.04 mg, 0.05 mg, 0.075 mg, 0.1 mg, 0.125 mg, 0.15 mg, 0.175 mg, 0.2 mg, 0.25 mg, 0.3 mg, 0.4 mg, 0.5 mg, 0.75 mg, 1 mg, 1.5 mg, 2 mg, 2.5 mg, 3 mg, 4 mg, 5 mg, 7.5 mg, 10 mg, 15 mg, and 20 mg being examples.

In specific embodiments, dosages may include amounts of a compound disclosed herein in the range of about, e.g., 0.001 to 20 mg, 0.001 to 10 mg, 0.001 to 5 mg, 0.001 to 2 mg, 0.001 to 1 mg, 0.001 to 0.5 mg, 0.001 to 0.25 mg, 0.001 to 0.15 mg, 0.001 to 0.1 mg, 0.001 to 0.075 mg, 0.001 to 0.05 mg, 0.001 to 0.025 mg, 0.001 to 0.015 mg, 0.001 to 0.01 mg, 0.01 to 5 mg, 0.01 to 2 mg, 0.01 to 1 mg, 0.01 to 0.5 mg, 0.01 to 0.25 mg, 0.01 to 0.15 mg, 0.01 to 0.1 mg, 0.01 to 0.075 mg, 0.01 to 0.05 mg, 0.01 to 0.025 mg, 0.01 to 0.015 mg, 0.025 to 2 mg, 0.025 to 1 mg, 0.025 to 0.5 mg, 0.025 to 0.25 mg, 0.025 to 0.15 mg, 0.025 to 0.1 mg, 0.025 to 0.075 mg, 0.025 to 0.05 mg, 0.05 to 2 mg, 0.05 to 1 mg, 0.05 to 0.5 mg, 0.05 to 0.25 mg, 0.05 to 0.15 mg, 0.05 to 0.1 mg, 0.05 to 0.075 mg, 0.1 to 2 mg, 0.1 to 1 mg, 0.1 to 0.5 mg, 0.1 to 0.25 mg, 0.1 to 0.15 mg, with doses of 0.001 mg, 0.0025 mg, 0.005 mg, 0.0075 mg, 0.01 mg, 0.015 mg, 0.02 mg, 0.025 mg, 0.03 mg, 0.04 mg, 0.05 mg, 0.075 mg, 0.1 mg, 0.125 mg, 0.15 mg, 0.175 mg, 0.2 mg, 0.25 mg, 0.3 mg, 0.4 mg, 0.5 mg, 0.75 mg, 1 mg, 1.5 mg, 2 mg, 2.5 mg, 3 mg, 4 mg, 5 mg, 7.5 mg, 10 mg, 15 mg, and 20 mg being specific examples of doses.

Typically, dosages of a compound disclosed herein are administered once, twice, three or four times daily, every other day, every three days, twice weekly, once weekly, twice monthly, or once monthly to a patient in need thereof. In embodiments, the dosage is about, e.g., 0.001-20 mg/day, or 0.001-10 mg/day, or 0.001-1 mg/day, or 0.001-0.25 mg/day, for example 20 mg/day, 5 mg/day, 1 mg/day, 0.5 mg/day, 0.25 mg/day, 0.15 mg/day, 0.1 mg/day, 0.05 mg/day, 0.025 mg/day, 0.01 mg/day, 0.005 mg/day, or 0.001 mg/day. In embodiments, the foregoing example dose ranges may be delivered over intervals longer than one day, e.g., 0.001-20 mg/week.

In embodiments, pharmaceutical compositions for parenteral or inhalation, e.g., a spray or mist, administration of a compound disclosed herein having a concentration of about 0.001 mg/mL to about 100 mg/mL. In embodiments, the compositions include a compound disclosed herein, at a concentration of, e.g., about 0.05 mg/mL to about 100 mg/mL, about 0.05 mg/mL to about 50 mg/mL, about 0.05 mg/mL to about 25 mg/mL, about 0.05 mg/mL to about 10 mg/mL, about 0.05 mg/mL to about 5 mg/mL, about 0.005 mg/mL to about 1 mg/mL, about 0.005 mg/mL to about 0.25 mg/mL, about 0.005 mg/mL to about 0.05 mg/mL, about 0.005 mg/mL to about 0.025 mg/mL, about 0.001 mg/mL to about 0.05 mg/mL, about 0.001 mg/mL to about 0.025 mg/mL, about 0.001 mg/mL to about 0.01 mg/mL, or about 0.001 mg/mL to about 0.005 mg/mL.

In embodiments, the composition of a compound disclosed herein at a concentration of, e.g., about 0.05 mg/mL to about 100 mg/mL, about 0.05 mg/mL to about 50 mg/mL, about 0.05 mg/mL to about 25 mg/mL, about 0.05 mg/mL to about 10 mg/mL, about 0.05 mg/mL to about 5 mg/mL, about 0.005 mg/mL to about 1 mg/mL, about 0.005 mg/mL to about 0.25 mg/mL, about 0.005 mg/mL to about 0.05 mg/mL, about 0.005 mg/mL to about 0.025 mg/mL, about 0.001 mg/mL to about 0.05 mg/mL, about 0.001 mg/mL to about 0.025 mg/mL, about 0.001 mg/mL to about 0.01 mg/mL, or about 0.001 mg/mL to about 0.005 mg/mL. In embodiments, the pharmaceutical compositions are formulated as a total volume of about, e.g., 0.1 mL, 0.25 mL, 0.5 mL, 1 mL, 2 mL, 5 mL, 10 mL, 20 mL, 25 mL, 50 mL, 100 mL, 200 mL, 250 mL, or 500 mL.

Typically, dosages may be administered to a subject once, twice, three times or four times daily, every other day, every three days, twice weekly, once weekly, twice monthly, once monthly, every 2 months, every 3 months, every 4 months, every 6 months, or every 12 months. In embodiments, a compound disclosed herein is administered to a subject once in the morning, or once in the evening. In embodiments, a compound disclosed herein is administered to a subject once in the morning, and once in the evening. In embodiments, a compound disclosed herein is administered to a subject three times a day (e.g., at breakfast, lunch, and dinner), at a dose, e.g., of 0.005 mg/administration (e.g., 0.015 mg/day).

In embodiments, an ergoline a compound disclosed herein is administered to a subject at a dose of 0.005 mg/day in one or more doses. In embodiments, a compound disclosed herein is administered to a subject at a dose of 0.01 mg/day in one or more doses. In embodiments, a compound disclosed herein is administered to a subject at a dose of 0.025 mg/day in one or more doses. In embodiments, a compound disclosed herein is administered to a subject at a dose of 0.05 mg/day in one or more doses. In embodiments, a compound disclosed herein is administered to a subject at a dose of 0.1 mg/day in one or more doses. In embodiments, a compound disclosed herein is administered to a subject at a dose of 0.15 mg/day in one or more doses. In embodiments, a compound disclosed herein is administered to a subject at a dose of 0.2 mg/day in one or more doses. In embodiments, a compound disclosed herein is administered to a subject at a dose of 0.25 mg/day in one or more doses. In embodiments, a compound disclosed herein is administered to a subject at a dose of 0.3 mg/day in one or more doses. In embodiments, a compound disclosed herein is administered to a subject at a dose of 0.4 mg/day in one or more doses. In embodiments, a compound disclosed herein is administered to a subject at a dose of 0.5 mg/day in one or more doses.

In embodiments, the dosage of a compound disclosed herein is 0.000025-0.25 mg/kg, 0.0001-0.1 mg/kg, 0.001-0.1 mg/kg, or 0.01-0.25 mg/kg once, twice, three times or four times daily. For example, in embodiments, the dosage is 0.000025 mg/kg, 0.00005 mg/kg, 0.0001 mg/kg, 0.0005 mg/kg, 0.001 mg/kg, 0.002 mg/kg, 0.003 mg/kg, 0.004 mg/kg, 0.005 mg/kg, 0.01 mg/kg, 0.05 mg/kg, once, twice, three times or four times daily. In embodiments, a subject is administered a total daily dose of 0.001 mg to 20 mg of a compound disclosed herein once, twice, three times, or four times daily. In embodiments, the total amount administered to a subject in 24-hour period is, e.g., 0.001 mg, 0.0025 mg, 0.005 mg, 0.0075 mg, 0.01 mg, 0.015 mg, 0.02 mg, 0.025 mg, 0.03 mg, 0.04 mg, 0.05 mg, 0.075 mg, 0.1 mg, 0.125 mg, 0.15 mg, 0.175 mg, 0.2 mg, 0.25 mg, 0.3 mg, 0.4 mg, 0.5 mg, 0.75 mg, 1 mg, 1.5 mg, 2 mg, 2.5 mg, 3 mg, 4 mg, 5 mg, 7.5 mg, 10 mg, 15 mg, or 20 mg. In embodiments, the subject may be started at a low dose and the dosage is escalated. In embodiments, the subject may be started at a high dose and the dosage is decreased.

In embodiments, a compound disclosed herein is administered to a patient under the supervision of a healthcare provider.

In embodiments, a compound disclosed herein is administered to a patient under the supervision of a healthcare provider at a clinic specializing in the delivery of psychoactive treatments.

In embodiments, a compound disclosed herein is administered to a patient under the supervision of a healthcare provider at a high dose intended to induce a psychedelic experience in the subject, e.g., 0.05 mg, 0.075 mg, 0.1 mg, 0.125 mg, 0.15 mg, 0.175 mg, 0.2 mg, 0.25 mg, 0.3 mg, 0.4 mg, 0.5 mg, 0.6 mg, 0.7 mg, 0.8 mg, 0.9 mg, or 1 mg.

In embodiments, the administration to a patient of a high dose under the supervision of a healthcare provider occurs periodically in order to maintain a therapeutic effect in the patient, e.g., once weekly, twice monthly, once monthly, every 2 months, every 3 months, every 4 months, every 6 months, or every 12 months.

In embodiments, a compound disclosed herein is administered by a patient on their own at home or otherwise away from the supervision of a healthcare provider.

In embodiments, a compound disclosed herein is administered by a patient on their own at home or otherwise away from the supervision of a healthcare provider at a low dose intended to be sub-perceptual or to induce threshold psychoactive effects, e.g., 0.001 mg, 0.0025 mg, 0.005 mg, 0.0075 mg, 0.01 mg, 0.015 mg, 0.02 mg, 0.025 mg, 0.03 mg, or 0.04 mg.

In embodiments, the administration by a patient of a low dose on their own occurs periodically in order to maintain a therapeutic effect in the patient, e.g., daily, every other day, every three days, twice weekly, once weekly, twice monthly, or once monthly.

The compounds of the present disclosure may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted above, a compound of this present disclosure may be administered orally, subcutaneously, topically, parenterally, by inhalation spray or rectally in dosage unit formulations containing conventional non-toxic pharmaceutically acceptable carriers, adjuvants, and vehicles. Parenteral administration may include subcutaneous injections, intravenous or intramuscular injections or infusion techniques.

Treatment can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result, for example a decrease in symptoms of a psychiatric disorder, is achieved. A treatment regimen can include a corrective phase, during which a dose sufficient to provide symptomatic relief is administered, and can be followed by a maintenance phase, during which a lower dose sufficient to prevent a return of symptoms is administered. A suitable maintenance dose is likely to be found in the lower parts of the dose ranges provided herein, but corrective and maintenance doses can readily be established for individual subjects by those of skill in the art without undue experimentation, based on the disclosure herein. Maintenance doses can be employed to maintain remission in subjects whose symptoms have been previously controlled by other means, including treatments employing other pharmacological agents.

### III. Pharmaceutical Compositions and Kits

Another aspect of the present disclosure provides pharmaceutical compositions comprising compounds as disclosed herein formulated together with a pharmaceutically acceptable carrier. In particular, the present disclosure provides pharmaceutical compositions comprising compounds as disclosed herein formulated together with one or more pharmaceutically acceptable carriers. These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions of this present disclosure may be used in the form of a pharmaceutical preparation, for example, in solid, semisolid, or liquid form, which contains one or more of the compounds of the present disclosure, as an active ingredient, in admixture with an organic or inorganic carrier or excipient suitable for external, enteral, or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills, and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills, and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous, or organic solvents, or mixtures thereof, and powders. Liquid dosage forms for oral administration include pharmaceutically acceptable emulsions, microemulsions, solutions, suspensions, syrups, and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax, or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required.

The ointments, pastes, creams, and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars, or sugar alcohols. Aerosols generally are prepared from isotonic solutions.

Pharmaceutical compositions of this present disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the present disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants

In another aspect, the present disclosure provides enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e.g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present disclosure.

Advantageously, the present disclosure also provides kits for use by a e.g., a consumer in need of treatment with a disclosed compound. Such kits include a suitable dosage form such as those described above and instructions describing the method of using such dosage form to treat a medical disorder, for example, a psychiatric disease or disorder. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

Also contemplated herein are methods and compositions that include a second active agent or administering a second active agent.

### EXEMPLIFICATION

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

At least some of the compounds identified as “Intermediates” herein are contemplated as compounds of the present disclosure.

### General Procedures

The compounds of the present disclosure may be prepared by techniques well known in organic synthesis and familiar to a practitioner ordinarily skilled in the art. For example, the compounds may be prepared by the chemical transformations described in the following examples. However, these may not be the only means by which to synthesize or obtain the desired compounds.

### Abbreviations

AcOH=acetic acid
DCM=dichloromethane
DMF=dimethyl formamide
TEA=triethylamine
T3P=propylphosphonic anhydride
mCPBA=meta-chloroperoxybenzoic acid
HFBA=heptafluorobutyric acid

### Example 1: Preparation of (6aR,9R)—N,N-diethyl-7-propyl-4,6,6a,7,8,9-hexahydroindolo[4,3-fg]quinoline-9-carboxamide (1)

### Reaction Scheme (Method 1):

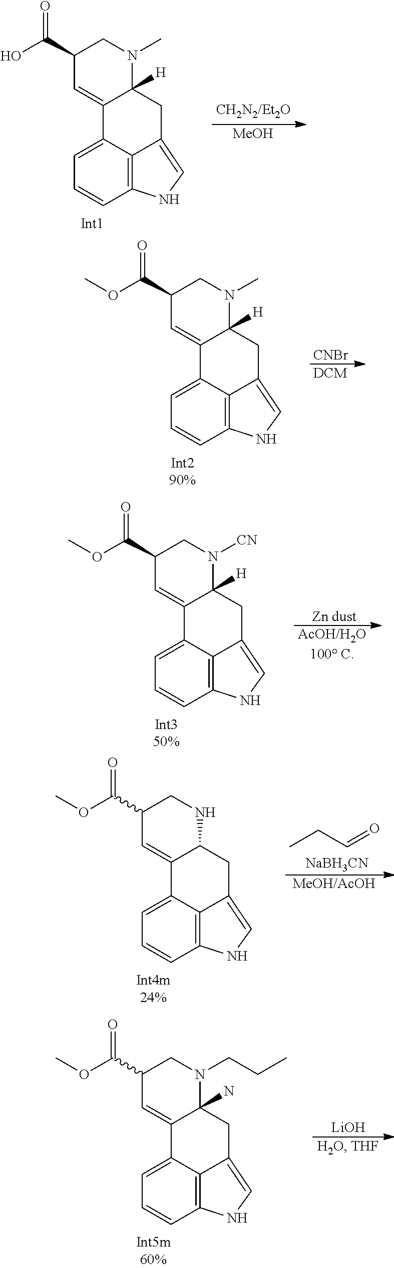

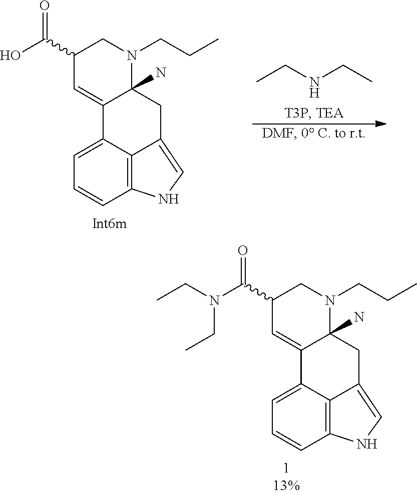

### Synthetic Protocols (Method 1):

To a suspension of (6aR,9R)-7-methyl-4,6,6a,7,8,9-hexahydroindolo[4,3-fg]quinoline-9-carboxylic acid (Int1, 2.01 g, 7.5 mmol) in absolute methanol (300 mL) was added a solution of diazomethane in diethyl ether (0.5 M, 75.0 mmol, 150 mL) under vigorous stirring. The resulting mixture was stirred until it became clear, then concentrated in vacuo and suspended in dichloromethane (100 mL). The solids were removed by filtration, the filter cake was washed with dichloromethane (3×30 mL), and the filtrate was concentrated in vacuo to provide methyl (6aR,9R)-7-methyl-4,6,6a,7,8,9-hexahydroindolo[4,3-fg]quinoline-9-carboxylate (Int2) as an off-white foam.

Yield: 1.92 g (90%).

LC-MS purity: 98% (ELSD).

LC-MS Rt (Sinergy Polar RP, 4.6 mm×150 mm, acetonitrile/water 20:80 to 100:0+0.1% FA in 10 min): 6.82 min.

LC-MS m/z: 283.2 (M+H)⁺.

Methyl (6aR,9R)-7-methyl-4,6,6a,7,8,9-hexahydroindolo[4,3-fg]quinoline-9-carboxylate (Int2, 564 mg, 2.0 mmol) was dissolved in dry dichloromethane (30 mL) and purged with argon. Cyanogen bromide (1.14 g, 10.72 mmol) was added in one portion and the obtained solution was stirred for 4.5 hours, at which point LC/MS showed full conversion. Silica gel (10 g) was added, and the resulting suspension was concentrated in vacuo. The product was purified by flash column chromatography (silica gel 60, 0.040-0.063 mm; eluent: cyclohexane/ethyl acetate 80:20 to 50:50) to give methyl (6aR,9R)-7-cyano-4,6,6a,7,8,9-hexahydroindolo[4,3-fg]quinoline-9-carboxylate (Int3) as a colorless foam.

Yield: 300 mg (50%).

LC-MS purity: 98% (ELSD).

LC-MS Rt (Sinergy Polar RP, 4.6 mm×150 mm, acetonitrile/water 05:95 to 100:0+0.1% FA in 10 min): 8.63 min.

LC-MS m/z: 294.1 (M+H)⁺.

Methyl (6aR,9R)-7-cyano-4,6,6a,7,8,9-hexahydroindolo[4,3-fg]quinoline-9-carboxylate (Int3, 205 mg, 0.70 mmol) was dissolved in glacial acetic acid (5 mL) and zinc dust (600 mg) and water (0.5 mL) were added. The resulting mixture was purged with argon, heated to 100° C., and stirred for 3 hours, at which point LC/MS showed full consumption of starting material. The reaction was cooled to 0° C., partitioned between saturated sodium bicarbonate (100 mL) and dichloromethane (100 mL), and extracted with dichloromethane (2×50 mL). The combined organic extracts were dried over anhydrous magnesium sulfate and concentrated in vacuo. The residue was purified by flash column chromatography (silica gel 60, 0.040-0.063 mm; eluent: dichloromethane/methanol 95:5 to 90:10) to afford methyl (6aR)-4,6,6a,7,8,9-hexahydroindolo[4,3-fg]quinoline-9-carboxylate (Int4m) (mixture of diastereomers; epimers at position 9) as an off-white foam.

Yield: 51 mg (24%).

LC-MS purity: 85% (ELSD).

LC-MS Rt (Sinergy Polar RP, 4.6 mm×150 mm, acetonitrile/water 05:95 to 100:0+0.1% FA in 10 min): 2.87 min.

LC-MS m/z: 269.2 (M+H)⁺.

A solution of methyl (6aR)-4,6,6a,7,8,9-hexahydroindolo[4,3-fg]quinoline-9-carboxylate (Int4m, 75 mg, 0.280 mmol; mixture of epimers at position 9) and propanal (88 μL, 1.40 mmol) in methanol (10 mL) was purged with argon and cooled to 0° C. Sodium cyanoborohydride (88.0 mg, 1.40 mmol) was added, the mixture was stirred for 5 minutes, and then acetic acid (300 μL) was introduced. After stirring at 0° C. for 1 hour, the solvent was evaporated, the residue was partitioned between dichloromethane (100 mL) and saturated sodium bicarbonate (100 mL), and the aqueous phase was extracted with ethyl acetate (3×50 mL). The combined organic phases were dried over sodium sulfate and concentrated in vacuo. The residue was purified by flash column chromatography (silica gel 60, 0.040-0.063 mm; eluent: dichloromethane/methanol 99:1 to 98:2) to afford methyl (6aR)-7-propyl-4,6,6a,7,8,9-hexahydroindolo[4,3-fg]quinoline-9-carboxylate (Int5m) (mixture of diastereomers; epimers at position 9) as an off-white foam.

Yield: 58 mg (67%).

LC-MS purity: 99% (ELSD), 95% (UV₃₁₀).

LC-MS Rt (Sinergy Polar RP, 4.6 mm×150 mm, acetonitrile/water 05:95 to 100:0+0.1% FA in 10 min): 2.95 min.

LC-MS m/z: 311.2 (M+H)⁺.

Methyl (6aR)-7-propyl-4,6,6a,7,8,9-hexahydroindolo[4,3-fg]quinoline-9-carboxylate (Int5m, 58.7 mg, 0.189 mmol; mixture of epimers at position 9) was dissolved in freshly distilled tetrahydrofuran (10 mL) and water (1 mL) and purged with argon. Lithium hydroxide (12.46 mg, 0.297 mmol) in water (500 μL) was added and resulting mixture was stirred overnight, at which point LC/MS showed full conversion. The reaction mixture was neutralized with ice-cold methanesulfonic acid (29.2 mg, 0.297 mmol) in water (1 mL), concentrated in vacuo, and the obtained off-white residue (Int6m) (mixture of diastereomers; epimers at position 9) was used in the next step without further purification.

Yield: 58 mg (crude).

LC-MS purity: 100% (ELSD), 95% (UV₃₁₀).

LC-MS Rt (Sinergy Polar RP, 4.6 mm×150 mm, acetonitrile/water 05:95 to 100:0+0.1% FA in 10 min): 7.08 min; 7.30 min.

LC-MS m/z: 297.2 (M+H)⁺.

Crude (6aR)-7-propyl-4,6,6a,7,8,9-hexahydroindolo[4,3-fg]quinoline-9-carboxylic acid (Int6m, 55 mg; mixture of epimers at position 9) was dissolved in dry N,N-dimethylformamide (3 mL) and the solution was purged with argon and cooled to 0° C. Triethylamine (106 μL, 0.760 mmol), diethylamine (60 μL, 0.570 mmol), and propanephosphonic acid anhydride (T3P, 332 μL, 0.570 mmol, 50% in DMF) were added and the resulting mixture was stirred for 1 hour. Ice-cold water (50 mL) was added, followed by ice-cold 1% ammonium hydroxide solution (5 mL), and the aqueous phase was extracted with dichloromethane (5×30 mL). The combined organic layers were dried over anhydrous sodium sulfate and concentrated in vacuo. The crude residue was purified by preparative LC/MS (Sinergy Polar RP C18, 5 μm, 21.2 mm×150 mm, acetonitrile/water 5:95+0.1% acetic acid) to give the title compound as a solution in acetonitrile/water. Freeze drying provided 10 mg of (6aR,9R)-9-(diethylcarbamoyl)-7-propyl-4,6,6a,7,8,9-hexahydroindolo[4,3-fg]quinolin-7-ium acetate (1) as a beige powder.

Yield: 10 mg (13% over two steps).

¹H NMR spectrum (of acetate salt; acetate peak obscured by solvent peak) (300 MHz, CD₃CN, δ_{H}): 9.00 (s, 1H), 7.22 (dd, J=6.8, 1.9 Hz, 1H), 7.14-7.05 (m, 2H), 6.98-6.84 (m, 2H), 6.30 (s, 1H), 3.78-3.68 (m, 1H), 3.56-3.30 (m, 6H), 3.13 (dd, J=10.9, 4.5 Hz, 1H), 2.93-2.82 (m, 1H), 2.69-2.42 (m, 4H), 1.67-1.43 (m, 2H), 1.21 (t, J=7.1 Hz, 3H), 1.11 (t, J=7.1 Hz, 3H), 0.94 (t, J=7.3 Hz, 3H).

LC-MS purity: 97% (ELSD), 96% (UV₃₁₀).

LC-MS Rt (Sinergy Polar RP, 4.6 mm×150 mm, acetonitrile/water 05:95 to 100:0+0.1% FA in 10 min): 8.20 min.

LC-MS m/z: 352.2 (M+H)⁺.

### Reaction Scheme (Method 2):

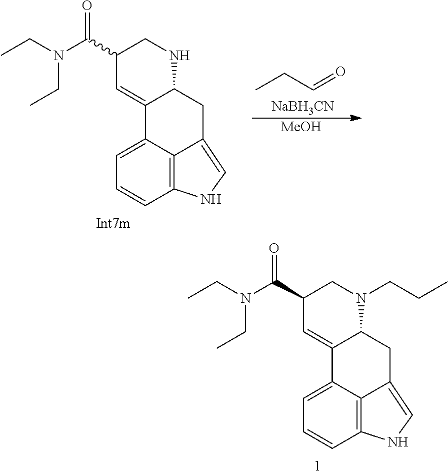

### Synthetic Protocols (Method 2):

A solution of (6aR)—N,N-diethyl-4,6,6a,7,8,9-hexahydroindolo[4,3-fg]quinoline-9-carboxamide (Int7m, 45.0 mg, 0.145 mmol; mixture of epimers at position 9) and propanal (52 μL, 0.72 mmol) in methanol (10 mL) was purged with argon and cooled to 0° C. Sodium cyanoborohydride (46.0 mg, 0.72 mmol) was added, the mixture was stirred for 5 minutes, and then acetic acid (160 μL) was added. The reaction mixture was stirred at 0° C. for 1 hour, the solvent was removed in vacuo, and the residue was partitioned between dichloromethane and a 1% solution of ammonium hydroxide. The aqueous phase was extracted with dichloromethane (3×50 mL) and the combined organic phases were dried over anhydrous sodium sulfate and evaporated. The residue was purified by flash column chromatography (silica gel 60, 0.040-0.063 mm; eluent: dichloromethane/methanol 99:1 to 98:2) to afford (6aR,9R)—N,N-diethyl-7-propyl-4,6,6a,7,8,9-hexahydroindolo[4,3-fg]quinoline-9-carboxamide (1) as colorless oil.

Yield: 6 mg (11%).

¹H NMR spectrum (of acetate salt; acetate peak obscured by solvent peak) (300 MHz, CD₃CN, δ_{H}): 9.00 (s, 1H), 7.22 (dd, J=6.8, 1.9 Hz, 1H), 7.14-7.05 (m, 2H), 6.98-6.84 (m, 2H), 6.30 (s, 1H), 3.78-3.68 (m, 1H), 3.56-3.30 (m, 6H), 3.13 (dd, J=10.9, 4.5 Hz, 1H), 2.93-2.82 (m, 1H), 2.69-2.42 (m, 4H), 1.67-1.43 (m, 2H), 1.21 (t, J=7.1 Hz, 3H), 1.11 (t, J=7.1 Hz, 3H), 0.94 (t, J=7.3 Hz, 3H).

LC-MS purity: 96% (ELSD), 93% (UV₃₁₀).

LC-MS Rt (Sinergy Polar RP, 4.6 mm×150 mm, acetonitrile/water 20:80 to 100:0+0.1% FA in 10 min): 5.66 min.

LC-MS m/z: 352.2 (M+H)⁺.

### Example 2: Preparation of (6aR,9R)—N,N-diethyl-7-(3-fluoropropyl)-4,6,6a,7,8,9-hexahydroindolo[4,3-fg]quinoline-9-carboxamide (2) and (6aR,9S)—N,N-diethyl-7-(3-fluoropropyl)-4,6,6a,7,8,9-hexahydroindolo[4,3-fg]quinoline-9-carboxamide (2a) Reaction Scheme (Method 1)

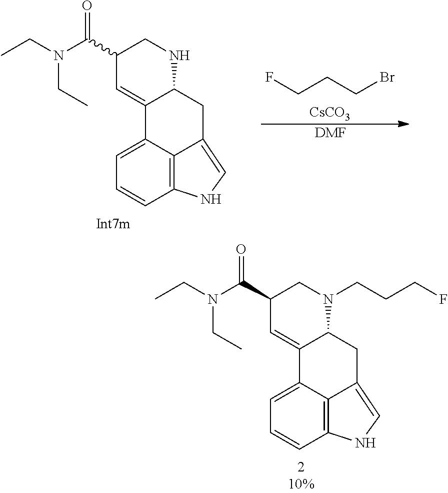

### Synthetic Protocols (Method 1):

A solution of (6aR)—N,N-diethyl-4,6,6a,7,8,9-hexahydroindolo[4,3-fg]quinoline-9-carboxamide (Int7m, 60.0 mg, 0.194 mmol; mixture of epimers at position 9), cesium carbonate (139 mg, 0.426 mmol), and 1-bromo-3-fluoropropane (30.2 mg, 0.214 mmol) in N,N-dimethylformamide (1 mL) was purged with argon and stirred for 96 hours at room temperature. The reaction mixture was diluted with water (50 mL), extracted with dichloromethane (3×50 mL), and the combined organic phases were dried over magnesium sulfate and concentrated in vacuo. The obtained crude product was purified by silica gel chromatography (silica gel 60, 0.040-0.063 mm; eluent: dichloromethane/methanol 98:2) to afford (6aR,9R)—N,N-diethyl-7-(3-fluoropropyl)-4,6,6a,7,8,9-hexahydroindolo[4,3-fg]quinoline-9-carboxamide (2, faster moving fluorescent band) as a colorless foam.

Yield: 6 mg (10%).

¹H NMR (300 MHz, CDCl₃, δ_{H}): 8.03 (s, 1H), 7.24-7.11 (m, 3H), 6.90 (s, 1H), 6.33 (s, 1H), 4.71-4.59 (m, 1H), 4.57-4.42 (m, 1H), 3.84 (s, 1H), 3.58-3.35 (m, 6H), 3.27-3.10 (m, 2H), 2.96 (t, J=13.2 Hz, 1H), 2.85-2.64 (m, 2H), 2.13-1.86 (m, J=23.6 Hz, 2H), 1.26 (t, J=7.0 Hz, 3H), 1.18 (t, J=7.1 Hz, 3H).

LC-MS purity: 90% (ELSD), 81% (UV, 310 nm).

LC-MS Rt (Sinergy Polar RP 4.6 mm×150 mm, acetonitrile/water 30:70 to 100:0+0.1% HFBA in 10 min): 5.68 min.

LC-MS m/z: 370.2 (M+H)⁺.

### Reaction Scheme (Method 2):

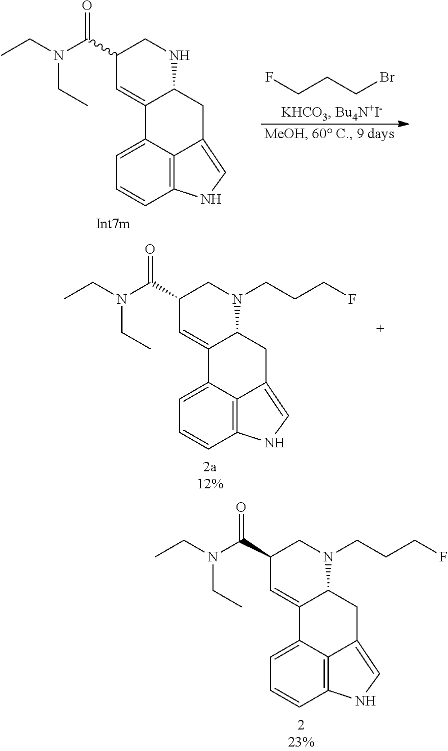

### Synthetic Protocols (Method 2):

To a stirred solution of (6aR)—N,N-diethyl-4,6,6a,7,8,9-hexahydroindolo[4,3-fg]quinoline-9-carboxamide (Int7m, 45 mg, 0.145 mmol; mixture of epimers at position 9) and potassium bicarbonate (30 mg, 0.29 mmol) in methanol (2 mL) was added a solution of 1-bromo-3-fluoropropane (50 mg, 0.348 mmol) in methanol (1 mL) dropwise under argon atmosphere. Tetrabutylammonium iodide (53.5 mg, 0.145 mmol) was then introduced in one portion and the reaction was heated to 60° C. and stirred for 9 days. After cooling to room temperature, the reaction mixture was diluted with dichloromethane (50 mL) and silica gel (10 g) was introduced. The obtained suspension was stripped of solvents in vacuo and subjected to silica gel chromatography (silica gel 60, 0.040-0.063 mm; eluent: dichloromethane/methanol/ammonia 98:2:0.1) to afford (6aR,9R)—N,N-diethyl-7-(3-fluoropropyl)-4,6,6a,7,8,9-hexahydroindolo[4,3-fg]quinoline-9-carboxamide (2, faster moving fluorescent band) as a colorless foam and (6aR,9S)—N,N-diethyl-7-(3-fluoropropyl)-4,6,6a,7,8,9-hexahydroindolo[4,3-fg]quinoline-9-carboxamide (2a, slower moving fluorescent band) as a dark brownish foam.

2:

Yield: 13.9 mg (23%).

¹H NMR spectrum (300 MHz, CD₃CN, δ_{H}): 8.99 (s, 1H); 7.22 (dd, J=J=6.9, 1.8, 1H); 7.14-7.03 (m, 2H); 6.95 (s, 1H); 6.30 (s, 1H); 4.75-4.58 (m, 1H); 4.57-4.41 (m, 1H); 3.79-3.65 (m, 1H); 3.57-3.29 (m, 6H); 3.18-3.01 (m, 2H); 2.71-2.45 (m, 3H); 2.02-1.82 (m, 2H); 1.20 (d, J=J=7.1, 3H); 1.11 (t, J=J=7.1, 3H).

LC-MS purity: 97% (ELSD), 100% (UV, 310 nm).

LC-MS Rt (Sinergy Polar RP 4.6 mm×150 mm, acetonitrile/water 30:70 to 100:0+0.1% HFBA in 10 min): 5.47 min.

LC-MS m/z: 370.2 (M+H)⁺.

2a:

Yield: 7.8 mg (12%).

¹H NMR spectrum (300 MHz, CD₃CN, δ_{H}): 8.98 (s, 1H); 7.20 (d, J=J=7.3, 1H); 7.12-6.98 (m, 2H); 6.91 (s, 1H); 6.24 (s, 1H); 4.68-4.59 (m, 1H); 4.51-4.43 (m, 1H); 3.76-3.66 (m, 1H); 3.55-3.27 (m, 6H); 3.12 (dd, J=14.6, 5.2, 1H); 3.07-2.75 (m, 5H); 1.92-1.77 (m, 2H); 1.23 (t, J=7.1, 3H); 1.06 (t, J=7.0, 3H).

LC-MS purity: 100% (ELSD), 96% (UV, 310 nm).

LC-MS Rt (Sinergy Polar RP 4.6 mm×150 mm, acetonitrile/water 30:70 to 100:0+0.1% HFBA in 10 min): 5.71 min.

LC-MS m/z: 370.2 (M+H)⁺.

### Example 3: Preparation of (6aR,9R)—N—((R)-sec-butyl)-7-propyl-4,6,6a,7,8,9-hexahydroindolo[4,3-fg]quinoline-9-carboxamide (3) and (6aR,9S)—N—((R)-sec-butyl)-7-propyl-4,6,6a,7,8,9-hexahydroindolo[4,3-fg]quinoline-9-carboxamide (3a)

### Reaction Scheme:

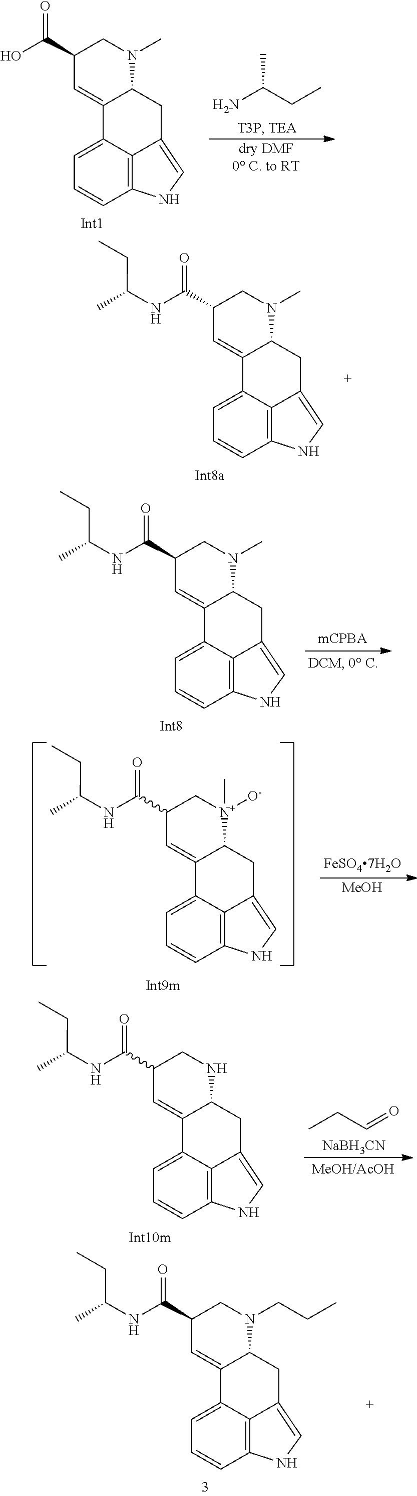

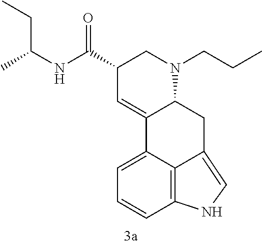 [SMILES: CCCN1C[C@@H](C(=O)N[C@H](C)CC)C=C2c3cccc4[nH]cc(c34)C[C@H]21]

### Synthetic Protocols:

A solution of (6aR,9R)-7-methyl-4,6,6a,7,8,9-hexahydroindolo[4,3-fg]quinoline-9-carboxylic acid (Int1, 805 mg, 3.00 mmol), triethylamine (1.69 mL, 12.0 mmol), and (R)-butan-2-amine (329 mg, 4.50 mmol) in dry N,N-dimethylformamide (30 mL) was cooled to 0° C. and propanephosphonic acid anhydride (T3P, 5.24 mL, 9.00 mmol, 50% solution in DMF) was added dropwise over 5 minutes. The resulting mixture was stirred for 1 hour at 0° C. and then diluted with water (200 mL) and washed with ethyl acetate (3×150 mL). The organic phases were discarded (product is in form of salt in aqueous phase) and the aqueous phase was basified to pH=12 by addition of a 30% solution of ammonium hydroxide. The mixture was then extracted with dichloromethane (3×200 mL) and the combined organic phases were dried over anhydrous sodium sulfate and concentrated in vacuo. The residue was purified by flash column chromatography (silica gel 60, 0.040-0.063 mm; eluent: dichloromethane/methanol 99:1 to 90:10) to afford (6aR,9S)—N—((R)-sec-butyl)-7-methyl-4,6,6a,7,8,9-hexahydroindolo[4,3-fg]quinoline-9-carboxamide (Int8a, faster, less polar isomer) as a dark-brown solid and (6aR,9R)—N—((R)-sec-butyl)-7-methyl-4,6,6a,7,8,9-hexahydroindolo[4,3-fg]quinoline-9-carboxamide (Int8, slower, more polar isomer) as a colorless solid.

### Int8a:

Yield: 0.28 g (28%).

¹H NMR spectrum (300 MHz, CDCl₃, δ_{H}): 8.28 (s, 1H); 8.01 (s, 1H); 7.25-7.10 (m, 3H); 6.93 (s, 1H); 6.60 (d, J=5.7, 1H); 3.93-3.76 (m, 1H); 3.59 (dd, J=14.5, 5.4, 1H); 3.27-3.04 (m, 2H); 2.78-2.61 (m, 2H); 2.58 (s, 3H); 1.56-1.33 (m, 2H); 1.01 (d, J=6.6, 3H); 0.91 (d, J=7.4, 3H).

LC-MS purity: 100% (ELSD).

LC-MS Rt (Sinergy Polar RP, 4.6 mm×150 mm, acetonitrile/water 30:70 to 100:0+0.1% HFBA in 10 min): 6.21 min.

LC-MS m/z: 324.2 (M+H)⁺.

### Int8:

Yield: 0.47 g (48%).

¹H NMR spectrum (300 MHz, CDCl₃, δ_{H}): 8.34 (s, 1H); 7.24-7.09 (m, 3H); 6.90 (s, 1H); 6.62 (d, J=8.0, 1H); 6.42 (dd, J=3.7, 1.9, 1H); 3.93 (dt, J=14.8, 6.7, 1H); 3.54-3.48 (m, 1H); 3.44-3.34 (m, 2H); 3.10 (dd, J=11.5, 4.7, 1H); 2.83-2.68 (m, 2H); 2.60 (s, 3H); 1.56-1.37 (m, 2H); 1.13 (d, J=6.6, 3H); 0.90 (t, J=7.4, 3H).

LC-MS purity: 100% (ELSD).

LC-MS Rt (Sinergy Polar RP, 4.6 mm×150 mm, acetonitrile/water 30:70 to 100:0+0.1% HFBA in 10 min): 5.29 min.

LC-MS m/z: 324.2 (M+H)⁺.

A solution of 3-chloroperbenzoic acid (361 mg, 1.61 mmol) in dry dichloromethane (20 mL) was added dropwise to a solution of (6aR)—N—((R)-sec-butyl)-7-methyl-4,6,6a,7,8,9-hexahydroindolo[4,3-fg]quinoline-9-carboxamide (Int8m, 526 mg, 1.63 mmol; mixture of epimers at position 9) in dry dichloromethane (40 mL) at 0° C. and the resulting mixture was stirred for 1 hour. A 10% solution of sodium hydroxide (50 mL) was then added, the phases were separated, and the aqueous phase was extracted with a 10% solution of isopropanol in dichloromethane (3×100 mL). The combined organic phases were dried and evaporated in vacuo to afford (6aR)-9-(((R)-sec-butyl)carbamoyl)-7-methyl-4,6,6a,7,8,9-hexahydroindolo[4,3-fg]quinoline 7-oxide (Int9m) (mixture of diastereomers; epimers at position 9) as a dark-brownish solid, which was used in the next step without further purification.

Yield: 0.52 g (100%).

LC-MS purity: 100% (UV, 310 nm).

LC-MS Rt (Sinergy Polar RP, 4.6 mm×150 mm, acetonitrile/water 50:50 to 100:0+0.1% FA in 10 min): 5.13 min.

LC-MS m/z: 340.2 (M+H)⁺.

Crude (6aR)-9-(((R)-sec-butyl)carbamoyl)-7-methyl-4,6,6a,7,8,9-hexahydroindolo[4,3-fg]quinoline 7-oxide (Int9m, 520 mg; mixture of epimers at position 9) was dissolved in methanol (20 mL), cooled to 0° C., and purged with argon. Iron (II) sulfate heptahydrate (Fe₂SO₄.7H₂O, 895 mg, 3.22 mmol) was then added in portions to this solution and the mixture was stirred for 3 hours at 0° C. The solvent was then removed in vacuo and the residue was partitioned between dichloromethane (150 mL) and a solution of EDTA (10 g) and 30% ammonium hydroxide (10 mL) in water (100 mL). The aqueous phase was further extracted with dichloromethane (3×100 mL) and the combined organic phases were dried and evaporated. The residue was purified by flash column chromatography (silica gel 60, 0.040-0.063 mm; eluent: dichloromethane/methanol 98:2 to 85:15) to afford (6aR)—N—((R)-sec-butyl)-4,6,6a,7,8,9-hexahydroindolo[4,3-fg]quinoline-9-carboxamide (Int10m) (mixture of diastereomers; epimers at position 9) as a dark-brownish solid.

Yield: 0.121 g (24% over 2 steps).

LC-MS purity: 100% (ELSD).

LC-MS Rt (Sinergy Polar RP, 4.6 mm×150 mm, acetonitrile/water 50:50 to 100:0+0.1% FA in 10 min): 4.78 min (diastereomer 1); 5.15 min (diastereomer 2).

LC-MS m/z: 340.2 (M+H)⁺.

A solution of (6aR)—N—((R)-sec-butyl)-4,6,6a,7,8,9-hexahydroindolo[4,3-fg]quinoline-9-carboxamide (Int10m, 55.0 mg, 0.178 mmol; mixture of epimers at position 9) and propanal (0.064 mL, 0.89 mmol) in methanol (10 mL) was purged with argon and cooled to 0° C. Sodium cyanoborohydride (56.0 mg, 0.89 mmol) was added, the mixture was stirred for 5 minutes, and acetic acid (160 μL) was then added. The reaction was then stirred at 0° C. for 1 hour. The solvent was evaporated, and the residue was partitioned between dichloromethane (50 mL) and a 1% solution of ammonium hydroxide (150 mL). The aqueous phase was further extracted with dichloromethane (3×50 mL) and the combined organic phases were dried over anhydrous magnesium sulfate and evaporated. The resulting residue was purified by flash column chromatography (silica gel 60, 0.040-0.063 mm; eluent: dichloromethane/methanol 99:1 to 98:2) to afford (6aR,9S)—N—((R)-sec-butyl)-7-propyl-4,6,6a,7,8,9-hexahydroindolo[4,3-fg]quinoline-9-carboxamide (3a, faster, less polar diastereomer) as a colorless oil and (6aR,9R)—N—((R)-sec-butyl)-7-propyl-4,6,6a,7,8,9-hexahydroindolo[4,3-fg]quinoline-9-carboxamide (3, slower, more polar diastereomer) as a colorless oil.

3a:

Yield: 16 mg (26%).

¹H NMR spectrum (300 MHz, CDCl₃, δ_{H}): 8.15 (br s, 1H); 7.99 (d, J=5.9, 1H); 7.26-7.09 (m, 3H); 6.93 (s, 1H); 6.61 (d, J=5.9, 1H); 3.85 (dt, J=14.9, 6.6, 1H); 3.57 (dd, J=14.5, 4.8, 1H); 3.46-3.34 (m, 1H); 3.27 (d, J=11.7, 1H); 3.12 (br s, 1H); 2.92 (ddd, J=13.3, 9.3, 4.6, 1H); 2.76-2.56 (m, 2H); 2.56-2.42 (m, 1H); 1.81-1.52 (m, 2H); 1.52-1.38 (m, 2H); 1.01 (d, J=7.4, 3H); 1.00 (t, J=7.4, 3H); 0.91 (t, J=7.5, 3H).

LC-MS purity: 100% (ELSD).

LC-MS Rt (Sinergy Polar RP, 4.6 mm×150 mm, acetonitrile/water 30:70 to 100:0+0.1% HBFA in 10 min): 6.81 min.

LC-MS m/z: 352.1 (M+H)⁺.

3:

Yield: 17 mg (27%).

¹H NMR spectrum (300 MHz, CDCl₃, δ_{H}): 8.15 (br s, 1H); 7.38 (br s, 1H); 7.23-7.12 (m, 2H); 7.08 (dd, J=6.9, 0.9, 1H); 6.91 (s, 1H); 6.41 (dd, J=5.3, 1.6, 1H); 4.03-3.80 (m, 2H); 3.26 (dd, J=14.0, 4.8, 2H); 3.03 (dd, J=11.9, 4.0, 1H); 2.97-2.79 (m, 3H); 2.76-2.59 (m, 1H); 1.75-1.55 (m, 2H); 1.54-1.34 (m, 2H); 1.13 (d, J=6.6, 3H); 0.98 (t, J=7.3, 3H); 0.88 (t, J=7.4, 3H).

LC-MS purity: 100% (ELSD).

LC-MS Rt (Sinergy Polar RP, 4.6 mm×150 mm, acetonitrile/water 30:70 to 100:0+0.1% HBFA in 10 min): 6.00 min.

LC-MS m/z: 352.1 (M+H)⁺.

### Example 4: Preparation of ((2S,4S)-2,4-dimethylazetidin-1-yl)((6aR,9R)-7-propyl-4,6,6a,7,8,9-hexahydroindolo[4,3-fg]quinolin-9-yl)methanone (4)

### Reaction Scheme:

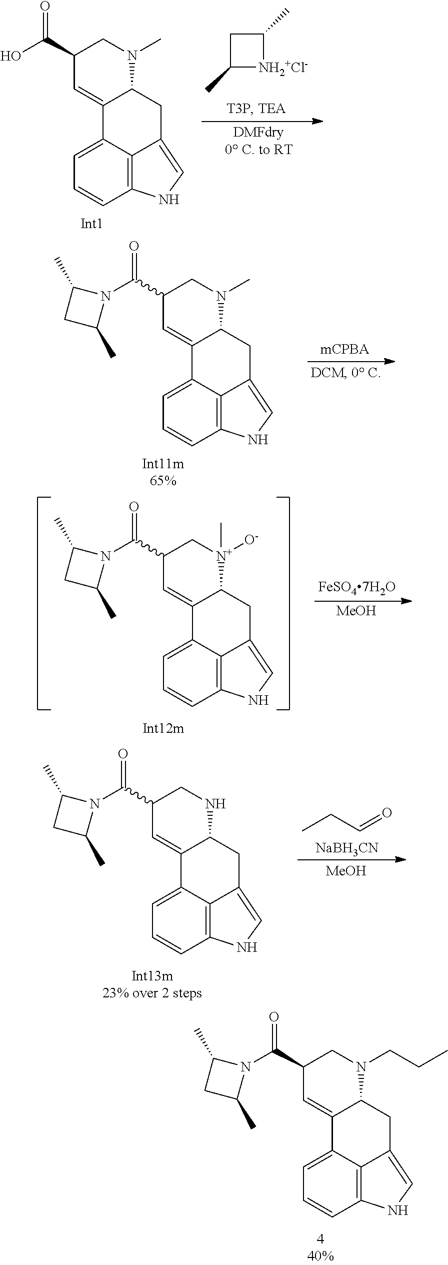

### Synthetic Protocols:

A solution of (6aR,9R)-7-methyl-4,6,6a,7,8,9-hexahydroindolo[4,3-fg]quinoline-9-carboxylic acid (Int1, 460 mg, 1.71 mmol), triethylamine (1.10 mL, 7.70 mmol), and (2S,4S)-2,4-dimethylazetidine hydrochloride (250 mg, 2.05 mmol) in dry N,N-dimethylformamide (10 mL) was cooled to 0° C. under an argon atmosphere. Propanephosphonic acid anhydride (T3P, 1.20 mL, 2.05 mmol, 50% solution in DMF) was then added dropwise over 5 minutes and the resulting mixture was stirred for 1 hour at 0° C. After the reaction was complete by LC/MS, it was quenched with ice-cold water (10 mL) and partitioned between 1M aqueous ammonium hydroxide solution (100 mL) and ethyl acetate (100 mL). The aqueous phase was further extracted with ethyl acetate (2×50 mL) and the combined organic phases were washed with 5% lithium chloride solution (4×50 mL), dried over anhydrous magnesium sulfate, and concentrated in vacuo. The residue was purified by flash column chromatography (silica gel 60, 0.040-0.063 mm; eluent: dichloromethane/methanol 100:0 to 98:2) to afford ((2S,4S)-2,4-dimethylazetidin-1-yl)((6aR, 9R)-7-methyl-4,6,6a,7,8,9-hexahydroindolo[4,3-fg]quinolin-9-yl)methanone (Int11, faster moving fluorescent band) as an off-white solid and ((2S,4S)-2,4-dimethylazetidin-1-yl)((6aR)-7-methyl-4,6,6a,7,8,9-hexahydroindolo[4,3-fg]quinolin-9-yl)methanone (Int11m; mixture of diastereomers; epimers at position 9) as a dark-brown solid.

Yield: 368 mg (65%), combined isomers.

¹H NMR spectrum (Int11, pure beta isomer) (300 MHz, CDCl₃, δ_{H}): 8.24 (br s, 1H); 7.24-7.09 (m, 3H); 6.88 (s, 1H); 6.36 (s, 1H); 4.52 (dt, J=7.5, 6.5, 2H); 3.60 (br s, 1H); 3.53 (dd, J=14.5, 5.4, 1H); 3.31-3.17 (m, 1H); 3.07 (dd, J=11.1, 4.9, 1H); 2.88 (t, J=10.9, 1H); 2.70 (t, J=12.0, 1H); 2.60 (s, 3H); 2.10-1.90 (m, 2H); 1.49 (t, J=6.3, 6H).

LC-MS purity: 100% (combined isomers, ELSD), 98% (combined isomers, UV₃₁₀).

LC-MS Rt (Sinergy Polar RP, 4.6 mm×150 mm, acetonitrile/water 20:80 to 100:0+0.1% FA in 10 min): 3.59 min (diastereomer 1); 3.95 min (diastereomer 2).

LC-MS m/z: 336.0 (M+H)⁺.

A solution of 3-chloroperbenzoic acid (189 mg, 1.10 mmol) in dry dichloromethane (5 mL) was added dropwise to a solution of ((2S,4S)-2,4-dimethylazetidin-1-yl)((6aR)-7-methyl-4,6,6a,7,8,9-hexahydroindolo[4,3-fg]quinolin-9-yl)methanone (Int11m, 368 mg, 1.10 mmol; mixture of epimers at position 9) in dry dichloromethane (30 mL) at 0° C. and the resulting mixture was stirred at 0° C. for 1 hour. A 10% solution of sodium hydroxide (100 mL) was then added to the reaction mixture and the aqueous phase was extracted with a 10% solution of isopropanol in dichloromethane (3×100 mL). The organic phases were combined, dried over anhydrous sodium sulfate, and concentrated in vacuo to afford (6aR)-9-((2S,4S)-2,4-dimethylazetidine-1-carbonyl)-4,6,6a,7,8,9-hexahydroindolo[4,3-fg]quinolone 7-oxide (Int12m) (mixture of diastereomers; epimers at position 9) as an off-white solid, which was used to the next step without further purification.

LC-MS purity: 100% (UV, 310 nm).

LC-MS Rt (Sinergy Polar RP, 4.6 mm×150 mm, acetonitrile/water 50:50 to 100:0+0.1% FA in 10 min): 1.92 min.

LC-MS m/z: 352.0 (M+H)⁺.

Crude (6aR)-9-((2S,4S)-2,4-dimethylazetidine-1-carbonyl)-4,6,6a,7,8,9-hexahydroindolo[4,3-fg]quinolone 7-oxide (Int12m; mixture of epimers at position 9) was dissolved in methanol (75 mL), cooled to 0° C., and purged with argon. Iron (II) sulfate heptahydrate (609 mg, 2.20 mmol) was then added and the mixture was stirred for 3 hours at 0° C. The solvent was removed in vacuo and the residue was partitioned between dichloromethane (150 mL) and a solution of EDTA (10 g) and 30% ammonium hydroxide (10 mL) in water (100 mL). The aqueous phase was further extracted with dichloromethane (3×100 mL) and the combined organic phases were dried over magnesium sulfate and concentrated in vacuo. The residue was purified by flash column chromatography (silica gel 60, 0.040-0.063 mm; eluent: dichloromethane/methanol 98:2 to 90:10) to afford ((2S,4S)-2,4-dimethylazetidin-1-yl)((6aR)-4,6,6a,7,8,9-hexahydroindolo[4,3-fg]quinolin-9-yl)methanone (Int13m) (mixture of diastereomers; epimers at position 9) as an off-white amorphous solid.

Yield: 81.5 mg (23% over 2 steps).

LC-MS purity: 88% (ELSD), 86% (UV₃₁₀).

LC-MS Rt (Sinergy Polar RP, 4.6 mm×150 mm, acetonitrile/water 05:95 to 100:0+0.1% FA in 10 min): 5.87 min.

LC-MS m/z: 322.2 (M+H)⁺.

A solution of ((2S,4S)-2,4-dimethylazetidin-1-yl)((6aR)-4,6,6a,7,8,9-hexahydroindolo[4,3-fg]quinolin-9-yl)methanone (Int13m, 81.5 mg, 0.242 mmol; mixture of epimers at position 9) and propanal (87 μL, 1.21 mmol) in methanol (10 mL) was purged with argon and cooled to 0° C. Sodium cyanoborohydride (76.0 mg, 1.21 mmol) was added, the mixture was stirred for 5 minutes, and then acetic acid (300 μL) was added. After stirring at 0° C. for 1 hour, the solvent was evaporated, and the residue was partitioned between dichloromethane and a 1% solution of ammonium hydroxide. The aqueous phase was extracted with dichloromethane (3×50 mL) and the combined organic phases were dried over sodium sulfate and concentrated in vacuo. The residue was purified by flash column chromatography (silica gel 60, 0.040-0.063 mm; eluent: dichloromethane/methanol 99:1 to 98:2) to afford ((2S,4S)-2,4-dimethylazetidin-1-yl)((6aR,9R)-7-propyl-4,6,6a,7,8,9-hexahydroindolo[4,3-fg]quinolin-9-yl)methanone (4, faster moving fluorescent band) as an off-white foam.

Yield: 35 mg (40%).

¹H NMR spectrum (300 MHz, CDCl₃, δ_{H}): 8.22 (br s, 1H); 7.24-7.06 (m, 3H); 6.89 (s, 1H); 6.34 (s, 1H); 4.66-4.45 (m, J=13.1, 6.2 Hz, 2H); 3.73-3.52 (m, J=18.2 Hz, 2H); 3.50-3.40 (m, 1H); 3.20 (dd, J=10.9, 4.4 Hz, 1H); 3.04-2.88 (m, J=12.8, 10.6 Hz, 2H); 2.86-2.64 (m, 2H); 2.14-1.89 (m, 2H); 1.74-1.56 (m, 2H); 1.49 (dd, 6H); 0.96 (t, J=7.3 Hz, 3H).

LC-MS purity: 99% (ELSD), 96% (UV, 310 nm).

LC-MS Rt (Sinergy Polar RP, 4.6 mm×150 mm, acetonitrile/water 30:70 to 100:0+0.1% HBFA in 10 min): 5.51 min.

LC-MS m/z: 364.1 (M+H)⁺.

### Example 5: Preparation of (6aR,9R)—N—((R)-sec-butyl)-7-ethyl-4,6,6a,7,8,9-hexahydroindolo[4,3-fg]quinoline-9-carboxamide (5) and (6aR,9S)—N—((R)-sec-butyl)-7-ethyl-4,6,6a,7,8,9-hexahydroindolo[4,3-fg]quinoline-9-carboxamide (5a)

### Reaction Scheme:

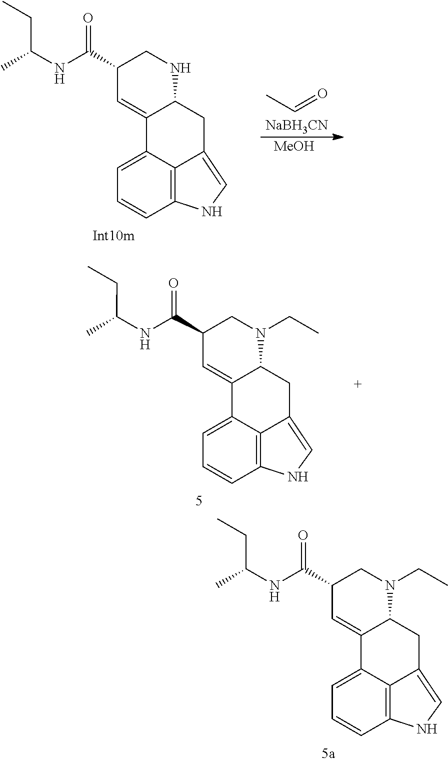

### Synthetic Protocols:

A solution of (6aR)—N—((R)-sec-butyl)-4,6,6a,7,8,9-hexahydroindolo[4,3-fg]quinoline-9-carboxamide (Int10m, 55.2 mg, 0.178 mmol; preparation is described in Example 3; mixture of epimers at position 9) and acetaldehyde (39.3 mg, 0.89 mmol) in methanol (10 mL) was purged with argon and cooled to 0° C. Sodium cyanoborohydride (56.1 mg, 0.89 mmol) was added, the mixture was stirred for 5 minutes, and acetic acid (200 μL) was then added. The reaction was stirred at 0° C. for 1 hour, the solvent was removed in vacuo, and the residue was partitioned between dichloromethane (50 mL) and a 1% solution of ammonium hydroxide (150 mL). The aqueous phase was further extracted with dichloromethane (3×50 mL) and the combined organic phases were dried over anhydrous sodium sulfate and evaporated. The resulting residue was purified by flash column chromatography (silica gel 60, 0.040-0.063 mm; eluent: dichloromethane/methanol 99:1 to 98:2) to afford (6aR,9S)—N—((R)-sec-butyl)-7-propyl-4,6,6a,7,8,9-hexahydroindolo[4,3-fg]quinoline-9-carboxamide (5a, faster moving, less polar diastereomer) as a colorless oil and (6aR,9R)—N—((R)-sec-butyl)-7-propyl-4,6,6a,7,8,9-hexahydroindolo[4,3-fg]quinoline-9-carboxamide (5, slower moving, more polar diastereomer) as a colorless solid. The separated isomers were each dissolved in absolute methanol (500 μL) and treated with an equimolar amount of 1M D-(−)-tartaric acid in absolute methanol. The obtained solutions were stripped of solvents in a flow of nitrogen and dried under high vacuum to yield (6aR,9S)-9-(((R)-sec-butyl)carbamoyl)-7-ethyl-4,6,6a,7,8,9-hexahydroindolo[4,3-fg]quinolin-7-ium (2S,3S)-3-carboxy-2,3-dihydroxypropanoate (5a tartrate) as a brown tinted amorphous solid and (6aR,9R)-9-(((R)-sec-butyl)carbamoyl)-7-ethyl-4,6,6a,7,8,9-hexahydroindolo[4,3-fg]quinolin-7-ium (2S,3S)-3-carboxy-2,3-dihydroxypropanoate (5 tartrate) as a colorless solid.

5a:

Yield (of freebase): 12.0 mg (20%).

¹H NMR spectrum (of freebase) (300 MHz, CDCl₃, δ_{H}): 8.50-7.90 (m, 2H); 7.26-7.21 (m, 1H); 7.20-7.06 (m, 2H); 6.92 (s, 1H); 6.59 (d, J=5.1 Hz, 1H); 3.93-3.75 (m, 1H); 3.67-3.38 (m, 2H); 3.33-3.01 (m, 3H); 2.91-2.41 (m, 3H); 1.52-1.38 (m, 2H); 1.24-1.12 (m, 3H); 1.09-0.97 (m, 3H); 0.91 (t, J=7.4 Hz, 3H).

¹H NMR spectrum (of tartrate) (300 MHz, MeOD, δ_{H}): 7.29 (dd, J=7.0, 1.6, 1H); 7.17 (t, J=6.9, 2H); 7.09 (s, 1H); 6.62 (d, J=5.7, 1H); 4.47 (s, 2H); 4.31 (dd, J=11.9, 5.3, 1H); 3.82 (dd, J=13.2, 6.4, 2H); 3.80-3.66 (m, 2H); 3.60-3.53 (m, 1H); 3.46-3.34 (m, 2H); 3.02 (t, J=13.0, 1H); 1.55-1.42 (m, 2H); 1.47 (t, J=7.3, 3H); 1.19 (d, J=7.0, 3H); 0.87 (t, J=7.4, 3H).

LC-MS purity (of freebase): 97% (ELSD), 91% (UV, 310 nm).

LC-MS purity (of tartrate): 99% (ELSD).

LC-MS Rt (of tartrate) (Sinergy Polar RP, 4.6 mm×150 mm, acetonitrile/water 30:70 to 100:0+0.1% HBFA in 10 min): 6.48 min.

LC-MS m/z: 338.1 (M+H)⁺.

5:

Yield (of freebase): 12.5 mg (21%).

¹H NMR spectrum (of tartrate) (300 MHz, MeOD, δ_{H}): 7.27 (dd, J=6.1, 2.5, 1H); 7.19-7.09 (m, 2H); 7.07 (s, 1H); 6.49 (s, 1H); 4.44 (s, 2H); 4.37-4.24 (m, 1H); 3.85 (dd, J=13.3, 6.6, 2H); 3.74-3.61 (m, 2H); 3.60-3.42 (m, 1H); 3.61-3.40 (m, 2H); 3.37-3.33 (m, 1H); 3.08 (t, J=12.9, 1H); 1.61-1.50 (m, 2H); 1.42 (t, J=7.2, 3H); 1.16 (d, J=7.0, 3H); 0.96 (t, J=7.4, 3H).

LC-MS purity (of tartrate): 91% (ELSD).

LC-MS Rt (of tartrate) (Sinergy Polar RP, 4.6 mm×150 mm, acetonitrile/water 30:70 to 100:0+0.1% HBFA in 10 min): 5.65 min.

LC-MS m/z: 338.1 (M+H)⁺.

### Example 6: Preparation of (6aR,9R)—N-(pentan-3-yl)-7-propyl-4,6,6a,7,8,9-hexahydroindolo[4,3-fg]quinoline-9-carboxamide (6)

### Reaction Scheme:

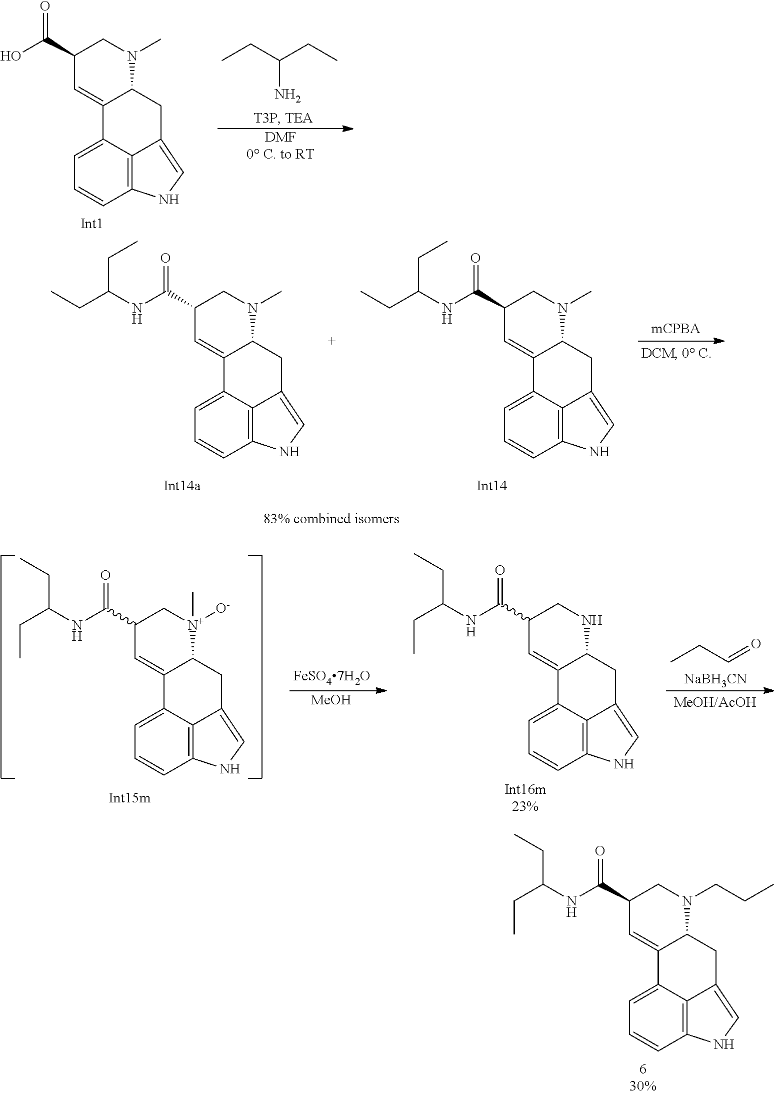

### Synthetic Protocols:

A solution of (6aR,9R)-7-methyl-4,6,6a,7,8,9-hexahydroindolo[4,3-fg]quinoline-9-carboxylic acid (Int1, 200 mg, 0.745 mmol), triethylamine (430 μL, 3.00 mmol), and 3-pentanamine (260 μL, 2.23 mmol) in dry N,N-dimethylformamide (10 mL) was cooled to 0° C. under argon atmosphere. Propanephosphonic acid anhydride (T3P®, 1.30 mL, 2.23 mmol, 50% solution in DMF) was added dropwise over 5 minutes, and the resulting mixture was stirred for 3 hours at 0° C. and then quenched with ice-cold water (10 mL). The reaction mixture was concentrated in vacuo along with silica gel (10 g) and the resulting solid was purified by flash column chromatography (silica gel 60, 0.040-0.063 mm; eluent: dichloromethane/methanol 100:0 to 98:2) to afford (6aR,9S)-7-methyl-N-(pentan-3-yl)-4,6,6a,7,8,9-hexahydroindolo[4,3-fg]quinoline-9-carboxamide (Int14a, faster moving, less polar diastereomer) as dark brown solid and (6aR,9R)-7-methyl-N-(pentan-3-yl)-4,6,6a,7,8,9-hexahydroindolo[4,3-fg]quinoline-9-carboxamide (Int14, slower moving, more polar diastereomer) as dark brown solid.

Yield: 208 mg (83%, combined isomers).

### Int14a:

¹H NMR spectrum (300 MHz, CD₃CN, δ_{H}): 9.03 (br s, 1H); 7.87 (br d, J=6.20 1H); 7.31-7.18 (m, 1H); 7.17-7.05 (m, 2H); 6.98 (s, 1H); 6.56 (d, J=6.2, 1H); 3.71-3.51 (m, 2H); 3.11 (d, J=11.7, 2H); 3.04-2.93 (m, 1H); 2.67 (dd, J=11.8, 3.8, 1H); 2.56 (dd, J=26.0, 1.6, 1H); 2.55 (s, 3H); 1.59-1.20 (m, 4H); 0.89 (t, J=7.4, 3H); 0.72 (t, J=7.4, 3H).

LC-MS purity: 100% (ELSD), 100% (UV₃₁₀).

LC-MS Rt (Sinergy Polar RP, 4.6 mm×150 mm, acetonitrile/water 30:70 to 100:0+0.1% HFBA in 10 min): 5.27 min.

LC-MS m/z: 338.2 (M+H)⁺.

### Int14:

¹H NMR spectrum (300 MHz, CD₃CN, δ_{H}): 9.03 (br s, 1H); 7.22 (dt, J=7.2, 3.6, 1H); 7.14-7.05 (m, 2H); 6.95 (s, 1H); 6.49 (br d, J=7.1, 1H); 6.40 (s, 1H); 3.67 (qd, J=8.5, 4.2, 1H); 3.48 (dd, J=14.6, 5.5, 1H); 3.37 (ddd, J=8.2, 5.6, 3.0, 1H); 3.24-3.14 (m, 1H); 3.07 (dd, J=11.2, 5.0, 1H); 2.69-2.44 (m, 4H); 2.53 (s, 3H); 1.62-1.46 (m, 2H); 1.46-1.31 (m, 2H); 0.96-0.84 (m, 6H).

LC-MS purity: 100% (ELSD), 100% (UV₃₁₀).

LC-MS Rt (Sinergy Polar RP, 4.6 mm×150 mm, acetonitrile/water 30:70 to 100:0+0.1% HFBA in 10 min): 4.95 min.

LC-MS m/z: 338.2 (M+H)⁺.

A solution of 3-chloroperbenzoic (77%, 138 mg, 800 μmol) acid in dry dichloromethane (5 mL) was added dropwise to a solution of (6aR)-7-methyl-N-(pentan-3-yl)-4,6,6a,7,8,9-hexahydroindolo[4,3-fg]quinoline-9-carboxamide (Int14m, 208 mg, 616 μmol; mixture of epimers at position 9) in dry dichloromethane (10 mL) at 0° C. and stirred for 1 hour under argon atmosphere. A 10% aqueous solution of sodium hydroxide (100 mL) was then added to the reaction mixture and the mixture was extracted with a 10% solution of isopropanol in dichloromethane (3×100 mL). The organic phases were combined, dried over anhydrous sodium sulfate, and concentrated in vacuo to afford (6aR)-7-methyl-9-(pentan-3-ylcarbamoyl)-4,6,6a,7,8,9-hexahydroindolo[4,3-fg]quinoline 7-oxide (Int15m) (mixture of diastereomers; epimers at position 9), which was used in the next step without further purification.

LC-MS purity: 100% (UV, 310 nm).

LC-MS Rt (Sinergy Polar RP, 4.6 mm×150 mm, acetonitrile/water 30:70 to 100:0+0.1% HBFA in 10 min): 5.09 min.

LC-MS m/z: 354.2 (M+H)⁺.

Crude (6aR)-7-methyl-9-(pentan-3-ylcarbamoyl)-4,6,6a,7,8,9-hexahydroindolo[4,3-fg]quinoline 7-oxide (Int15m, entire quantity obtained in above procedure; mixture of epimers at position 9) was dissolved in methanol (20 mL) and the solution was cooled to 0° C. under argon. Iron (II) sulfate heptahydrate (343 mg, 1.23 mmol) was then added and the resulting mixture stirred for 3 hours at 0° C. At this time, the solvent was removed in vacuo and the residue partitioned between dichloromethane (150 mL) and a solution of EDTA (10 g) and 30% ammonium hydroxide (10 mL) in water (100 mL). The aqueous phase was further extracted with dichloromethane (3×100 mL) and the combined organic phases were dried over magnesium sulfate and concentrated in vacuo. The crude residue was purified by flash column chromatography (silica gel 60, 0.040-0.063 mm; eluent: dichloromethane/methanol 98:2 to 90:10) to yield (6aR)—N-(pentan-3-yl)-4,6,6a,7,8,9-hexahydroindolo[4,3-fg]quinoline-9-carboxamide (Int16m) (mixture of diastereomers; epimers at position 9) as an amorphous beige solid.

Yield: 50.0 mg (25% over 2 steps from Int14m).

LC-MS purity: 95% (UV₃₁₀).

LC-MS Rt (Sinergy Polar RP, 4.6 mm×150 mm, acetonitrile/water 30:70 to 100:0+0.1% HBFA in 10 min): 4.77 min.

LC-MS m/z: 324.2 (M+H)⁺.

A solution of (6aR)—N-(pentan-3-yl)-4,6,6a,7,8,9-hexahydroindolo[4,3-fg]quinoline-9-carboxamide (Int16m, 50.0 mg, 0.154 mmol; mixture of epimers at position 9) and propanal (55 μL, 0.771 mmol) in methanol (10 mL) was purged with argon and cooled to 0° C. Sodium cyanoborohydride (48.0 mg, 0.77 mmol) was then added and the resulting mixture stirred for 5 minutes followed by addition of glacial acetic acid (100 uL). After stirring at 0° C. for 1 hour, solvents were removed in vacuo, the residue partitioned between dichloromethane (200 mL) and 1% ammonium hydroxide (150 mL), and the aqueous phase was further extracted with dichloromethane (3×50 mL). The combined organic phases were dried over sodium sulfate and concentrated in vacuo. Residue was purified by flash column chromatography (Silica gel 60, 0.040-0.063 mm; eluent: dichloromethane/methanol 99:1 to 98:2) to afford (6aR,9R)—N-(pentan-3-yl)-7-propyl-4,6,6a,7,8,9-hexahydroindolo[4,3-fg]quinoline-9-carboxamide (6, slower moving fluorescent band) as a colorless foam.

Yield: 20 mg (30%).

¹H NMR spectrum (300 MHz, CD₃CN, δ_{H}): 9.00 (br s, 1H); 7.21 (dd, J=7.6, 0.8, 1H); 7.14-7.00 (m, 2H); 6.99-6.84 (m, 2H); 6.37 (dd, J=3.8, 1.7, 1H); 3.75-3.61 (m, 1H); 3.60-3.50 (m, 1H); 3.37 (dd, J=14.4, 5.0, 1H); 3.23-3.13 (m, 1H); 3.07 (dd, J=11.4, 4.5, 1H); 2.79-2.58 (m, 4H); 1.65-1.45 (m, 4H); 1.44-1.24 (m, 2H); 0.98-0.81 (m, 9H).

LC-MS purity: 98% (ELSD), 97% (UV, 310 nm).

LC-MS Rt (Sinergy Polar RP, 4.6 mm×150 mm, acetonitrile/water 30:70 to 100:0+0.1% HBFA in 10 min): 5.72 min.

LC-MS m/z: 366.2 (M+H)⁺.

### Example 7: Preparation of (6aR,9R)—N—((R)-pentan-2-yl)-7-propyl-4,6,6a,7,8,9-hexahydroindolo[4,3-fg]quinoline-9-carboxamide (7)

### Reaction Scheme:

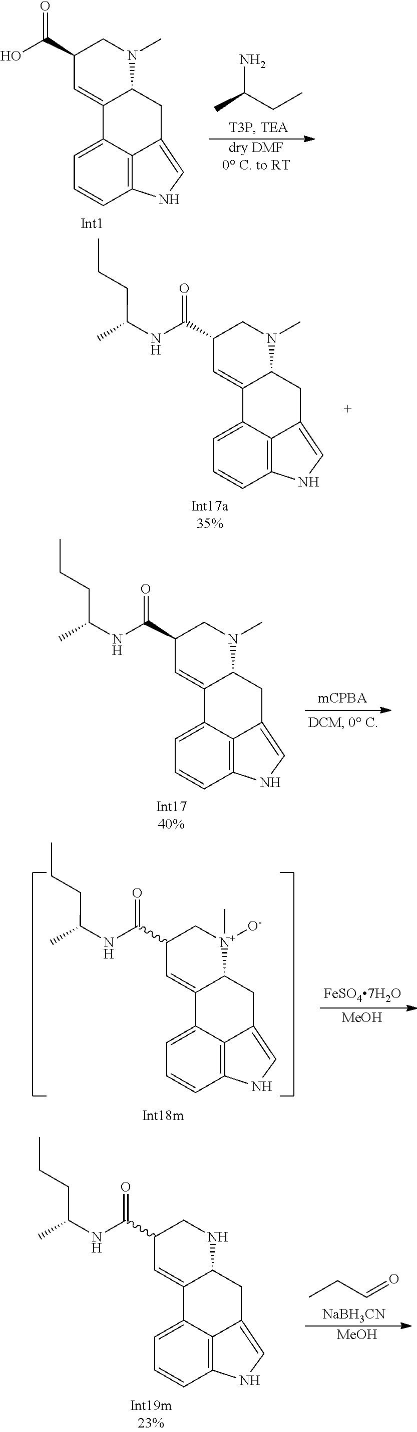

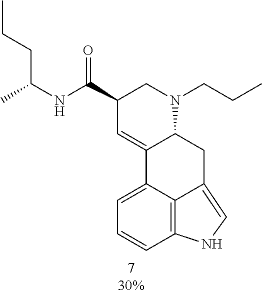 [SMILES: CCC[C@@H](C)NC(=O)[C@@H]1C=C2c3cccc4[nH]cc(c34)C[C@H]2N(CCC)C1]

### Synthetic Protocols:

A solution of (6aR,9R)-7-methyl-4,6,6a,7,8,9-hexahydroindolo[4,3-fg]quinoline-9-carboxylic acid (Int1, 200 mg, 0.745 mmol), triethylamine (430 μL, 3.00 mmol), and (R)-pentan-2-amine hydrochloride (250 μg, 1.50 mmol) in dry N,N-dimethylformamide (10 mL) was cooled to 0° C. under argon atmosphere. Propanephosphonic acid anhydride (T3P®, 875 μL, 1.50 mmol, 50% solution in DMF) was then added dropwise over 5 minutes. The resulting mixture was stirred for 3 hours at 0° C. and quenched with ice-cold water (1 mL). The resulting mixture was concentrated in vacuo along with silica gel (10 g) and purified by flash column chromatography (silica gel 60, 0.040-0.063 mm; eluent: dichloromethane/methanol 100:0 to 98:2) to afford (6aR,9S)-7-methyl-N—((R)-pentan-2-yl)-4,6,6a,7,8,9-hexahydroindolo[4,3-fg]quinoline-9-carboxamide (Int17a, faster fluorescent band) as a dark-brown solid and (6aR,9R)-7-methyl-N—((R)-pentan-2-yl)-4,6,6a,7,8,9-hexahydroindolo[4,3-fg]quinoline-9-carboxamide (Int17, slower fluorescent band) as a dark-brown solid.

### Int17a:

Yield: 89 mg (35%).

¹H NMR spectrum (300 MHz, CD₃CN, δ_{H}): 9.07 (br s, 1H); 7.79 (br d, J=6.10, 1H); 7.28-7.21 (m, 1H); 7.14-7.09 (m, 2H); 6.98 (s, 1H); 6.53 (d, J=6.1, 1H); 3.89-3.73 (m, 1H); 3.61 (dd, J=14.6, 5.5, 1H); 3.15 (br s, 1H); 3.12 (d, J=12.0, 1H); 3.00 (br s, 1H); 2.69 (dd, J=11.6, 3.4, 1H); 2.65-2.51 (m, 1H); 2.56 (s, 3H); 1.45-1.31 (m, 4H); 0.98 (d, J=6.5, 3H); 0.90 (t, J=6.94, H).

LC-MS purity: 95% (ELSD), 100% (UV₃₁₀).

LC-MS Rt (Sinergy Polar RP, 4.6 mm×150 mm, acetonitrile/water 30:70 to 100:0+0.1% HFBA in 10 min): 5.317 min.

LC-MS m/z: 338.2 (M+H)⁺.

### Int17:

Yield: 102 mg (40%).

¹H NMR spectrum (300 MHz, CD₃CN, δ_{H}): 9.02 (br s, 1H); 7.23 (p, J=3.8, 1H); 7.13-7.07 (m, 2H); 6.95 (s, 1H); 6.53 (br d, J=6.81, 1H); 6.38 (s, 1H); 3.89 (dt, J=15.0, 6.6, 1H); 3.48 (dd, J=14.6, 5.5, 1H); 3.38-3.27 (m, 1H); 3.21-3.11 (m, 1H); 3.05 (dd, J=11.1, 5.0, 1H); 2.64-2.54 (m, 2H); 2.51 (s, 3H); 1.48-1.29 (m, 4H); 1.11 (d, J=6.6, 3H); 0.91 (t, J=7.1, 3H).

LC-MS purity: 91% (ELSD), 100% (UV₃₁₀).

LC-MS Rt (Sinergy Polar RP, 4.6 mm×150 mm, acetonitrile/water 30:70 to 100:0+0.1% HFBA in 10 min): 5.06 min.

LC-MS m/z: 338.2 (M+H)⁺.

A solution of 3-chloroperbenzoic (77%, 142.7 mg, 827 μmol) acid in dry dichloromethane (5 mL) was added dropwise to a solution of (6aR)-7-methyl-N—((R)-pentan-2-yl)-4,6,6a,7,8,9-hexahydroindolo[4,3-fg]quinoline-9-carboxamide (Int17m, 215 mg, 630 μmol; mixture of epimers at position 9) in dry dichloromethane (10 mL) at 0° C. and the mixture was stirred at the given temperature for 1 hour under argon atmosphere. A 10% aqueous solution of sodium hydroxide (100 mL) was then added to the reaction mixture and the aqueous phase was extracted with a 10% solution of isopropanol in dichloromethane (3×100 mL). The combined organic phases were combined, dried over anhydrous sodium sulfate, and concentrated in vacuo to afford (6aR)-7-methyl-9-(((R)-pentan-2-yl)carbamoyl)-4,6,6a,7,8,9-hexahydroindolo[4,3-fg]quinoline 7-oxide (Int18m) (mixture of diastereomers, epimers at position 9), which was used in the next step without further purification.

LC-MS purity: 100% (UV, 310 nm).

LC-MS Rt (Sinergy Polar RP, 4.6 mm×150 mm, acetonitrile/water 30:70 to 100:0+0.1% HBFA in 10 min): 5.23 min.

LC-MS m/z: 354.1 (M+H)⁺.

Crude (6aR)-7-methyl-9-(((R)-pentan-2-yl)carbamoyl)-4,6,6a,7,8,9-hexahydroindolo[4,3-fg]quinoline 7-oxide (Int18m, entire quantity obtained in above procedure; mixture of epimers at position 9) was dissolved in methanol (20 mL) and cooled to 0° C. under argon. Iron (II) sulfate heptahydrate (351 mg, 1.26 mmol) was then added and the resulting mixture was stirred for 3 hours at 0° C. The solvent was removed in vacuo and the residue partitioned between dichloromethane (150 mL) and a solution of EDTA (10 g) and 30% ammonium hydroxide (10 mL) in water (100 mL). The aqueous phase was further extracted with dichloromethane (3×100 mL) and the combined organic phases were dried over magnesium sulfate and concentrated in vacuo. The crude residue was purified by flash column chromatography (silica gel 60, 0.040-0.063 mm; eluent: dichloromethane/methanol 98:2 to 90:10) to yield (6aR)—N—((R)-pentan-2-yl)-4,6,6a,7,8,9-hexahydroindolo[4,3-fg]quinoline-9-carboxamide (Int19m) (mixture of diastereomers, epimers at position 9) as an amorphous beige solid.

Yield: 47.0 mg (23% over 2 steps from Int17m).

LC-MS purity: 97% (combined diastereomers, UV₃₁₀).

LC-MS Rt (Sinergy Polar RP, 4.6 mm×150 mm, acetonitrile/water 30:70 to 100:0+0.1% HBFA in 10 min): 4.94 min, 5.19 min.

LC-MS m/z: 324.2 (M+H)⁺.

A solution of (6aR)—N-(pentan-3-yl)-4,6,6a,7,8,9-hexahydroindolo[4,3-fg]quinoline-9-carboxamide (Int19m, 47.0 mg, 0.145 mmol; mixture of epimers at position 9) and propanal (53 μL, 0.74 mmol) in methanol (10 mL) was cooled to 0° C. under argon. Sodium cyanoborohydride (46.0 mg, 0.74 mmol) was added and the resulting mixture was stirred for 5 minutes followed by addition of glacial acetic acid (100 μL). After stirring at 0° C. for 1 hour, the solvents were removed in vacuo and the residue partitioned between dichloromethane (200 mL) and a 1% solution of ammonium hydroxide (150 mL). The aqueous phase was further extracted with dichloromethane (3×50 mL) and the combined organic phases were dried over sodium sulfate and concentrated in vacuo. The residue was purified by flash column chromatography (silica gel 60, 0.040-0.063 mm; eluent: dichloromethane/methanol 99:1 to 98:2) to afford (6aR,9R)-7-propyl-N—((R)-pentan-2-yl)-4,6,6a,7,8,9-hexahydroindolo[4,3-fg]quinoline-9-carboxamide (7, slower moving fluorescent band) as a dark brownish foam.

Yield: 20 mg.

LC-MS purity: 100% (ELSD), 89% (UV, 310 nm).

LC-MS Rt (Sinergy Polar RP, 4.6 mm×150 mm, acetonitrile/water 30:70 to 100:0+0.1% HBFA in 10 min): 5.88 min.

LC-MS m/z: 366.2 (M+H)⁺.

### Example 8: Preparation of (6aR,9R)-7-allyl-N—((R)-sec-butyl)-4,6,6a,7,8,9-hexahydroindolo[4,3-fg]quinoline-9-carboxamide (8) and (6aR,9S)-7-allyl-N—((R)-sec-butyl)-4,6,6a,7,8,9-hexahydroindolo[4,3-fg]quinoline-9-carboxamide (8a)

### Reaction Scheme:

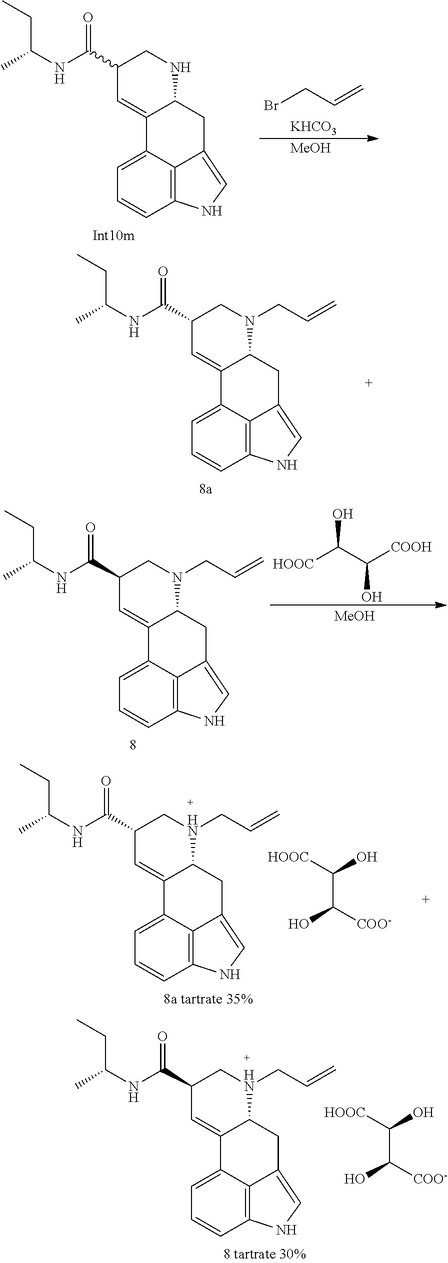

### Synthetic Protocols:

To a stirred solution of (6aR)—N—((R)-sec-butyl)-4,6,6a,7,8,9-hexahydroindolo[4,3-fg]quinoline-9-carboxamide (Int10m, 35 mg, 0.113 mmol; preparation is described in Example 3; mixture of epimers at position 9) and potassium bicarbonate (23 mg, 0.226 mmol) in methanol (2 mL) was added a solution of allylbromide (20 μL, 0.226 mmol) in methanol (1 mL) dropwise under argon. The resulting mixture was stirred for 72 hours at ambient temperature, diluted with dichloromethane (50 mL), and silica gel (10 g) was introduced. The obtained suspension was stripped of solvents in vacuo and subjected to silica gel chromatography (silica gel 60, 0.040-0.063 mm; eluent: dichloromethane/methanol/ammonia 98:2:0.1) to afford (6aR,9S)—N—((R)-sec-butyl)-7-allyl-4,6,6a,7,8,9-hexahydroindolo[4,3-fg]quinoline-9-carboxamide (8a, faster moving fluorescent band) as a brownish amorphous solid and (6aR,9R)—N—((R)-sec-butyl)-7-allyl-4,6,6a,7,8,9-hexahydroindolo[4,3-fg]quinoline-9-carboxamide (8, slower moving fluorescent band) as a brownish foam. The separated isomers were each dissolved in absolute methanol (500 μL) and treated with an equimolar amount of 1M D-(−)-tartaric in absolute methanol. The resulting solutions were stripped of solvents under a flow of nitrogen and dried under high vacuum to yield (6aR,9S)-7-allyl-9-(((R)-sec-butyl)carbamoyl)-4,6,6a,7,8,9-hexahydroindolo[4,3-fg]quinolin-7-ium (2S,3S)-3-carboxy-2,3-dihydroxypropanoate (8a tartrate) as a light-brown solid and (6aR,9R)-7-allyl-9-(((R)-sec-butyl)carbamoyl)-4,6,6a,7,8,9-hexahydroindolo[4,3-fg]quinolin-7-ium (2S,3S)-3-carboxy-2,3-dihydroxypropanoate (8 tartrate) as a light-brown solid.

8a:

Yield (of freebase): 14.1 mg (35%).

¹H NMR spectrum (of tartrate) (300 MHz, MeOD, δ_{H}): 7.26 (dt, J=7.3, 3.6, 1H); 7.18-7.08 (m, 2H); 7.05 (d, J=1.1, 1H); 6.57 (d, J=5.6, 1H); 6.18-5.99 (m, 1H); 5.63-5.46 (m, 2H); 4.47 (s, 2H); 4.14 (dd, J=13.7, 5.6, 1H); 4.02 (d, J=6.8, 1H); 3.87-3.62 (m, 4H); 3.40 (br s, 1H); 3.18 (dd, J=12.1, 3.5, 1H); 2.93 (t, J=13.0, 1H); 1.58-1.40 (m, 2H); 1.18 (t, J=7.0, 1H); 1.13 (d, J=6.6, 3H); 0.89 (t, J=7.4, 3H).

LC-MS purity (of freebase): 98% (ELSD), 97% (UV, 310 nm).

LC-MS purity (of tartrate): 99% (ELSD).

LC-MS Rt (of tartrate) (Sinergy Polar RP 4.6 mm×150 mm, acetonitrile/water 30:70 to 100:0+0.1% HFBA in 10 min): 6.65 min.

LC-MS m/z: 350.1 (M+H)⁺.

8:

Yield (of freebase): 12 mg (30%).

¹H NMR spectrum (of tartrate) (300 MHz, MeOD, δ_{H}): 7.25 (dd, J=6.6, 2.0, 1H); 7.18-7.07 (m, 2H); 7.04 (s, 1H); 6.46 (s, 1H); 6.20-5.99 (m, 1H); 5.67-5.47 (m, 2H); 4.46 (s, 2H); 4.16-4.09 (m, 1H); 4.06 (dd, J=21.2, 7.4, 1H); 3.91-3.64 (m, 4H); 3.57 (dd, J=12.0, 4.8, 1H); 3.29 (t, J=12.6, 1H); 3.00 (t, J=12.6, 1H); 1.62-1.44 (m, 2H); 1.20 (t, J=3.3, 1H); 1.17 (d, J=7.0, 3H); 0.94 (t, J=7.4, 3H).

LC-MS purity (of tartrate): 98% (ELSD).

LC-MS Rt (of tartrate) (Sinergy Polar RP 4.6 mm×150 mm, acetonitrile/water 30:70 to 100:0+0.1% HFBA in 10 min): 5.47 min.

LC-MS m/z: 350.1 (M+H)⁺.

### Example 9: Preparation of (6aR,9R)-5-bromo-N,N-diethyl-7-propyl-4,6,6a,7,8,9-hexahydroindolo[4,3-fg]quinoline-9-carboxamide (9)

### Reaction Scheme:

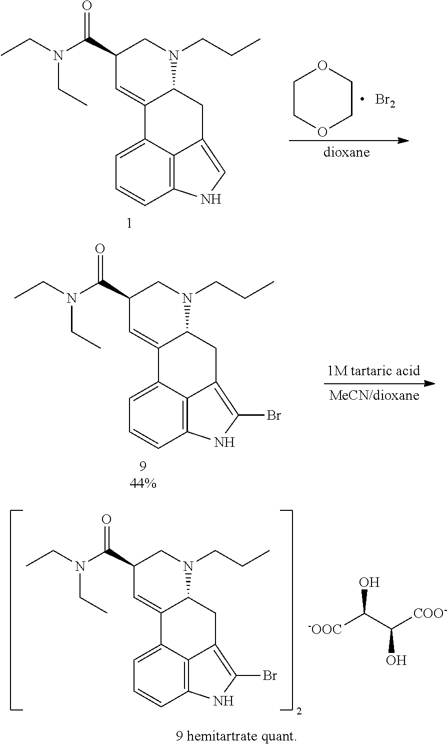

### Synthetic Protocols:

A solution of (6aR,9R)—N,N-Diethyl-7-propyl-4,6,6a,7,8,9-hexahydroindolo[4,3-fg]quinoline-9-carboxamide (1, 53.0 mg, 0.151 mmol) in anhydrous dioxane (2.0 mL) was flushed with argon. To this solution was added a 10% v/v solution of bromine in dioxane (754 μL, 0.151 mmol) in a dropwise fashion and the resulting mixture was stirred for 48 h. The reaction mixture was filtered through a pad of silica gel. The filtrate was concentrated in vacuo and the residue was purified by flash column chromatography (silica gel 60, 0.040-0.063 mm; eluent: dichloromethane/methanol 98:2) to give (6aR,9R)-5-bromo-N,N-diethyl-7-propyl-4,6,6a,7,8,9-hexahydroindolo[4,3-fg]quinoline-9-carboxamide (9) as a dark-tinted amorphous solid.

Yield: 28.4 mg (44%).

¹H NMR spectrum (300 MHz, CD₃CN, δ_{H}): 9.36 (s, 1H), 7.19-7.11 (m, 1H), 7.11-7.06 (m, 2H), 6.32 (s, 1H), 3.75-3.65 (m, 1H), 3.45 (dt, J=10.8, 3.6, 2H), 3.41-3.29 (m, 4H), 3.13 (ddd, J=11.2, 4.8, 1.0, 1H), 2.89 (ddd, J=13.3, 9.0, 7.1, 1H), 2.61 (t, J=10.8, 1H), 2.48 (ddd, J=13.4, 8.7, 4.9, 1H), 2.40 (dd, J=16.3, 12.6, 1H), 1.67-1.46 (m, 2H), 1.21 (t, J=7.1, 3H), 1.10 (t, J=7.1, 3H), 0.94 (t, J=7.4, 3H).

LC-MS purity: 100% (ELSD), 97% (UV, 310 nm).

LC-MS Rt (Sinergy Polar RP, 4.6 mm×150 mm, acetonitrile/water 30:70 to 100:0+0.1% HBFA in 10 min): 6.20 min.

LC-MS m/z: 431.9 (M+H)⁺.

A solution of (6aR,9R)-5-bromo-N,N-diethyl-7-propyl-4,6,6a,7,8,9-hexahydroindolo[4,3-fg]quinoline-9-carboxamide (9, 28.4 mg, 66 μmol) in gradient-grade acetonitrile (5.0 mL) was treated with aqueous 1M D-(−)-tartaric acid solution (33 μL, 66 μmol) and stirred for 5 minutes. The solvent was removed in vacuo and the residue was re-dissolved in dioxane (5.0 mL) and then freeze dried at 0° C. to yield (6aR,9R)-5-bromo-N,N-diethyl-7-propyl-4,6,6a,7,8,9-hexahydroindolo[4,3-fg]quinoline-9-carboxamide hemitartrate (9 hemitartrate) as a fluffy light-brown solid.

Yield: 33.2 mg (quantitative).

¹H NMR spectrum (300 MHz, MeOD, δ_{H}): 7.18 (dd, J=7.0, 1.9, 1H), 7.16-7.06 (m, 2H), 6.39 (s, 1H), 4.40 (s, 1H), 4.07-3.99 (m, 1H), 3.99-3.86 (m, 1H), 3.58 (dt, J=14.1, 7.2, 2H), 3.51-3.36 (m, 5H), 3.27-3.12 (m, 2H), 3.09-2.93 (m, 1H), 2.77 (t, J=12.4, 1H), 1.89-1.69 (m, 2H), 1.31 (t, J=7.1, 3H), 1.19 (t, J=7.1, 3H), 1.06 (t, J=7.3, 3H).

LC-MS purity: 100% (ELSD), 97% (UV, 310 nm).

LC-MS Rt (Sinergy Polar RP, 4.6 mm×150 mm, acetonitrile/water 30:70 to 100:0+0.1% HBFA in 10 min): 6.20 min.

LC-MS m/z: 431.9 (M+H)⁺.

### Example 10: Preparation of (6aR,9R)—N,N-diethyl-7-(2-methoxybenzyl)-4,6,6a,7,8,9-hexahydroindolo[4,3-fg]quinoline-9-carboxamide (10)

### Reaction Scheme:

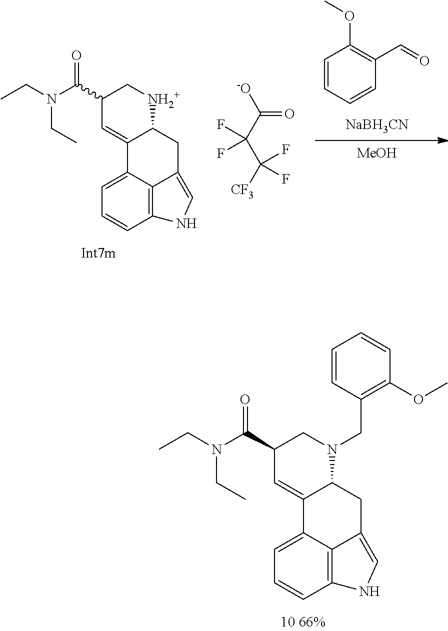

### Synthetic Protocols:

A solution of (6aR)—N,N-diethyl-4,6,6a,7,8,9-hexahydroindolo[4,3-fg]quinoline-9-carboxamide heptafluorobutanoate (Int7m, 40.0 mg, 0.077 mmol; mixture of epimers at position 9) and 2-methoxybenzaldehyde (32.0 mg, 0.23 mmol) in methanol (10 mL) was cooled to 0° C. under argon. Sodium cyanoborohydride (15.0 mg, 0.23 mmol) was added, the resulting mixture was stirred for 5 minutes, and then glacial acetic acid (100 μL) was added and stirring was continued at room temperature. After 48 hours, the solvents were removed in vacuo, the residue was partitioned between dichloromethane (200 mL) and a 1% solution of ammonium hydroxide (150 mL), and the aqueous phase was further extracted with dichloromethane (3×50 mL). The combined organic phases were dried over sodium sulfate and concentrated in vacuo. The residue was purified by flash column chromatography (silica gel 60, 0.040-0.063 mm; eluent: dichloromethane/methanol 98:2) to afford (6aR,9R)—N,N-diethyl-7-(2-methoxybenzyl)-4,6,6a,7,8,9-hexahydroindolo[4,3-fg]quinoline-9-carboxamide (10) as a colorless solid.

Yield: 22 mg (66%).

¹H NMR spectrum (300 MHz, CD₃CN, δ_{H}): 8.99 (br s, 1H); 7.48 (d, J=7.4, 1H); 7.30-7.19 (m, 2H); 7.14-7.05 (m, 2H); 7.00-6.90 (m, 3H); 6.32 (s, 1H); 4.14 (d, J=14.6, 1H); 3.80 (s, 3H); 3.75-3.63 (m, 3H); 3.47-3.26 (m, 5H); 3.05 (dd, J=11.1, 4.4, 1H); 2.70-2.47 (m, 2H); 1.08 (dt, J=9.4, 7.1, 6H).

LC-MS purity: 99% (ELSD), 100% (UV, 310 nm).

LC-MS Rt (Sinergy Polar RP, 4.6 mm×150 mm, acetonitrile/water 30:70 to 100:0+0.1% HFBA in 10 min): 7.64 min.

LC-MS m/z: 430.2 (M+H)⁺.

### Example 11: Preparation of (6aR,9R)—N,N-diethyl-7-(2-methoxyphenethyl)-4,6,6a,7,8,9-hexahydroindolo[4,3-fg]quinoline-9-carboxamide (11)

### Reaction Scheme:

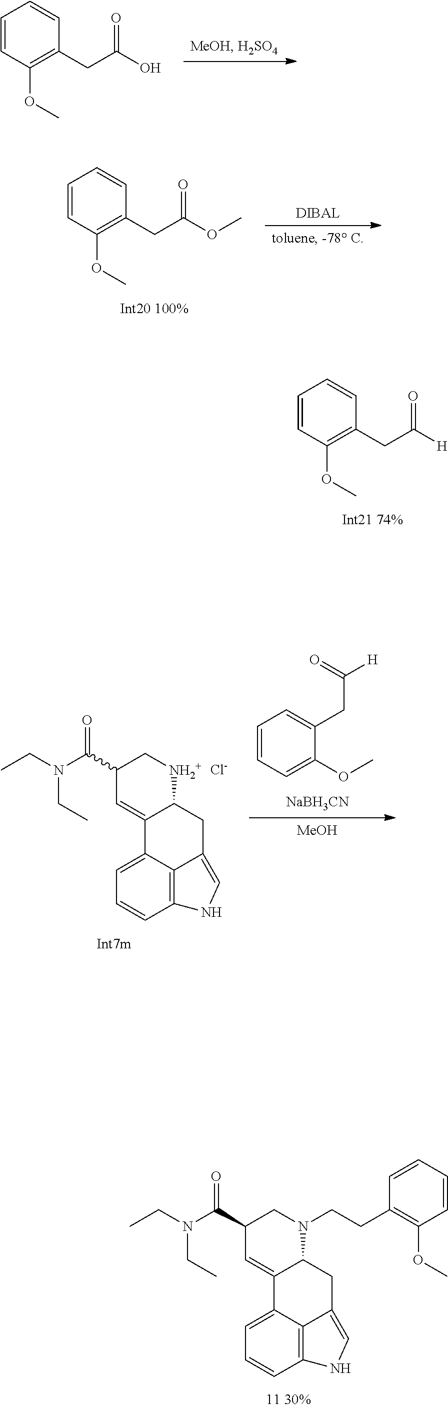

### Synthetic Protocols:

2-(2-methoxyphenyl)acetic acid (1.66 g, 10.0 mmol) was dissolved in dry methanol (10 mL), sulfuric acid 96% (1.0 mL) was added, and the mixture was refluxed for 3 hours. The solvent was then evaporated and the residue was partitioned between ethyl acetate (50 mL) and a saturated solution of sodium bicarbonate (50 mL). The organic phase was dried over anhydrous sodium sulfate and concentrated in vacuo to yield methyl 2-(2-methoxyphenyl)acetate (Int20) as a colorless oil.

Yield: 1.80 g (100%).

¹H NMR spectrum (300 MHz, CDCl₃, δ_{H}): 7.30-7.14 (m, 2H); 6.96-6.84 (m, 2H); 3.82 (s, 3H); 3.69 (s, 3H); 3.64 (s, 2H).

2-(2-methoxyphenyl)acetate (Int20, 1.80 g, 10.0 mmol) was dissolved in dry toluene (20 mL) and cooled to −78° C. A solution of diisobutylaluminium hydride (15.0 mL, 15 mmol, 1 M solution in hexanes) was introduced dropwise and the resulting mixture was stirred for 2 hours at −78° C. The reaction was quenched by slow addition of methanol (5 mL), followed by addition of a 10% solution of sodium potassium tartarate (20 mL) and ethylacetate (50 mL). The resulting mixture was then stirred for 1 hour at room temperature. The phases were separated, the aqueous phase was further extracted with ethyl acetate (2×50 mL), and the combined organic phases were dried over anhydrous sodium sulfate and evaporated. The residue was purified by flash column chromatography (silica gel 60, 0.040-0.063 mm; eluent: cyclohexane/ethylacetate 9:1) to afford 2-(2-methoxyphenyl)acetaldehyde (Int21) as a colorless oil.

Yield: 1.11 g (74%).

¹H NMR spectrum (300 MHz, CDCl₃, δ_{H}): 9.68 (t, J=2.1 Hz, 1H); 7.30 (td, J=8.1, 1.6 Hz, 1H); 7.15 (dd, J=7.3, 1.2 Hz, 1H); 7.01-6.87 (m, 2H); 3.83 (s, 3H); 3.65 (d, J=2.0 Hz, 2H).

A solution of (6aR)—N,N-diethyl-4,6,6a,7,8,9-hexahydroindolo[4,3-fg]quinoline-9-carboxamide hydrochloride (Int7m, 51.0 mg, 0.148 mmol; HCl salt; mixture of epimers at position 9) and 2-(2-methoxyphenyl)acetaldehyde (Int21, 111 mg, 0.74 mmol) in methanol (10 mL) was cooled to 0° C. under argon. Sodium cyanoborohydride (46.0 mg, 0.74 mmol) was added, the mixture was stirred for 5 minutes, and then glacial acetic acid (100 μL) was added and stirring was continued at 0° C. After 1 hour, the solvents were removed in vacuo, the residue was partitioned between dichloromethane (200 mL) and a 1% solution of ammonium hydroxide (150 mL), and the aqueous phase was further extracted with dichloromethane (3×50 mL). The combined organic phases were dried over sodium sulfate and concentrated in vacuo. The residue was purified by flash column chromatography (silica gel 60, 0.040-0.063 mm; eluent: dichloromethane/methanol 98:2) to afford (6aR,9R)—N,N-diethyl-7-(2-methoxyphenethyl)-4,6,6a,7,8,9-hexahydroindolo[4,3-fg]quinoline-9-carboxamide (11) as a colorless foam.

Yield: 20 mg (30%).

¹H NMR spectrum (300 MHz, CD₃CN, δ_{H}): 9.01 (br s, 1H); 7.25-7.15 (m, 3H); 7.13-7.06 (m, 2H); 6.99-6.83 (m, 3H); 6.31 (s, 1H); 3.85 (s, 3H); 3.76-3.67 (m, 1H); 3.60 (dd, J=14.4, 5.4, 1H); 3.53-3.33 (m, 4H); 3.18 (dd, J=11.1, 4.1, 1H); 3.13-3.02 (m, 1H); 2.97-2.69 (m, 4H); 2.46 (ddd, J=14.2, 11.1, 1.5, 1H); 1.22 (t, J=7.1, 3H); 1.12 (t, J=7.1, 3H).

LC-MS purity: 99% (ELSD), 97% (UV, 310 nm).

LC-MS Rt (Sinergy Polar RP, 4.6 mm×150 mm, acetonitrile/water 30:70 to 100:0+0.1% HBFA in 10 min): 6.61 min.

LC-MS m/z: 444.3 (M+H)⁺.

### Example 12: Preparation of (6aR,9R)—N,N-diethyl-7-(3,3,3-trifluoropropyl)-4,6,6a,7,8,9-hexahydroindolo[4,3-fg]quinoline-9-carboxamide (12)

### Reaction Scheme:

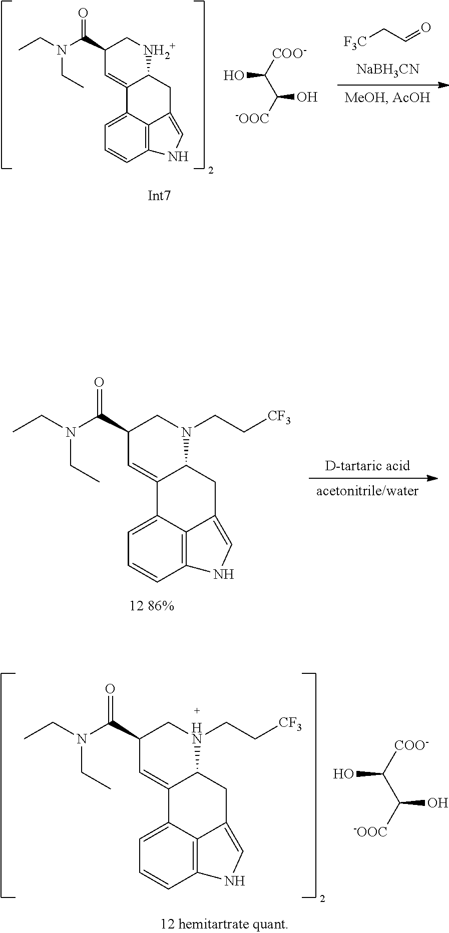

### Synthetic Protocols:

A solution of (6aR,9R)—N,N-diethyl-4,6,6a,7,8,9-hexahydroindolo[4,3-fg]quinoline-9-carboxamide hemitartrate (Int7, 30.0 mg, 78.0 μmol; 2 moles Int7 per mole tartrate) and 3,3,3-trifluoropropanal (27.0 μL, 0.31 mmol) in methanol (2 mL) was purged with argon and cooled to 0° C. Sodium cyanoborohydride (20.0 mg, 0.32 mmol) was added and the resulting mixture stirred for 5 minutes followed by addition of glacial acetic acid (20 μL). After stirring at 0° C. for 3 h, the solvents were removed in vacuo, the residue was partitioned between dichloromethane (100 mL) and a 1% aqueous solution of ammonium hydroxide (150 mL), and the aqueous phase was further extracted with dichloromethane (3×50 mL). The combined organic phases were dried over sodium sulfate and concentrated in vacuo. The residue was purified by flash column chromatography (silica gel 60, 0.040-0.063 mm; eluent: dichloromethane/methanol 98:2) to provide (6aR,9R)—N,N-diethyl-7-(3,3,3-trifluoropropyl)-4,6,6a,7,8,9-hexahydroindolo[4,3-fg]quinoline-9-carboxamide (12) as a colorless foam.

Yield: 27.2 mg (86%).

¹H NMR spectrum (300 MHz, CD₃CN, δ_{H}): 9.01 (s, 1H), 7.23 (dd, J=6.8, 1.9, 1H), 7.15-7.03 (m, 2H), 6.96 (t, J=1.8, 1H), 6.31 (s, 1H), 3.79-3.66 (m, 1H), 3.51 (dd, J=14.5, 5.5, 1H), 3.46 (dd, J=7.3, 3.2, 1H), 3.37 (m, 4H), 3.22 (ddd, J=14.0, 9.1, 6.8, 1H), 3.09 (ddd, J=11.1, 4.8, 1.0, 1H), 2.86 (ddd, J=14.0, 8.9, 5.3, 1H), 2.73 (t, 1H), 2.57 (dd, J=11.1, 1.7, 1H), 2.54-2.40 (m, 2H), 1.22 (t, J=7.1, 3H), 1.11 (t, J=7.1, 3H).

LC-MS purity: 99% (ELSD), 99% (UV, 310 nm).

LC-MS Rt (Sinergy Polar RP, 4.6 mm×150 mm, acetonitrile/water 30:70 to 100:0+0.1% HBFA in 10 min): 5.85 min.

LC-MS m/z: 406.0 (M+H)⁺.

(6aR,9R)—N,N-diethyl-7-(3,3,3-trifluoropropyl)-4,6,6a,7,8,9-hexahydroindolo[4,3-fg]quinoline-9-carboxamide (12, 27.2 mg, 67.1 μmol) was dissolved in gradient-grade acetonitrile (5.0 mL) and treated with aqueous 1M D-(−)-tartaric acid solution (33.6 μL, 33.6 μmol). Solvents were removed in vacuo, and the obtained material was redissolved in dioxane (5.0 mL) and subjected to freeze drying at 0° C. to yield (6aR,9R)—N,N-diethyl-7-(3,3,3-trifluoropropyl)-4,6,6a,7,8,9-hexahydroindolo[4,3-fg]quinoline-9-carboxamide hemitartrate (12 hemitartrate) as a fluffy white solid.

Yield: 32.2 mg (quantitative).

¹H NMR spectrum (300 MHz, MeOD, δ_{H}): 7.20 (dd, J=6.3, 2.4, 1H), 7.14-7.05 (m, 2H), 6.98 (d, J=1.2, 1H), 6.31 (s, 1H), 4.50 (s, 1H), 3.99-3.88 (m, 1H), 3.63-3.40 (m, 7H), 3.20 (dd, J=11.1, 4.5, 1H), 3.11-2.99 (m, 1H), 2.94 (t, J=10.3, 1H), 2.73 (t, J=12.0, 1H), 2.58 (ddd, J=16.1, 10.2, 5.5, 2H), 1.30 (t, J=7.1, 3H), 1.18 (t, J=7.1, 3H).

LC-MS purity: 97% (ELSD), 92% (UV, 310 nm).

LC-MS Rt (Sinergy Polar RP, 4.6 mm×150 mm, acetonitrile/water 30:70 to 100:0+0.1% HBFA in 10 min): 5.85 min.

LC-MS m/z: 406.0 (M+H)⁺.

### Example 13: Preparation of (6aR,9R)—N,N-diethyl-7-(cyclopropylmethyl)-4,6,6a,7,8,9-hexahydroindolo[4,3-fg]quinoline-9-carboxamide (13)

### Reaction Scheme:

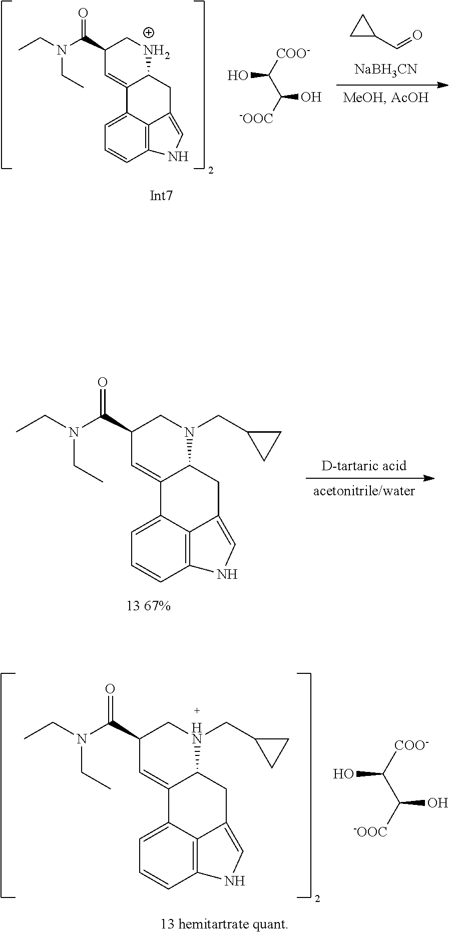

### Synthetic Protocols:

A solution of (6aR,9R)—N,N-diethyl-4,6,6a,7,8,9-hexahydroindolo[4,3-fg]quinoline-9-carboxamide hemitartrate (Int7, 30.0 mg, 78.0 μmol; 2 moles Int7 per mole tartrate) and cyclopropanecarbaldehyde (23.0 μL, 0.31 mmol) in methanol (2 mL) was purged with argon and cooled to 0° C. Sodium cyanoborohydride (20.0 mg, 0.32 mmol) was added and the resulting mixture was stirred for 5 minutes followed by the addition of glacial acetic acid (20 uL). After stirring at 0° C. for 3 h, the solvents were removed in vacuo, the residue was partitioned between dichloromethane (100 mL) and a 1% aqueous solution of ammonium hydroxide (150 mL), and the aqueous phase was further extracted with dichloromethane (3×50 mL). The combined organic phases were dried over sodium sulfate and concentrated in vacuo. The residue was purified by flash column chromatography (silica gel 60, 0.040-0.063 mm; eluent: dichloromethane/methanol 98:2) to provide (6aR,9R)-7-(cyclopropylmethyl)-N,N-diethyl-4,6,6a,7,8,9-hexahydroindolo[4,3-fg]quinoline-9-carboxamide (13) as a colorless foam.

Yield: 19.0 mg (67%).

¹H NMR spectrum (300 MHz, CD₃CN, δ_{H}): 9.17 (s, 1H), 7.27 (dd, J=6.8, 1.9, 1H), 7.17-7.05 (m, 2H), 7.00 (s, 1H), 6.37 (s, 1H), 3.98-3.79 (m, 2H), 3.62-3.30 (m, 6H), 3.18 (dd, J=11.3, 7.7, 1H), 3.01-2.84 (m, 2H), 2.78 (t, J=13.1, 1H), 1.24 (t, J=7.1, 3H), 1.13 (t, J=7.1, 3H), 1.15-1.00 (m, 1H), 0.65-0.56 (m, 2H), 0.34-0.26 (m, 2H).

LC-MS purity: 99% (ELSD), 99% (UV, 310 nm).

LC-MS Rt (Sinergy Polar RP, 4.6 mm×150 mm, acetonitrile/water 30:70 to 100:0+0.1% HBFA in 10 min): 5.56 min.

LC-MS m/z: 364.1 (M+H)⁺.

(6aR,9R)-7-(cyclopropylmethyl)-N,N-diethyl-4,6,6a,7,8,9-hexahydroindolo[4,3-fg]quinoline-9-carboxamide (13, 19.0 mg, 52.2 μmol) was dissolved in gradient-grade acetonitrile (5.0 mL) and treated with aqueous 1M D-(−)-tartaric acid solution (26.2 μL, 26.2 μmol). The solvents were removed in vacuo, and the obtained material was redissolved in dioxane (5.0 mL) and subjected to freeze drying at 0° C. to yield (6aR,9R)-7-(cyclopropylmethyl)-N,N-diethyl-4,6,6a,7,8,9-hexahydroindolo[4,3-fg]quinoline-9-carboxamide hemitartrate (13 hemitartrate) as a fluffy white solid.

Yield: 22.9 mg (quantitative).

¹H NMR spectrum (300 MHz, MeOD, δ_{H}): 7.27 (p, J=3.8, 1H), 7.17-7.10 (m, 2H), 7.07 (d, J=0.8, 1H), 6.43 (dd, J=2.8, 1.7, 1H), 4.40 (s, 1H), 4.36-4.24 (m, 1H), 4.15 (s, 1H), 3.77-3.36 (m, 8H), 3.31-3.22 (m, 1H), 3.02 (t, J=12.9, 1H), 1.34 (t, J=7.1, 3H), 1.34-1.16 (m, 1H), 1.20 (t, J=7.1, 3H), 0.78 (q, J=5.4, 2H), 0.48 (d, J=4.3, 2H).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 5.56 min.

LC-MS m/z: 364.1 (M+H)⁺.

### Example 14: Preparation of ((6aR,9R)-7-propyl-4,6,6a,7,8,9-hexahydroindolo[4,3-fg]quinolin-9-yl)(pyrrolidin-1-yl)methanone (14)

### Reaction Scheme:

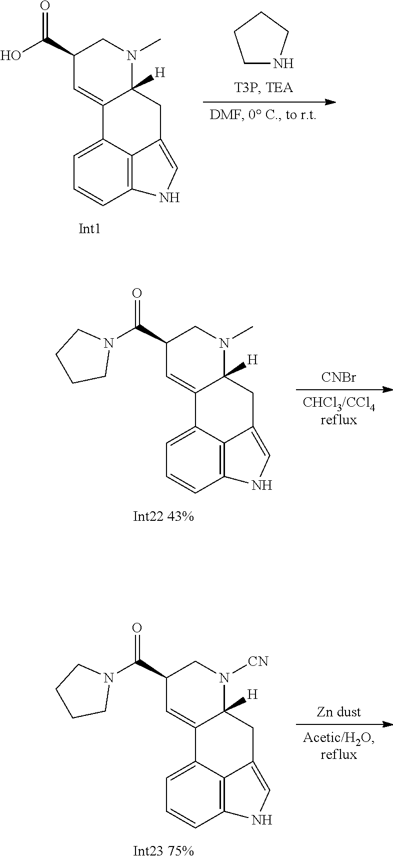

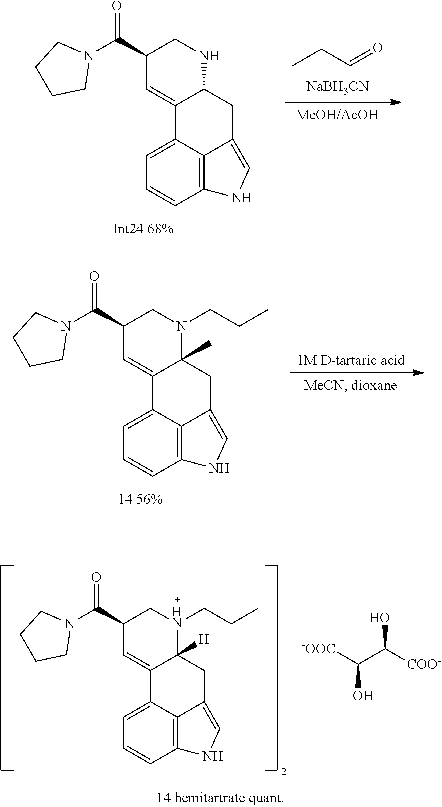

### Synthetic Protocols:

A solution of (6aR,9R)-7-methyl-4,6,6a,7,8,9-hexahydroindolo[4,3-fg]quinoline-9-carboxylic acid (Int1, 500 mg, 1.86 mmol), triethylamine (1.05 mL, 7.44 mmol), and pyrrolidine (460 μl, 5.59 mmol) in dry N,N-dimethylformamide (10 mL) was cooled to 0° C. under an argon atmosphere. Propanephosphonic acid anhydride (T3P®, 3.26 mL, 5.59 mmol, 50% solution in DMF) was added dropwise over 5 minutes. The resulting mixture was stirred for 1 h at 0° C. The reaction was judged to be complete by LC-MS and it was then quenched with ice-cold water (10 mL). The mixture was partitioned between 1M aqueous ammonium hydroxide solution (200 mL) and ethyl acetate (100 mL). The aqueous phase was re-extracted with ethyl acetate (2×150 mL). The organic phases were combined and then washed with 10% aq. lithium chloride solution (4×150 mL), dried over anhydrous magnesium sulfate, and concentrated in vacuo. The residue was purified by flash column chromatography (silica gel 60, 0.040-0.063 mm; eluent: dichloromethane/methanol 100:0 to 98:2) to afford ((6aR,9R)-7-methyl-4,6,6a,7,8,9-hexahydroindolo[4,3-fg]quinolin-9-yl)(pyrrolidin-1-yl)methanone (Int22) as a dark-brown solid.

Yield: 255 mg (43%).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 4.59 min.

LC-MS m/z: 322.0 (M+H)⁺.

A solution of ((6aR,9R)-7-methyl-4,6,6a,7,8,9-hexahydroindolo[4,3-fg]quinolin-9-yl)(pyrrolidin-1-yl)methanone (Int22, 52.7 mg, 0.164 mmol) in gradient-grade acetonitrile (5.0 mL) was treated with aqueous 1M D-(−)-tartaric acid solution (81.4 μL, 0.081 mmol) and stirred for 5 min at room temperature. The solvent was removed in vacuo. The residue was re-dissolved in dioxane (5.0 mL) and subjected to freeze drying at 0° C. to yield ((6aR,9R)-7-methyl-4,6,6a,7,8,9-hexahydroindolo[4,3-fg]quinolin-9-yl)(pyrrolidin-1-yl)methanone hemitartrate (Int22 hemitartrate) as a fluffy white solid.

Yield: 69.0 mg (quantitative).

¹H NMR spectrum (300 MHz, MeOD, δ_{H}): 7.24 (d, J=7.7, 1H), 7.20-7.07 (m, 2H), 7.03 (s, 1H), 6.46 (s, 1H), 4.40 (s, 1H), 4.07 (dd, J=6.5, 4.0, 1H), 3.90-3.79 (m, 1H), 3.76-3.67 (m, 1H), 3.71 (dd, J=13.8, 6.3, 2H), 3.54-3.44 (m, 1H), 3.49 (dd, J=12.7, 5.9, 2H), 3.28-3.16 (m, 1H), 2.94 (s, 3H), 2.95-2.83 (m, 1H), 2.10-1.90 (m, 4H).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 4.59 min.

LC-MS m/z: 322.0 (M+H)⁺.

To a solution of cyanogen bromide (380 mg, 3.60 mmol) in carbon tetrachloride (30 mL) under reflux was added a solution of ((6aR,9R)-7-methyl-4,6,6a,7,8,9-hexahydroindolo[4,3-fg]quinolin-9-yl)(pyrrolidin-1-yl)methanone (Int22, 255 mg, 0.795 mmol) in chloroform (10 mL) and carbon tetrachloride (70 mL) at a rate sufficient to maintain reflux. The reaction mixture was then heated under reflux for an additional 4 h. The mixture was then allowed to cool to room temperature and silica gel (silica gel 0.063-0.200 mm, 10 g) was added. The mixture was concentrated in vacuo. The resulting powder was added to the top of a flash chromatography column previously filled with silica gel and the product was eluted as follows (silica gel 60, 0.040-0.063 mm; eluent: cyclohexane/ethyl acetate 100:0 to 50:50) to afford (6aR,9R)-9-(pyrrolidine-1-carbonyl)-6,6a,8,9-tetrahydroindolo[4,3-fg]quinoline-7(4H)-carbonitrile (Int23) as a colorless amorphous solid.

Yield: 200 mg (75%).

¹H NMR spectrum (300 MHz, MeOD, δ_{H}): 8.10 (s, 1H), 7.33-7.23 (m, 1H), 7.22-7.13 (m, 2H), 6.98 (s, 1H), 6.36 (s, 1H), 4.32-4.17 (m, 1H), 3.92-3.80 (m, 1H), 3.76-3.69 (m, 2H), 3.68-3.50 (m, 5H), 3.11-2.98 (m, 1H), 2.11-1.89 (m, 4H).

LC-MS purity: 98% (ELSD), 98% (UV, 310 nm).

LC-MS Rt (Sinergy Polar RP, 4.6 mm×150 mm, acetonitrile/water 30:70 to 100:0+0.1% HBFA in 10 min): 5.87 min.

LC-MS m/z: 333.0 (M+H)⁺.

A solution of (6aR,9R)-9-(pyrrolidine-1-carbonyl)-6,6a,8,9-tetrahydroindolo[4,3-fg]quinoline-7(4H)-carbonitrile (Int23, 200 mg, 0.601 mmol) in acetic acid (15 mL) and water (1.5 mL) was treated with zinc dust (1000 mg). The resulting suspension was heated under reflux for 1 h. After cooling, the mixture was filtered through cotton, and the solution was basified with 10% aqueous ethylenediamine (100 mL) and stirred for 1 h. The mixture was diluted with water (100 mL) and extracted with dichloromethane (3×100 mL). The combined organic extracts were dried over anhydrous sodium sulfate and then filtered. The filtrate was treated with silica gel (silica gel 0.063-0.200 mm, 10 g) and evaporated in vacuo. The powder was added to the top of a flash chromatography column previously filled with silica gel and the product was eluted as follows (silica gel 60, 0.040-0.063 mm; eluent: dichloromethane/methanol 100:0 to 98:2) to afford ((6aR,9R)-4,6,6a,7,8,9 hexahydroindolo[4,3-fg]quinolin-9-yl)(pyrrolidin-1-yl)methanone (Int24) as a dark amorphous solid.

Yield: 125 mg (68%).

¹H NMR spectrum (300 MHz, CD₃CN, δ_{H}): 9.08 (s, 1H), 7.28-7.17 (m, 1H), 7.16-7.06 (m, 2H), 6.95 (s, 1H), 6.39 (s, 1H), 3.71 (ddd, J=11.4, 5.7, 2.4, 1H), 3.66-3.50 (m, 3H), 3.39 (td, J=6.8, 3.7, 2H), 3.25 (dd, J=12.9, 4.8, 1H), 3.14 (dd, J=14.8, 5.8, 1H), 2.99 (dd, J=12.5, 9.3, 1H), 2.67-2.53 (m, 1H), 2.14 (s, 1H), 1.98-1.91 (m, 2H), 1.90-1.77 (m, 2H).

LC-MS purity: 95% (ELSD), 95% (UV, 310 nm).

LC-MS Rt (Sinergy Polar RP, 4.6 mm×150 mm, acetonitrile/water 30:70 to 100:0+0.1% HBFA in 10 min): 4.22 min.

LC-MS m/z: 308.0 (M+H)⁺.

A solution of ((6aR,9R)-4,6,6a,7,8,9-hexahydroindolo[4,3-fg]quinolin-9-yl)(pyrrolidin-1-yl)methanone hemitartrate (Int24, 40 mg, 105 μmol; 2 moles Int24 per mole tartrate; salt prepared as described for other hemitartrates) and propanal (15 μL, 210 μmol) in methanol (2 mL) was purged with argon gas and cooled to 0° C. Sodium cyanoborohydride (14 mg, 210 μmol) was added. The resulting mixture was stirred for 5 minutes, and then acetic acid (50 μL) was introduced. After stirring at 0° C. for 1 hour, silica gel (silica gel 0.063-0.200 mm, 10 g) was added, and the mixture was concentrated in vacuo. The powder was added to the top of a flash chromatography column previously filled with silica gel and the product was eluted as follows (silica gel 60, 0.040-0.063 mm; eluent: dichloromethane/methanol 99:1 to 98:2) to afford ((6aR,9R)-7-propyl-4,6,6a,7,8,9-hexahydroindolo[4,3-fg]quinolin-9-yl)(pyrrolidin-1-yl)methanone (14) as a colorless foam.

Yield: 20.5 mg (56%).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 5.28 min.

LC-MS m/z: 350.1 (M+H)⁺.

A solution of ((6aR,9R)-7-propyl-4,6,6a,7,8,9-hexahydroindolo[4,3-fg]quinolin-9-yl)(pyrrolidin-1-yl)methanone (14, 20.5 mg, 58.7 μmol) in gradient-grade acetonitrile (5.0 mL) was treated with aqueous 1M D-(−)-tartaric acid solution (29 μL, 29 μmol). After stirring at room temperature for 5 minutes, the solvent was removed in vacuo. The residue was re-dissolved in dioxane (5.0 mL) and subjected to freeze drying at 0° C. to afford ((6aR,9R)-7-propyl-4,6,6a,7,8,9-hexahydroindolo[4,3-fg]quinolin-9-yl)(pyrrolidin-1-yl)methanone hemitartrate (14 hemitartrate) as a fluffy white solid.

Yield: 23.2 mg (quantitative).

¹H NMR spectrum (300 MHz, MeOD, δ_{H}): 7.26 (dd, J=7.2, 1.4, 1H), 7.19-7.09 (m, 2H), 7.06 (d, J=1.1, 1H), 6.47 (s, 1H), 4.39 (s, 1H), 4.25-4.10 (m, 1H), 4.08-3.95 (m, 1H), 3.74 (dd, J=6.3, 4.7, 2H), 3.70-3.63 (m, 2H), 3.59-3.47 (m, 3H), 3.47-3.38 (m, 1H), 3.25-3.11 (m, 1H), 2.99 (t, J=12.0, 1H), 2.07 (dt, J=11.5, 5.8, 2H), 1.97 (dt, J=9.0, 4.6, 2H), 1.90-1.77 (m, 2H), 1.07 (t, J=7.4, 3H).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 5.28 min.

LC-MS m/z: 350.1 (M+H)⁺.

### Example 15: Preparation of (6aR,9R)—N,N-diethyl-7-(2-hydroxybenzyl)-4,6,6a,7,8,9-hexahydroindolo[4,3-fg]quinoline-9-carboxamide (16)

### Reaction Scheme:

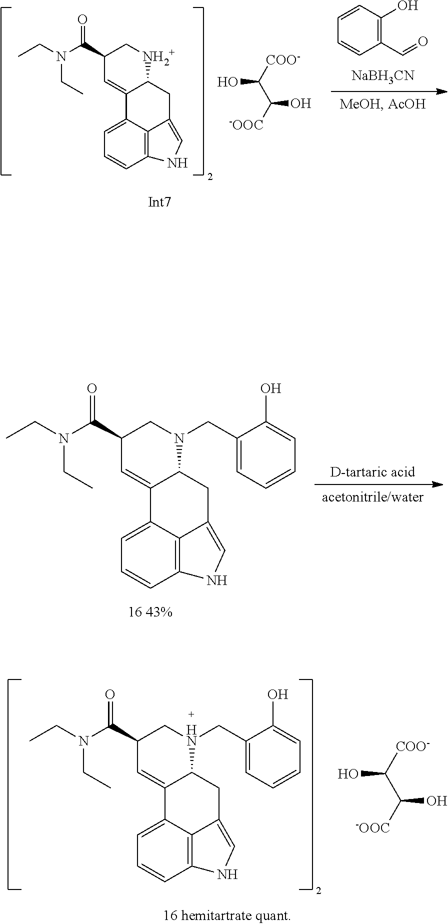

### Synthetic Protocols:

A solution of (6aR,9R)—N,N-diethyl-4,6,6a,7,8,9-hexahydroindolo[4,3-fg]quinoline-9-carboxamide hemitartrate (Int7, 20.0 mg, 52.0 μmol; 2 moles Int7 per mole tartrate) and 2-hydroxybenzaldehyde (22.0 μL, 0.208 mmol) in methanol (2 mL) was purged with argon and cooled to 0° C. Sodium cyanoborohydride (13.0 mg, 0.208 mmol) was introduced and the resulting mixture was stirred for 5 minutes followed by addition of glacial acetic acid (20 μL). After stirring at 0° C. for 3 h, the solvents were removed in vacuo, the residue was partitioned between dichloromethane (100 mL) and an aqueous 1% solution of ammonium hydroxide (150 mL), and the aqueous phase was further extracted with dichloromethane (3×50 mL). The combined organic phases were dried over sodium sulfate and concentrated in vacuo. The residue was purified by flash column chromatography (silica gel 60, 0.040-0.063 mm; eluent: dichloromethane/methanol 98:2) to give a material still containing impurities. This crude material was dissolved in 1M hydrochloric acid (25 mL) and methanol (5 mL), washed with diethyl ether (3×50 mL), basified with 24% aqueous ammonium hydroxide, and extracted with dichloromethane (3×50 mL). The combined organic extracts were dried over anhydrous sodium sulfate and concentration in vacuo to provide (6aR,9R)—N,N-diethyl-7-(2-hydroxybenzyl)-4,6,6a,7,8,9-hexahydroindolo[4,3-fg]quinoline-9-carboxamide (16) as a colorless solid.

Yield: 9.2 mg (43%).

¹H NMR spectrum (300 MHz, CD₃CN, δ_{H}): 9.03 (s, 1H), 7.25 (dd, J=6.1, 2.6, 1H), 7.20-7.06 (m, 3H), 6.96 (t, J=1.7, 1H), 6.87-6.71 (m, 3H), 6.37 (s, 1H), 4.61 (d, J=14.2, 1H), 3.79-3.73 (m, 1H), 3.68 (dd, J=14.3, 5.1, 1H), 3.57 (d, J=14.3, 1H), 3.49 (ddd, J=11.5, 4.6, 2.5, 1H), 3.41 (dd, J=15.0, 7.4, 1H), 3.36-3.25 (m, 4H), 3.08 (dd, J=11.5, 4.4, 1H), 2.79 (dd, J=12.0, 2.2, 1H), 2.72 (dd, J=11.6, 9.2, 1H), 1.08 (dt, J=14.3, 7.1, 6H).

LC-MS purity: 99% (ELSD), 99% (UV, 310 nm).

LC-MS Rt (Sinergy Polar RP, 4.6 mm×150 mm, acetonitrile/water 30:70 to 100:0+0.1% HBFA in 10 min): 5.85 min.

LC-MS m/z: 416.1 (M+H)⁺.

(6aR,9R)—N,N-diethyl-7-(2-hydroxybenzyl)-4,6,6a,7,8,9-hexahydroindolo[4,3-fg]quinoline-9-carboxamide (16, 9.20 mg, 22.1 μmol) was dissolved in gradient-grade acetonitrile (5.0 mL) and treated with aqueous 1M D-(−)-tartaric acid solution (11.0 μL, 11.0 μmol). The solvents were removed in vacuo, and the obtained material was redissolved in dioxane (5.0 mL) and subjected to freeze drying at 0° C. to yield (6aR,9R)—N,N-diethyl-7-(2-hydroxybenzyl)-4,6,6a,7,8,9-hexahydroindolo[4,3-fg]quinoline-9-carboxamide hemitartrate (16 hemitartrate) as a fluffy off-white solid.

Yield: 13.3 mg (quantitative).

¹H NMR spectrum (300 MHz, MeOD, δ_{H}): 7.33-7.18 (m, 3H), 7.16-7.07 (m, 2H), 7.03 (d, J=1.0, 1H), 6.87 (t, J=7.7, 2H), 6.37 (s, 1H), 4.67 (d, J=13.6, 1H), 4.42 (s, 1H), 4.05 (d, J=13.6, 1H), 4.00-3.91 (m, 2H), 3.86 (dd, J=13.8, 5.0, 1H), 3.66-3.33 (m, J=7.9, 1.9, 6H), 3.12-2.91 (m, 2H), 1.18 (t, J=7.1, 3H), 1.12 (t, J=7.1, 3H).

LC-MS purity: 97% (ELSD), 90% (UV, 310 nm).

LC-MS Rt (Sinergy Polar RP, 4.6 mm×150 mm, acetonitrile/water 30:70 to 100:0+0.1% HBFA in 10 min): 5.84 min.

LC-MS m/z: 416.1 (M+H)⁺.

### Example 16: Preparation of (6aR,9R)—N,N-diethyl-7-(3-methoxybenzyl)-4,6,6a,7,8,9-hexahydroindolo[4,3-fg]quinoline-9-carboxamide (18)

### Reaction Scheme:

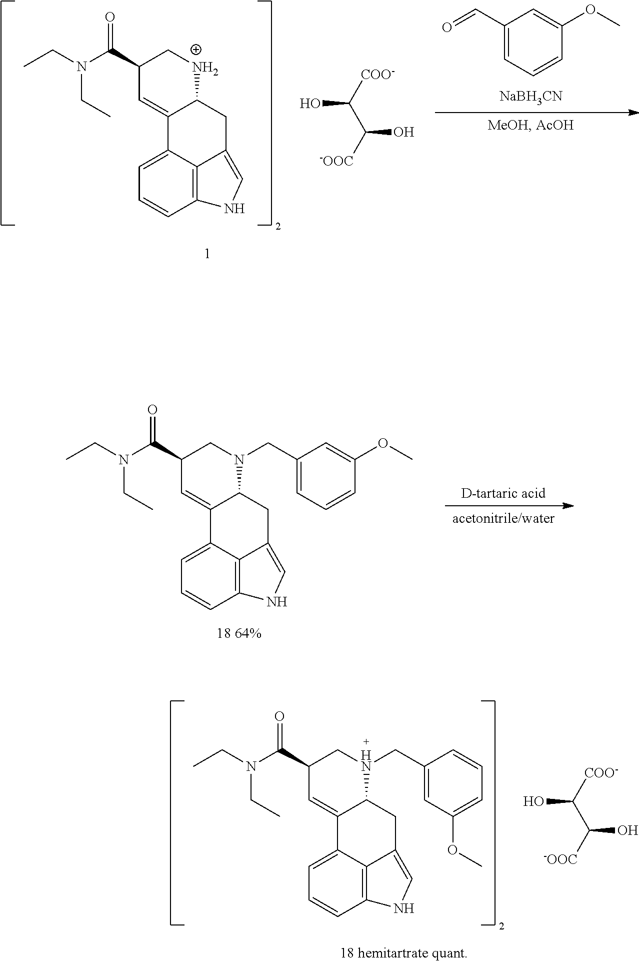

### Synthetic Protocols:

A solution of (6aR,9R)—N,N-diethyl-4,6,6a,7,8,9-hexahydroindolo[4,3-fg]quinoline-9-carboxamide hemitartrate (Int7, 20.0 mg, 52.0 μmol; 2 moles Int7 per mole tartrate) and 3-methoxybenzaldehyde (28.3 μL, 0.208 mmol) in methanol (2 mL) was purged with argon and cooled to 0° C. Sodium cyanoborohydride (13.0 mg, 0.208 mmol) was introduced and the resulting mixture was stirred for 5 minutes followed by addition of glacial acetic acid (20 μL). After stirring at 0° C. for 3 h, the solvents were removed in vacuo, the residue was partitioned between dichloromethane (100 mL) and a 1% aqueous solution of ammonium hydroxide (150 mL), and the aqueous phase was further extracted with dichloromethane (3×50 mL). The combined organic phases were dried over sodium sulfate and concentrated in vacuo. The residue was purified by flash column chromatography (silica gel 60, 0.040-0.063 mm; eluent: dichloromethane/methanol 98:2) to provide a material still containing impurities. This crude material was dissolved in 1M hydrochloric acid (25 mL) and methanol (5 mL), washed with diethyl ether (3×50 mL), basified with 24% aqueous ammonium hydroxide, and extracted with dichloromethane (3×50 mL). The combined organic extracts were dried over anhydrous sodium sulfate and concentrated in vacuo to give (6aR,9R)—N,N-diethyl-7-(3-methoxybenzyl)-4,6,6a,7,8,9-hexahydroindolo[4,3-fg]quinoline-9-carboxamide (18) as a colorless solid.

Yield: 14.2 mg (64%).

¹H NMR spectrum (300 MHz, CD₃CN, δ_{H}): 9.02 (s, 1H), 7.33-7.21 (m, 2H), 7.18-7.08 (m, 2H), 7.06-7.00 (m, 2H), 6.98 (t, J=1.7, 1H), 6.88-6.81 (m, 1H), 6.37 (s, 1H), 4.33 (d, J=14.1, 1H), 3.81 (s, 3H), 3.74-3.68 (m, 1H), 3.68 (dd, J=14.6, 5.4, 1H), 3.49-3.25 (m, 6H), 3.02 (ddd, J=11.1, 4.7, 1.0, 1H), 2.69 (ddd, J=14.6, 11.3, 1.7, 1H), 2.56 (t, J=10.5, 1H), 1.08 (td, J=7.1, 2.5, 6H).

LC-MS purity: 97% (ELSD), 99% (UV, 310 nm).

LC-MS Rt (Sinergy Polar RP, 4.6 mm×150 mm, acetonitrile/water 30:70 to 100:0+0.1% HBFA in 10 min): 6.31 min.

LC-MS m/z: 430.1 (M+H)⁺.

(6aR,9R)—N,N-diethyl-7-(3-methoxybenzyl)-4,6,6a,7,8,9-hexahydroindolo[4,3-fg]quinoline-9-carboxamide (18, 14.2 mg, 33.0 μmol) was dissolved in gradient-grade acetonitrile (5.0 mL) and treated with aqueous 1M D-(−)-tartaric acid solution (16.4 μL, 16.4 μmol). The solvents were removed in vacuo, and the obtained material was redissolved in dioxane (5.0 mL) and subjected to freeze drying at 0° C. to yield (6aR,9R)—N,N-diethyl-7-(3-methoxybenzyl)-4,6,6a,7,8,9-hexahydroindolo[4,3-fg]quinoline-9-carboxamide hemitartrate (18 hemitartrate) as a fluffy white solid.

Yield: 16.8 mg (quantitative).

¹H NMR spectrum (300 MHz, MeOD, δ_{H}): 7.30 (t, J=7.9, 1H), 7.22 (dd, J=6.5, 2.2, 1H), 7.15-7.00 (m, 5H), 6.91 (dd, J=8.2, 1.9, 1H), 6.35 (s, 1H), 4.46 (d, J=13.1, 1H), 4.44 (s, 1H), 3.91-3.84 (m, 2H), 3.81 (s, 3H), 3.83-3.78 (m, 2H), 3.51-3.33 (m, 4H), 3.21 (dd, J=11.4, 4.2, 1H), 2.92 (t, J=13.8, 1H), 2.82 (t, J=10.2, 1H), 1.13 (dt, J=14.4, 7.2, 6H).

LC-MS purity: 99% (ELSD), 96% (UV, 310 nm).

LC-MS Rt (Sinergy Polar RP, 4.6 mm×150 mm, acetonitrile/water 30:70 to 100:0+0.1% HBFA in 10 min): 6.31 min.

LC-MS m/z: 430.1 (M+H)⁺.

### Example 17: Preparation of (6aR,9R)—N,N-diethyl-7-(4-methoxybenzyl)-4,6,6a,7,8,9-hexahydroindolo[4,3-fg]quinoline-9-carboxamide (19)

### Reaction Scheme:

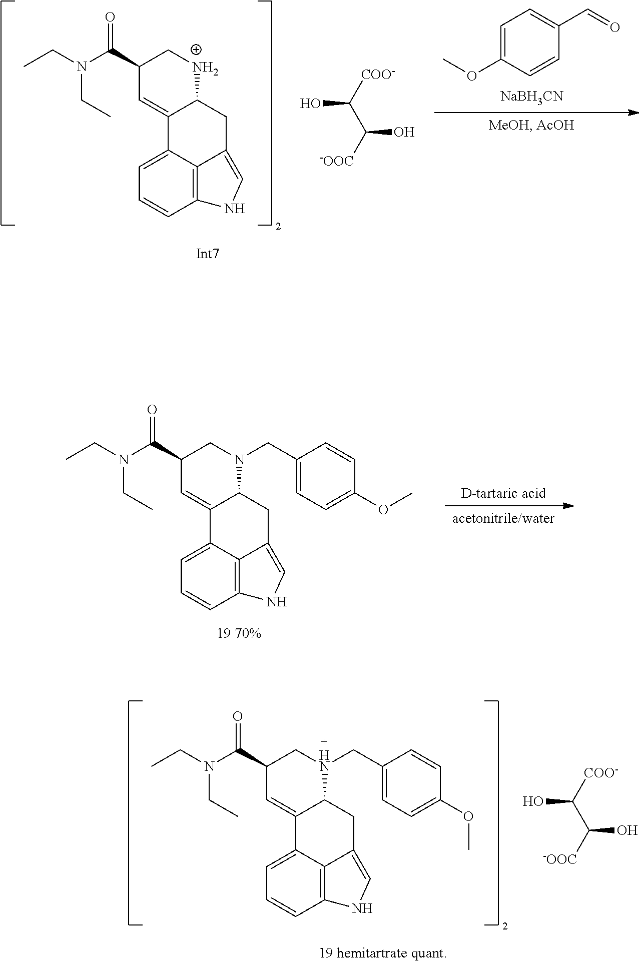

### Synthetic Protocols:

A solution of (6aR,9R)—N,N-diethyl-4,6,6a,7,8,9-hexahydroindolo[4,3-fg]quinoline-9-carboxamide hemitartrate (Int7, 20.0 mg, 52.0 μmol; 2 moles Int7 per mole tartrate) and p-anisaldehyde (22.0 μL, 0.208 mmol) in methanol (2 mL) was purged with argon and cooled to 0° C. Sodium cyanoborohydride (13.0 mg, 0.208 mmol) was introduced and the resulting mixture was stirred for 5 minutes followed by addition of glacial acetic acid (20 μL). After stirring at 0° C. for 3 h, the solvents were removed in vacuo, the residue was partitioned between dichloromethane (100 mL) and a 1% aqueous solution of ammonium hydroxide (150 mL), and the aqueous phase was further extracted with dichloromethane (3×50 mL). The combined organic phases were dried over sodium sulfate and concentrated in vacuo. The residue was purified by flash column chromatography (silica gel 60, 0.040-0.063 mm; eluent: dichloromethane/methanol 98:2) to provide a material still containing impurities. This crude material was dissolved in 1M hydrochloric acid (25 mL) and methanol (5 mL), washed with diethyl ether (3×50 mL), basified with 24% aqueous ammonium hydroxide, and extracted with dichloromethane (3×50 mL). The combined organic extracts were dried over anhydrous sodium sulfate and concentration in vacuo to provide (6aR,9R)—N,N-diethyl-7-(4-methoxybenzyl)-4,6,6a,7,8,9-hexahydroindolo[4,3-fg]quinoline-9-carboxamide (19) as a colorless solid.

Yield: 15.6 mg (70%).

¹H NMR spectrum (300 MHz, CD₃CN, δ_{H}): 9.01 (s, 1H), 7.31 (d, J=8.6, 2H), 7.23 (dd, J=6.7, 1.9, 1H), 7.14-7.05 (m, 2H), 6.96 (t, J=1.7, 1H), 6.89 (d, J=10.9, 2H), 6.32 (s, 1H), 4.24 (d, J=13.7, 1H), 3.77 (s, 3H), 3.69 (dd, J=14.7, 5.3, 1H), 3.63-3.58 (m, 1H), 3.45-3.18 (m, 6H), 2.99 (ddd, J=11.1, 4.7, 0.9, 1H), 2.66 (ddd, J=14.5, 11.3, 1.6, 1H), 2.49 (t, J=10.6, 1H), 1.05 (t, J=7.1, 6H).

LC-MS purity: 97% (ELSD), 99% (UV, 310 nm).

LC-MS Rt (Sinergy Polar RP, 4.6 mm×150 mm, acetonitrile/water 30:70 to 100:0+0.1% HBFA in 10 min): 6.24 min.

LC-MS m/z: 430.1 (M+H)⁺.

(6aR,9R)—N,N-diethyl-7-(4-methoxybenzyl)-4,6,6a,7,8,9-hexahydroindolo[4,3-fg]quinoline-9-carboxamide (19, 15.6 mg, 36.3 μmol) was dissolved in gradient-grade acetonitrile (5.0 mL) and treated with aqueous 1M D-(−)-tartaric acid solution (18.2 μL, 18.2 μmol). The solvents were removed in vacuo, and the obtained material was redissolved in dioxane (5.0 mL) and subjected to freeze drying at 0° C. to yield (6aR,9R)—N,N-diethyl-7-(4-methoxybenzyl)-4,6,6a,7,8,9-hexahydroindolo[4,3-fg]quinoline-9-carboxamide hemitartrate (19 hemitartrate) as a fluffy white solid.

Yield: 18.4 mg (quantitative).

¹H NMR spectrum (300 MHz, MeOD, δ_{H}): 7.44 (d, J=8.6, 2H), 7.25 (dd, J=5.4, 3.4, 1H), 7.17-7.10 (m, 2H), 7.07 (d, J=1.1, 1H), 6.98 (d, J=8.7, 2H), 6.36 (s, 1H), 4.48 (d, J=13.5, 1H), 4.45 (s, 1H), 4.02-3.97 (m, 1H), 3.97-3.88 (m, 3H), 3.81 (s, 3H), 3.48 (dd, J=14.8, 7.6, 2H), 3.45 (ddd, J=14.6, 13.5, 7.4, 2H), 3.30-3.27 (m, 1H), 3.09-2.83 (m, 2H), 1.17 (dt, J=14.2, 7.1, 6H).

LC-MS Rt (Sinergy Polar RP, 4.6 mm×150 mm, acetonitrile/water 30:70 to 100:0+0.1% HBFA in 10 min): 6.24 min.

LC-MS m/z: 430.1 (M+H)⁺.

### Example 18: Preparation of (6aR,9R)—N,N-diethyl-7-(3-methoxyphenethyl)-4,6,6a,7,8,9-hexahydroindolo[4,3-fg]quinoline-9-carboxamide (20)

### Reaction Scheme:

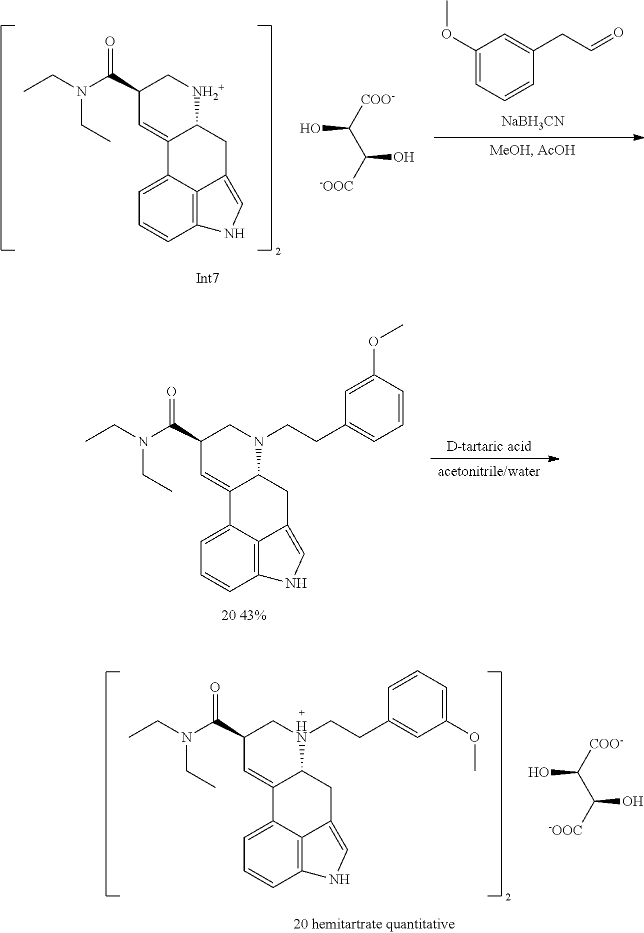

### Synthetic Protocols:

A solution of (6aR,9R)—N,N-diethyl-4,6,6a,7,8,9-hexahydroindolo[4,3-fg]quinoline-9-carboxamide hemitartrate (Int7, 30.0 mg, 78.0 μmol; 2 moles Int7 per mole tartrate) and 2-(3-methoxyphenyl)acetaldehyde (50.0 mg, 0.33 mmol) in methanol (2 mL) was purged with argon and cooled to 0° C. Sodium cyanoborohydride (20.0 mg, 0.32 mmol) was added and the resulting mixture was stirred for 5 minutes followed by addition of glacial acetic acid (20 μL). After stirring at 0° C. for 3 h, the solvents were removed in vacuo, the residue was partitioned between dichloromethane (100 mL0 and a 1% aqueous solution of ammonium hydroxide (150 mL), and the aqueous phase was further extracted with dichloromethane (3×50 mL). The combined organic phases were dried over sodium sulfate and concentrated in vacuo. The residue was purified by flash column chromatography (silica gel 60, 0.040-0.063 mm; eluent: dichloromethane/methanol 98:2) to provide a material still containing impurities. This crude material was dissolved in 1M hydrochloric acid (25 mL) and methanol (5 mL), washed with diethyl ether (3×50 mL), basified with 24% aqueous ammonium hydroxide, and extracted with dichloromethane (3×50 mL). The combined organic extracts were dried over anhydrous sodium sulfate and concentrated in vacuo to provide (6aR,9R)—N,N-diethyl-7-(3-methoxyphenethyl)-4,6,6a,7,8,9-hexahydroindolo[4,3-fg]quinoline-9-carboxamide (20) as a colorless foam.

Yield: 14.9 mg (43%).

¹H NMR spectrum (300 MHz, CD₃CN, δ_{H}): 8.99 (s, 1H), 7.27-7.16 (m, 2H), 7.14-7.05 (m, 2H), 6.95 (t, J=1.7, 1H), 6.87 (dd, J=4.0, 2.2, 2H), 6.76 (ddd, J=8.3, 2.5, 0.9, 1H), 6.30 (s, 1H), 3.77 (s, 3H), 3.74-3.65 (m, 1H), 3.55 (dd, J=14.5, 5.3, 1H), 3.50-3.29 (m, 5H), 3.22-3.10 (m, 2H), 2.92-2.69 (m, 4H), 2.48 (ddd, J=14.3, 11.0, 1.6, 1H), 1.21 (t, J=7.1, 3H), 1.11 (t, J=7.1, 3H).

LC-MS purity: 99% (ELSD), 99% (UV, 310 nm).

LC-MS Rt (Sinergy Polar RP, 4.6 mm×150 mm, acetonitrile/water 30:70 to 100:0+0.1% HBFA in 10 min): 6.58 min.

LC-MS m/z: 444.2 (M+H)⁺.

(6aR,9R)—N,N-diethyl-7-(3-methoxyphenethyl)-4,6,6a,7,8,9-hexahydroindolo[4,3-fg]quinoline-9-carboxamide (20, 14.9 mg, 33.6 μmol) was dissolved in gradient-grade acetonitrile (5.0 mL) and treated with aqueous 1M D-(−)-tartaric acid solution (16.8 μL, 16.8 μmol). The solvents were removed in vacuo, and the obtained material was redissolved in dioxane (5.0 mL) and subjected to freeze drying at 0° C. to yield (6aR,9R)—N,N-diethyl-7-(3-methoxyphenethyl)-4,6,6a,7,8,9-hexahydroindolo[4,3-fg]quinoline-9-carboxamide hemitartrate (20 hemitartrate) as a fluffy off-white solid.

Yield: 17.4 mg (quantitative).

¹H NMR spectrum (300 MHz, MeOD, δ_{H}): 7.28-7.20 (m, 2H), 7.14-7.08 (m, 2H), 7.02 (d, J=1.0, 1H), 6.92-6.87 (m, 2H), 6.80 (dd, J=8.3, 1.5, 1H), 6.37 (s, 1H), 4.41 (s, 1H), 4.08-3.96 (m, 2H), 3.79 (s, 3H), 3.68-3.49 (m, 5H), 3.49-3.38 (m, 4H), 3.10-2.98 (m, 2H), 2.91 (t, J=12.9, 1H), 1.32 (t, J=7.2, 3H), 1.19 (t, J=7.1, 3H).

LC-MS purity: 96% (ELSD), 90% (UV, 310 nm).

LC-MS Rt (Sinergy Polar RP, 4.6 mm×150 mm, acetonitrile/water 30:70 to 100:0+0.1% HBFA in 10 min): 6.58 min.

LC-MS m/z: 444.1 (M+H)⁺.

### Example 19: Preparation of (6aR,9R)—N,N-diethyl-7-(4-methoxyphenethyl)-4,6,6a,7,8,9-hexahydroindolo[4,3-fg]quinoline-9-carboxamide (21)

### Reaction Scheme:

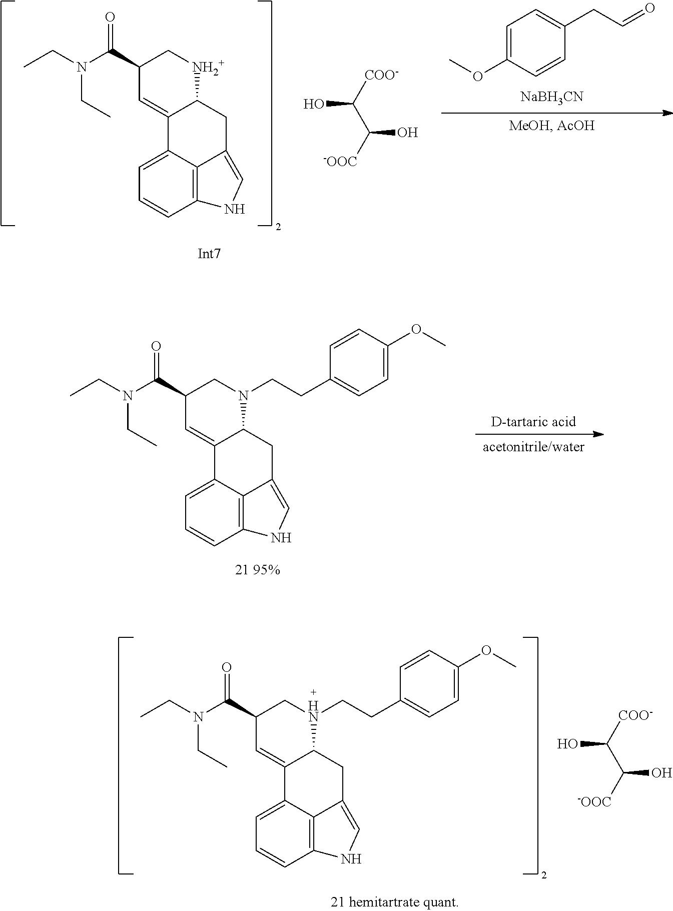

### Synthetic Protocols:

A solution of (6aR,9R)—N,N-diethyl-4,6,6a,7,8,9-hexahydroindolo[4,3-fg]quinoline-9-carboxamide hemitartrate (Int7, 33.0 mg, 86.0 μmol; 2 moles Int7 per mole tartrate) and 2-(4-methoxyphenyl)acetaldehyde (50.0 mg, 0.33 mmol) in methanol (2 mL) was purged with argon and cooled to 0° C. Sodium cyanoborohydride (20.0 mg, 0.32 mmol) was added and the resulting mixture was stirred for 5 minutes followed by addition of glacial acetic acid (20 μL). After stirring at 0° C. for 3 h, the solvents were removed in vacuo, the residue was partitioned between dichloromethane (100 mL) and a 1% aqueous solution of ammonium hydroxide (150 mL), and the aqueous phase was further extracted with dichloromethane (3×50 mL). The combined organic phases were dried over sodium sulfate and concentrated in vacuo. The residue was purified by flash column chromatography (silica gel 60, 0.040-0.063 mm; eluent: dichloromethane/methanol 98:2) to provide a material still containing impurities. This crude material was dissolved in 1M hydrochloric acid (25 mL) and methanol (5 mL), washed with diethyl ether (3×50 mL), basified with 24% aqueous ammonium hydroxide, and extracted with dichloromethane (3×50 mL). The combined organic extracts were dried over anhydrous sodium sulfate and concentrated in vacuo to provide (6aR,9R)—N,N-diethyl-7-(4-methoxyphenethyl)-4,6,6a,7,8,9-hexahydroindolo[4,3-fg]quinoline-9-carboxamide (21) as a colorless foam.

Yield: 36.2 mg (95%).

¹H NMR spectrum (300 MHz, CD₃CN, δ_{H}): 9.04 (s, 1H), 7.25-7.17 (m, 3H), 7.13-7.05 (m, 2H), 6.95 (t, J=1.6, 1H), 6.90-6.81 (m, 2H), 6.32 (s, 1H), 3.80-3.75 (m, 1H), 3.75 (s, J=3.0, 3H), 3.53 (dd, J=15.8, 5.4, 1H), 3.48-3.32 (m, 5H), 3.23-3.07 (m, 2H), 2.93-2.74 (m, 4H), 2.55 (ddd, J=15.6, 12.6, 1.6, 1H), 1.21 (t, J=7.1, 3H), 1.11 (t, J=7.1, 3H).

LC-MS purity: 99% (ELSD), 99% (UV, 310 nm).

LC-MS Rt (Sinergy Polar RP, 4.6 mm×150 mm, acetonitrile/water 30:70 to 100:0+0.1% HBFA in 10 min): 6.55 min.

LC-MS m/z: 444.2 (M+H)⁺.

(6aR,9R)—N,N-diethyl-7-(4-methoxyphenethyl)-4,6,6a,7,8,9-hexahydroindolo[4,3-fg]quinoline-9-carboxamide (21, 36.2 mg, 81.6 μmol) was dissolved in gradient-grade acetonitrile (5.0 mL) and treated with aqueous 1M D-(−)-tartaric acid solution (40.8 μL, 40.80 μmol). The solvents were removed in vacuo, and the obtained material was redissolved in dioxane (5.0 mL) and subjected to freeze drying at 0° C. to yield (6aR,9R)—N,N-diethyl-7-(4-methoxyphenethyl)-4,6,6a,7,8,9-hexahydroindolo[4,3-fg]quinoline-9-carboxamide hemitartrate (21 hemitartrate) as a fluffy white solid.

Yield: 42.3 mg (quantitative).

¹H NMR spectrum (300 MHz, MeOD, δ_{H}): 7.28-7.19 (m, 3H), 7.16-7.08 (m, 2H), 7.03 (d, J=0.8, 2H), 6.89 (d, J=8.6, 2H), 6.39 (s, 1H), 4.42 (s, 1H), 4.18-4.03 (m, 2H), 3.77 (s, 3H), 3.68-3.54 (m, 2H), 3.54-3.40 (m, 4H), 3.10-2.89 (m, 3H), 1.32 (t, J=7.2, 3H), 1.20 (t, J=7.1, 3H).

LC-MS Rt (Sinergy Polar RP, 4.6 mm×150 mm, acetonitrile/water 30:70 to 100:0+0.1% HBFA in 10 min): 6.57 min.

LC-MS m/z: 444.1 (M+H)⁺.

### Example 20: Preparation of (6aR,9R)—N,N-diethyl-7-(pyridin-2-ylmethyl)-4,6,6a,7,8,9-hexahydroindolo[4,3-fg]quinoline-9-carboxamide (22)

### Reaction Scheme:

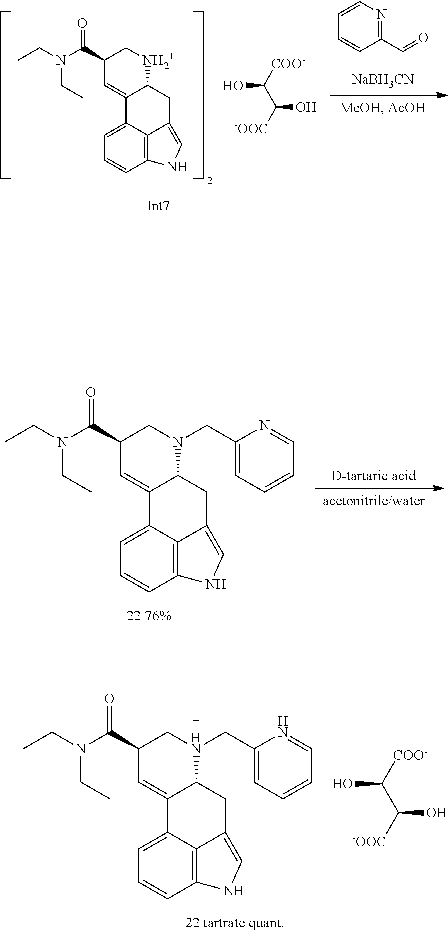

### Synthetic Protocols:

A solution of (6aR,9R)—N,N-diethyl-4,6,6a,7,8,9-hexahydroindolo[4,3-fg]quinoline-9-carboxamide hemitartrate (Int7, 20.0 mg, 52.0 μmol; 2 moles Int7 per mole tartrate) and pyridine-2-carbaldehyde (20.0 μL, 0.208 mmol) in methanol (2 mL) was purged with argon and cooled to 0° C. Sodium cyanoborohydride (13.0 mg, 0.208 mmol) was introduced and the resulting mixture was stirred for 5 minutes followed by addition of glacial acetic acid (20 μL). After stirring at 0° C. for 3 h, the solvents were removed in vacuo, the residue was partitioned between dichloromethane (100 mL) and a 1% aqueous solution of ammonium hydroxide (150 mL), and the aqueous phase was further extracted with dichloromethane (3×50 mL). The combined organic phases were dried over sodium sulfate and concentrated in vacuo. The residue was purified by flash column chromatography (silica gel 60, 0.040-0.063 mm; eluent: dichloromethane/methanol 98:2) to give a material still containing impurities. This crude material was dissolved in 1M hydrochloric acid (25 mL) and methanol (5 mL), washed with diethyl ether (3×50 mL), basified with 24% aqueous ammonium hydroxide, and extracted with dichloromethane (3×50 mL). The combined organic extracts were dried over anhydrous sodium sulfate and concentrated in vacuo to provide (6aR,9R)—N,N-diethyl-7-(pyridin-2-ylmethyl)-4,6,6a,7,8,9-hexahydroindolo[4,3-fg]quinoline-9-carboxamide (22) as a dark-tinted amorphous solid.

Yield: 15.8 mg (76%).

¹H NMR spectrum (300 MHz, CD₃CN, δ_{H}): 9.02 (s, 1H), 8.51 (ddd, J=4.8, 1.6, 0.8, 1H), 7.73 (td, J=7.7, 1.8, 1H), 7.57 (d, J=7.8, 1H), 7.22 (dt, J=7.7, 3.9, 2H), 7.15-7.05 (m, 2H), 6.94 (t, J=1.7, 1H), 6.33 (s, 1H), 4.31 (d, J=14.8, 1H), 3.76-3.68 (m, 1H), 3.72 (d, J=14.8, 1H), 3.66 (dd, J=14.4, 5.1, 1H), 3.57-3.46 (m, 1H), 3.44-3.27 (m, 4H), 3.05 (dd, J=10.8, 4.3, 1H), 2.69 (t, J=10.5, 1H), 2.63 (ddd, J=14.4, 11.2, 1.7, 1H), 1.08 (dt, J=12.3, 7.1, 6H).

LC-MS purity: 99% (ELSD), 99% (UV, 310 nm).

LC-MS Rt (Sinergy Polar RP, 4.6 mm×150 mm, acetonitrile/water 30:70 to 100:0+0.1% HBFA in 10 min): 5.74 min.

LC-MS m/z: 401.1 (M+H)⁺.

(6aR,9R)—N,N-diethyl-7-(pyridin-2-ylmethyl)-4,6,6a,7,8,9-hexahydroindolo[4,3-fg]quinoline-9-carboxamide (22, 15.8 mg, 39.5 μmol) was dissolved in gradient-grade acetonitrile (5.0 mL) and treated with aqueous 1M D-(−)-tartaric acid solution (39.4 μL, 39.4 μmol). The solvents were removed in vacuo, and the obtained material was redissolved in dioxane (5.0 mL) and subjected to freeze drying at 0° C. to yield (6aR,9R)—N,N-diethyl-7-(pyridin-2-ylmethyl)-4,6,6a,7,8,9-hexahydroindolo[4,3-fg]quinoline-9-carboxamide tartrate (22 tartrate) as a fluffy off-white solid.

Yield: 21.8 mg (quantitative).

¹H NMR spectrum (300 MHz, MeOD, δ_{H}): 8.60 (d, J=4.2, 1H), 7.91 (td, J=7.7, 1.7, 1H), 7.67 (d, J=7.8, 1H), 7.41 (dd, J=7.0, 5.5, 1H), 7.23 (dd, J=6.8, 1.9, 1H), 7.16-7.07 (m, 2H), 7.00 (d, J=1.2, 1H), 6.39 (s, 1H), 4.63 (d, J=14.7, 1H), 4.49 (s, 2H), 4.21 (d, J=14.6, 1H), 4.08-3.93 (m, 2H), 3.73 (dd, J=14.0, 5.2, 1H), 3.57-3.34 (m, 5H), 3.13 (dd, J=11.6, 8.9, 1H), 3.03-2.90 (m, 1H), 1.22 (t, J=7.1, 3H), 1.15 (t, J=7.1, 3H).

LC-MS purity: 99% (ELSD), 96% (UV, 310 nm).

LC-MS Rt (Sinergy Polar RP, 4.6 mm×150 mm, acetonitrile/water 30:70 to 100:0+0.1% HBFA in 10 min): 5.74 min.

LC-MS m/z: 401.1 (M+H)⁺.

### Example 21: Preparation of (6aR,9R)—N,N-diethyl-7-(2-(pyridin-2-yl)ethyl)-4,6,6a,7,8,9-hexahydroindolo[4,3-fg]quinoline-9-carboxamide (23)

### Reaction Scheme:

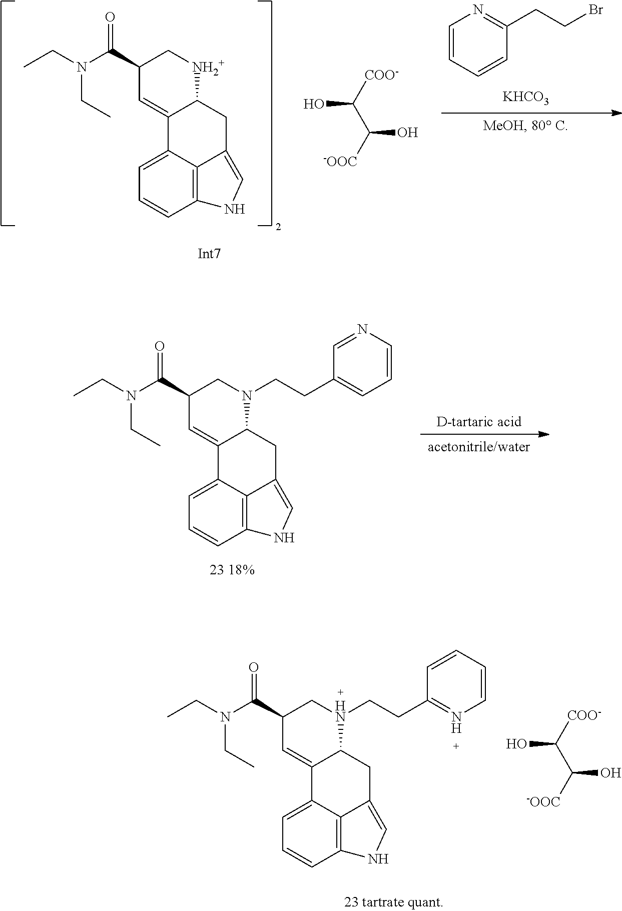

### Synthetic Protocols:

A solution of (6aR,9R)—N,N-diethyl-4,6,6a,7,8,9-hexahydroindolo[4,3-fg]quinoline-9-carboxamide hemitartrate (Int7, 20.0 mg, 53.0 μmol; 2 moles Int7 per mole tartrate), potassium bicarbonate (32 mg, 0.32 mmol), and 2-(2-bromoethyl)pyridin-1-ium bromide (28.0 mg, 0.33 mmol) in methanol (2 mL) was purged with argon and stirred at 80° C. for 4 days. After LC-MS analysis showed complete consumption of starting material, the reaction mixture was concentrated on silica gel and subjected to flash column chromatography (silica gel 60, 0.040-0.063 mm; eluent: dichloromethane/methanol 98:2). The combined product fractions were stripped of solvents in vacuo to yield (6aR,9R)—N,N-diethyl-7-(2-(pyridin-2-yl)ethyl)-4,6,6a,7,8,9-hexahydroindolo[4,3-fg]quinoline-9-carboxamide (23) as a dark-tinted amorphous solid.

Yield: 3.8 mg (18%).

¹H NMR spectrum (300 MHz, CD₃CN, δ_{H}): 8.97 (s, 1H), 8.53-8.49 (m, 1H), 7.65 (td, J=7.7, 1.9, 1H), 7.29 (d, J=7.8, 1H), 7.22 (dd, J=6.7, 2.0, 1H), 7.18-7.13 (m, 1H), 7.11-7.07 (m, 2H), 6.95 (t, J=1.8, 1H), 6.29 (s, 1H), 3.71-3.62 (m, 1H), 3.53 (dd, J=14.4, 5.3, 1H), 3.48-3.26 (m, 6H), 3.19 (dd, J=11.1, 3.8, 1H), 3.06-2.93 (m, 3H), 2.73 (t, J=10.7, 1H), 2.41 (ddd, J=14.3, 11.0, 1.6, 1H), 1.22 (t, J=7.1, 3H), 1.11 (t, J=7.1, 3H).

LC-MS purity: 98% (ELSD), 97% (UV, 310 nm).

LC-MS Rt (Sinergy Polar RP, 4.6 mm×150 mm, acetonitrile/water 30:70 to 100:0+0.1% HBFA in 10 min): 4.88 min.

LC-MS m/z: 415.1 (M+H)⁺.

(6aR,9R)—N,N-diethyl-7-(2-(pyridin-2-yl)ethyl)-4,6,6a,7,8,9-hexahydroindolo[4,3-fg]quinoline-9-carboxamide (23, 3.8 mg, 9.17 μmol) was dissolved in gradient-grade acetonitrile (5.0 mL) and treated with aqueous 1M D-(−)-tartaric acid solution (9.2 μL, 9.2 μmol). The solvents were removed in vacuo, and the obtained material was redissolved in dioxane (5.0 mL) and subjected to freeze drying at 0° C. to yield (6aR,9R)—N,N-diethyl-7-(2-(pyridin-2-yl)ethyl)-4,6,6a,7,8,9-hexahydroindolo[4,3-fg]quinoline-9-carboxamide tartrate (23 tartrate) as a fluffy light-brown solid.

Yield: 5.2 mg (quantitative).

¹H NMR spectrum (300 MHz, MeOD, δ_{H}): 8.52 (dd, J=4.9, 0.8, 1H), 7.80 (td, J=7.7, 1.8, 1H), 7.43 (d, J=7.8, 1H), 7.31 (ddd, J=7.5, 5.0, 0.9, 1H), 7.28-7.21 (m, 1H), 7.13 (dd, J=6.7, 5.6, 2H), 7.05 (d, J=1.0, 1H), 6.41 (dd, J=3.6, 1.7, 1H), 4.45 (s, 2H), 4.29-4.18 (m, 1H), 4.13-4.03 (m, 1H), 3.81-3.35 (m, 9H), 3.01 (t, J=12.9, 1H), 1.33 (t, J=7.1, 3H), 1.19 (t, J=7.1, 3H).

LC-MS purity: 95% (ELSD), 92% (UV, 310 nm).

LC-MS Rt (Sinergy Polar RP, 4.6 mm×150 mm, acetonitrile/water 30:70 to 100:0+0.1% HBFA in 10 min): 4.88 min.

LC-MS m/z: 415.1 (M+H)⁺.

### Example 22: Preparation of (6aR)—N—((R)-sec-butyl)-7-(2-methoxybenzyl)-4,6,6a,7,8,9-hexahydroindolo[4,3-fg]quinoline-9-carboxamide (24m)

### Reaction Scheme:

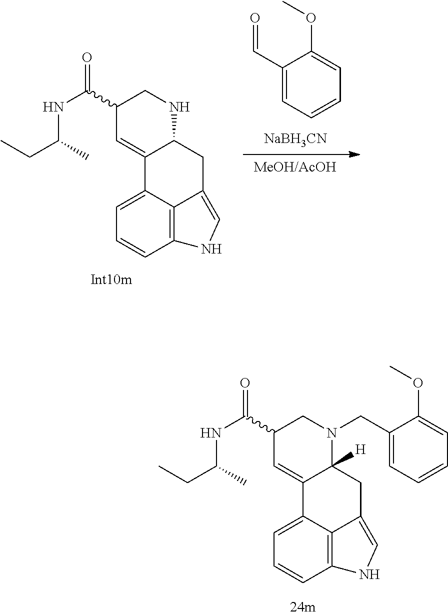

### Synthetic Protocols:

A solution of (6aR)—N—((R)-sec-butyl)-4,6,6a,7,8,9-hexahydroindolo[4,3-fg]quinoline-9-carboxamide (Int10m, 20 mg, 46.7 μmol; mixture of epimers at position 9) and 2-methoxybenzaldehyde (27 mg, 194 μmol) in methanol (0.5 mL) was purged with argon and cooled to 0° C. Sodium cyanoborohydride (12 mg, 194 μmol) was added and the resulting mixture was stirred for 5 minutes. Acetic acid (20 μL) was then introduced, and the reaction mixture was stirred at 0° C. for 24 h. There was obtained a diastereomeric mixture of (6aR)—N—((R)-sec-butyl)-7-(2-methoxybenzyl)-4,6,6a,7,8,9-hexahydroindolo[4,3-fg]quinoline-9-carboxamide (24m; mixture of diastereomers; epimers at position 9) as confirmed via LC-MS analysis.

Composition by LC-MS: 87% (ELSD, faster moving isomer), 13% (ELSD, slower moving isomer).

LC-MS Rt (Sinergy Polar RP, 4.6 mm×150 mm, acetonitrile/water 30:70 to 100:0+0.1% HBFA in 10 min): 6.54 min (isomer A), 6.57 min (isomer B).

LC-MS m/z: 430.2 (M+H)⁺.

### Example 23: Preparation of (6aR)—N—((R)-sec-butyl)-7-(2-methoxyphenethyl)-4,6,6a,7,8,9-hexahydroindolo[4,3-fg]quinoline-9-carboxamide (25m)

### Reaction Scheme:

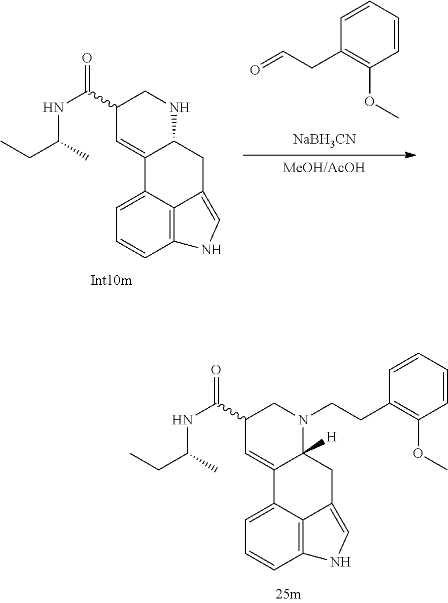

### Synthetic Protocols:

A solution of (6aR)—N—((R)-sec-butyl)-4,6,6a,7,8,9-hexahydroindolo[4,3-fg]quinoline-9-carboxamide (Int10m, 20 mg, 46.7 μmol; mixture of epimers at position 9) and 2-(2-methoxyphenyl)acetaldehyde (29 mg, 194 μmol) in methanol (0.5 mL) was purged with argon and cooled to 0° C. Sodium cyanoborohydride (12 mg, 194 μmol) was added and the resulting mixture was stirred for 5 minutes. Acetic acid (20 μL) was introduced, and the reaction mixture was then stirred at 0° C. for 24 h. There was obtained a diastereomeric mixture of (6aR)—N—((R)-sec-butyl)-7-(2-methoxyphenethyl)-4,6,6a,7,8,9-hexahydroindolo[4,3-fg]quinoline-9-carboxamide (25m; mixture of diastereomers; epimers at position 9) as confirmed via LC-MS analysis.

Composition by LC-MS: 87% (ELSD, faster moving isomer), 13% (ELSD, slower moving isomer).

LC-MS Rt (Sinergy Polar RP, 4.6 mm×150 mm, acetonitrile/water 30:70 to 100:0+0.1% HBFA in 10 min): 6.49 min (isomer A), 6.79 min (isomer B).

LC-MS m/z: 444.2 (M+H)⁺.

### Example 24: Preparation of ((2S,4S)-2,4-dimethylazetidin-1-yl)((6aR,9R)-7-(3-fluoropropyl)-4,6,6a,7,8,9-hexahydroindolo[4,3-fg]quinolin-9-yl)methanone (26)

### Reaction Scheme:

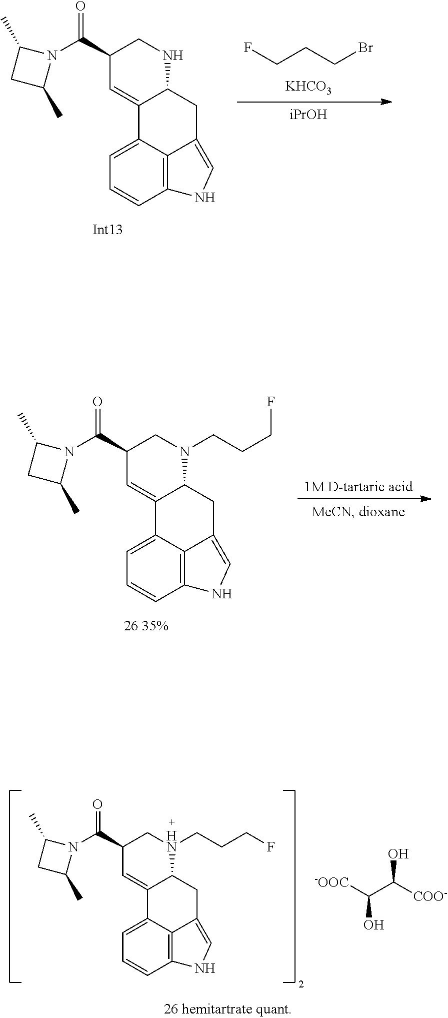

### Synthetic Protocols:

A solution of ((2S,4S)-2,4-dimethylazetidin-1-yl)((6aR,9R)-4,6,6a,7,8,9-hexahydroindolo[4,3-fg]quinolin-9-yl)methanone (Int13, 40 mg, 0.117 mmol), potassium bicarbonate (47 mg, 0.468 mg), and 1-bromo-3-fluoropropane (33 mg, 0.234 mmol) in isopropanol (1.0 mL) was purged with argon and heated to 90° C. in a sealed glass vial. The reaction mixture was stirred for 20 hours. The vial was opened, and the solvent was removed and evaporated in vacuo. The crude material was redissolved in dichloromethane (25 mL), treated with silica gel (silica gel 0.063-0.200 mm, 10 g), and concentrated. The resulting powder was added to a flash column and was purified by flash column chromatography (silica gel 60, 0.040-0.063 mm; eluent: dichloromethane/methanol 99:1 to 98:2) to afford ((2S,4S)-2,4-dimethylazetidin-1-yl)((6aR,9R)-7-(3-fluoropropyl)-4,6,6a,7,8,9-hexahydroindolo[4,3-fg]quinolin-9-yl)methanone (26) as a colorless foam.

Yield: 15.6 mg (35%).

¹H NMR spectrum (300 MHz, CDCl₃, δ_{H}): 8.99 (s, 1H), 7.22 (dd, J=6.6, 2.1, 1H), 7.16-7.03 (m, 2H), 6.94 (t, J=1.7, 1H), 6.30 (s, 1H), 4.73-4.55 (m, 2H), 4.55-4.44 (m, 1H), 4.43-4.33 (m, 1H), 3.50 (dd, J=14.6, 5.1, 1H), 3.41-3.29 (m, 2H), 3.18-3.02 (m, 2H), 2.66-2.43 (m, 3H), 2.01-1.91 (m, 2H), 2.05-1.86 (m, 2H), 1.46 (d, J=6.3, 3H), 1.39 (d, J=6.3, 3H).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 5.53 min.

LC-MS m/z: 382.1 (M+H)⁺.

((2S,4S)-2,4-dimethylazetidin-1-yl)((6aR,9R)-7-(3-fluoropropyl)-4,6,6a,7,8,9-hexahydroindolo[4,3-fg]quinolin-9-yl)methanone (26, 15.6 mg, 40.9 μmol) was dissolved in gradient-grade acetonitrile (5.0 mL) and treated with aqueous 1M D-(−)-tartaric acid solution (19.6 μL, 19.6 μmol). The resulting solution was stirred for 5 min. The solvent was removed in vacuo and the residue was re-dissolved in dioxane (5.0 mL) and subjected to freeze drying at 0° C. to afford ((2S,4S)-2,4-dimethylazetidin-1-yl)((6aR,9R)-7-(3-fluoropropyl)-4,6,6a,7,8,9-hexahydroindolo[4,3-fg]quinolin-9-yl)methanone hemitartrate (26 hemitartrate) as a fluffy white solid.

Yield: 18.6 mg (quantitative).

¹H NMR spectrum (300 MHz, MeOD, δ_{H}): 7.22 (dd, J=6.9, 1.8, 1H), 7.16-7.06 (m, 2H), 7.00 (d, J=1.2, 1H), 6.32 (s, 1H), 4.73 (dd, J=13.2, 6.2, 1H), 4.70-4.61 (m, 1H), 4.50 (dd, J=12.7, 6.3, 2H), 4.43 (s, 1H), 3.88-3.74 (m, 1H), 3.70-3.64 (m, 2H), 3.62 (dd, J=14.7, 5.4, 1H), 3.38 (dd, J=15.3, 4.2, 1H), 3.38-3.28 (m, 2H), 3.05 (t, J=9.8, 1H), 2.81 (t, J=12.6, 1H), 2.16-2.06 (m, 2H), 2.22-1.95 (m, 2H), 1.57 (d, J=6.3, 3H), 1.47 (d, J=6.3, 3H).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 5.53 min.

LC-MS m/z: 382.1 (M+H)⁺.

### Example 25: Preparation of ((6aR,9R)-7-allyl-4,6,6a,7,8,9-hexahydroindolo[4,3-fg]quinolin-9-yl)((2S,4S)-2,4-dimethylazetidin-1-yl)methanone (27)

### Reaction Scheme:

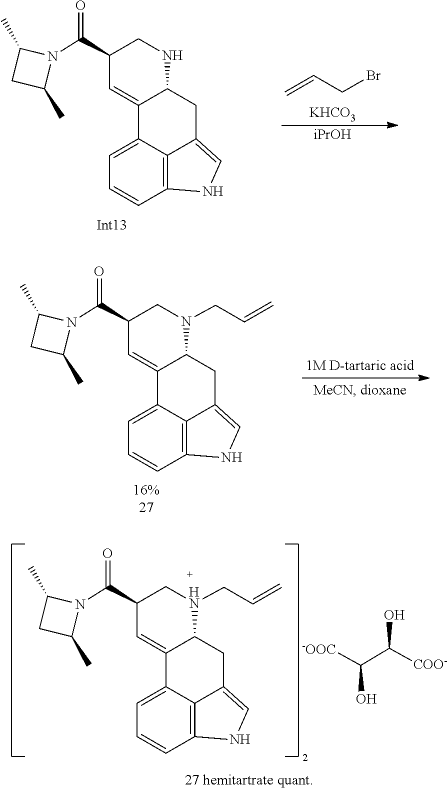

### Synthetic Protocol:

A solution of ((2S,4S)-2,4-dimethylazetidin-1-yl)((6aR,9R)-4,6,6a,7,8,9-hexahydroindolo[4,3-fg]quinolin-9-yl)methanone (4, 40.0 mg, 0.117 mmol), potassium bicarbonate (47 mg, 0.468 mg), and allyl bromide (20 μL, 0.234 mmol) in isopropanol (1.0 mL) was purged with argon and heated to 90° C. The reaction mixture was stirred for 20 h. The solvent was removed in vacuo and the crude material was redissolved in dichloromethane (25 mL). Silica gel (silica gel 0.063-0.200 mm, 10 g) was added, and the solvent was removed in vacuo. The resulting powder was placed on a preloaded silica gel flash column and eluted (silica gel 60, 0.040-0.063 mm; eluent: dichloromethane/methanol 99:1 to 98:2) to afford ((6aR,9R)-7-allyl-4,6,6a,7,8,9-hexahydroindolo[4,3-fg]quinolin-9-yl)((2S,4S)-2,4-dimethylazetidin-1-yl)methanone (27) as a colorless foam.

Yield: 14.2 mg (33%).

¹H NMR spectrum (300 MHz, CDCl₃, δ_{H}): 9.03 (s, 1H), 7.29-7.20 (m, 1H), 7.17-7.08 (m, 2H), 6.97 (t, J=1.8, 1H), 6.33 (s, 1H), 6.11-5.93 (m, 1H), 5.32 (dd, J=17.2, 1.1, 1H), 5.21 (d, J=10.1, 1H), 4.68-4.53 (m, 1H), 4.41 (dq, J=12.8, 6.2, 1H), 3.74-3.63 (m, 1H), 3.57 (dd, J=14.7, 5.2, 1H), 3.45-3.34 (m, 2H), 3.23-3.12 (m, 2H), 2.62 (t, J=12.0, 1H), 2.59-2.47 (m, 1H), 2.09-1.99 (m, 2H), 1.48 (d, J=6.3, 3H), 1.41 (d, J=6.3, 3H).

LC-MS purity: 98% (ELSD), 91% (UV, 310 nm).

LC-MS Rt (Sinergy Polar RP, 4.6 mm×150 mm, acetonitrile/water 30:70 to 100:0+0.1% HBFA in 10 min): 5.42 min.

LC-MS m/z: 362.1 (M+H)⁺.

((6aR,9R)-7-allyl-4,6,6a,7,8,9-hexahydroindolo[4,3-fg]quinolin-9-yl)((2S,4S)-2,4-dimethylazetidin-1-yl)methanone (27, 14.2 mg, 39.3 μmol) was dissolved in gradient-grade acetonitrile (5.0 mL) and treated with aqueous 1M D-(−)-tartaric acid solution (19.6 μL, 19.6 μmol). The resulting solution was stirred for 5 min and then the solvent was removed in vacuo. The residue was re-dissolved in dioxane (5.0 mL) and subjected to freeze drying at 0° C. to afford ((6aR,9R)-7-allyl-4,6,6a,7,8,9-hexahydroindolo[4,3-fg]quinolin-9-yl)((2S,4S)-2,4-dimethylazetidin-1-yl)methanone hemitartrate (27 hemitartrate) as a fluffy off-white solid.

Yield: 17.1 mg (quantitative).

¹H NMR spectrum (300 MHz, MeOD, δ_{H}): 7.23 (dd, J=7.2, 1.5, 1H), 7.16-7.07 (m, 2H), 7.01 (d, J=1.2, 1H), 6.33 (s, 1H), 6.14-5.96 (m, 1H), 5.49 (d, J=17.0, 1H), 5.42 (d, J=10.3, 1H), 4.77-4.63 (m, 1H), 4.56-4.44 (m, 1H), 4.44 (s, 1H), 3.92 (dd, J=14.1, 5.5, 1H), 3.86-3.76 (m, 1H), 3.73-3.53 (m, 4H), 3.40 (dd, J=11.5, 4.5, 1H), 3.00 (t, J=10.8, 1H), 2.82 (t, J=10.8, 1H), 2.20-2.01 (m, 2H), 1.57 (d, J=6.3, 3H), 1.46 (d, J=6.3, 3H).

LC-MS purity: 98% (ELSD), 91% (UV, 310 nm).

LC-MS Rt (Sinergy Polar RP, 4.6 mm×150 mm, acetonitrile/water 30:70 to 100:0+0.1% HBFA in 10 min): 5.42 min.

LC-MS m/z: 362.1 (M+H)⁺.

### Example 26: Preparation of (6aR,9R)-5-bromo-N,N-diethyl-7-methyl-4,6,6a,7,8,9-hexahydroindolo[4,3-fg]quinoline-9-carboxamide (35)

### Reaction Scheme:

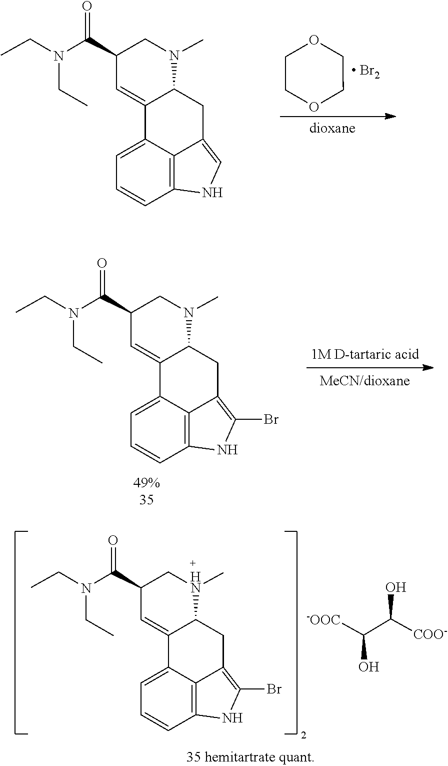

### Synthetic Protocols:

(6aR,9R)—N,N-Diethyl-7-methyl-4,6,6a,7,8,9-hexahydroindolo[4,3-fg]quinoline-9-carboxamide (54.0 mg, 0.167 mmol) was dissolved in anhydrous dioxane (2.0 mL) and flushed with argon. A solution of bromine in dioxane (10% v/v, 834 μL, 0.151 mmol) was added dropwise and the resulting mixture was stirred for 2 h. The mixture was then treated with silica gel (silica gel 0.063-0.200 mm, 10 g) and evaporated in vacuo. The resulting powder was added to the top of a flash chromatography column previously filled with silica gel and the product was eluted as follows (silica gel 60, 0.040-0.063 mm; eluent: dichloromethane/methanol 98:2) to give (6aR,9R)-5-bromo-N,N-diethyl-7-methyl-4,6,6a,7,8,9-hexahydroindolo[4,3-fg]quinoline-9-carboxamide (35) as a dark-tinted amorphous solid.

Yield: 32.8 mg (49%).

¹H NMR spectrum (300 MHz, CD₃CN, δ_{H}): 9.48 (s, 1H), 7.21-7.05 (m, 3H), 6.33 (s, 1H), 3.88-3.73 (m, 1H), 3.45 (q, J=7.1, 2H), 3.36 (ddd, J=9.0, 6.4, 2.2, 3H), 3.11-3.05 (m, 1H), 3.02 (dd, J=11.7, 4.4, 1H), 2.64 (t, J=10.8, 1H), 2.51 (s, 3H), 2.41 (dd, J=14.9, 11.3, 1H), 1.20 (t, J=7.0, 3H), 1.10 (t, J=7.1, 3H).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 5.37 min.

LC-MS m/z: 403.9 (M+H)⁺.

(6aR,9R)-5-bromo-N,N-diethyl-7-methyl-4,6,6a,7,8,9-hexahydroindolo[4,3-fg]quinoline-9-carboxamide (35, 32.8 mg, 81.5 μmol) was dissolved in gradient-grade acetonitrile (5.0 mL) and treated with aqueous 1M D-(−)-tartaric acid solution (40.8 μL, 40.8 μmol). The mixture was stirred at room temperature and then evaporated in vacuo. The residue was re-dissolved in dioxane (5.0 mL) and subjected to freeze drying at 0° C. to afford (6aR,9R)-5-bromo-N,N-diethyl-7-methyl-4,6,6a,7,8,9-hexahydroindolo[4,3-fg]quinoline-9-carboxamide hemitartrate (35 hemitartrate) as a fluffy light-brown solid.

Yield: 38.8 mg (quantitative).

¹H NMR spectrum (300 MHz, MeOD, δ_{H}): 7.21-7.06 (m, 3H), 6.39 (s, 1H), 4.42 (s, 1H), 4.16-4.04 (m, 1H), 3.76-3.66 (m, 1H), 3.62-3.41 (m, 5H), 3.41-3.33 (m, 1H), 3.20-3.09 (m, 1H), 2.87 (s, 3H), 2.72 (dd, J=14.5, 11.6, 1H), 1.31 (t, J=7.1, 3H), 1.18 (t, J=7.1, 3H).

LC-MS purity: 100% (ELSD), 97% (UV, 310 nm).

LC-MS Rt (Sinergy Polar RP, 4.6 mm×150 mm, acetonitrile/water 30:70 to 100:0+0.1% HBFA in 10 min): 5.37 min.

LC-MS m/z: 403.9 (M+H)⁺.

### Example 27: (6aR,9R)—N,N-diethyl-7-propyl-4,5,5a,6,6a,7,8,9-octahydroindolo[4,3-fg]quinoline-9-carboxamide (36)

### Reaction Scheme:

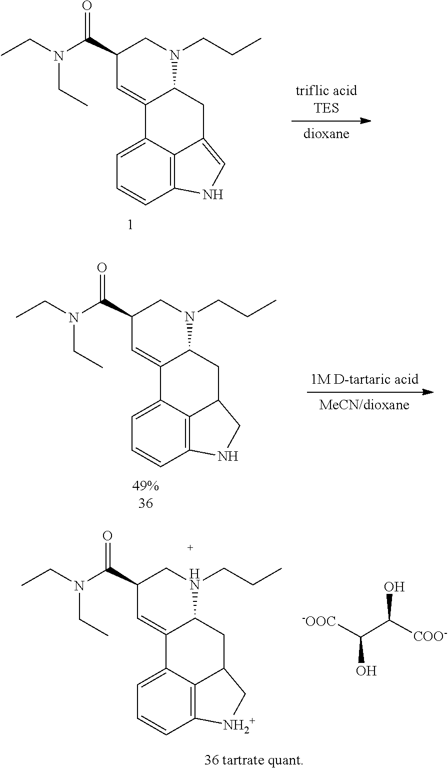

### Synthetic Protocols:

(6aR,9R)—N,N-Diethyl-7-propyl-4,6,6a,7,8,9-hexahydroindolo[4,3-fg]quinoline-9-carboxamide (1, 200 mg, 0.567 mmol) was dissolved in anhydrous dioxane (10 mL) followed by the addition of triethylsilane (1 mL) and triflic acid (500 μL), and the resulting mixture was stirred at 40° C. for 96 h. The mixture was then allowed to cool to room temperature and silica gel (silica gel 0.063-0.200 mm, 10 g) was added. The mixture was concentrated in vacuo and the resulting powder was added to the top of a flash chromatography column previously filled with silica gel and the product was eluted as follows (silica gel 60, 0.040-0.063 mm; eluent: dichloromethane/methanol 98:2) to give (6aR,9R)—N,N-diethyl-7-propyl-4,5,5a,6,6a,7,8,9-octahydroindolo[4,3-fg]quinoline-9-carboxamide (36; mixture of diastereomers; epimers at position 5a) as a dark-tinted amorphous solid.

Yield: 97.6 mg (49%).

LC-MS purity: 100% (ELSD), 98% (UV, 310 nm). LC-MS Rt (Sinergy Polar RP, 4.6 mm×150 mm, acetonitrile/water 30:70 to 100:0+0.1% HBFA in 10 min): 3.85 min.

LC-MS m/z: 354.2 (M+H)⁺.

(6aR,9R)—N,N-diethyl-7-propyl-4,5,5a,6,6a,7,8,9-octahydroindolo[4,3-fg]quinoline-9-carboxamide (36, 16.3 mg, 46.1 μmol; mixture of epimers at position 5a) was dissolved in gradient-grade acetonitrile (5.0 mL) and treated with aqueous 1M D-(−)-tartaric acid solution (50 μL, 50.0 μmol). The solvent was removed in vacuo. The residue was re-dissolved in dioxane (5.0 mL) and subjected to freeze drying at 0° C. to yield (6aR,9R)—N,N-diethyl-7-propyl-4,5,5a,6,6a,7,8,9-octahydroindolo[4,3-fg]quinoline-9-carboxamide tartrate (36 tartrate; mixture of diastereomers; epimers at position 5a) as a fluffy brown solid.

Yield: 24.0 mg (quantitative).

¹H NMR spectrum (300 MHz, MeOD, δ_{H}): 7.11-6.94 (m, 2H), 6.57 (dd, J=5.3, 2.9, 1H), 6.37 (s, 1H), 4.42 (s, 2H), 4.22-4.06 (m, 2H), 3.75-3.67 (m, 1H), 3.66-3.61 (m, 2H), 3.56 (dd, J=14.8, 7.4, 2H), 3.50-3.40 (m, 1H), 3.44 (dt, J=11.5, 6.3, 2H), 3.31-3.22 (m, 2H), 3.21-3.06 (m, 2H), 2.81-2.67 (m, 1H), 1.93-1.73 (m, 2H), 1.65 (dd, J=23.6, 11.7, 1H), 1.31 (t, J=7.1, 3H), 1.17 (t, J=7.1, 3H), 1.05 (t, J=7.3, 3H).

LC-MS purity: 100% (ELSD), 98% (UV, 310 nm).

LC-MS Rt (Sinergy Polar RP, 4.6 mm×150 mm, acetonitrile/water 30:70 to 100:0+0.1% HBFA in 10 min): 3.85 min.

LC-MS m/z: 354.2 (M+H)⁺.

### Example 28: Preparation of (6aR,9R)—N,N-diethyl-7-methyl-4,5,5a,6,6a,7,8,9-octahydroindolo[4,3-fg]quinoline-9-carboxamide (37)

### Reaction Scheme:

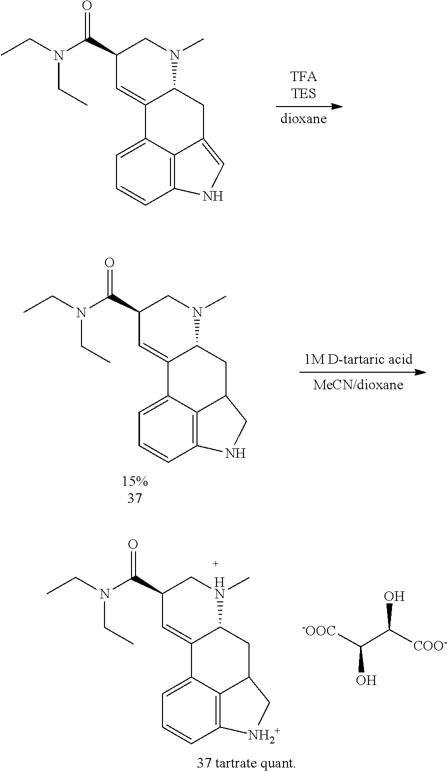

### Synthetic Protocols:

(6aR,9R)—N,N-Diethyl-7-methyl-4,6,6a,7,8,9-hexahydroindolo[4,3-fg]quinoline-9-carboxamide (108 mg, 0.334 mmol) was dissolved in trifluoroacetic acid (10 mL) followed by the addition of triethylsilane (1 mL). The resulting mixture was stirred at 40° C. for 72 h. The mixture was then allowed to cool to room temperature and silica gel (silica gel 0.063-0.200 mm, 10 g) was added. The mixture was concentrated in vacuo. The resulting powder was added to the top of a flash chromatography column previously filled with silica gel and the product was eluted as follows (silica gel 60, 0.040-0.063 mm; eluent: dichloromethane/methanol 98:2) to give (6aR,9R)—N,N-diethyl-7-methyl-4,5,5a,6,6a,7,8,9-octahydroindolo[4,3-fg]quinoline-9-carboxamide (37; mixture of diastereomers; epimers at position 5a) as a dark-tinted amorphous solid.

Yield: 16.3 mg (15%).

LC-MS purity: 92% (ELSD), 77% (UV, 310 nm).

LC-MS Rt (Sinergy Polar RP, 4.6 mm×150 mm, acetonitrile/water 30:70 to 100:0+0.1% HBFA in 10 min): 3.46 min.

LC-MS m/z: 326.1 (M+H)⁺.

(6aR,9R)—N,N-diethyl-7-methyl-4,5,5a,6,6a,7,8,9-octahydroindolo[4,3-fg]quinoline-9-carboxamide (37, 16.3 mg, 50.0 μmol; mixture of epimers at position 5a) was dissolved in gradient-grade acetonitrile (5.0 mL) and treated with aqueous 1M D-(−)-tartaric acid solution (50 μL, 50.0 μmol). The solvents were removed in vacuo, and the material was re-dissolved in dioxane (5.0 mL) and subjected to freeze drying at 0° C. to afford (6aR,9R)—N,N-diethyl-7-methyl-4,5,5a,6,6a,7,8,9-octahydroindolo[4,3-fg]quinoline-9-carboxamide tartrate (37 tartrate; mixture of diastereomers; epimers at position 5a) as a fluffy brown solid.

Yield: 24.0 mg (quantitative).

LC-MS purity: 92% (ELSD), 77% (UV, 310 nm).

LC-MS Rt (Sinergy Polar RP, 4.6 mm×150 mm, acetonitrile/water 30:70 to 100:0+0.1% HBFA in 10 min): 3.46 min.

LC-MS m/z: 326.1 (M+H)⁺.

### Example 29: Preparation of (6aR,9R)—N,N-bis(2-fluoroethyl)-7-methyl-4,6,6a,7,8,9-hexahydroindolo[4,3-fg]quinoline-9-carboxamide (38)

### Reaction Scheme:

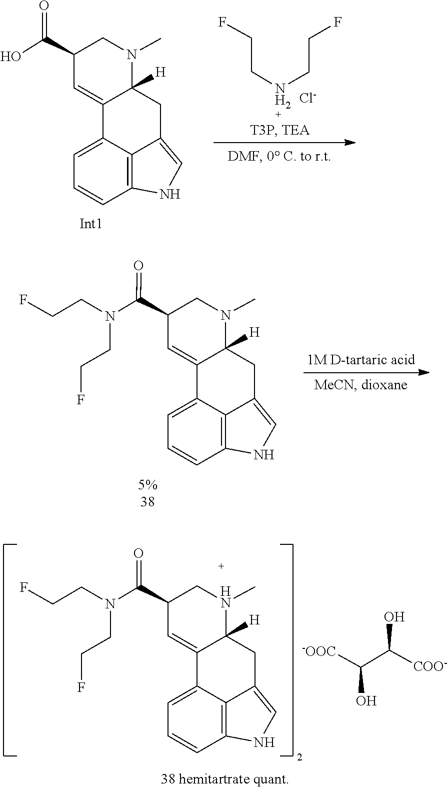

### Synthetic Protocols:

A solution of (6aR,9R)-7-methyl-4,6,6a,7,8,9-hexahydroindolo[4,3-fg]quinoline-9-carboxylic acid (Int1, 384 mg, 1.43 mmol), triethylamine (0.93 mL, 6.43 mmol), and bis(2-fluoroethyl)amine hydrochloride (250 mg, 1.71 mmol) in dry N,N-dimethylformamide (10 mL) was cooled to 0° C. under an argon atmosphere. Propanephosphonic acid anhydride (T3P®, 0.998 mL, 1.71 mmol, 50% solution in DMF) was added dropwise over 5 minutes. The resulting mixture was stirred for 3 h at 0° C. The reaction was judged to be complete by LC-MS and then it was quenched with ice-cold water (10 mL). The mixture was partitioned between 1M ammonium hydroxide solution (250 mL) and ethyl acetate (200 mL). The aqueous phase was re-extracted with ethyl acetate (2×200 mL), and the combined organic phases were washed with 10% aq. lithium chloride solution (4×150 mL) and dried over anhydrous magnesium sulfate. The solvent was filtered and concentrated in vacuo. The residue was purified by flash column chromatography (silica gel 60, 0.040-0.063 mm; eluent: dichloromethane/methanol 100:0 to 98:2). Leading fractions were cut to afford (6aR,9R)—N,N-bis(2-fluoroethyl)-7-methyl-4,6,6a,7,8,9-hexahydroindolo[4,3-fg]quinoline-9-carboxamide (38) as a colorless amorphous solid after evaporation of solvents.

Yield: 25 mg (5%).

¹H NMR spectrum (300 MHz, CD₃CN, δ_{H}): 9.01 (s, 1H), 7.23 (dd, J=6.0, 2.7, 1H), 7.14-7.06 (m, 2H), 6.95 (t, J=1.7, 1H), 6.32 (s, 1H), 4.74-4.62 (m, 2H), 4.59-4.47 (m, 2H), 3.98-3.86 (m, 2H), 3.80 (dd, J=9.2, 4.6, 1H), 3.75 (td, J=4.9, 1.9, 1H), 3.70-3.63 (m, 1H), 3.53 (dd, J=14.7, 5.6, 1H), 3.12-3.07 (m, 1H), 3.07-2.98 (m, 1H), 2.63 (t, J=10.7, 1H), 2.58-2.47 (m, 1H), 2.48 (s, 3H).

LC-MS purity: 100% (ELSD), 95% (UV, 310 nm).

LC-MS Rt (Sinergy Polar RP, 4.6 mm×150 mm, acetonitrile/water 30:70 to 100:0+0.1% HBFA in 10 min): 4.90 min.

LC-MS m/z: 360.1 (M+H)⁺.

(6aR,9R)—N,N-bis(2-fluoroethyl)-7-methyl-4,6,6a,7,8,9-hexahydroindolo[4,3-fg]quinoline-9-carboxamide (38, 25.0 mg, 69.6 μmol) was dissolved in gradient-grade acetonitrile (5.0 mL) and treated with aqueous 1M D-(−)-tartaric acid solution (34.8 μL, 34.8 μmol). The resulting mixture was stirred for an additional 5 minutes. The solvent was removed in vacuo, and the remaining material was re-dissolved in dioxane (5.0 mL) and subjected to freeze drying at 0° C. This afforded (6aR,9R)—N,N-bis(2-fluoroethyl)-7-methyl-4,6,6a,7,8,9-hexahydroindolo[4,3-fg]quinoline-9-carboxamide hemitartrate (38 hemitartrate) as a fluffy white solid.

Yield: 30.2 mg (quantitative).

¹H NMR spectrum (300 MHz, MeOD, δ_{H}): 7.24 (dd, J=6.8, 1.9, 1H), 7.16-7.07 (m, 2H), 7.02 (d, J=1.3, 1H), 6.42 (s, 1H), 4.77 (t, J=4.6, 1H), 4.70 (t, J=4.9, 1H), 4.61 (t, J=4.6, 1H), 4.55 (t, J=4.9, 1H), 4.40 (s, 1H), 4.34-4.24 (m, 1H), 4.02 (dd, J=9.7, 4.7, 1H), 3.93 (dd, J=10.2, 4.8, 1H), 3.84 (t, J=4.9, 1H), 3.81-3.73 (m, 2H), 3.72-3.63 (m, 2H), 3.43 (dd, J=11.7, 4.8, 1H), 3.19 (t, J=10.4, 1H), 2.89 (s, 3H), 2.93-2.82 (m, 1H).

LC-MS purity: 100% (ELSD), 95% (UV, 310 nm).

LC-MS Rt (Sinergy Polar RP, 4.6 mm×150 mm, acetonitrile/water 30:70 to 100:0+0.1% HBFA in 10 min): 4.90 min.

LC-MS m/z: 360.1 (M+H)⁺.

### Example 30. Preparation of (6aR,9R)—N,N-diethyl-7-(2-fluoroethyl)-4,6,6a,7,8,9-hexahydroindolo[4,3-fg]quinoline-9-carboxamide (39)

### Reaction Scheme:

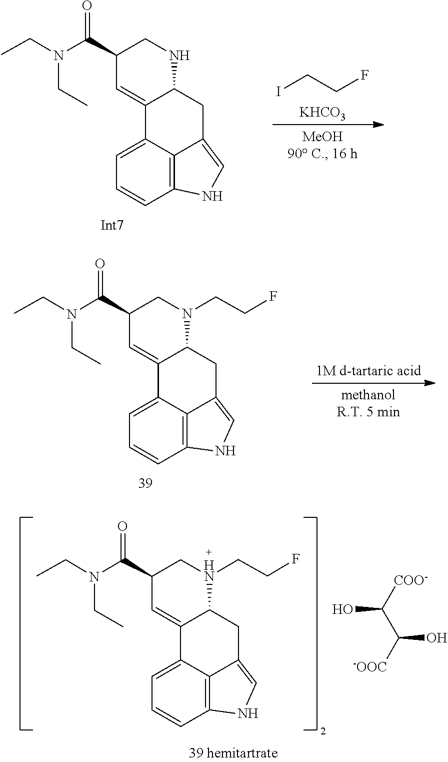

### Synthetic Protocols:

(6aR,9R)—N,N-diethyl-4,6,6a,7,8,9-hexahydroindolo[4,3-fg]quinoline-9-carboxamide (Int7, 30 mg, 96.0 μmol), potassium bicarbonate (33 mg, 323 μmol), and 1-iodo-2-fluoroethane (53 mg, 323 μmol) were mixed in 2-propanol (1 mL) under argon atmosphere in a sealed pressure vessel. The reaction was stirred at 90° C. for 16 h. After cooling to room temperature, the reaction was diluted with dichloromethane (20 mL) and silica gel (4 g) was added. This suspension was stripped of solvents in vacuo and subjected to silica gel chromatography (silica gel 60, 0.040-0.063 mm; eluent: dichloromethane/methanol/ammonia 98:2:0.1) to afford (6aR,9R)—N,N-diethyl-7-(2-fluoroethyl)-4,6,6a,7,8,9-hexahydroindolo[4,3-fg]quinoline-9-carboxamide (39, upper fluorescent band on TLC) as a colorless foam.

Yield: 30.8 mg (23%).

LC-MS purity: 99% (ELSD), 99% (UV, 310 nm).

LC-MS Rt (Sinergy Polar RP 4.6 mm×150 mm, acetonitrile/water 30:70 to 100:0+0.1% HFBA in 10 min): 5.21 min.

LC-MS m/z (ESI+, Cone Voltage 30V, Centroid): 356.1 (M+H)⁺.

(6aR,9R)—N,N-diethyl-7-(2-fluoroethyl)-4,6,6a,7,8,9-hexahydroindolo[4,3-fg]quinoline-9-carboxamide (39, 30.8 mg, 86.6 μmol) was dissolved in gradient-grade methanol (5.0 mL) and treated with 2N d-tartaric acid aqueous solution (43.3 μL, 43.3 μmol). Resulting mixture was stirred 5 min at room temperature at room temperature, solvents were removed in vacuo to yield (6aR,9R)—N,N-diethyl-7-(2-fluoroethyl)-4,6,6a,7,8,9-hexahydroindolo[4,3-fg]quinoline-9-carboxamide hemitartrate (39 hemitartrate) as an amorphous off-white solid.

Yield: 37.2 mg (quantitative).

¹H NMR spectrum (300 MHz, MeOD, δ_{H}): 7.21 (dd, J=6.7, 1.9, 1H), 7.16-7.05 (m, 2H), 6.98 (s, 1H), 6.34 (s, 1H), 4.84-4.76 (m, 1H), 4.75-4.62 (m, 1H), 4.46 (s, 1H), 4.07-3.95 (m, 1H), 3.86-3.72 (m, 1H), 3.57 (dt, J=11.4, 7.4, 3H), 3.45 (dd, J=14.1, 7.0, 3H), 3.42-3.35 (m, 1H), 3.28-3.17 (m, 1H), 3.11 (t, J=10.5, 1H), 2.79 (t, J=12.8, 1H), 1.30 (t, J=7.1, 3H), 1.18 (t, J=7.1, 3H).

LC-MS purity: 99% (ELSD), 99% (UV, 310 nm).

LC-MS Rt (Sinergy Polar RP 4.6 mm×150 mm, acetonitrile/water 30:70 to 100:0+0.1% HFBA in 10 min): 5.21 min.

LC-MS m/z (ESI+, Cone Voltage 30V, Centroid): 356.1 (M+H)⁺.

### Example 31. Preparation of (6aR,9R)—N—((R)-sec-butyl)-7-(3,3,3-trifluoropropyl)-4,6,6a,7,8,9-hexahydroindolo[4,3-fg]quinoline-9-carboxamide (40)

### Reaction Scheme:

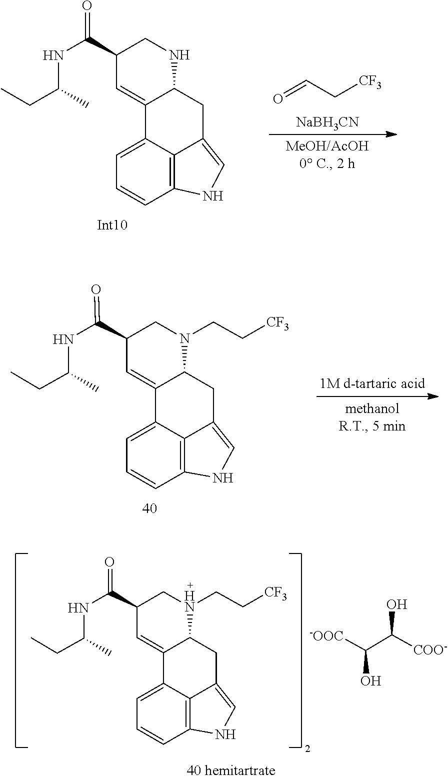

### Synthetic Protocols:

A solution of (6aR,9R)—N—((R)-sec-butyl)-4,6,6a,7,8,9-hexahydroindolo[4,3-fg]quinoline-9-carboxamide (Int10, 15.0 mg, 48.5 μmol) and 3,3,3-trifluoropropanal (21.8 μL, 194 μmol) in methanol (2.0 mL) was purged with argon and cooled to 0° C. Sodium cyanoborohydride (12.2 mg, 194 μmol) was added and the mixture was stirred for 5 minutes. Acetic acid (50 μL) was then added, and the mixture was stirred at 0° C. for 2 hours. Reaction mixture was diluted with dichloromethane (10 mL) then silica gel (4 g), and resulting suspension was stripped from solvents in vacuo. This residue was purified via flash column chromatography (silica gel 60, 0.040-0.063 mm; eluent: dichloromethane/methanol 99:1 to 98:2) to afford (6aR,9R)—N—((R)-sec-butyl)-7-(3,3,3-trifluoropropyl)-4,6,6a,7,8,9-hexahydroindolo[4,3-fg]quinoline-9-carboxamide (40, faster moving spot on TLC, less polar diastereomer) as a colorless oil.

Yield: 18.8 mg (96%)

LC-MS purity: 99% (ELSD), 100% (UV, 310 nm)

LC-MS Rt (Sinergy Polar RP, 4.6 mm×150 mm, acetonitrile/water 30:70 to 100:0+0.1% HBFA in 10 min): 5.78 min

LC-MS m/z (ESI+, Cone Voltage 30V, Centroid): 406.1 (M+H)⁺

(6aR,9R)-5-bromo-N,N-diethyl-7-(3,3,3-trifluoropropyl)-4,6,6a,7,8,9-hexahydroindolo[4,3-fg]quinoline-9-carboxamide (40, 18.8 mg, 46.5 μmol) was dissolved in gradient-grade methanol (5.0 mL) and treated with 2N d-tartaric acid aqueous solution (23.2 μL, 23.2 μmol). Resulting mixture was stirred at room temperature 5 min before solvents were removed in vacuo to yield (6aR,9R)-5-bromo-N,N-diethyl-7-(3,3,3-trifluoropropyl)-4,6,6a,7,8,9-hexahydroindolo[4,3-fg]quinoline-9-carboxamide hemitartrate (40 hemitartrate) as an off-white solid.

Yield: 22.2 mg (quantitative).

¹H NMR spectrum (300 MHz, MeOD, δ_{H}): 7.19 (dd, J=6.7, 1.9, 1H), 7.14-7.04 (m, 2H), 6.97 (s, 1H), 6.38 (s, 1H), 4.51 (s, 1H), 3.93-3.79 (m, 1H), 3.68-3.58 (m, 1H), 3.48 (dd, J=14.0, 5.3, 2H), 3.28-3.17 (m, 2H), 3.11-2.98 (m, 1H), 2.90 (dd, J=11.0, 8.9, 1H), 2.81-2.67 (m, 2H), 2.55 (ddd, J=16.3, 10.7, 5.7, 2H), 1.59-1.46 (m, 2H), 1.18 (d, J=6.6, 3H), 0.94 (t, J=7.4, 3H).

LC-MS purity: 99% (ELSD), 100% (UV, 310 nm)

LC-MS Rt (Sinergy Polar RP, 4.6 mm×150 mm, acetonitrile/water 30:70 to 100:0+0.1% HBFA in 10 min): 5.78 min

LC-MS m/z (ESI+, Cone Voltage 30V, Centroid): 406.1 (M+H)⁺

### Example 32. Preparation of (6aR,9R)-5-bromo-N,N-diethyl-7-(3-fluoropropyl)-4,6,6a,7,8,9-hexahydroindolo[4,3-fg]quinoline-9-carboxamide (41)

### Reaction Scheme:

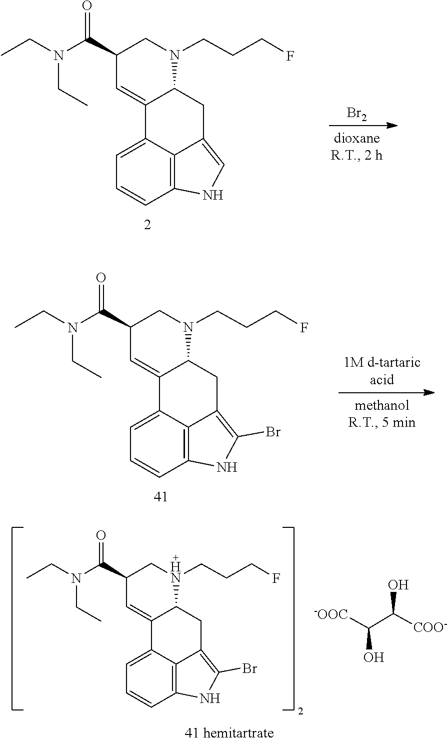

### Synthetic Protocols:

(6aR,9R)—N,N-diethyl-7-(3-fluoropropyl)-4,6,6a,7,8,9-hexahydroindolo[4,3-fg]quinoline-9-carboxamide (2, 30.0 mg, 0.081 mmol) was dissolved anhydrous dioxane (3.0 mL) and flushed with argon. A solution of bromine in dioxane (28.5 mg/mL, 455.2 μL, 0.081 mmol) was added dropwise and the mixture stirred for 2 h at room temperature. Reaction mixture was basified with triethylamine (100 μL), poured onto silica gel (4 g), and concentrated in vacuo. Purification via flash column chromatography (silica gel 60, 0.040-0.063 mm; eluent: cyclohexane/ethyl acetate 100:0 to 70:30+0.5%_{v/v} triethylamine) provided (6aR,9R)-5-bromo-N,N-diethyl-7-(3-fluoropropyl)-4,6,6a,7,8,9-hexahydroindolo[4,3-fg]quinoline-9-carboxamide (41) as an off-white amorphous solid.

Yield: 16.5 mg (45%).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 5.91 min.

LC-MS m/z (ESI+, Cone Voltage 30V, Centroid): 448.1 (⁷⁹Br, M+1), 450.0 (⁸¹Br, M+1)

(6aR,9R)-5-bromo-N,N-diethyl-7-(3-fluoropropyl)-4,6,6a,7,8,9-hexahydroindolo[4,3-fg]quinoline-9-carboxamide (41, 16.5 mg, 37.0 μmol) was dissolved in gradient-grade methanol (5.0 mL) and treated with 2N d-tartaric acid aqueous solution (18.5 μL, 18.5 μmol). Resulting mixture was stirred 5 min at room temperature, solvents were removed in vacuo to yield (6aR,9R)-5-bromo-N,N-diethyl-7-(3-fluoropropyl)-4,6,6a,7,8,9-hexahydroindolo[4,3-fg]quinoline-9-carboxamide hemitartrate (41 hemitartrate) as an off-white solid.

Yield: 19.25 mg (quantitative).

¹H NMR spectrum (300 MHz, MeOD, δ_{H}): 7.24-7.02 (m, 1H), 6.37 (s, 1H), 4.74-4.63 (m, 1H), 4.58-4.48 (m, 1H), 4.44 (s, 1H), 4.05-3.94 (m, 1H), 3.89-3.74 (m, 1H), 3.63-3.51 (m, 2H), 3.46 (q, J=7.4, 3H), 3.40-3.33 (m, 2H), 3.18-2.92 (m, 2H), 2.70 (t, J=12.0, 1H), 2.23-1.99 (m, 2H), 1.31 (t, J=7.0, 3H), 1.18 (t, J=7.1, 3H).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 5.91 min.

LC-MS m/z (ESI+, Cone Voltage 30V, Centroid): 448.1 (⁷⁹Br, M+1), 450.0 (⁸¹Br, M+1).

### Example 33. Preparation of (6aR,9R)-5-bromo-N,N-diethyl-7-(3,3,3-trifluoropropyl)-4,6,6a,7,8,9-hexahydroindolo[4,3-fg]quinoline-9-carboxamide (42)

### Reaction Scheme:

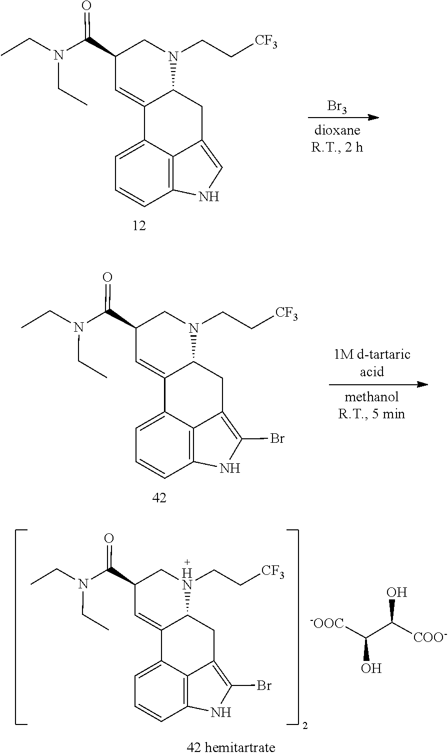

### Synthetic Protocols:

(6aR,9R)—N,N-Diethyl-7-(3,3,3-trifluoropropyl)-4,6,6a,7,8,9-hexahydroindolo[4,3-fg]quinoline-9-carboxamide (12, 35.8 mg, 0.088 mmol) was dissolved in anhydrous dioxane (3.0 mL) and flushed with argon. Solution of bromine in dioxane (28.5 mg/mL, 495.1 μL, 0.081 mmol) was added dropwise and resulting mixture stirred for 2 h at room temperature. The reaction mixture was basified with triethylamine (100 μL), poured onto silica gel (4.0 g), and concentrated in vacuo. Purification via flash column chromatography (silica gel 60, 0.040-0.063 mm; eluent: cyclohexane/ethyl acetate 100:0 to 70:30+0.5%ᵥ/v triethylamine) provided (6aR,9R)-5-bromo-N,N-diethyl-7-(3,3,3-trifluoropropyl)-4,6,6a,7,8,9-hexahydroindolo[4,3-fg]quinoline-9-carboxamide (42) as an off-white amorphous solid.

Yield: 19.3 mg (45%).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 6.28 min.

LC-MS m/z (ESI+, Cone Voltage 30V, Centroid): 484.1 (⁷⁹Br, M+1), 486.0 (⁸¹Br, M+1).

(6aR,9R)-5-bromo-N,N-diethyl-7-(3,3,3-trifluoropropyl)-4,6,6a,7,8,9-hexahydroindolo[4,3-fg]quinoline-9-carboxamide (42, 19.3 mg, 39.8 μmol) was dissolved in gradient-grade methanol (5.0 mL) and treated with 2N d-tartaric acid aqueous solution (19.9 μL, 19.9 μmol). Resulting mixture was stirred 5 min at room temperature before solvents were removed in vacuo to yield (6aR,9R)-5-bromo-N,N-diethyl-7-(3,3,3-trifluoropropyl)-4,6,6a,7,8,9-hexahydroindolo[4,3-fg]quinoline-9-carboxamide hemitartrate (42 hemitartrate) as an off-white solid.

Yield: 22.2 mg (quantitative).

¹H NMR spectrum (300 MHz, MeOD, δ_{H}): 7.22-7.00 (m, 3H), 6.31 (s, 1H), 4.51 (s, 1H), 3.97-3.83 (m, 1H), 3.55 (q, J=7.1, 3H), 3.45 (ddd, J=14.0, 7.0, 3.7, 2H), 3.38 (dd, J=5.8, 2.8, 1H), 3.28 (dd, J=12.5, 6.5, 1H), 3.19 (dd, J=11.2, 4.1, 1H), 3.05-2.92 (m, 1H), 2.86 (t, J=9.0, 1H), 2.56 (ddt, J=15.9, 10.5, 5.2, 3H), 1.29 (t, J=7.1, 3H), 1.18 (t, J=7.1, 3H).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 6.28 min.

LC-MS m/z (ESI+, Cone Voltage 30V, Centroid): 484.1 (⁷⁹Br, M+1), 486.0 (⁸¹Br, M+1).

### Example 34. Preparation of (6aR,9R)-5-bromo-N—((R)-sec-butyl)-7-methyl-4,6,6a,7,8,9-hexahydroindolo[4,3-fg]quinoline-9-carboxamide (43)

### Reaction Scheme:

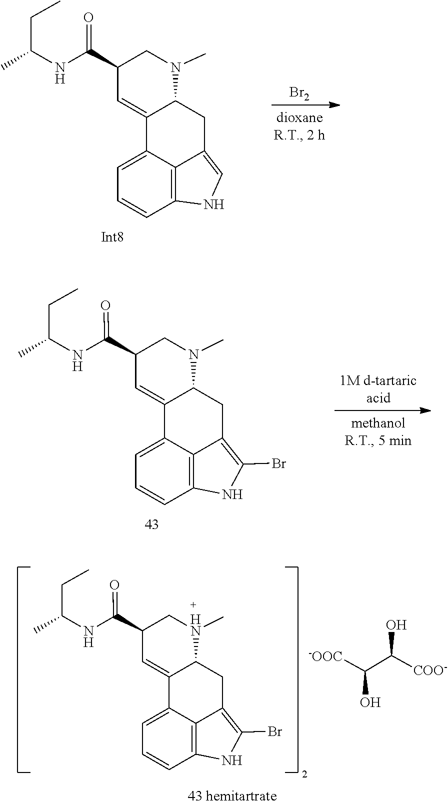

### Synthetic Protocols:

(6aR,9R)—N—((R)-sec-butyl)-7-methyl-4,6,6a,7,8,9-hexahydroindolo[4,3-fg]quinoline-9-carboxamide (Int8, 56.3 mg, 174 μmol) was dissolved in anhydrous dioxane (5.0 mL) and vessel was flushed with argon. A solution of bromine in dioxane (28.5 mg/mL, 976 μL, 174 μmol) was added dropwise and resulting mixture stirred for 2 h at room temperature. Reaction mixture was basified with trimethylamine (100 μL), poured onto silica gel (4 g), and concentrated in vacuo. Purification via flash column chromatography (silica gel 60, 0.040-0.063 mm; eluent: cyclohexane/ethyl acetate 100:0 to 70:30+0.5% v/v triethylamine) provided (6aR,9R)-5-bromo-N—((R)-sec-butyl)-7-methyl-4,6,6a,7,8,9-hexahydroindolo[4,3-fg]quinoline-9-carboxamide (43) as an off-white amorphous solid.

Yield: 15.1 mg (45%).

LC-MS purity: 100% (ELSD), 98% (UV, 310 nm).

LC-MS Rt (Sinergy Polar RP, 4.6 mm×150 mm, acetonitrile/water 30:70 to 100:0+0.1% HBFA in 10 min): 5.16 min.

LC-MS m/z (ESI+, Cone Voltage 30V, Centroid): 402.9 (⁷⁹Br, M+1), 403.9 (⁸¹Br, M+1).

(6aR,9R)-5-bromo-N—((R)-sec-butyl)-7-methyl-4,6,6a,7,8,9-hexahydroindolo[4,3-fg]quinoline-9-carboxamide (43, 15.1 mg, 37.6 μmol) was dissolved in gradient-grade methanol (5.0 mL) and treated with 2N d-tartaric acid aqueous solution (18.8 μL, 18.8 μmol). Resulting mixture was stirred 5 min at room temperature, then solvents were removed in vacuo to yield (6aR,9R)-5-bromo-N—((R)-sec-butyl)-7-methyl-4,6,6a,7,8,9-hexahydroindolo[4,3-fg]quinoline-9-carboxamide hemitartrate (43 hemitartrate) as an off-white solid.

Yield: 18.0 mg (quantitative).

¹H NMR spectrum (300 MHz, MeOD, δ_{H}): 7.23-7.05 (m, 3H), 6.46 (s, 1H), 4.43 (s, 1H), 3.85 (dd, J=13.3, 6.7, 1H), 3.79-3.67 (m, 2H), 3.49 (dd, J=14.5, 5.3, 1H), 3.44 (dd, J=14.4, 4.6, 1H), 3.14 (t, J=11.0, 1H), 2.89 (s, 3H), 2.73 (dd, J=14.1, 11.7, 1H), 1.60-1.46 (m, 2H), 1.20 (d, J=6.6, 3H), 0.94 (t, J=7.4, 3H).

LC-MS purity: 100% (ELSD), 98% (UV, 310 nm).

LC-MS Rt (Sinergy Polar RP, 4.6 mm×150 mm, acetonitrile/water 30:70 to 100:0+0.1% HBFA in 10 min): 5.16 min.

LC-MS m/z (ESI+, Cone Voltage 30V, Centroid): 402.9 (⁷⁹Br, M+1), 403.9 (⁸¹Br, M+1).

### Example 35. Preparation of (6aR,9R)-5-bromo-N-(pentan-3-yl)-7-methyl-4,6,6a,7,8,9-hexahydroindolo[4,3-fg]quinoline-9-carboxamide (44)

### Reaction Scheme:

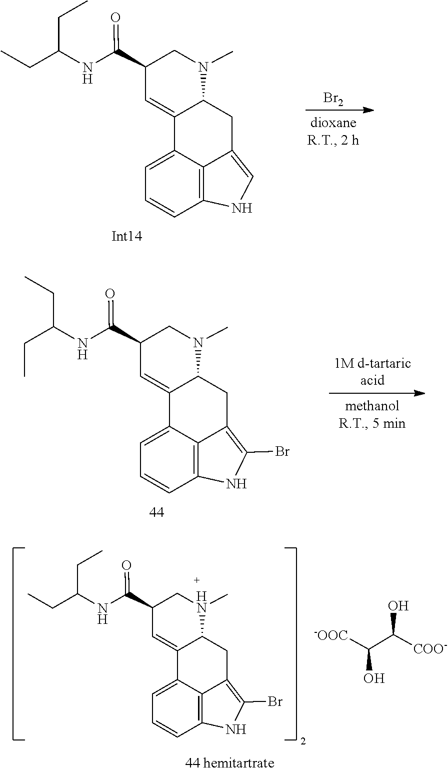

### Synthetic Protocols:

(6aR,9R)—N-(pentan-3-yl)-7-methyl-4,6,6a,7,8,9-hexahydroindolo[4,3-fg]quinoline-9-carboxamide (Int14, 58.7 mg, 174 μmol) was dissolved in anhydrous dioxane (6.0 mL) and flushed with argon. A solution of bromine in dioxane (28.5 mg/mL, 976 μL, 174 μmol) was added dropwise and resulting mixture stirred for 2 h at room temperature. The reaction mixture was basified with trimethylamine (100 μL), poured onto silica gel (4 g), and concentrated in vacuo. Purification via flash column chromatography (silica gel 60, 0.040-0.063 mm; eluent: cyclohexane/ethyl acetate 100:0 to 70:30+0.5%ᵥ/v triethylamine) provided (6aR,9R)-5-bromo-N-(pentan-3-yl)-7-methyl-4,6,6a,7,8,9-hexahydroindolo[4,3-fg]quinoline-9-carboxamide (44) as an off-white amorphous solid.

Yield: 29.1 mg (40%).

LC-MS purity: 99% (ELSD), 100% (UV, 310 nm).

LC-MS Rt (Sinergy Polar RP, 4.6 mm×150 mm, acetonitrile/water 30:70 to 100:0+0.1% HBFA in 10 min): 5.43 min.

LC-MS m/z (ESI+, Cone Voltage 30V, Centroid): 416.0 (⁷⁹Br, M+1), 418.0 (⁸¹Br, M+1).

(6aR,9R)-5-bromo-N-(pentan-3-yl)-7-methyl-4,6,6a,7,8,9-hexahydroindolo[4,3-fg]quinoline-9-carboxamide (44, 29.1 mg, 69.8 μmol) was dissolved in gradient-grade methanol (5.0 mL) and treated with 2N d-tartaric acid aqueous solution (34.9 μL, 34.9 μmol). Resulting mixture was stirred 5 min at room temperature, then solvents were removed in vacuo to yield (6aR,9R)-5-bromo-N-(pentan-3-yl)-7-methyl-4,6,6a,7,8,9-hexahydroindolo[4,3-fg]quinoline-9-carboxamide hemitartrate (44 hemitartrate) as an off-white solid.

Yield: 34.2 mg (quantitative).

¹H NMR spectrum (300 MHz, MeOD, δ_{H}): 7.22-7.06 (m, 3H), 6.48 (s, 1H), 4.43 (s, 1H), 3.86-3.75 (m, 2H), 3.75-3.67 (m, 1H), 3.50 (dd, J=15.0, 5.2, 2H), 3.21 (t, J=11.1, 1H), 2.92 (s, 3H), 2.77 (dd, J=14.3, 11.8, 1H), 1.62 (dt, J=13.5, 7.7, 2H), 1.46 (dt, J=14.1, 7.8, 2H), 0.96 (dt, J=15.1, 7.4, 6H).

LC-MS purity: 99% (ELSD), 100% (UV, 310 nm).

LC-MS Rt (Sinergy Polar RP, 4.6 mm×150 mm, acetonitrile/water 30:70 to 100:0+0.1% HBFA in 10 min): 5.43 min.

LC-MS m/z (ESI+, Cone Voltage 30V, Centroid): 416.0 (⁷⁹Br, M+1), 418.0 (⁸¹Br, M+1).

### Example 36. Preparation of (6aR,9R)-5-bromo-N—((R)-pentan-2-yl)-7-methyl-4,6,6a,7,8,9-hexahydroindolo[4,3-fg]quinoline-9-carboxamide (45)

### Reaction Scheme:

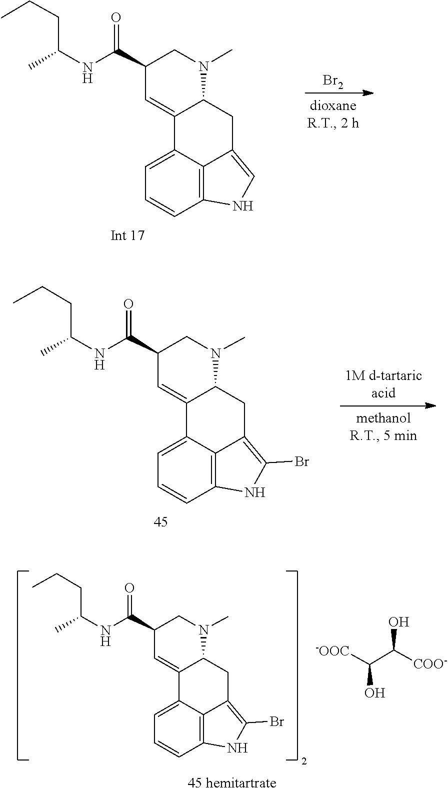

### Synthetic Protocols:

(6aR,9R)—N—((R)-pentan-2-yl)-7-propyl-4,6,6a,7,8,9-hexahydroindolo[4,3-fg]quinoline-9-carboxamide (Int17, 39.3 mg, 116 μmol) was dissolved in anhydrous dioxane (5.0 mL) and flushed with argon. A solution of bromine in dioxane (28.5 mg/mL, 653 μL, 116 μmol) was added dropwise and resulting mixture stirred for 2 h at room temperature. Reaction mixture was basified with trimethylamine (100 μL), poured onto silica gel (4 g) and concentrated in vacuo. Purification via flash column chromatography (silica gel 60, 0.040-0.063 mm; eluent: cyclohexane/ethyl acetate 100:0 to 70:30+0.5%_{v/v} triethylamine) provided (6aR,9R)-5-bromo-N—((R)-pentan-2-yl)-7-methyl-4,6,6a,7,8,9-hexahydroindolo[4,3-fg]quinoline-9-carboxamide (45) as an off-white amorphous solid.

Yield: 12.8 mg (61%).

LC-MS purity: 100% (ELSD), 98% (UV, 310 nm).

LC-MS Rt (Sinergy Polar RP, 4.6 mm×150 mm, acetonitrile/water 30:70 to 100:0+0.1% HBFA in 10 min): 5.54 min.

LC-MS m/z (ESI+, Cone Voltage 30V, Centroid): 416.0 (⁷⁹Br, M+1), 418.0 (⁸¹Br, M+1).

(6aR,9R)-5-bromo-N—((R)-pentan-2-yl)-7-methyl-4,6,6a,7,8,9-hexahydroindolo[4,3-fg]quinoline-9-carboxamide (45, 12.8 mg, 30.9 μmol) was dissolved in gradient-grade methanol (5.0 mL) and treated with 2N d-tartaric acid aqueous solution (15.4 μL, 15.4 μmol). Resulting mixture was stirred 5 min at room temperature, then solvents were removed in vacuo to yield (6aR,9R)-5-bromo-N—((R)-pentan-2-yl)-7-methyl-4,6,6a,7,8,9-hexahydroindolo[4,3-fg]quinoline-9-carboxamide hemitartrate (45 hemitartrate) as an off-white solid.

Yield: 15.3 mg (quantitative).

¹H NMR spectrum (300 MHz, MeOD, δ_{H}): 7.24-7.06 (m, 3H), 6.46 (s, 1H), 4.43 (s, 1H), 3.95 (dd, J=13.1, 6.4, 1H), 3.75-3.63 (m, 2H), 3.49 (dd, J=14.3, 5.3, 1H), 3.40 (dd, J=11.7, 3.7, 1H), 3.10 (t, J=10.8, 1H), 2.86 (s, 3H), 2.70 (dd, J=14.2, 11.9, 1H), 1.55-1.32 (m, 4H), 1.19 (d, J=6.6, 3H), 0.95 (t, J=7.1, 3H).

LC-MS purity: 100% (ELSD), 98% (UV, 310 nm).

LC-MS Rt (Sinergy Polar RP, 4.6 mm×150 mm, acetonitrile/water 30:70 to 100:0+0.1% HBFA in 10 min): 5.54 min.

LC-MS m/z (ESI+, Cone Voltage 30V, Centroid): 416.0 (⁷⁹Br, M+1), 418.0 (⁸¹Br, M+1).

### Example 37. Preparation of (6aR,9R)-5-bromo-N—((R)-sec-butyl)-7-propyl-4,6,6a,7,8,9-hexahydroindolo[4,3-fg]quinoline-9-carboxamide (46)

### Reaction Scheme:

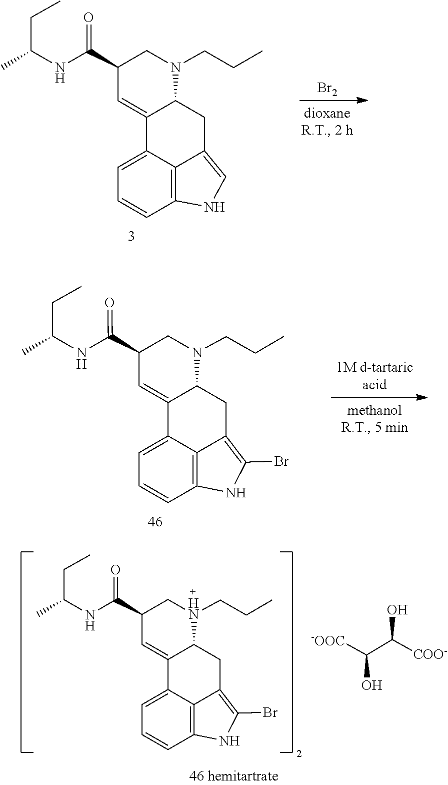

### Synthetic Protocols:

(6aR,9R)—N—((R)-sec-butyl)-7-propyl-4,6,6a,7,8,9-hexahydroindolo[4,3-fg]quinoline-9-carboxamide (3, 22.5 mg, 64.0 μmol) was dissolved anhydrous dioxane (3.0 mL) and flushed with argon. A solution of bromine in dioxane (26.43 mg/mL, 359.8 μL, 64.0 μmol) was added dropwise and resulting mixture stirred for 2 h at room temperature. The reaction mixture was basified with trimethylamine (100 μL), poured onto silica gel (4 g), and concentrated in vacuo. Purification via flash column chromatography (silica gel 60, 0.040-0.063 mm; eluent: cyclohexane/ethyl acetate 100:0 to 70:30+0.5%_{v/v} triethylamine) provided (6aR,9R)-5-bromo-N—((R)-sec-butyl)-7-propyl-4,6,6a,7,8,9-hexahydroindolo[4,3-fg]quinoline-9-carboxamide freebase (46) as an off-white amorphous solid.

Yield: 16.7 mg (61%).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 5.86 min.

LC-MS m/z (ESI+, Cone Voltage 30V, Centroid): 430.1 (⁷⁹Br, M+1), 432.0 (⁸¹Br, M+1).

(6aR,9R)-5-bromo-N—((R)-sec-butyl)-7-propyl-4,6,6a,7,8,9-hexahydroindolo[4,3-fg]quinoline-9-carboxamide (46, 16.7 mg, 38.8 μmol) was dissolved in gradient-grade methanol (5.0 mL) and treated with 2N d-tartaric acid aqueous solution (19.4 μL, 19.4 μmol). Resulting mixture was stirred 5 min at room temperature, then solvents were removed in vacuo to yield (6aR,9R)-5-bromo-N—((R)-sec-butyl)-7-propyl-4,6,6a,7,8,9-hexahydroindolo[4,3-fg]quinoline-9-carboxamide hemitartrate (46 hemitartrate) as an off-white solid.

Yield: 19.6 mg (quantitative).

¹H NMR spectrum (300 MHz, MeOD, δ_{H}): 7.23-7.05 (m, 3H), 6.45 (s, 1H), 4.41 (s, 1H), 4.01-3.90 (m, 1H), 3.85 (dd, J=13.4, 6.7, 1H), 3.66-3.56 (m, 1H), 3.50-3.41 (m, 1H), 3.41 (dd, J=14.6, 5.2, 1H), 3.25-3.12 (m, 1H), 3.25-3.11 (m, 1H), 3.09-2.95 (m, 1H), 2.77 (dd, J=14.2, 11.9, 1H), 1.87-1.67 (m, 2H), 1.61-1.46 (m, 2H), 1.19 (d, J=6.6, 3H), 1.05 (t, J=7.3, 3H), 0.94 (t, J=7.4, 3H).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 5.86 min.

LC-MS m/z (ESI+, Cone Voltage 30V, Centroid): 430.1 (⁷⁹Br, M+1), 432.0 (⁸¹Br, M+1).

### Example 38. Preparation of ((6aR,9R)-5-bromo-7-propyl-4,6,6a,7,8,9-hexahydroindolo[4,3-fg]quinolin-9-yl)((2S,4S)-2,4-dimethylazetidin-1-yl)methanone (47)

### Reaction Scheme:

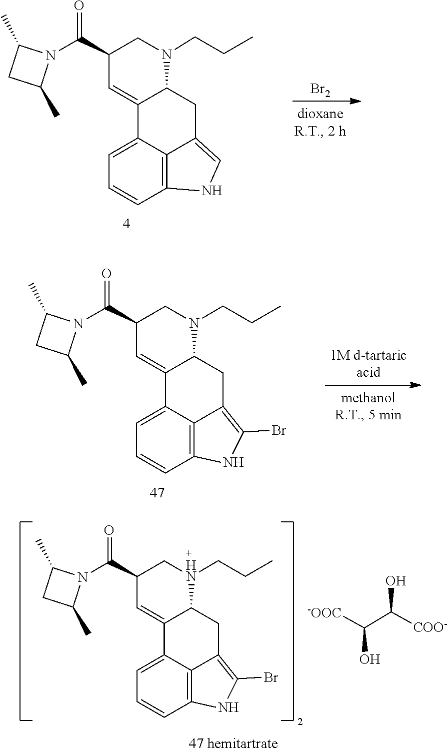

### Synthetic Protocols:

((6aR,9R)-7-propyl-4,6,6a,7,8,9-hexahydroindolo[4,3-fg]quinolin-9-yl)((2S,4S)-2,4-dimethylazetidin-1-yl)methanone (4, 46.3 mg, 127 μmol) was dissolved anhydrous dioxane (6.0 mL) and flushed with argon. A solution of bromine in dioxane (28.5 mg/mL, 715 μL, 127 μmol) was added dropwise and resulting mixture stirred for 2 h at room temperature. Reaction mixture was basified with trimethylamine (100 μL), poured onto silica gel (4 g), and concentrated in vacuo. Purification via flash column chromatography (silica gel 60, 0.040-0.063 mm; eluent: cyclohexane/ethyl acetate 100:0 to 80:20+0.5%_{v/v} triethylamine) provided ((6aR,9R)-5-bromo-7-propyl-4,6,6a,7,8,9-hexahydroindolo[4,3-fg]quinolin-9-yl)((2S,4S)-2,4-dimethylazetidin-1-yl)methanone (47) as an off-white amorphous solid.

Yield: 5.2 mg (61%).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 5.99 min.

LC-MS m/z (ESI+, Cone Voltage 30V, Centroid): 442.0 (⁷⁹Br, M+1), 444.0 (⁸¹Br, M+1).

((6aR,9R)-5-bromo-7-propyl-4,6,6a,7,8,9-hexahydroindolo[4,3-fg]quinolin-9-yl)((2S,4S)-2,4-dimethylazetidin-1-yl)methanone (47, 5.2 mg, 11.7 μmol) was dissolved in gradient-grade methanol (5.0 mL) and treated with 2N d-tartaric acid aqueous solution (5.9 μL, 5.9 μmol). Resulting mixture was stirred 5 min at room temperature, then solvents were removed in vacuo to yield ((6aR,9R)-5-bromo-7-propyl-4,6,6a,7,8,9-hexahydroindolo[4,3-fg]quinolin-9-yl)((2S,4S)-2,4-dimethylazetidin-1-yl)methanone hemitartrate (47 hemitartrate) as an off-white solid.

Yield: 6.05 mg (quantitative).

¹H NMR spectrum (300 MHz, MeOD, δ_{H}): 7.24-7.05 (m, 3H), 6.35 (s, 1H), 4.81-4.64 (m, 1H), 4.57-4.43 (m, 1H), 4.42 (s, 1H), 3.99-3.76 (m, 1H), 3.69-3.59 (m, 1H), 3.51-3.38 (m, 2H), 3.25-3.12 (m, 1H), 3.01-2.87 (m, 2H), 2.69 (t, J=13.2, 1H), 2.20-2.05 (m, 2H), 1.88-1.66 (m, 2H), 1.58 (d, J=6.2, 3H), 1.47 (d, J=6.3, 3H), 1.05 (t, J=7.3, 3H).

LC-MS purity: 100% (ELSD), 100% (UV, 310 nm).

LC-MS Rt (Sinergy Polar RP, 4.6 mm×150 mm, acetonitrile/water 30:70 to 100:0+0.1% HBFA in 10 min): 5.99 min.

LC-MS m/z (ESI+, Cone Voltage 30V, Centroid): 442.0 (⁷⁹Br, M+1), 444.0 (⁸¹Br, M+1).

### Example 39: 5-HT2A and 5-HT2B Receptor Binding

The binding affinities of disclosed compounds at the ketanserin binding site of the 5-HT2A receptor and the LSD binding site of the 5-HT2B receptor were determined in radioligand binding experiments.

### Methods:

Affinity of the test compounds for the 5-HT2A receptor was determined in radioligand binding experiments with [³H]ketanserin by WuXi AppTec (Hong Kong) Limited, using methods adapted from the literature and under conditions described in Table 1.

TABLE 1
Assay conditions for 5-HT2A receptor radioligand binding.

| | |
|---|---|
| Receptor Source | HEK293 stable cell line |
| Incubation Vehicle | 0.5% DMSO |
| Incubation Time | l h |
| Incubation Temperature | 25° C. |
| Incubation Buffer | 50 mM Tris-HCl, pH 7.4 |
| Ligand | 1 nM [³H]ketanserin |
| Non-Specific Ligand | 1 μM ketanserin |

Affinity of the test compounds for the 5-HT2B receptor was determined in radioligand binding experiments with [³H]LSD by WuXi AppTec (Hong Kong) Limited, using methods adapted from the literature and under conditions described in Table 2.

TABLE 2
Assay conditions for 5-HT2B receptor radioligand binding.

| | |
|---|---|
| Receptor Source | HEK293 stable cell line |
| Vehicle | 1.0% DMSO |
| Incubation Time | 1 h |
| Incubation Temperature | 25° C. |
| Incubation Buffer | 50 mM Tris-HCl, pH 7.4 |
| Ligand | 1 nM [³H]LSD |
| Non-Specific Ligand | 50 μM serotonin |

### Results:

Results of the radioligand binding assays are shown in Table 3. Tested compounds showed substantial binding affinity for the 5-HT2A and 5-HT2B receptor. Compounds having the R configuration at position 9 were much more potent at the 5-HT2A receptor than those having the S configuration at this position. Tested compounds were more selective for the 5-HT2A receptor over the 5-HT2B receptor compared to the reference compound LSD. Compounds bearing an arylalkyl or heteroarylalkyl substituent on the amine nitrogen (position 7) tended to be much more potent in binding at the 5-HT2A receptor than in the Ca²⁺ signaling assay (see Table 4). Similarly, compounds bearing a bromine on the indole (position 5) also tended to be much more potent in binding at the 5-HT2A receptor than in the Ca²⁺ signaling assay (see Table 4). Lastly, dihydro derivatives 36 and 37 were reduced in potency compared to their dehydro counterparts 1 and LSD, respectively.

TABLE 3
Results of 5-HT2A and 5-HT2B receptor binding
affinity experiments. NT = not tested.

| | 5-HT2A Receptor | 5-HT2B Receptor |
|---|---|---|
| | Kᵢ (nM) | Kᵢ (nM) |
| Compound | ([³H]ketanserin) | ([³H]LSD) |
| LSD | 0.89 | 0.42 |
| 1 | 1.06 | NT |
| 2a | 115.05 | NT |
| 2 | 2.79 | 4.1 |
| 3a | 27.6 | NT |
| 3 | 1.3615 | 4.09 |
| 6 | 1.16 | 3.33 |
| 7 | 0.85 | NT |
| 4 | 3.07 | 17.7 |
| 5a | 74.53 | NT |
| 5 | 2.1836 | NT |
| 8a | >1225 | NT |
| 8 | 2.3775 | NT |
| 9 | 1.11 | NT |
| 10 | 32.69 | NT |
| 11 | 0.69 | NT |
| Int11 | 2.22 | NT |
| Int8a | 84.34 | NT |
| Int8 | 1.0707 | NT |
| Int14a | 56.23 | NT |
| Int14 | 0.47 | NT |
| Int17a | 41.3 | NT |
| Int17 | 0.41 | NT |
| 12 | 5.61 | NT |
| 13 | 2.23 | NT |
| 16 | 2.18 | NT |
| 18 | 3.41 | NT |
| 19 | 5.58 | NT |
| 20 | 1.03 | NT |
| 21 | 1.93 | NT |
| 22 | 28.44 | NT |
| 23 | 26.57 | NT |
| 26 | 11.30 | NT |
| 27 | 8.96 | NT |
| 35 | 0.73 | NT |
| Int22 | 14.88 | NT |
| 36 | 10.67 | NT |
| 37 | 11.42 | NT |
| 38 | 0.84 | NT |
| 39 | 4.54 | NT |
| 40 | 37.74 | NT |
| 41 | 0.58 | NT |
| 42 | 1.84 | NT |
| 43 | 1.13 | NT |
| 44 | 0.76 | NT |
| 45 | 0.38 | NT |
| 46 | 1.03 | NT |
| 47 | 1.38 | NT |

### Example 40: Functional Activity at Serotonin Receptors

Disclosed compounds were tested for agonist activity at several serotonin receptor subtypes (5-HT2A, 2-HT2B, 5-HT2C, and 5-HT1A) using Ca²⁺ flux functional assays and the 5-HT1B receptor using a cAMP accumulation assay, with the results summarized in Table 4. Most compounds exhibited potent agonist activity at the 5-HT2A receptor, suggestive of potential hallucinogenic activity as well as possible therapeutic effects. Compounds having the R configuration at position 9 were much more potent at the 5-HT2A receptor than those having the S configuration at this position. Potent agonist activity was also observed at the other serotonin receptors tested, although the selectivity profile among the receptors varied across compounds. For example, compounds with longer alkyl chains on the amine nitrogen (e.g., 2, 3, 4, and 6) tended to exhibit greater selectivity for 5-HT2A over 5-HT1B compared to the N-methyl prototype LSD. Similarly, compounds with longer N-alkyl chains (e.g., the N-propyl compounds 3, 4, and 7), tended to be more selective for 5-HT2A over 5-HT2B than their closest N-methyl counterparts (e.g., Int8, Int11, and Int17, respectively). However, at the same time, the N-propyl compounds were more efficacious agonists at 5-HT2B than the corresponding N-methyl compounds. The selectivity of disclosed compounds for 5-HT2A over 5-HT2C and 5-HT1A was less predictable and varied widely across compounds, ranging from ˜1:6 to ˜20:1 (2C/2A) in the case of 5-HT2C and from ˜1:4 to >100:1 (1A/2A) in the case of 5-HT1A. Compounds bearing an arylalkyl or heteroarylalkyl substituent on the amine nitrogen (position 7) tended to be much more selective for the 5-HT2A receptor in terms of agonist activity and did not exhibit substantial agonist activity at other serotonin receptors. Compound 40 was also highly selective for 5-HT2A compared to the other serotonin receptors tested. An azetidinyl amide substituent tended to increase potency at 5-HT1A relative to 5-HT2A. In contrast, bromination on the indole (position 5) tended to increase selectivity for 5-HT2A relative to 5-HT1A, while also attenuating the maximal efficacy at 5-HT2A to yield partial agonists. These bromo compounds were also much less potent as functional agonists compared to their binding affinities (see Table 3). Interestingly, compound 42 exhibited no agonist activity at any of the receptors tested, despite potent binding to 5-HT2A (see Table 3), suggesting that this compound may act as an antagonist at this receptor.

Functional Assays at 5-HT2A, 5-HT2B, and 5-HT1A. Agonist activity at 5-HT2A, 5-HT2B, and 5-HT1A receptors was determined using a FLIPR Ca²⁺ flux assay at WuXi AppTec (Hong Kong) Limited according to their standard protocols. Briefly, stably transfected cells expressing the receptor of interest (HEK293 for 5-HT2A and 5-HT2B; CHO cells for 5-HT1A) were grown and plated in a 384 well plate and incubated at 37° C. and 5% CO₂ overnight. A solution of 250 mM probenecid in 1 mL FLIPR assay buffer was prepared fresh. This was combined with a fluorescent dye (Fluo-4 Direct™) to make a final assay concentration of 2.5 mM. Compounds were diluted 1:3.16 for 10 points and 750 nL was added to a 384 well compound plate using ECHO along with 30 μL assay buffer. The fluorescent dye was then added to the assay plate along with assay buffer to a final volume of 40 μL. The cell plate was incubated for 50 min at 37° C. and 5% CO₂ and placed into the FLIPR Tetra along with the compound plate. 10 μL of references and compounds were then transferred from the compound plate into the cell plate and the fluorescent signal was read.

Functional Assay at 5-HT2C. Agonist activity at 5-HT2C was determined using a FLIPR Ca²⁺ flux assay at Eurofins DiscoverX (Fremont, Calif.) according to their standard protocols. Briefly, stably transfected cells expressing the human 5-HT2C receptor were grown and plated in a 384 well plate and incubated at 37° C. and 5% CO₂ overnight. Assays were performed in 1× Dye Loading Buffer consisting of 1× Dye, 1× Additive A, and 2.5 mM Probenecid in HBSS/20 mM Hepes. Probenecid was prepared fresh. Cells were loaded with dye prior to testing and incubated at 37° C. for 30-60 minutes. After dye loading, cells were removed from the incubator and 10 μL HBSS/20 mM Hepes was added. 3× vehicle was included in the assay buffer. Cells were incubated for 30 mins at room temperature in the dark to equilibrate plate temperature. Intermediate dilution of sample stocks was performed to generate 4× sample in assay buffer. Compound agonist activity was measured on a FLIPR Tetra (MDS). Calcium mobilization was monitored for 2 minutes and 10 μL 4× sample in HBSS/20 mM Hepes was added to the cells 5 seconds into the assay.

Functional Assay at 5-HT1B. Agonist activity at 5-HT1B was determined using a cAMP accumulation protocol at WuXi AppTec (Hong Kong) Limited according to their standard protocols. Briefly, stably transfected cells were plated in an OptiPlate-384 well plate, incubated at RT for 60 mins, and cAMP standard solution (800 nM, 10 μL) was added to the blank well. Then, 10 μL detection reagent was added to each well, the plate incubated for 60 mins at RT, and the plate read using EnVision.

TABLE 4
Agonist activity of compounds at select serotonin receptors in Ca²⁺ flux (5-HT2A, 2B, 2C, and 1A) and cAMP (5-HT1B) functional assays.

| | 5-HT2A | 5-HT2A | 5-HT2B | 5-HT2B | 5-HT2C | 5-HT2C | 5-HT1A | 5-HT1A | 5-HT1B | 5-HT1B |
|---|---|---|---|---|---|---|---|---|---|---|
| | EC₅₀ | % Act @ | EC₅₀ | % Act @ | EC₅₀ | % Act @ | EC₅₀ | % Act @ | EC₅₀ | % Act @ |
| Compound | (nM) | Max Dose | (nM) | Max Dose | (nM) | Max Dose | (nM) | Max Dose | (nM) | Max Dose |
| LSD | 4.332 | 107.7 | 59.4 | 19.26 | 88.2551 | 92.119 | 25.13 | 91.39 | 2.065 | 98.03 |
| 1 | 18.49 | 96.96 | 104.3 | 103.75 | 11.8806 | 106.79 | 44.4 | 95.6 | 3.816 | 96.6 |
| 2a | 168.6 | 90.29 | 2165 | 40.13 | 256.46 | 118.85 | 542.7 | 96.77 | NT | NT |
| 2 | 11.1 | 97.75 | 201.7 | 101.16 | 4.3513 | 105.48 | 25.36 | 92.42 | 193.5 | 89.97 |
| 3a | 145.5 | 92.04 | 1825 | 52.81 | 131.758 | 108.705 | 155.2 | 108.14 | NT | NT |
| 3 | 1.568 | 102.64 | 61.98 | 94.11 | 0.82239 | 119.49 | 1.794 | 107.6 | 68.85 | 98.66 |
| 6 | 11.32 | 94.64 | 252.7 | 92.93 | 6.183 | 116.77 | 36.11 | 101.82 | 201 | 94.62 |
| 7 | 13.21 | 94.52 | 364 | 94.75 | 1.9931 | 114.78 | 12.83 | 107.16 | NT | NT |
| 4 | 21.28 | 97.6 | 511.3 | 87.46 | 5.60015 | 110.845 | 5.882 | 123.1 | 97.56 | 96 |
| 5a | 118 | 89.59 | 2507 | 43.7 | 810.599 | 99.4405 | 130.7 | 111.65 | NT | NT |
| 5 | 0.7176 | 102.81 | 35.39 | 84.77 | 1.28585 | 114.5 | 0.9598 | 107.87 | NT | NT |
| 8a | 395.6 | 87.45 | 3588 | 35.71 | >10000 | 13.314 | 1559 | 90.34 | NT | NT |
| 8 | 0.6784 | 99.25 | 22.23 | 91.56 | 4.0451 | 108.26 | 2.533 | 116.41 | NT | NT |
| 9 | 227.7 | 75.81 | 399.8 | 89.6 | NT | NT | 96.5 | 101.39 | 264.4 | 81.46 |
| 10 | 642.6 | 49.44 | >10000 | 2.005 | >10000 | 13.417 | 30554 | 52.96 | 3192 | 87.29 |
| 11 | 216.1 | 63.46 | >10000 | 2.5 | 74.502 | 107.89 | 29841 | 57.91 | 447.7 | 47.01 |
| Int11 | 37 | 110.03 | 96.56 | 38.15 | 7.68228 | 93.265 | 8.879 | 89.9 | NT | NT |
| Int8a | 178.4 | 72.6 | >10000 | 6.87 | 159.918 | 93.73 | 163.5 | 103.65 | NT | NT |
| Int8 | 10.26 | 96.25 | 71.91 | 17.7 | 7.3198 | 98.245 | 9.694 | 90.25 | NT | NT |
| Int14a | 468 | 32.98 | >10000 | 1.86 | 508.08 | 119.28 | 1550 | 70.27 | NT | NT |
| Int14 | 15.9 | 93.33 | >10000 | 9.84 | 3.6318 | 99.149 | 46.04 | 102.46 | NT | NT |
| Int17a | 425.3 | 63.31 | >10000 | 5.63 | 962.16 | 112.06 | 265.6 | 88.52 | NT | NT |
| Int17 | 9.67 | 91.47 | 70.76 | 25.49 | 2.5348 | 102.19 | 7.007 | 113.04 | NT | NT |
| 12 | 52.89 | 78.69 | 154.3 | 29.55 | NT | NT | 2138 | 71.42 | 193.8 | 95.51 |
| 13 | 25.95 | 92.60 | 182.5 | 91.75 | NT | NT | 15.33 | 83.08 | 98.61 | 90.43 |
| 16 | 546.9 | 62.05 | >10000 | 26.497 | NT | NT | >10000 | 22.29 | 682.2 | 90.1 |
| 18 | 226.9 | 61.09 | >10000 | 4.69 | NT | NT | >10000 | 28.93 | 1780 | 83.31 |
| 19 | 2964 | 70.54 | >10000 | 2.44 | NT | NT | >10000 | −0.65 | 5850 | 71.08 |
| 20 | 264.8 | 77.19 | >10000 | 3.60 | NT | NT | >10000 | 3.85 | >10000 | 34.59 |
| 21 | 619 | 51.23 | >10000 | 1.73 | NT | NT | >10000 | 8.2 | 2751 | 79.13 |
| 22 | 747.8 | 64.47 | >10000 | 7.74 | NT | NT | 7839 | 57.33 | 846.7 | 89.15 |
| 23 | 511.9 | 85.397 | >10000 | 12.50 | NT | NT | >10000 | 46.19 | 449.8 | 83.68 |
| 26 | 15.56 | 83.23 | 430.5 | 100.66 | NT | NT | 3.63 | 97.94 | 734.1 | 77.16 |
| 27 | 12.0 | 81.1 | 351.0 | 96.0 | NT | NT | 2.97 | 90.8 | 88.71 | 96.92 |
| 35 | 66.4 | 67.74 | >10000 | −0.13 | NT | NT | >3160 | −1.58 | 213.5 | 76.8 |
| Int22 | 21.1 | 89.65 | 55.24 | 21.35 | NT | NT | 8.82 | 103.7 | 7.38 | 94.25 |
| 36 | 50.38 | 77.54 | 626.5 | 110.53 | NT | NT | 4.84 | 88.68 | 22.75 | 97.51 |
| 37 | 76.57 | 76.37 | 465.3 | 24.06 | NT | NT | 17.19 | 108.98 | 19.42 | 90.74 |
| 38 | 11.82 | 93.39 | 22.14 | 28.13 | NT | NT | 3.45 | 90.5 | 4.65 | 94.72 |
| 39 | 16.15 | 95.52 | 136.8 | 85.46 | NT | NT | 238.8 | 91.21 | 1095 | 99.00 |
| 40 | 169.1 | 45.12 | >10000 | 8.46 | NT | NT | >10000 | 22.27 | 8305 | 62.14 |
| 41 | 115.5 | 75.44 | 509 | 84.51 | NT | NT | >10000 | 39.75 | >10000 | 61.49 |
| 42 | >1000 | 8.29 | >10000 | 2.58 | NT | NT | >10000 | 9.13 | >10000 | 33.13 |
| 43 | 118.2 | 39.32 | >10000 | 1.00 | NT | NT | >10000 | 6.30 | 310.8 | 83.76 |
| 44 | 129.0 | 29.90 | >10000 | 15.68 | NT | NT | >10000 | 16.56 | 1268 | 68.53 |
| 45 | 133.2 | 22.28 | >10000 | −0.31 | NT | NT | >10000 | 12.53 | 245.0 | 87.10 |
| 46 | 110.5 | 69.48 | 788.3 | 79.00 | NT | NT | 707.5 | 69.48 | 1181 | 78.76 |
| 47 | 117.3 | 57.01 | 1440 | 87.01 | NT | NT | 93.60 | 62.15 | 4963 | 76.22 |

NT = not tested

### Example 41: Functional Activity at the 5-HT2A Receptor in a Beta-Arrestin Recruitment Assay

Disclosed compounds were tested for agonist activity at the 5-HT2A receptor using a beta-arrestin (arrestin) recruitment functional assay, with the results summarized in Table 5. All compounds tested were agonists in this assay and many were highly potent. Compounds having the R configuration at position 9 were much more potent at the 5-HT2A receptor than those having the S configuration at this position. It was found that the size and nature of the substituent on the amine nitrogen (position 7) was an important determinant of maximal efficacy in this assay. Compounds with longer alkyl chains at this position (e.g., 1, 2, 3, 4, 5, 6, 7, and 8) were all full agonists, whereas compounds with a methyl substituent on the amine (e.g., LSD, Int11, Int8, Int14, and Int17) were all partial agonists with an Eₘₐₓ<60%. Interestingly, compounds 10 and 11, with much larger aryl substituents at this position, were also partial agonists. These compounds (10 and 11) were also unique in that they exhibited substantial arrestin bias in signaling, as they were much more potent (>50-fold) in this assay compared to the G protein-dependent Ca²⁺ signaling assay (see Table 4). The degree of arrestin bias varied substantially across the other compounds tested as well.

Arrestin Functional Assay at 5-HT2A. Recruitment of beta-arrestin was determined using a PathHunter assay at Eurofins DiscoverX (Fremont, Calif.) according to their standard protocols. PathHunter GPCR beta-arrestin assays take advantage of DiscoverX's proprietary Enzyme Fragment Complementation technology. The GPCR is fused in frame with a small enzyme donor fragment ProLink™ (PK) and co-expressed in cells stably expressing a fusion protein of beta-arrestin and the larger, N-terminal deletion mutant of beta-galactosidase. Activation of the GPCR (5-HT2A receptor in this case) stimulates binding of beta-arrestin to the PK-tagged GPCR and forces complementation of the two enzyme fragments, resulting in the formation of an active beta-galactosidase enzyme. This interaction leads to an increase in enzyme activity that can be measured using chemiluminescent PathHunter Detection Reagents. Briefly, PathHunter cells expressing 5-HT2A receptors were seeded in a volume of 20 μL into 384-well plates and incubated at 37° C. for the appropriate time prior to testing. For agonist activity determination, cells were incubated with 5 μL of 5× sample in assay buffer and incubated at 37° C. for 90-180 min with a vehicle concentration of 1%. Plates were then imaged on a microplate reader and the agonist activity was calculated using the following formula: % Activity=100%×(mean RLU of test sample−mean RLU of vehicle control)/(mean MAX control ligand−mean RLU of vehicle control), where RLU=relative light units.

TABLE 5
Agonist activity of compounds at the 5-HT2A
receptor in an arrestin recruitment assay.

| | 5-HT2A Arrestin | 5-HT2A Arrestin |
|---|---|---|
| Compound | EC₅₀ (nM) | % Act @ Max Dose |
| LSD | 2.1839 | 52.646 |
| 1 | 2.0083 | 128.71 |
| 2a | 50.62 | 83.328 |
| 2 | 2.2682 | 114.55 |
| 3a | 53.9995 | 112.91 |
| 3 | <0.509 | 109.57 |
| 6 | 1.9128 | 102.75 |
| 7 | 1.5951 | 110.2 |
| 4 | 1.44168 | 116.8 |
| 5a | 66.7575 | 62.444 |
| 5 | 1.91871 | 105.72 |
| 8a | 1184.02 | 81.117 |
| 8 | 1.70581 | 112.42 |
| 10 | 8.47423 | 47.06 |
| 11 | 1.2686 | 54.255 |
| Int11 | 1.56921 | 49.269 |
| Int8a | 19.3215 | 35.52 |
| Int8 | 4.04977 | 46.06 |
| Int14a | 35.713 | 44.321 |
| Int14 | 1.475 | 44.377 |
| Int17a | 75.772 | 43.729 |
| Int17 | 1.187 | 38.809 |

### Example 42: Functional Activity at Other Monoamine Receptors

Disclosed compounds were tested for agonist activity at several adrenergic (Alpha1A and Alpha2A) and dopamine (D1 and D2) receptor subtypes using Ca²⁺ flux functional assays, with the results summarized in Table 6. Selectivity for the 5-HT2A receptor over these other targets varied depending on the specific target and compound. In many cases, the disclosed compounds were more selective than LSD for the 5-HT2A receptor over the tested adrenergic and dopamine receptors. In particular, compound 3 showed exceptional selectivity. It was also found that the size and nature of the substituent on the amine nitrogen (position 7) was an important determinant of maximal efficacy at these receptors. Compounds with longer alkyl chains at this position (e.g., 1, 2, 3, 4, and 6) showed substantially higher maximal efficacy at Alpha1A, Alpha2A, and D2 compared to the N-methyl compound LSD. Interestingly, compounds 10 and 11, with much larger aryl substituents at this position, were very low efficacy agonists at Alpha1A and Alpha2A.

Functional Assays at Adrenergic and Dopamine Receptors. Agonist activity at Alpha1A, Alpha2A, D1, and D2 receptors was determined using a FLIPR Ca²⁺ flux assay at WuXi AppTec (Hong Kong) Limited according to their standard protocols. Briefly, stably transfected cells expressing the receptor of interest were grown and plated in a 384 well plate and incubated at 37° C. and 5% CO₂ overnight. A solution of 250 mM probenecid in 1 mL FLIPR assay buffer was prepared fresh. This was combined with a fluorescent dye (Fluo-4 Direct™) to make a final assay concentration of 2.5 mM. Compounds were diluted 1:3.16 for 10 points and 750 nL was added to a 384 well compound plate using ECHO along with 30 μL assay buffer. The fluorescent dye was then added to the assay plate along with assay buffer to a final volume of 40 μL. The cell plate was incubated for 50 min at 37° C. and 5% CO₂ and placed into the FLIPR Tetra along with the compound plate. 10 μL of references and compounds were then transferred from the compound plate into the cell plate and the fluorescent signal was read.

TABLE 6
Agonist activity of compounds at other monoamine receptors in Ca²⁺ flux functional assays.

| | Alpha1A | Alpha 1A | Alpha 2A | Alpha 2A | D1 | D1 | D2 | D2 |
|---|---|---|---|---|---|---|---|---|
| | EC₅₀ | % Act @ | EC₅₀ | % Act @ | EC₅₀ | % Act @ | EC₅₀ | % Act @ |
| Compound | (nM) | Max Dose | (nM) | Max Dose | (nM) | Max Dose | (nM) | Max Dose |
| LSD | 91.06 | 60.3 | 201.2 | 21.3 | 4.866 | 94.9 | 4.928 | 78.33 |
| 1 | 8.818 | 80.6 | 42.23 | 58.3 | 4.641 | 108.68 | 2.168 | 94.51 |
| 2 | 237.1 | 109.68 | 1003 | 64.96 | 86.39 | 113.6 | 27.67 | 91.2 |
| 3 | 42.27 | 124.7 | 3639 | 96.82 | 47.86 | 121.3 | 27.18 | 116.9 |
| 6 | NT | NT | NT | NT | 18.1 | 93.13 | 7.009 | 91.67 |
| 4 | 115.8 | 118.64 | 1078 | 102.86 | 81.75 | 113.6 | 21.04 | 116.6 |
| 10 | >31600 | 26.56 | >31600 | 36.52 | 2778 | 114.19 | 983.3 | 86.87 |
| 11 | >31600 | 28.23 | >31600 | 14.18 | 1592 | 90.9 | 853.8 | 98.49 |
| 12 | >3160 | 2.08 | NT | NT | NT | NT | NT | NT |
| 13 | 44.59 | 80.79 | NT | NT | NT | NT | NT | NT |

NT = not tested.

### Example 43: Effects of Compound 1 on the Head Twitch Response (HTR) in Mice

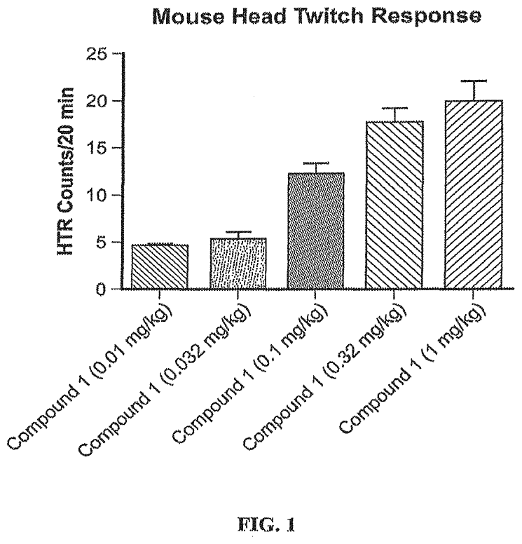

Compound 1 was tested for its ability to induce a head twitch response (HTR) in mice, with the results summarized in FIG. 1. Agonists of the 5-HT2A receptor are well known to induce this effect in rodents and the potency of this HTR is correlated with hallucinogenic potency in humans. Compound 1 induced a robust and dose-dependent HTR.

### Methods:

Animals. Adult male C57BL/6 mice, aged 6-8 weeks (body weight 20-25 g) were used in this experiment. Animals were housed under controlled temperatures and 12-hour light/dark cycles (lights on between 07:00-19:00 h), with ad libitum food and water. This study was carried out in strict accordance with the requirements of the Committee for the Purpose of Control and Supervision of Experiments on Animals (CPCSEA), India. All efforts were made to minimize suffering.

Drugs and Drug Administration. Compound 1 was synthesized as described above. It was administered subcutaneously (SC) dissolved in saline vehicle at a volume of 10 mL/kg. Five doses were tested, with n=6 animals/group. Doses were calculated on the basis of the freebase.

Procedure. Mice were administered one dose of the drug and immediately placed into a small open field for behavioral observation. Animals were observed continuously for 20 mins and the number of head twitches (HTs) were counted by an observer blind to the treatment condition.

Statistical Analysis. The data points shown are the mean±standard error of the mean (SEM). Analysis was performed using GraphPad Prism 6. Curves were fit using a non-linear gaussian distribution to calculate ED₅₀ and Eₘₐₓ values.

### Example 44: Effects of Compound 1 in the Forced Swim Test in Rats

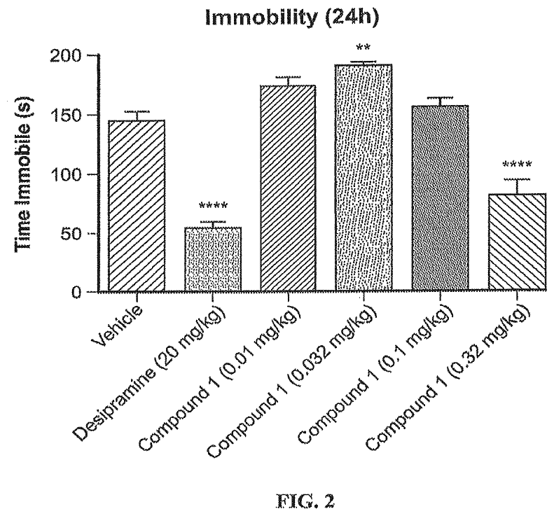

Compound 1 induced antidepressant-like effects in the forced swim test (FST) in rats with a 23.5-h pre-treatment time (FIG. 2). Specifically, the compound at the highest dose reduced immobility time relative to vehicle control, indicative of an antidepressant-like effect. This effect on immobility was observed 23.5 hours after a single compound administration, a time point at which most or all of the drug has been cleared from the systemic circulation.

### Methods:

Animals. Male Sprague Dawley rats, aged 9-10 weeks, were used in this experiment. Animals were housed in groups of 2 under controlled temperature (22±3° C.) and relative humidity (30-70%) conditions, with 12-hour light/dark cycles, and with ad libitum food and water. This study was carried out in strict accordance with the requirements of the Committee for the Purpose of Control and Supervision of Experiments on Animals (CPCSEA), India. All efforts were made to minimize suffering.

Drugs and Drug Administration. Compound 1 was synthesized as described above. Desipramine HCl was commercially obtained. The test compound, saline vehicle, and the positive control desipramine were administered subcutaneously (SC), with doses calculated based on the freebase. Normal saline was used as the vehicle. All compounds were administered at a volume of 5 mL/kg. The test compound and vehicle were administered 0.5 h after the start of the training swim (Swim 1) and 23.5 h before the test swim (Swim 2). Desipramine was administered 3 times, at 23.5 h, 5 h, and 1 h before the test swim (Swim 2), each time at a dose of 20 mg/kg. Group size was n=10 per treatment.

Forced Swim Test (FST). Animals were randomized based on body weight, and it was ensured that inter-group variations were minimal and did not exceed ±20% of the mean body weight across the groups. Rats were handled for about 2 min daily for the 5 days prior to the beginning of the experimental procedure. On the first day of the experiment (i.e., Day 0), post randomization, training swim sessions (Swim 1) were conducted between 12:00 and 18:00 h with all animals by placing rats in individual glass cylinders (46 cm tall×20 cm in diameter) containing 23-25° C. water 30 cm deep for 15 minutes. At the conclusion of Swim 1, animals were dried with paper towels, placed in heated drying cages for 15 minutes, and then returned to their home cages. Animals were then administered the appropriate drug or vehicle treatment(s), as described above. For clarity, a compound administration time of 23.5 h before Swim 2 means 0.5 h after the start of Swim 1 and 0.25 h after the completion of Swim 1 (i.e., immediately after return to the home cage). On Day 1 (i.e., 24 h after start of Swim 1), animals performed the test swim (Swim 2) for a period of 5 min but otherwise under the same conditions as Swim 1. During all swim sessions, the water was changed between each animal.

Behavioral scoring was conducted by observers who were blind to the treatment groups. Animals were continuously observed during Swim 2 and the total time spent engaging in the following behaviors was recorded: immobile, swimming, and climbing. A rat was judged to be immobile when it remained floating in the water without struggling and was making only those movements necessary to keep its head above water. A rat was judged to be swimming when it made active swimming motions, more than necessary to merely maintain its head above water (e.g., moving around in the cylinder). A rat was judged to be climbing when it made active movements with its forepaws in and out of the water, usually directed against the walls.

Statistical Analysis. The data points shown represent the mean±standard error of the mean (SEM). Analysis was performed using GraphPad Prism 6. Comparisons between groups were performed using the one-way analysis of variance (ANOVA), followed by Dunnett's test for comparisons to vehicle.

### Example 45: Effects of Compound 1 on Marble Burying in Mice

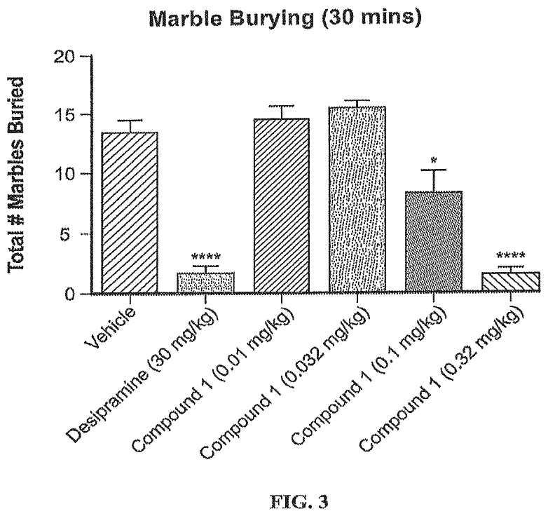

Compound 1 produced an anxiolytic-like effect in the marble burying test (MBT) in C57BL/6 mice (FIG. 3). Specifically, Compound 1 reduced the number of marbles buried in a 30-minute period compared to vehicle.

### Methods:

Animals. Adult male C57BL/6 mice, aged 8-10 weeks (body weight 20-25 g) were used in these experiments. Animals were housed under controlled temperatures and 12-hour light/dark cycles (lights on between 07:00-19:00 h), with ad libitum food and water. This study was carried out in strict accordance with the requirements of the Committee for the Purpose of Control and Supervision of Experiments on Animals (CPCSEA), India. All efforts were made to minimize suffering.

Drugs and Drug Administration. Compound 1 was synthesized as described above. Desipramine HCl was commercially obtained. The test compound, vehicle, and the positive control desipramine were administered subcutaneously (SC), with doses calculated based on the freebase. Normal saline was used as the vehicle. All compounds were administered at a volume of 10 mL/kg. All treatments were administered 30 minutes prior to the start of behavioral testing. Group size was n=9-10 per treatment.

Marble Burying Test (MBT). Animals were randomized based on body weight, and it was ensured that inter-group variations were minimal and did not exceed ±20% of the mean body weight across the groups. Mice were handled for about 2 min daily for the 3 days prior to the beginning of the experimental procedure. Twenty glass marbles (16 mm diameter) were placed with equal distances in a 5×4 pattern on a 5-cm layer of corn-cob bedding, with marbles at least 2 cm from the borders of the cage. The total number of marbles buried were counted in three 10-minute time bins (total 30 minutes). A marble was considered buried when it was >⅔ covered by bedding material.

Statistical Analysis. The data points shown are the mean±standard error of the mean (SEM). Analysis was performed using GraphPad Prism 6. Comparisons between groups were performed using the one-way analysis of variance (ANOVA), followed by Dunnett's test for comparisons to vehicle.

### Example 46. Effects of Additional Compounds in the Mouse Head Twitch Assay

Additional disclosed compounds were tested in the mouse head twitch response (HTR) assay according to the procedure described in Example 43. The compounds induced an HTR, with the results summarized in Table 7. Interestingly, the compounds varied substantially in the maximal effect (Eₘₐₓ) induced in this assay, and the size and nature of the substituent on the amine nitrogen (position 7) and of the amide substituent(s) were found to be important determinants of efficacy in this assay as quantified by the number of HTR elicited at the most efficacious dose. For example, compounds with larger alkyl substituents on the amine (e.g., 1, 2, 3, 5, 6, 7, and 8; all >15 HTR at most efficacious dose) tended to be more efficacious in inducing a HTR compared to compounds with a methyl group at this position (e.g., Int8, Int14, and Int17; all <15 HTR at most efficacious dose). However, some compounds showed the opposite trend. For example, N-propyl derivative 4 was less efficacious than its N-methyl counterpart compound Int11. Further, both of these azetidinyl amide compounds (4 and Int11) were lower efficacy than analogous compounds bearing other amide substituents, suggesting that they may have a decreased propensity to induce hallucinogenic effects. Lastly, compounds with much larger benzyl and phenethyl substituents on the amine nitrogen (e.g., 10 and 11) were low efficacy in this assay, consistent with their lower maximal efficacy in the arrestin functional assay (see Example 41). Other compounds of the disclosure with low maximal efficacy in 5-HT2A receptor functional assays and/or higher potency for 5-HT2A receptor binding compared to functional agonist activity (e.g., compounds with a bromine substituent on the indole) are expected to exhibit an attenuated maximal response in the HTR assay, suggestive of a lack of hallucinogenic effects in humans.

TABLE 7
Effects of disclosed compounds in the mouse HTR assay.

| | | | # HTR at Most |
|---|---|---|---|
| | | | Efficacious Dose |
| | | | (Most Efficacious |
| Compound | ED₅₀ (95% CI)* | Eₘₐₓ (95% CI)* | Dose) |
| 1 | 0.09456 (0.04319-0.2157) | 21.63 (18.45-25.78) | 19.5 (1 mg/kg) |
| 2 | 0.07031 (0.05272-0.09029) | 20.47 (18.43-22.6) | 21.833 (0.32 mg/kg) |
| 3 | 0.04009 (0.02296-0.06228) | 25.56 (22.08-29.36) | 27.0 (0.32 mg/kg) |
| 6 | 0.1387 (0.072-0.2822) | 23.06 (19.86-27.47) | 20.5 (1 mg/kg) |
| 7 | 58.85 (0.5552-???) | 720.4 (???-???) | 16.667 (1 mg/kg) |
| 4 | 0.3757 (0.1774-1.543) | 4.034 (2.965-5.217) | 4.667 (0.1 mg/kg) |
| 5 | 0.03057 (0.01093-0.06217) | 15.22 (11.77-19.03) | 17.667 (0.32 mg/kg) |
| 8 | 0.0282 (0.002515-0.1076) | 12.03 (9.571-14.68) | 15.75 (1 mg/kg) |
| 10 | 1.257 (???-???) | 7.749 (4.126-???) | 6.5 (3.2 mg/kg) |
| 11 | 2.205 (0.7678 - 19.75) | 19.79 (12.66-80.7) | 12.0 (3.2 mg/kg) |
| Int11 | 0.0154 (0.006325-0.02682) | 10.93 (9.181-12.76) | 10.667 (0.1 mg/kg) |
| Int8 | 0.1063 (0.004925-2.219) | 14.34 (10.41-31.47) | 13.5 (1 mg/kg) |
| Int14 | 1.473 (0.22-???) | 21.63 (10.59-???) | 10.5 (1 mg/kg) |
| Int17 | 0.004054 (4.78E-07-0.01872) | 4.96 (3.764-6.286) | 6.0 (0.32 mg/kg) |

*In some cases, curve fitting was poor due to low maximal response and/or incomplete dose range tested.

### Example 47. Effects of Additional Compounds in the Forced Swim Test in Rats

Additional disclosed compounds were tested in the forced swim test (FST) in rats according to the procedure described in Example 44. The compounds decreased time immobile in a dose-dependent manner, indicative of an antidepressant-like effect, with the results summarized in Table 8. All compounds tested reduced immobility time in a dose-dependent manner 23.5 hours after a single dose, suggesting that the compounds rapidly induce durable antidepressant-like effects. Compounds 2 and 4 were the most potent tested in this assay, being as efficacious as the positive control desipramine at a dose of 0.032 mg/kg, SC.

TABLE 8
Effects of disclosed compounds in the rat FST 23.5
hours after a single dose. Significant values compared
to vehicle (p < 0.05) are in bold.

| Compound | |
|---|---|
| (dose, mg/kg, SC) | Time Immobile (s) |
| Vehicle* | 140.6 |
| Desipramine (20)* | 60.93 |
| 1 (0.032) | 161.6 |
| 1 (0.32) | 68.2 |
| 2 (0.032) | 76.44 |
| 2 (0.32) | 41.33 |
| 3 (0.032) | 137.5 |
| 3 (0.32) | 50.4 |
| 6 (0.032) | 93.6 |
| 6 (0.32) | 76.6 |
| 4 (0.01) | 110.4 |
| 4 (0.032) | 68.89 |
| 4 (0.32) | 117.9 |

*Values are the average of 3 independent experiments

### Example 48. Effects of Additional Compounds in the Mouse Marble Burying Assay

Additional compounds of the present invention are tested in the marble burying test (MBT) in mice according to the procedure described in Example 45. The compounds decrease the number of marbles buried in a dose-dependent manner, indicative of an anxiolytic-like effect.

### Example 49: Metabolic Stability in Human Liver Microsomes

Disclosed compounds were tested for stability in human liver microsomes (HLM), with the results summarized in Table 9. The compounds varied in stability in this assay. It was found that the size and nature of the substituent on the amine nitrogen (position 7) was an important determinant of stability in this assay. Among compounds having the R configuration at position 9, those with longer alkyl chains or bulky substituents on the amine (e.g., 1, 2, 3, 4, 5, 6, 7, 8, 10, and 11) exhibited much lower stability (higher clearance) than compounds with a methyl substituent at this position (e.g., LSD, Int11, Int8, Int14, and Int17), suggesting that the former might exhibit shorter half-lives in vivo.

### Methods:

HLM Stability. Pooled HLM from adult male and female donors (Corning 452117) were used. Microsomal incubations were carried out in multi-well plates. Liver microsomal incubation medium consisted of PBS (100 mM, pH 7.4), MgCl₂ (1 mM), and NADPH (1 mM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/mL labetalol as internal standards (IS), followed by shaking for 10 minutes, and then protein sedimentation by centrifugation at 4,000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}), and intrinsic clearance (Clᵢₙₜ) were determined in a plot of ln(AUC) versus time, using linear regression analysis.

TABLE 9
Intrinsic clearance (Clᵢₙₜ) and half-life (t_{1/2}) of
compounds in the presence of HLM.

| Compound | Clᵢₙₜ (μL/min/mg) | t_{1/2} (min) |
|---|---|---|
| LSD | 19.4 | 71.6 |
| 1 | 91.6 | 15.1 |
| 2a | 81.445 | 17.018 |
| 2 | 72.715 | 19.061 |
| 3a | 63.4 | 2.2 |
| 3 | 330.9 | 4.2 |
| 6 | 862.385 | 1.607 |
| 7 | 839.957 | 1.65 |
| 4 | 130.2 | 10.6 |
| 5a | 250.9 | 5.5 |
| 5 | 62.3 | 22.2 |
| 8a | 408.9 | 3.4 |
| 8 | 174.4 | 7.9 |
| 9 | 140.4 | 9.9 |
| 10 | 190.3 | 7.3 |
| 11 | 584.336 | 2.372 |
| Int11 | 53.9 | 25.7 |
| Int8a | 124.6 | 11.1 |
| Int8 | 21.3 | 64.9 |
| Int14a | 413.701 | 3.35 |
| Int14 | 78.783 | 17.593 |
| Int17a | 302.78 | 4.578 |
| Int17 | 87.363 | 15.865 |
| 12 | 103.0 | 13.5 |
| 13 | 65.7 | 21.1 |
| 16 | 285.8 | 4.9 |
| 18 | 101.7 | 13.6 |
| 19 | 82.7 | 16.8 |
| 20 | 649.2 | 2.1 |
| 21 | 367.7 | 3.8 |
| 22 | 58.2 | 23.8 |
| 23 | 193.3 | 7.2 |
| 26 | 67.7 | 20.5 |
| 27 | 86.1 | 16.1 |
| 35 | 57.4 | 24.2 |
| Int22 | <9.6 | >145 |
| 36 | 42.2 | 32.8 |
| 37 | <9.6 | >145 |
| 38 | 21.6 | 64.0 |
| 39 | 43.8 | 31.7 |
| 40 | 434.9 | 3.2 |
| 41 | 130.1 | 10.7 |
| 42 | 125.2 | 11.1 |
| 43 | 56.0 | 24.8 |
| 44 | 153.3 | 9.0 |
| 45 | 175.6 | 7.9 |
| 46 | 427.4 | 3.2 |
| 47 | 249.3 | 5.6 |

### Example 50: Metabolic Stability in Mouse Liver Microsomes

Disclosed compounds were tested for stability in mouse liver microsomes (MLM), with the results summarized in Table 10. The compounds varied in stability in this assay. It was found that the size and nature of the substituent on the amine nitrogen (position 7) was an important determinant of stability in this assay. Among compounds having the R configuration at position 9, those with longer alkyl chains or bulky substituents on the amine (e.g., 1, 2, 3, 4, 5, 6, 7, 8, 10, and 11) exhibited much lower stability (higher clearance) than compounds with a methyl substituent at this position (e.g., LSD, Int11, Int8, Int14, and Int17), suggesting that the former might exhibit shorter half-lives in vivo.

### Methods:

MLM Stability Pooled MLM from CD-1 mice (BIOIVT M00501) were used. Microsomal incubations were carried out in multi-well plates. Liver microsomal incubation medium consisted of PBS (100 mM, pH 7.4), MgCl₂ (1 mM), and NADPH (1 mM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/mL labetalol as internal standards (IS), followed by shaking for 10 minutes, and then protein sedimentation by centrifugation at 4,000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}) and intrinsic clearance (Clᵢₙₜ) were determined in a plot of ln(AUC) versus time, using linear regression analysis.

TABLE 10
Intrinsic clearance (Clᵢₙₜ) and half-life (t_{1/2}) of
compounds in the presence of MLM.

| Compound Number | Clᵢₙₜ (μL/min/mg) | t_{1/2} (min) |
|---|---|---|
| LSD | 44.2 | 31.3 |
| 1 | 615.3 | 2.3 |
| 2a | 780.349 | 1.776 |
| 2 | 519.811 | 2.666 |
| 3a | 2487.8 | 0.6 |
| 3 | 893.8 | 1.6 |
| 6 | 2419.6 | 0.6 |
| 7 | 2560.375 | 0.541 |
| 4 | 1672.5 | 0.8 |
| 5a | 834 | 1.7 |
| 5 | 170.4 | 8.1 |
| 8a | 1663.2 | 0.8 |
| 8 | 377.2 | 3.7 |
| 10 | 1396.8 | 1 |
| 11 | 1512.047 | 0.917 |
| Int11 | 100 | 13.9 |
| Int8a | 842.8 | 1.6 |
| Int8 | 34.7 | 39.9 |
| Int14a | 815.747 | 1.699 |
| Int14 | 228.32 | 6.07 |
| Int17a | 879.576 | 1.576 |
| Int17 | 167.954 | 8.252 |
| 12 | 654.8 | 2.1 |
| 13 | 375.7 | 3.7 |
| 16 | 1053.2 | 1.3 |
| 18 | 523.7 | 2.6 |
| 19 | 192.7 | 7.2 |
| 20 | 1548.2 | 0.9 |
| 21 | 717.4 | 1.9 |
| 22 | 150.0 | 9.2 |
| 23 | 768.7 | 1.8 |

### Example 51: Metabolic Stability in Rat Liver Microsomes

Disclosed compounds were tested for stability in rat liver microsomes (RLM), with the results summarized in Table 11. The compounds varied in stability in this assay. It was found that the size and nature of the substituent on the amine nitrogen (position 7) was an important determinant of stability in this assay. Among compounds having the R configuration at position 9, those with longer alkyl chains or bulky substituents on the amine (e.g., 1, 2, 3, 4, 5, 6, 7, 8, 10, and 11) exhibited much lower stability (higher clearance) than compounds with a methyl substituent at this position (e.g., LSD, Int11, Int8, Int14, and Int17), suggesting that the former might exhibit shorter half-lives in vivo.

### Methods:

RLM Stability Pooled RLM from adult male and female donors (Xenotech R1000) were used. Microsomal incubations were carried out in multi-well plates. Liver microsomal incubation medium consisted of PBS (100 mM, pH 7.4), MgCl₂ (1 mM), and NADPH (1 mM), with 0.50 mg of liver microsomal protein per mL. Control incubations were performed by replacing the NADPH-cofactor system with PBS. Test compounds (1 ptM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/mL labetalol as internal standards (IS), followed by shaking for 10 minutes, and then protein sedimentation by centrifugation at 4,000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}) and intrinsic clearance (Clᵢₙₜ) were determined in a plot of ln(AUC) versus time, using linear regression analysis.

TABLE 11
Intrinsic clearance (Clᵢₙₜ) and half-life (t_{1/2}) of
compounds in the presence of RLM.

| Compound | Clᵢₙₜ (μL/min/mg) | t_{1/2} (min) |
|---|---|---|
| LSD | 23.0 | 60.2 |
| 1 | 85.4 | 16.2 |
| 2a | 143.287 | 9.673 |
| 2 | 115.768 | 11.972 |
| 3a | 960.1 | 1.4 |
| 3 | 317.5 | 4.4 |
| 6 | 837.443 | 1.655 |
| 7 | 859.914 | 1.612 |
| 4 | 108 | 12.8 |
| 5a | 453.2 | 3.1 |
| 5 | 72.0 | 19.3 |
| 8a | 835.1 | 1.7 |
| 8 | 180.1 | 7.7 |
| 10 | 407.4 | 3.4 |
| 11 | 1195.423 | 1.159 |
| Int11 | 25.3 | 54.9 |
| Int8a | 394 | 3.5 |
| Int8 | 13.9 | 99.5 |
| Int14a | 770.023 | 1.8 |
| Int14 | 36.486 | 37.988 |
| Int17a | 706.862 | 1.961 |
| Int17 | 49.688 | 27.894 |
| 12 | 236.2 | 5.9 |
| 13 | 50.0 | 27.7 |
| 16 | 616.3 | 2.2 |
| 18 | 262.8 | 5.3 |
| 19 | 139.0 | 10.0 |
| 20 | 1147.6 | 1.2 |
| 21 | 323.2 | 4.3 |
| 22 | 104.8 | 13.2 |
| 23 | 331.7 | 4.2 |

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the present disclosure have been discussed, the above specification is illustrative and not restrictive. Many variations of the present disclosure will become apparent to those skilled in the art upon review of this specification. The full scope of the present disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1. A compound of Formula (I): 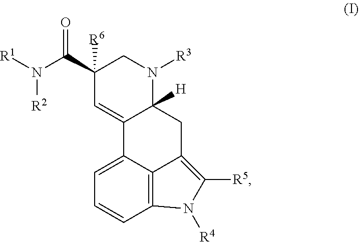
or a pharmaceutically acceptable salt thereof,
wherein
R¹ is C₁-C₆ alkyl or 3-7 membered carbocyclyl, wherein R¹ is optionally substituted with one or more halogen or C₁-C₆ alkyl;
R² is hydrogen or C₁-C₆ alkyl, wherein R² is optionally substituted with one or more halogen or C₁-C₆ alkyl; or
wherein R¹ and R² can be taken together with the atom on which they are attached to form an optionally substituted 3-7 membered heterocyclyl comprising 1-3 heteroatoms selected from the group consisting of N, O, and S, wherein the heterocyclyl is optionally substituted with one or more fluoro or C₁-C₆ alkyl;
R³ is selected from the group consisting of C₂-C₆ alkyl, C₂-C₆ alkenyl, C₂-C₆ alkynyl, —CH₂-(cyclopropyl), and 3-7 membered cycloalkyl,
wherein R³ may be substituted with one or more substituents each independently selected from the group consisting of fluoro, hydroxyl, and —OMe;
or
R³ is selected from the group consisting of —(C₁-C₂ alkyl)-phenyl and —(C₁-C₂ alkyl)-(6-membered heteroaryl),
wherein C₁-C₂ alkyl is optionally substituted with one or more fluoro, hydroxyl, and —OMe, and wherein phenyl and 6-membered heteroaryl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₄ alkyl, C₃-C₅ cycloalkyl, and C₁-C₄ alkoxy;
R⁴ is hydrogen or —C(O)(C₁-C₈ alkyl);
R⁵ is hydrogen, Me, Et, —CH₂F, CHF₂, —CF₃, or halogen;
R⁶ is hydrogen or deuterium; with the provisos that
(a) when R¹ and R² are both ethyl, and R⁴ and R⁵ are both hydrogen, R³ is not unsubstituted linear C₂-C₆ alkyl, isopropyl, —CH₂CH═CH₂, —CH₂CH₂F, or —CH₂CH₂Ph;
(b) when R¹ and R² are both ethyl, R⁴ is —C(O)(C₂ alkyl), and R⁵ is hydrogen, R³ is not unsubstituted ethyl; and
(c) when R¹ is ethyl and R² is H, R³ is not unsubstituted ethyl, unsubstituted n-propyl, or —CH₂CH═CH₂.

2. The compound of claim 1, wherein
R¹ is C₁-C₆ alkyl or 3-5 membered carbocyclyl, wherein R¹ is optionally substituted with one or more fluoro or C₁-C₄ alkyl;
R² is hydrogen or C₁-C₃ alkyl, wherein R² is optionally substituted with one or more fluoro or C₁-C₄ alkyl; or
wherein R¹ and R² can be taken together with the atom on which they are attached to form an optionally substituted 3-6 membered heterocyclyl comprising 1-3 heteroatoms selected from the group consisting of N, O, and S, wherein the heterocyclyl is optionally substituted with one or more fluoro or C₁-C₃ alkyl;
R³ is selected from the group consisting of C₂-C₄ alkyl, C₂-C₄ alkenyl, C₂-C₄ alkynyl, —CH₂-(cyclopropyl), and 3-5 membered cycloalkyl,
wherein R³ may be substituted with one or more substituents each independently selected from the group consisting of fluoro, hydroxyl, and —OMe;
or R³ is selected from the group consisting of —(C₁-C₂ alkyl)-phenyl and —(C₁-C₂ alkyl)-(6-membered heteroaryl),
wherein C₁-C₂ alkyl is optionally substituted with one or more fluoro, and wherein phenyl and 6-membered heteroaryl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₃ alkyl, cyclopropyl, and C₁-C₃ alkoxy;
R⁴ is hydrogen or —C(O)(C₁-C₈ alkyl);
R⁵ is hydrogen, Me, Et, —CH₂F, CHF₂, —CF₃, or halogen;
R⁶ is hydrogen or deuterium; or a pharmaceutically acceptable salt thereof.

3. The compound of claim 2, wherein R⁴ is hydrogen.

4. The compound of claim 2, wherein R⁵ is hydrogen.

5. The compound of claim 2, wherein R⁵ is Me, Et, —CH₂F, CHF₂, —CF₃, or halogen.

6. The compound of claim 2, wherein the compound is a compound of formula (Ia): 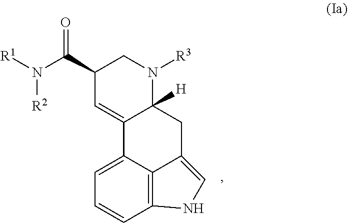
or a pharmaceutically acceptable salt thereof.

7. The compound of claim 2, wherein the compound is a compound of formula (Ib), formula (Ic), formula (1d), or formula (1e): 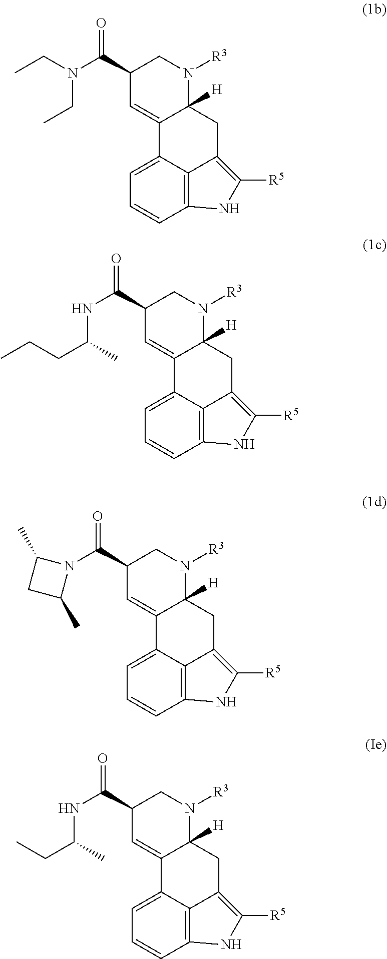
or a pharmaceutically acceptable salt thereof.

8. The compound of claim 7, wherein R⁵ is hydrogen.

9. The compound of claim 7, wherein R⁵ is Me, Et, or halogen.

10. The compound of claim 7, wherein R⁵ is Me, Et, or bromo.

11. The compound of claim 7, wherein R³ is selected from the group consisting of ethyl, n-propyl, —CH₂CH═CH₂, cyclopropyl, and —CH₂-(cyclopropyl),
wherein R³ may be substituted with one to three instances of fluoro.

12. The compound of claim 7, wherein R³ is selected from the group consisting of ethyl, n-propyl, —CH₂CH═CH₂, cyclopropyl, —CH₂-(cyclopropyl), —CH₂CF₃, —CH₂CH₂CH₂F, and —CH₂CH₂CF₃.

13. The compound of claim 7, wherein R³ is selected from the group consisting of —(C₁-C₂ alkyl)-phenyl and —(C₁-C₂ alkyl)-(6-membered heteroaryl),
wherein C₁-C₂ alkyl is optionally substituted with one or more fluoro, and wherein phenyl and 6-membered heteroaryl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₃ alkyl, cyclopropyl, and C₁-C₃ alkoxy.

14. The compound of claim 7, wherein R³ is selected from the group consisting of —(C₁-C₂ alkyl)-phenyl and —(C₁-C₂ alkyl)-pyridinyl,
wherein phenyl and pyridinyl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₃ alkyl, cyclopropyl, and C₁-C₃ alkoxy.

15. The compound of claim 7, wherein R³ is selected from the group consisting of

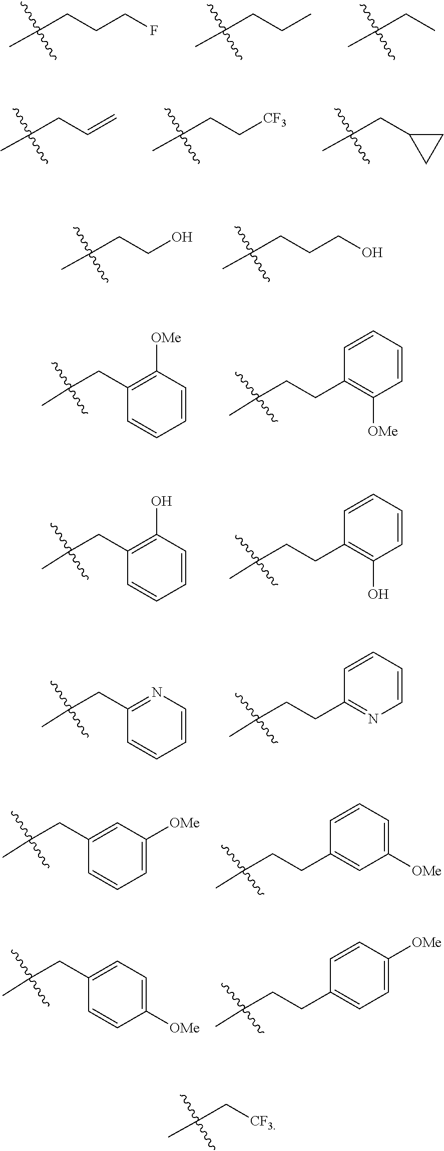

16. A compound selected from the group consisting of: 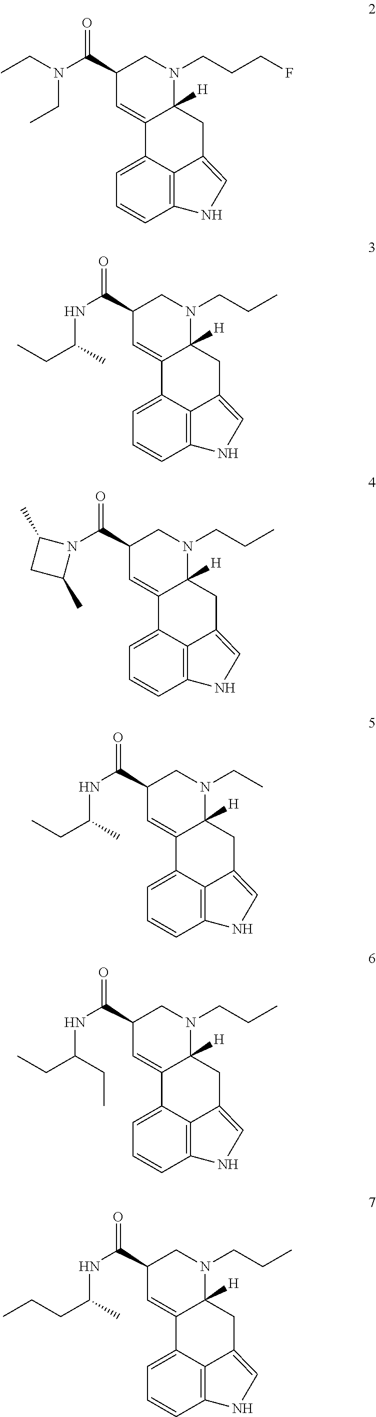 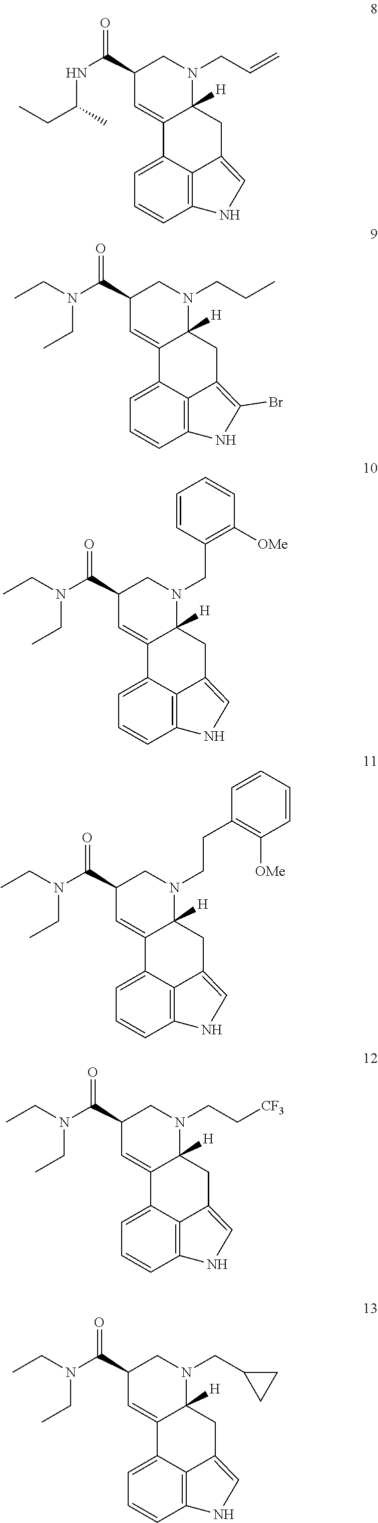 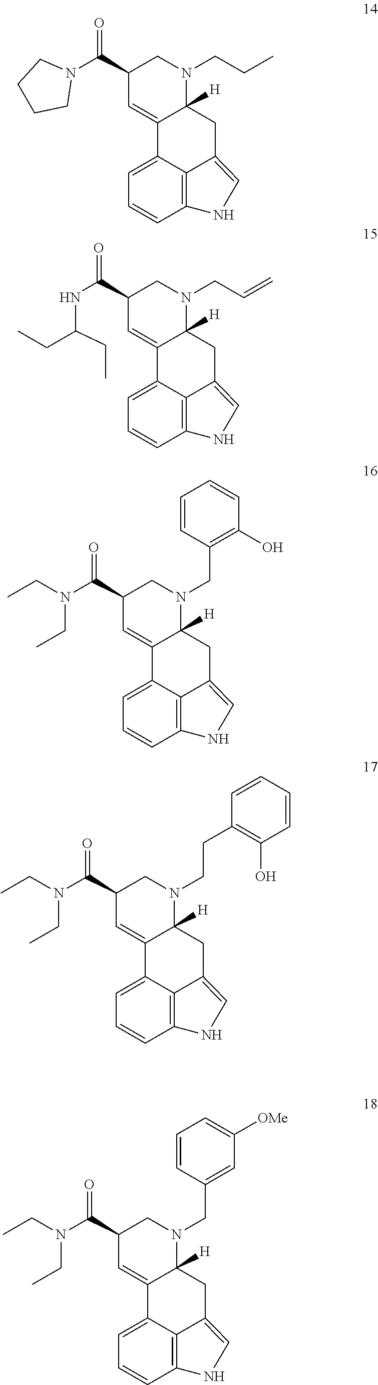 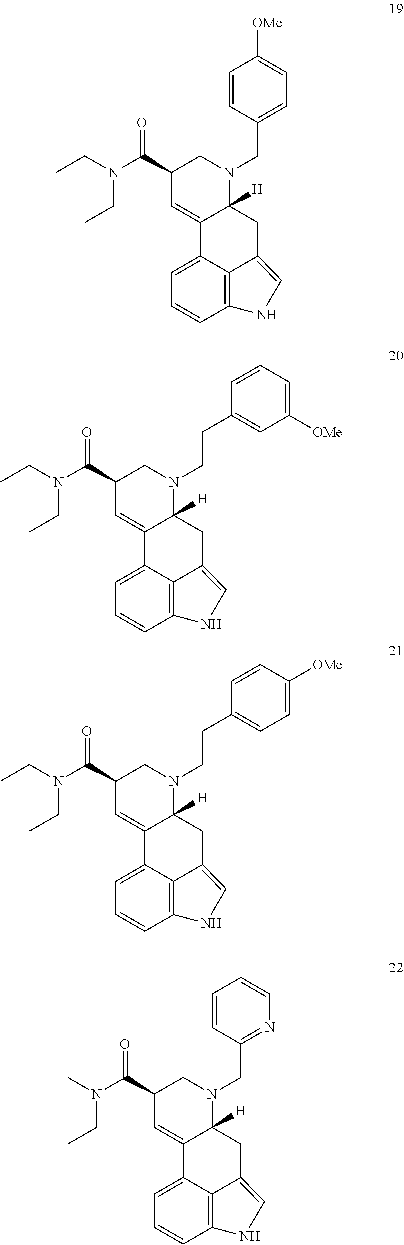 [SMILES: CCN(CC)C(=O)[C@@H]1C=C2c3cccc4[nH]cc(c34)C[C@H]2N(Cc2ccc(OC)cc2)C1 ; CCN(CC)C(=O)[C@@H]1C=C2c3cccc4[nH]cc(c34)C[C@H]2N(CCc2cccc(OC)c2)C1 ; CCN(CC)C(=O)[C@@H]1C=C2c3cccc4[nH]cc(c34)C[C@H]2N(CCc2ccc(OC)cc2)C1 ; CCN(C)C(=O)[C@@H]1C=C2c3cccc4[nH]cc(c34)C[C@H]2N(Cc2ccccn2)C1] 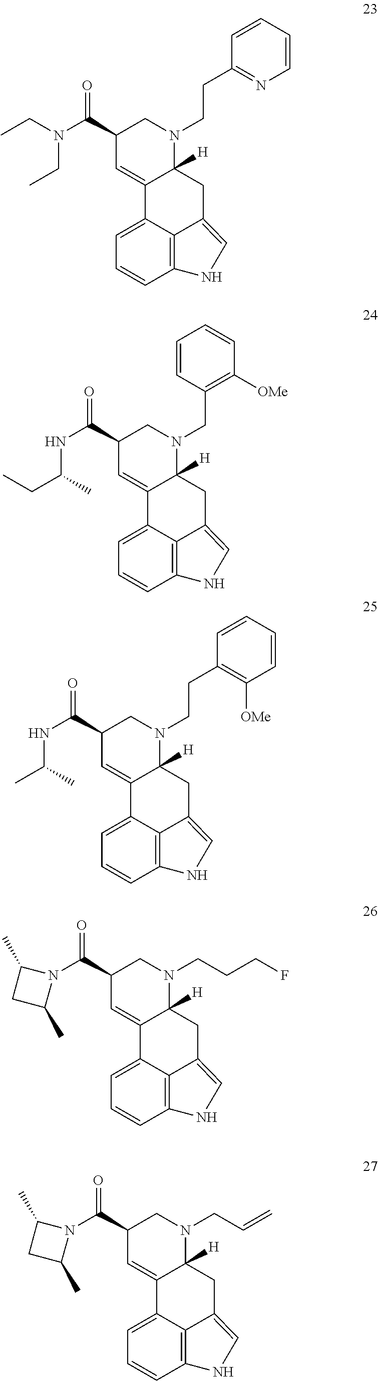 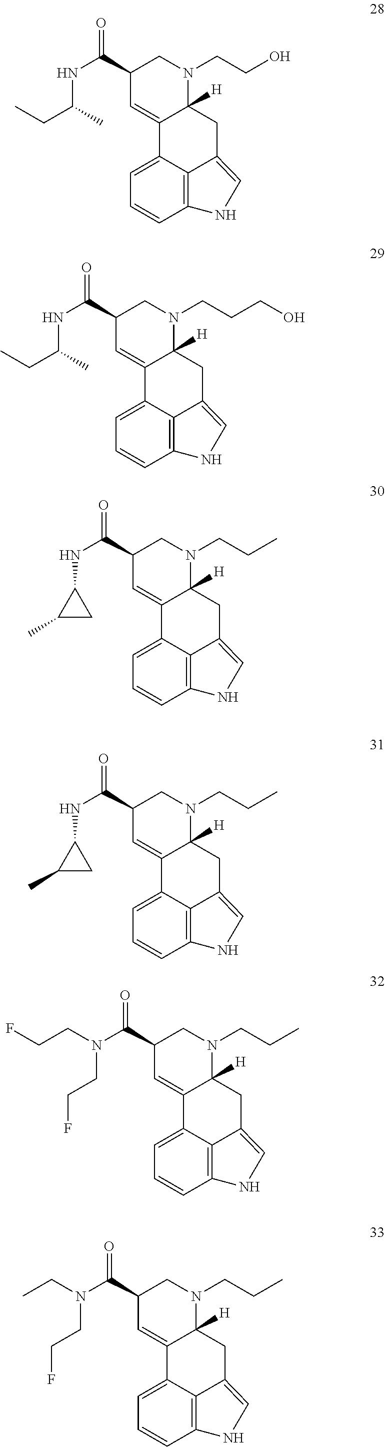 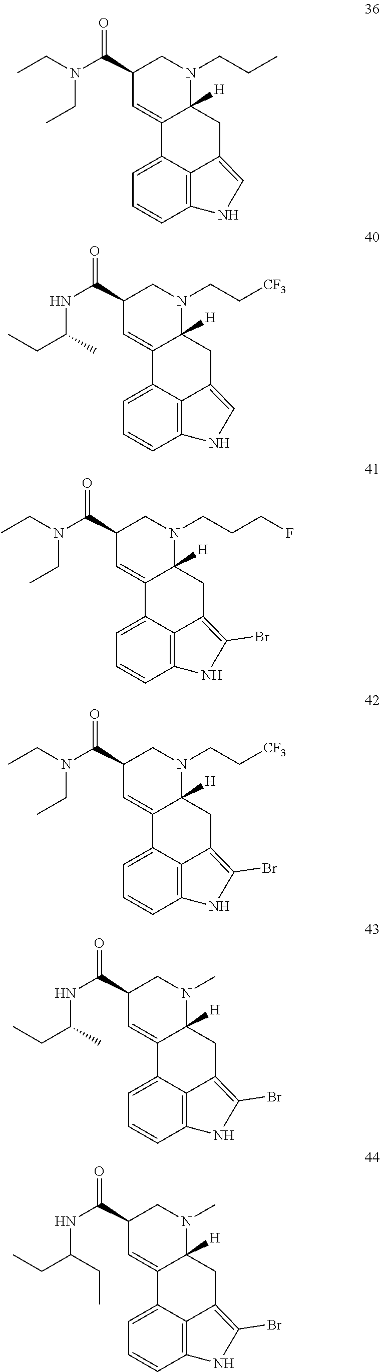 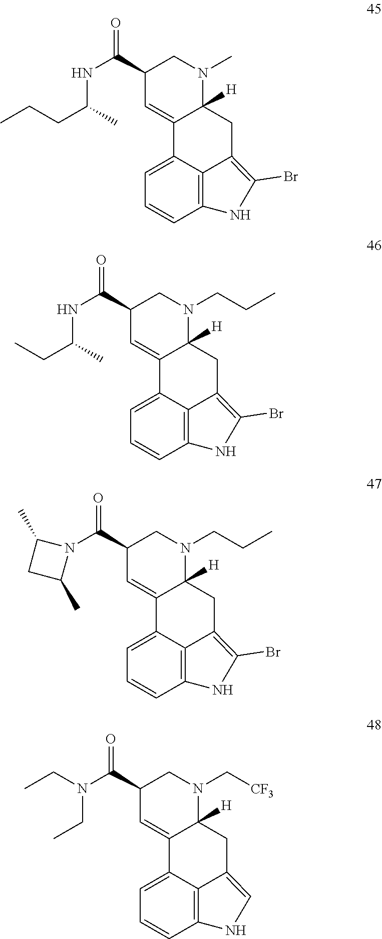
or a pharmaceutically acceptable salt thereof.

17. A compound of Formula (II): 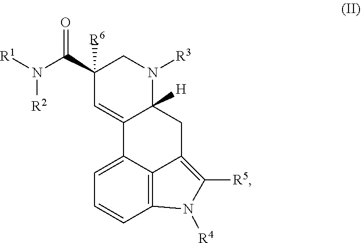
or a pharmaceutically acceptable salt thereof,
wherein
R¹ is C₁-C₆ alkyl or 3-7 membered carbocyclyl, wherein R¹ is optionally substituted with one or more halogen or C₁-C₆ alkyl;
R² is hydrogen or C₁-C₆ alkyl, wherein R² is optionally substituted with one or more halogen or C₁-C₆ alkyl; or
wherein R¹ and R² can be taken together with the atom on which they are attached to form an optionally substituted 3-7 membered heterocyclyl comprising 1-3 heteroatoms selected from the group consisting of N, O, and S, wherein the heterocyclyl is optionally substituted with one or more fluoro or C₁-C₆ alkyl;
R³ is selected from the group consisting of C₁-C₆ alkyl, C₂-C₆ alkenyl, C₂-C₆ alkynyl, —CH₂-(cyclopropyl), and 3-7 membered cycloalkyl,
wherein R³ may be substituted with one or more substituents each independently selected from the group consisting of fluoro, hydroxyl, and —OMe;
or
R³ is selected from the group consisting of —(C₁-C₂ alkyl)-phenyl and —(C₁-C₂ alkyl)-(6-membered heteroaryl),
wherein C₁-C₂ alkyl is optionally substituted with one or more fluoro, hydroxyl, and —OMe, and wherein phenyl and 6-membered heteroaryl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₄ alkyl, C₃-C₅ cycloalkyl, and C₁-C₄ alkoxy;
R⁴ is hydrogen or —C(O)(C₁-C₈ alkyl);
R⁵ is Me, Et, —CH₂F, —CHF₂, —CF₃, or halogen;
R⁶ is hydrogen or deuterium; with the provisos that
(a) when R¹ and R² are both ethyl, R³ is methyl, and R⁴ is hydrogen, then R⁵ is not chloro, bromo, iodo, or unsubstituted methyl;
(b) when R² is hydrogen, R³ is methyl, R⁴ is hydrogen, and R⁵ is bromo, then R¹ is not ethyl, isopropyl, or propargyl; and
(c) when R² is methyl, R³ is methyl, R⁴ is hydrogen, and R⁵ is bromo, then R¹ is not propargyl or cyclopropyl.

18. The compound of claim 17, wherein
R¹ is C₁-C₆ alkyl or 3-5 membered carbocyclyl, wherein R¹ is optionally substituted with one or more fluoro or C₁-C₄ alkyl;
R² is hydrogen or C₁-C₃ alkyl, wherein R² is optionally substituted with one or more fluoro or C₁-C₄ alkyl; or
wherein R¹ and R² can be taken together with the atom on which they are attached to form an optionally substituted 3-6 membered heterocyclyl comprising 1-3 heteroatoms selected from the group consisting of N, O, and S, wherein the heterocyclyl is optionally substituted with one or more fluoro or C₁-C₃ alkyl;
R³ is selected from the group consisting of C₁-C₄ alkyl, C₂-C₄ alkenyl, C₂-C₄ alkynyl, —CH₂-(cyclopropyl), and 3-5 membered cycloalkyl,
wherein R³ may be substituted with one or more substituents each independently selected from the group consisting of fluoro, hydroxyl, and —OMe;
or R³ is selected from the group consisting of —(C₁-C₂ alkyl)-phenyl and —(C₁-C₂ alkyl)-(6-membered heteroaryl),
wherein C₁-C₂ alkyl is optionally substituted with one or more fluoro, and wherein phenyl and 6-membered heteroaryl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₃ alkyl, cyclopropyl, and C₁-C₃ alkoxy;
R⁴ is hydrogen or —C(O)(C₁-C₈ alkyl);
R⁵ is Me, Et, —CH₂F, —CHF₂, —CF₃, or halogen;
R⁶ is hydrogen or deuterium.

19. The compound of claim 18, wherein R⁴ is hydrogen.

20. The compound of claim 18, wherein the compound is a compound of formula (IIb), formula (IIc), formula (IId), or formula (IIe): 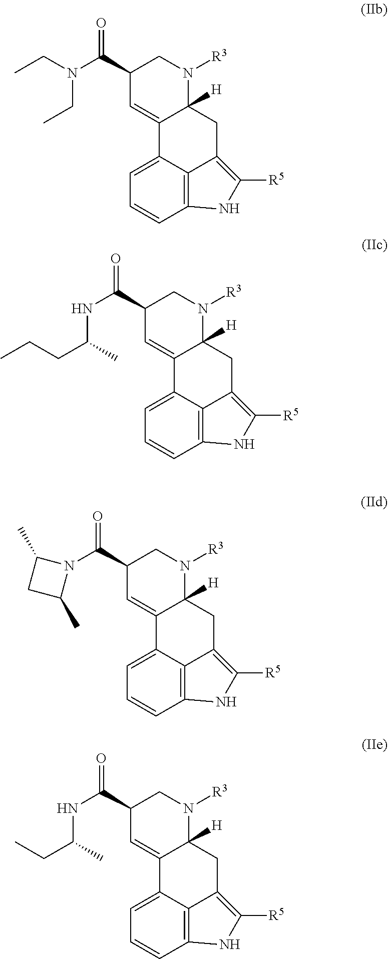
or a pharmaceutically acceptable salt thereof.

21. The compound of claim 20, wherein R⁵ is Me, Et, or halogen.

22. The compound of claim 20, wherein R⁵ is Me, Et, or bromo.

23. The compound of claim 20, wherein R⁵ is bromo.

24. The compound of claim 20, wherein R³ is selected from the group consisting of methyl, ethyl, n-propyl, —CH₂CH═CH₂, cyclopropyl, and —CH₂-(cyclopropyl),
wherein R³ may be substituted with one to three instances of fluoro.

25. The compound of claim 20, wherein R³ is selected from the group consisting of ethyl, n-propyl, —CH₂CH═CH₂, cyclopropyl, and —CH₂-(cyclopropyl),
wherein R³ may be substituted with one to three instances of fluoro.

26. The compound of claim 20, wherein R³ is selected from the group consisting of methyl, ethyl, n-propyl, —CH₂CH═CH₂, cyclopropyl, —CH₂-(cyclopropyl), —CH₂CF₃, —CH₂CH₂CH₂F, and —CH₂CH₂CF₃.

27. The compound of any of claim 20, wherein R³ is selected from the group consisting of ethyl, n-propyl, —CH₂CH═CH₂, cyclopropyl, —CH₂-(cyclopropyl), —CH₂CF₃, —CH₂CH₂CH₂F, and —CH₂CH₂CF₃.

28. The compound of claim 20, wherein R³ is selected from the group consisting of —(C₁-C₂ alkyl)-phenyl and —(C₁-C₂ alkyl)-(6-membered heteroaryl),
wherein C₁-C₂ alkyl is optionally substituted with one or more fluoro, and wherein phenyl and 6-membered heteroaryl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₃ alkyl, cyclopropyl, and C₁-C₃ alkoxy.

29. The compound of claim 20, wherein R³ is selected from the group consisting of —(C₁-C₂ alkyl)-phenyl and —(C₁-C₂ alkyl)-pyridinyl,
wherein phenyl and pyridinyl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₃ alkyl, cyclopropyl, and C₁-C₃ alkoxy.

30. The compound of claim 20, wherein R³ is selected from the group consisting of

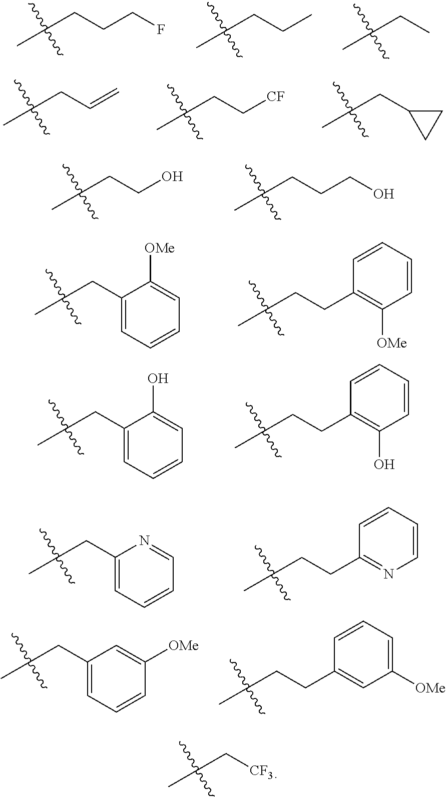

31. The compound of claim 20, selected from the group consisting of: 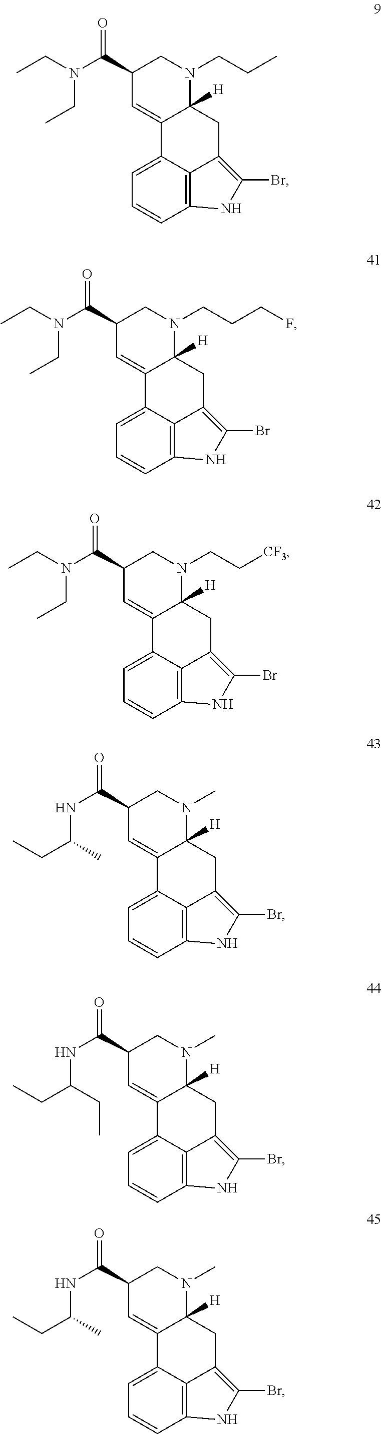 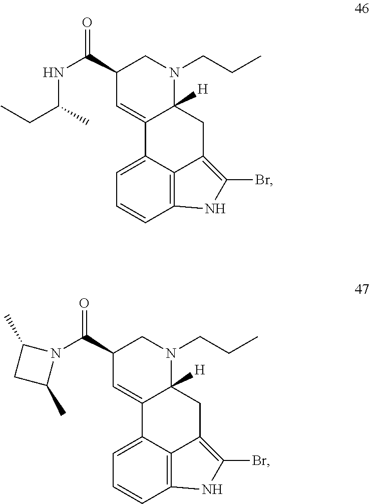
or a pharmaceutically acceptable salt thereof.

32. A compound having the structure: 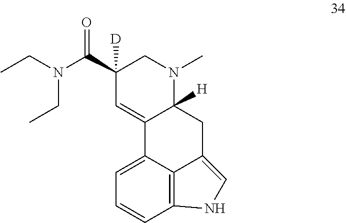 [SMILES: [2H][C@@]1(C(=O)N(CC)CC)C=C2c3cccc4[nH]cc(c34)C[C@H]2N(C)C1]
or a pharmaceutically acceptable salt thereof.

33. A pharmaceutical composition comprising a compound of claim 1 or claim 17 and a pharmaceutically acceptable adjuvant or carrier.

34. A method of treating a mood disorder comprising administering to a patient in need thereof a pharmaceutical composition comprising an effective amount of a compound of claim 1 or claim 17.

35. The method of claim 34, wherein the mood disorder is selected from the group consisting of depressive disorders and bipolar disorders.

36. The method of claim 34, wherein the mood disorder is a depressive disorder.

37. The method of claim 34, wherein the mood disorder is a treatment-resistant depressive disorder.

38. The method of claim 34, wherein the mood disorder is selected from the group consisting of major depressive disorder, persistent depressive disorder, postpartum depression, premenstrual dysphoric disorder, seasonal affective disorder, psychotic depression, disruptive mood dysregulation disorder, substance/medication-induced depressive disorder, and depressive disorder due to another medical condition.

39. The method of claim 34, wherein the mood disorder is selected from the group consisting of bipolar disorder I, bipolar disorder II, cyclothymic disorder, substance/medication-induced bipolar and related disorder, and bipolar and related disorder due to another medical condition.

40. The method of claim 34, wherein the mood disorder is a substance-related disorder.

41. The method of claim 34, wherein the mood disorder is a substance-use disorder.

42. The method of claim 34, wherein the mood disorder is an anxiety disorder.

43. The method of claim 34, wherein the mood disorder is selected from the group consisting of obsessive-compulsive and related disorders, trauma- and stressor-related disorders, feeding and eating disorders, borderline personality disorder, attention-deficit/hyperactivity disorder, and autism spectrum disorder.

44. The method of claim 34, wherein the mood disorder is a neurocognitive disorder.

45. A method of treating a mood disorder comprising administering to a patient in need thereof a pharmaceutical composition comprising an effective amount of a compound according to the Formula 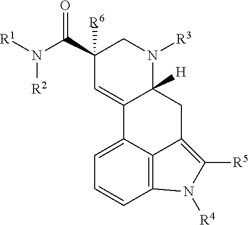
or a pharmaceutically acceptable salt thereof,
wherein
R¹ is C₁-C₆ alkyl or 3-7 membered carbocyclyl, wherein R¹ is optionally substituted with one or more halogen or C₁-C₆ alkyl;
R² is hydrogen or C₁-C₆ alkyl, wherein R² is optionally substituted with one or more halogen or C₁-C₆ alkyl; or
wherein R¹ and R² can be taken together with the atom on which they are attached to form an optionally substituted 3-7 membered heterocyclyl comprising 1-3 heteroatoms selected from the group consisting of N, O, and S, wherein the heterocyclyl is optionally substituted with one or more fluoro or C₁-C₆ alkyl;
R³ is selected from the group consisting of C₁-C₆ alkyl, C₂-C₆ alkenyl, C₂-C₆ alkynyl, —CH₂-(cyclopropyl), and 3-7 membered cycloalkyl,
wherein R³ may be substituted with one or more substituents each independently selected from the group consisting of fluoro, hydroxyl, and —OMe;
or
R³ is selected from the group consisting of —(C₁-C₂ alkyl)-phenyl and —(C₁-C₂ alkyl)-(6-membered heteroaryl),
wherein C₁-C₂ alkyl is optionally substituted with one or more fluoro, hydroxyl, and —OMe, and wherein phenyl and 6-membered heteroaryl are optionally substituted with one or more substitutents each independently selected from the group consisting of halogen, hydroxyl, —OC(O)(C₁-C₈ alkyl), —CN, —NO₂, —NH₂, —C(O)NH₂, C₁-C₄ alkyl, C₃-C₅ cycloalkyl, and C₁-C₄ alkoxy;
R⁴ is hydrogen or —C(O)(C₁-C₈ alkyl);
R⁵ is hydrogen, Me, Et, —CH₂F, CHF₂, —CF₃, or halogen;
R⁶ is hydrogen or deuterium.

46. The method of claim 45, wherein the mood disorder is selected from the group consisting of depressive disorders and bipolar disorders.

47. The method of claim 45, wherein the mood disorder is a depressive disorder.

48. The method of claim 45, wherein the mood disorder is a treatment-resistant depressive disorder.

49. The method of claim 45, wherein the mood disorder is selected from the group consisting of major depressive disorder, persistent depressive disorder, postpartum depression, premenstrual dysphoric disorder, seasonal affective disorder, psychotic depression, disruptive mood dysregulation disorder, substance/medication-induced depressive disorder, and depressive disorder due to another medical condition.

50. The method of claim 45, wherein the mood disorder is selected from the group consisting of bipolar disorder I, bipolar disorder II, cyclothymic disorder, substance/medication-induced bipolar and related disorder, and bipolar and related disorder due to another medical condition.

51. The method of claim 45, wherein the mood disorder is a substance-related disorder.

52. The method of claim 45, wherein the mood disorder is a substance-use disorder.

53. The method of claim 45, wherein the mood disorder is an anxiety disorder.

54. The method of claim 45, wherein the mood disorder is selected from the group consisting of obsessive-compulsive and related disorders, trauma- and stressor-related disorders, feeding and eating disorders, borderline personality disorder, attention-deficit/hyperactivity disorder, and autism spectrum disorder.

55. The method of claim 45, wherein the mood disorder is a neurocognitive disorder.

56. The method of claim 45, wherein the compound has the structure: 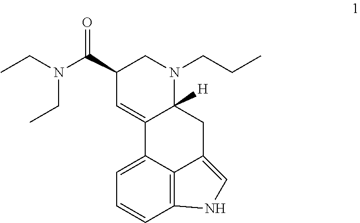 [SMILES: CCCN1C[C@H](C(=O)N(CC)CC)C=C2c3cccc4[nH]cc(c34)C[C@H]21]
or a pharmaceutically acceptable salt thereof.
